SEQUENCE LISTING (18 sequences)

SEQ ID NO: 1 - protein, 447 aa, Artificial Sequence
  Brentuximab/cAC10 heavy chain
QIQLQQSGPEVVKPGASVKISCKASGYTFTDYYITWVKQKPGQGLEWIGWIYPGSGNTKYNEKFKGKATLTVDTSSSTAFMQLSSLTSEDTAVYFCANYGNYWFAYWGQGTQVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 218 aa, Artificial Sequence
  Brentuximab/cAC10 light chain
DIVLTQSPASLAVSLGQRATISCKASQSVDFDGDSYMNWYQQKPGQPPKVLIYAASNLESGIPARFSGSGSGTDFTLNIHPVEEEDAATYYCQQSNEDPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 4 aa, Artificial Sequence
  TTL recognition sequence
  misc_feature (1)..(2): Xaa can be any naturally occurring amino acid
  MISC_FEATURE (3)..(3): X can be E, D or C
XXXE

SEQ ID NO: 4 - protein, 4 aa, Artificial Sequence
  TTL recognition sequence
EGEE

SEQ ID NO: 5 - protein, 14 aa, Artificial Sequence
  TTL recognition sequence
VDSVEGEGEEEGEE

SEQ ID NO: 6 - protein, 12 aa, Artificial Sequence
  TTL recognition sequence
SVEGEGEEEGEE

SEQ ID NO: 7 - protein, 10 aa, Artificial Sequence
  TTL recognition sequence
SADGEDEGEE

SEQ ID NO: 8 - protein, 11 aa, Artificial Sequence
  TTL recognition sequence
SVEAEAEEGEE

SEQ ID NO: 9 - protein, 10 aa, Artificial Sequence
  TTL recognition sequence
SYEDEDEGEE

SEQ ID NO: 10 - protein, 10 aa, Artificial Sequence
  TTL recognition sequence
SFEEENEGEE

SEQ ID NO: 11 - protein, 237 aa, Artificial Sequence
  Brentuximab light chain comprising TTL recognition sequence at C-terminus
DIVLTQSPASLAVSLGQRATISCKASQSVDFDGDSYMNWYQQKPGQPPKVLIYAASNLESGIPARFSGSGSGTDFTLNIHPVEEEDAATYYCQQSNEDPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSVDSVEGEGEEEGEE

SEQ ID NO: 12 - protein, 461 aa, Artificial Sequence
  Brentuximab heavy chain fused to TTL recognition sequence at C-terminus
QIQLQQSGPEVVKPGASVKISCKASGYTFTDYYITWVKQKPGQGLEWIGWIYPGSGNTKYNEKFKGKATLTVDTSSSTAFMQLSSLTSEDTAVYFCANYGNYWFAYWGQGTQVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKVDSVEGEGEEEGEE

SEQ ID NO: 13 - protein, 377 aa, Canis lupus
MYTFVVRDENSSVYAEVSRLLLATGHWKRLRRDNPRFNLMLGERNRLPFGRLGHEPGLMQLVNYYRGADKLCRKASLVKLIKTSPDLAESCTWFPESYVIYPTNLKTPVAPAQNGIHPPIHNSRTDEREFFLASYNRKKEDGEGNVWIAKSSAGAKGEGILISSEATELLDFIDNQGQVHVIQKYLEHPLLLEPGHRKFDIRSWVLVDHQYNIYLYKEGVLRTASEPYHVDNFQDKTCHLTNHCIQKEYSKNYGKYEEGNEMFFEEFNQYLISALNITLESSILLQIKHIIRSCLMSVEPAISTRYLPYQSFQLFGFDFMVDETLKVWLIEVNGAPACAQRLYAELCQGIVDIAISSVFPPPDVEPQHIQPAAFIKL

SEQ ID NO: 14 - protein, 232 aa, Artificial sequence
  Brentuximab light chain comprising TTL recognition sequence at C-terminus
DIVLTQSPASLAVSLGQRATISCKASQSVDFDGDSYMNWYQQKPGQPPKVLIYAASNLESGIPARFSGSGSGTDFTLNIHPVEEEDAATYYCQQSNEDPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECVDSVEGEGEEEGEE

SEQ ID NO: 15 - protein, 4 aa, Artificial sequence
  recognition sequence for tubulin-tyrosine ligase
  misc_feature (1)..(4): Xaa can be any naturally occurring amino acid
XXXX

SEQ ID NO: 16 - protein, 5 aa, Artificial sequence
  Gly Ser linker
GGGGS

SEQ ID NO: 17 - protein, 10 aa, Artificial sequence
  Gly Ser linker
GGGGSGGGGS

SEQ ID NO: 18 - protein, 15 aa, Artificial sequence
  Gly Ser linker
GGGGSGGGGSGGGGS

(19) United States
(12) Patent Application Publication
(10) Pub. No.: US 2022/0193251 A1
(43) Pub. Date: Jun. 23, 2022

(54) CD30 TARGETING ANTIBODY DRUG CONJUGATES AND USES THEREOF

(71) Applicants: Ludwig-Maximilians-Universitaet Muenchen, Munich (DE); Forschungsverbund Berlin e.V., Berlin (DE)

(72) Inventors: Heinrich LEONHARDT, Munich (DE); Jonas HELMA-SMETS, Munich (DE); Dominik SCHUMACHER, Munich (DE); Marcus GERLACH, Munich (DE); Christian PR HACKENBERGER, Berlin (DE); Marc-André KASPER, Berlin (DE)

(21) Appl. No.: 17/559,664

(22) Filed: Dec. 22, 2021

(30) Foreign Application Priority Data
Dec. 23, 2020 (EP) 20216838.1

(51) Int. Cl. A61K 47/68

(52) U.S. Cl. CPC A61K 47/6851; A61K 47/6811

(57) ABSTRACT

The present invention relates to an antibody-drug conjugate (ADC) comprising: (a) Brentuximab, wherein Brentuximab comprises at the C-terminus of the light chains, the heavy chains or all of the heavy and light chains of the Brentuximab a recognition sequence for tubulin tyrosine ligase and a non-natural amino acid; and (b) at least one drug moiety; wherein a drug moiety is coupled to each of the non-natural amino acids via a linker. The present invention further relates to methods of producing same, pharmaceutical compositions comprising same as well as uses thereof.

### CROSS-REFERENCE TO RELATED APPLICATIONS

The present application claims the benefit of priority of European Patent Application No. 20216838.1 filed 23 Dec. 2020, the content of which is hereby incorporated by reference in its entirety for all purposes.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 350066.405_SEQUENCE_LISTING.txt. The text file is 21,000 bytes, was created on Dec. 20, 2021, and is being submitted electronically via EFS-Web.

### TECHNICAL FIELD OF THE INVENTION

The present invention relates to an antibody-drug conjugate (ADC) comprising Brentuximab and at least one drug moiety, methods of producing same, pharmaceutical compositions comprising same as well as uses thereof.

### BACKGROUND

Lymphomatic malignanices account for about 4-5% of all cancer cancers and derive from the lymphatic system. In classical Hodgkin's lymphoma and several non-Hodgkin lymphomas such as Anaplastic large cell lymphoma and Peripheral T cell lymphoma, CD30 has proven as a valuable tumor biomarker for targeted treatment in recent years. With the First-in-class approval of brentuximab vedotin (Brentuximab vedotin) in 2011, the first CD30-specific biological drug has entered the market and has since become an indispensable treatment option with good overall response. Brentuximab Vedotin consists of the tumour-targeting chimeric IgG1 antibody component brentuximab and a linker-payload component, comprising a Cathepsin B cleavable Valine-Citrulline linker moiety chemically connected with a potent payload moiety Monomethyl-Auristatin E, which efficiently induces apoptosis upon intracellular delivery and release.

Brentuximab vedotin is an Antibody Drug Conjugate (ADC), a relatively novel therapeutic modality that has gained major interest in recent years. These biopharmaceuticals deliver the potent cytotoxic drugs directly to the tumor-cells and therefore have the potential to broaden the therapeutic window compared to conventional chemotherapy. To date, nine stochastically conjugated ADCs have been approved. These molecules are either based on conjugation with activated carboxylic acids (e.g. Mylotarg, Besponsa, Kadcyla) which randomly react with exposed lysine residues. Alternatively, maleimides have become the most important conjugation reagent to conjugate the linker-payload moiety to free cysteines following intrachain-disulfide reduction (e.g. Brentuximab vedotin (available as Adcetris®), Polivy, Padcev, Enhertu, Trodelvy, Blenrep).

Brentuximab vedotin is however known to cause sensitive and long-lasting toxicities as side effects such as thrombocytopenia in the clinical routine.

Accordingly, there is still a need for an improved Brentuximab-comprising ADC, preferably having fewer side effects. The present invention aims to address this need.

### SUMMARY OF THE INVENTION

This need is solved by the subject-matter as defined in the claims and in the embodiments described herein.

Accordingly, the present invention relates to an antibody-drug conjugate (ADC) comprising:

(a) Brentuximab, wherein Brentuximab comprises at the C-terminus of the light chains, the heavy chains or all of the heavy and light chains of the Brentuximab a recognition sequence for tubulin tyrosine ligase and a non-natural amino acid; and
(b) at least one drug moiety;
wherein a drug moiety is coupled to each of the non-natural amino acids via a linker.

The heavy chains of Brentuximab may have an amino acid sequence that comprises SEQ ID NO: 1 or have a sequence identity of at least 95% to SEQ ID NO: 1 and/or wherein the light chains of Brentuximab have an amino acid sequence that comprises SEQ ID NO: 2 or have a sequence identity of at least 95% to SEQ ID NO: 2, preferably, Brentuximab consists of heavy chains consisting of the amino acid sequence of SEQ ID NO: 1 and light chains consisting of the amino acid sequence of SEQ ID NO: 2.

The drug moiety may be selected from the group consisting of camptothecins, maytansinoids, calicheamycins, duocarmycins, tubulysins, amatoxins, dolastatins and auristatins such as monomethyl auristatin E (MMAE), pyrrolobenzodiazepine dimers, indolino-benzodiazepine dimers, radioisotopes, therapeutic proteins and peptides (or fragments thereof), nucleic acids, PROTACs, kinase inhibitors, MEK inhibitors, KSP inhibitors, and analogues or prodrugs thereof, preferably the drug moiety is MMAE.

The recognition sequence for tubulin tyrosine ligase may have at least the amino acid sequence X₁X₂X₃X₄ (SEQ ID NO: 3), wherein X₁ and X₂ is any amino acid, X₃ is E, D or C and X₄ is E, preferably wherein X₂ is G, S, A, V, or F and/or wherein X₁ is E, D, A, K, or P.

The recognition sequence may be EGEE (SEQ ID No. 4), preferably the recognition sequence is VDSVEGEGEEEGEE (SEQ ID No. 5), SVEGEGEEEGEE (SEQ ID No. 6), SADGEDEGEE (SEQ ID No. 7), SVEAEAEEGEE (SEQ ID No. 8), SYEDEDEGEE (SEQ ID No. 9), or SFEEENEGEE (SEQ ID No. 10).

The unnatural amino acid may be a 2-substituted, 3-substituted or 4-substituted tyrosine or a tyrosine derivative substituted at the benzylic position. The 3- or 4-substituted tyrosine derivative may be 3-nitrotyrosine, 3-aminotyrosine, 3-azidotyrosine, 3-formyltyrosine, 3-acetyltyrosine, or 4-aminophenylalanine, preferably the unnatural amino acid is 3-formyltyrosine.

The linker may be cleavable, preferably by a protease, more preferably by a cathepsin such as cathepsin B. The linker may comprise a valine-citrulline moiety. The linker may comprise a hydroxylamine group and the unnatural amino acid comprises a formyl group ortho of a hydroxyl group in an aromatic ring such as 3-formyltyrosine, and wherein the hydroxylamine group of the linker forms an oxime with the formyl group of the unnatural amino acid after conjugation.

Brentuximab may be conjugated to two, four, six, or eight, preferably two or four drug moieties, more preferably two drug moieties.

The linker may have a structure as depicted in structure 1 before being coupled to the unnatural amino acid:

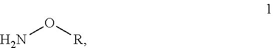

wherein R is one or more drug moieties, which are optionally coupled to the hydroxylamine of structure 1 by one or more cleavage sites, preferably wherein the hydroxylamine of structure 1 is conjugated to the unnatural amino acid.

The linker may have a structure as depicted in structure 2 or 3 before being coupled to the unnatural amino acid:

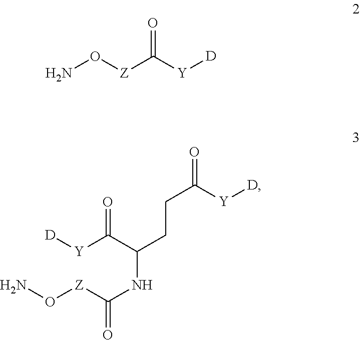

wherein Z is selected from the group consisting of substituted or unsubstituted alkyl, substituted or unsubstituted alkenyl, substituted or unsubstituted alkynyl, substituted or unsubstituted cycloalkyl, substituted or unsubstituted aryl, substituted or unsubstituted arylalkyl, substituted or unsubstituted heteroaryl, substituted or unsubstituted heteroarylalkyl, substituted or unsubstituted heterocyclyl, substituted or unsubstituted heteroalkyl, substituted or unsubstituted heteroalkenyl and substituted or unsubstituted heteroalkynyl;
wherein D is one or more drug moieties; and
wherein Y is a cleavage site such as a cleavage site for a cathepsin such as cathepsin B; preferably wherein the hydroxylamine of structure 2 or 3 is conjugated to the unnatural amino acid.

The linker may have a structure as depicted in structure 4 or 5 before being coupled to the unnatural amino acid, wherein D is a drug moiety, preferably MMAE:

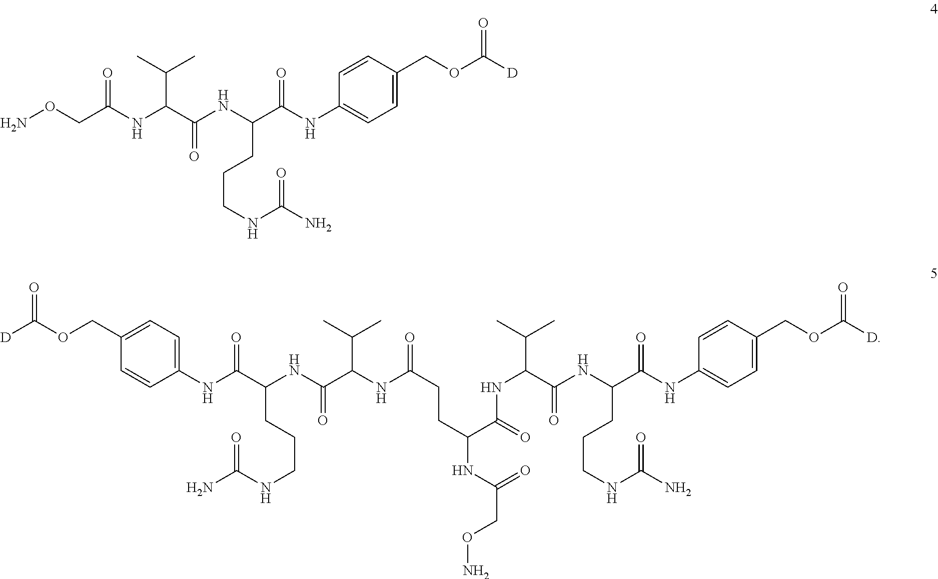

The unnatural amino acid may be 3-formyltyrosine and the hydroxylamine group of the linker may form an oxime with the 3-formyl group of the unnatural amino acid.

The present invention further relates to a method of producing an ADC as defined herein, comprising

(a) introducing or adding at the C-terminus of the light chain, the heavy chain or both the light chain and the heavy chain of Brentuximab a recognition sequence for tubulin tyrosine ligase;
(b) contacting the Brentuximab obtained in step (a) in the presence of tubulin tyrosine ligase and a non-natural amino acid under conditions suitable for the tubulin tyrosine ligase to ligate said Brentuximab with said non-natural amino acid; and
(c) conjugating an optionally cleavable linker comprising a drug moiety to said ligated Brentuximab obtained in step (b).

The present invention further relates to an ADC obtainable by the method of producing an ADC as defined herein. The present invention further relates to an ADC obtained by the method of producing an ADC as defined herein.

The present invention further relates to a pharmaceutical composition comprising the ADC of the invention.

The present invention further relates to the ADC of the invention or the pharmaceutical composition of the invention for use in a method of treating a disease. Preferably, the disease is associated with overexpression of CD30. More preferably, the disease is selected from the group consisting of lymphoma, such as Hodgkin's lymphoma (HL), non-Hodgkin lymphoma (NHL), anaplastic large-cell lymphoma (ALCL), large B-cell lymphoma, paediatric lymphoma, T-cell lymphoma and enteropathy-associated T-cell lymphoma (EATL), leukaemia, such as acute myeloid leukaemia (AML), acute lymphoblastic leukaemia (ALL) and mast cell leukaemia, germ cell cancer, graft-versus-host disease (GvHD) and lupus, in particular systemic lupus erythematosus (SLE), preferably Hodgkin Lymphoma (HL) or anaplastic large cell lymphoma (ALCL).

### BRIEF DESCRIPTION OF THE DRAWINGS

The invention will be better understood with reference to the detailed description when considered in conjunction with the non-limiting examples and the accompanying drawings, in which:

FIGS. 1A-1D show analytical size exclusion chromatography (SEC) chromatograms, with FIG. 1A showing SEC of unmodified Brentuximab and FIGS. 1B-1D showing SEC of Tub-tag variants of Brentuximab after Protein A chromatography (PAC). The monoclonal antibodies (mAbs) are highly monomeric after expression and purification.

FIGS. 2A-2B show an analysis of Brentuximab (black) and the Tub-tag analogs of Brentuximab (Brentuximab is abbreviated as “Bren.”) denoted as Bren. LC-Tub (grey, dotted), Bren. HC-Tub (grey), Bren. LCHC-Tub (black, dotted). FIG. 2A is a hydrophilic interaction chromatography (HIC) chromatogram showing normalized absorption spectra recorded at 220 nm. The retention time is a measure for hydrophobicity. FIG. 2B shows differential scanning fluorimetry (DSF) for determination of the melting point (Tₘ).

FIGS. 3A-3F show an analysis of Brentuximab Tub-tag ADCs via analytical SEC and HIC. Each of the Brentuximab variants (Brentuximab is abbreviated as “Bren.”, FIGS. 3A and 3D show Bren. HC-vc-PAB-MMAE DAR2 (also denoted herein as “Bren. HC-2”), FIGS. 3B and 3E show Bren. LC-vc-PAB-MMAE DAR2 (also denoted herein as “Bren. LC-2”), and FIGS. 3C and 3F show Bren. LCHC-vc-PAB-MMAE DAR4 (also denoted herein as “Bren. LCHC-2”) was expressed, purified with PAC, and conjugated to payload 2 for generation of DAR 2 and payload 2 or payload 3 for generation of DAR 4 ADCs. After final polishing with HIC and buffer exchange, Tub-tag ADCs were analyzed in terms of aggregate content and DAR homogeneity. All Tub-tag ADCs contain a very low content of HMWS (<1%) and displayed an excellent DAR homogeneity.

FIGS. 4A-4F show an analysis of Brentuximab Tub-tag ADCs by middle-up protein MS after deconvolution of the crude spectra. FIG. 4A shows the result for Bren. HC-Tub, FIG. 4B shows the result for Bren. LCHC-Tub, FIG. 4C shows the result for Bren-LC-Tub, FIG. 4D shows the result for Bren. HC-vc-PAB-MMAE (also denoted herein as “Bren. HC-2”), FIG. 4E shows the result for Bren-LCHC-vc-PAB-MMAE (also denoted herein as “Bren. LCHC-2”) and FIG. 4F shows the result for Bren. LC-2(vc-PAB-MMAE) (also denoted herein as “Bren. LC-3”). The mass shift of 1369 m/z results from the incorporation of 3-formyl-L-tyrosine 1 and oxime ligation with payload 2. After oxime ligation with payload 3 a mass difference of 2604 m/z was observed. These results are in accordance with the calculated values.

FIGS. 5A-5C show in FIG. 5A RP-HPLC analysis of the valine-citrulline (vc) containing payloads 2 and 3 during digestion reaction with the protease cathepsin B (1:1000 for each vc moiety). Prior to analysis the reaction was stopped with E-64. Chromatogram after a reaction time of 150 min recorded at 220 nm. The plot of FIG. 5B shows the increase of free MMAE over time (black) and the decrease of payload 2 (grey, circle). FIG. 5C shows cleavage of payload 3. During the reaction with cathepsin B an intermediate is formed (grey, triangle) which contains one MMAE moiety.

FIGS. 6A-6C show the results of the storage of Brentuximab vedotin and Brentuximab Tub-tag ADCs at elevated temperatures. FIG. 6A displays the increase of HMWS during the course of the study (Bren. LC-2(vc-PAB-MMAE) (also denoted herein as “Bren. LC-3”); Bren. LC-vc-PAB-MMAE (also denoted herein as “Bren. LC-2”); Bren. HC-vc-PAB-MMAE (also denoted herein as “Bren. HC-2”). The highest increase of aggregate content can be seen for Brentuximab vedotin. In comparison, the HMWS content of Tub-tag ADCs remains almost constant. FIGS. 6B and 6C show SEC chromatograms showing normalized absorption spectra recorded at 220 nm for Brentuximab vedotin (average drug to antibody ratio (DARₐᵥ) 4) and Bren. LC conjugated with payload 3 (Bren. LC-3, DAR 4).

FIGS. 7A-7D show an exemplarily illustration of Brentuximab Tub-tag ADC stability in mouse plasma. FIGS. 7A and 7B show the result of storage of Brentuximab vedotin at 37° C. in mouse-plasma and FIGS. 7C and 7D show the result of storage of Bren. LC-3 DAR 4 at 37° C. in mouse-plasma.

FIG. 8 shows the in vitro efficacy of Brentuximab Tub-tag ADCs in the CD30-overexpressing cell line Karpas299 and the CD30-negative cell line HL60.

FIG. 9 shows the in vivo efficacy of Bren. LC-2 expressed in terms of tumor volume (cm³). For assessment of the in vivo efficacy of Bren. LC-2, mice, bearing a tumor volume between 100-150 mm³ were randomized and treatment was conducted with two injections of 1.5 mg/kg at day 7 and 10 after tumor transplantation.

FIG. 10 shows the in vivo efficacy of Bren. LC-2 expressed in terms of tumor volume (cm³) and percentage of survival of treated animals. For assessment of the in vivo efficacy of Bren. LC-2, mice, bearing a tumor volume between 100-150 mm³ were randomized and treatment was conducted with one single injection of 1.0 mg/kg.

FIG. 11 shows the in vivo efficacy of Bren. LC-3 expressed in terms of tumor volume (cm³) and percentage of survival of treated animals (Kaplan-Meier-Plot). For assessment of the in vivo efficacy of Bren. LC-3, mice, bearing a tumor volume between 100-150 mm³ were randomized and treatment was conducted with two injections of 0.5 mg/kg at day 8 and 11 after tumor transplantation.

FIGS. 12A-12B show a PK analysis of Brentuximab Tub-tag MMAE (Bren LC-2). In FIG. 12A the amount of intact ADC in comparison to Brentuximab vedotin is shown. In FIG. 12B, the amount of transferred MMAE to blood proteins analyzed by MS-Analysis is shown.

FIG. 13 shows the mean serum concentrations of intact ADC and total antibody in male and female rats following an intravenous (bolus) administration at 10 mg/kg at day 1, 8, 15 and 22 for Brentuximab Tub-tag ADCs compared to that of Brentuximab vedotin.

FIG. 14 shows an exemplary toxicity profile of Brentuximab Tub-tag ADCs (Bren LC-2) (grey bar) compared to that of Brentuximab vedotin (black bar) in male and female rats. Parameters depicted in FIG. 14 are Reticulocytes in counts per microliter (RET in K/μl), Red Blood Cells in millions per microliter (RBC in M/μL), Hemoglobin in millions per microliter (Hb in M/μL), Hematocrit in volume percent (HTC in %), Eosinophils in count per microliter (EOS in K/μl), activated partial thromboplastin time in seconds (APPT) (seconds), glucose levels in millimole per liter (mmol/L), thymus weight in gram (g) and testicle weight in gram (g).

FIG. 15 shows a chromatogram of HA-VC-PAB-MMAE after purification.

FIG. 16 shows a chromatogram of Boc-Glu-(VC-PAB-MMAE)₂ after purification.

FIG. 17 shows a chromatogram of Boc-HA-Glu-(VC-PAB-MMAE)₂ after purification.

FIG. 18 shows a chromatogram of HA-Glu-(VC-PAB-MMAE)₂ after purification.

FIG. 19 shows a toxicokinetic analysis of Brentuximab Tub-tag MMAE in cynomolgus monkey dosed with 12 & 15 mg/kg. Total amount of mAb and intact ADC was assessed by ELISA.

High overlap of intact ADC and total mAb curves show high stability of Brentuximab Tub-tag MMAE at both dose levels.

FIG. 20 shows the body weight and a selection of different blood values that have been collected throughout a repeated dose study of Brentuximab Tub-tag MMAE (Bren. LC-2) in cynomolgus monkey. FIG. 20, upper panel on the left shows the body weight over time. FIG. 20, upper panel on the right shows the lymphocytes concentration over time. FIG. 20, lower panel on the left shows the neutrophiles concentration over time. FIG. 20, lower panel on the right shows the white blood cells concentration over time. The Data is shown, in each case, as mean and error of three groups of two female animals dosed repeatedly with 6, 12 and 15 mg/kg Brentuximab Tub-tag MMAE (Bren. LC-2) in comparison with literature values of a similar study with Brentuximab vedotin (Adcetris). Bodyweight is depicted in kilogram, lymphocytes in billion per liter, neutrophiles in billion per liter and white blood cells in billion per liter.

FIG. 21 shows the amino acid sequences of the heavy chain and the light chains of Brentuximab, as described in the 2013 Report of the Evaluation and Licensing Division, Pharmaceutical and Food Safety Bureau Ministry of Health, Labour and Welfare on the Deliberation Results for Brentuximab Vedotin available under the link https://www.pmda.go.jp/files/000229787.pdf.

### DETAILED DESCRIPTION OF THE INVENTION

The present invention is described in detail in the following and will also be further illustrated by the appended examples and figures.

Lymphomatic malignancies account for about 4-5% of all cancer cancers and derive from the lymphatic system. In classical Hodgkin's lymphoma and several non-Hodgkin lymphomas such as Anaplastic large cell lymphoma and Peripheral T cell lymphoma, CD30 has proven as a valuable tumor biomarker for targeted treatment in recent years. With the First-in-class approval of Brentuximab vedotin (Adcetris®) in 2011, the first CD30-specific biological drug has entered the market and has since become an indispensable treatment option with good overall response. Brentuximab vedotin consists of the tumour-targeting chimeric IgG1 antibody component brentuximab and a linker-payload component, comprising a Cathepsin B cleavable Valin-Citrulline linker moiety chemically connected with a potent payload moiety Monomethyl-Auristatin E, which efficiently induces apoptosis upon intracellular delivery and release.

Brentuximab vedotin is however known to cause sensitive and long-lasting toxicities as side effects such as thrombocytopenia in the clinical routine.

The present Inventors could show that the use of the tubulin tyrosine ligase surprisingly allows a highly specific and stoichiometrically defined conjugation of a drug moiety to Brentuximab, which has been modified by adding TTL recognition sequence at the C-terminus of the light and/or heavy chains. The TTL can be used to conjugate an unnatural amino acid such as 3-formyl tyrosine to the TTL recognition sequence. The drug moiety, e.g. MMAE, can be specifically conjugated to the unnatural amino acid by a linker, e.g. by a linker comprising an active group, which can react with an active group on the unnatural amino acid. E.g., the linker may comprise a hydroxylamine, which reacts with a formyl group of the unnatural amino acid to form an oxime (see Example 1). In sum, the Inventors showed that Brentuximab vedotin can surprisingly be improved by combining Brentuximab vedotin's functional components Brentuximab and MMAE by the conjugation strategy described herein. The ADCs of the invention surprisingly show a higher in vitro stability (see Example 2), higher in vivo efficacy (see Example 4), higher in vivo stability (see Example 5) and have an improved toxicology profile (see Example 6). Thereby, the side effects of Brentuximab vedotin could be surprisingly reduced.

Accordingly, the present invention relates to an antibody-drug conjugate (ADC) comprising:

(a) Brentuximab, wherein Brentuximab comprises at the C-terminus of the light chains, the heavy chains or all of the heavy and light chains of the Brentuximab a recognition sequence for tubulin tyrosine ligase and a non-natural amino acid; and
(b) at least one drug moiety;
wherein a drug moiety is coupled to each of the non-natural amino acids via a linker.

The term “antibody-drug conjugate” (ADC) as used herein in general refers to the linkage of an antibody such as Brentuximab or an antigen binding fragment thereof with another agent, such as a chemotherapeutic agent, a toxin, an immunotherapeutic agent, an imaging probe, and the like. The linkage can be covalent bonds, or non-covalent interactions such as through electrostatic forces, preferably covalent bonds. Various linkers, known in the art and described herein, can be employed in order to form the antibody drug conjugate. An ADC of the invention may also be described by having the formula Ab-(L-(D)ₓ)_{y}. “Ab” stands for an antibody such as Brentuximab or antigen binding fragment thereof, “L” stands for a linker and “D” stands for a drug moiety. “x” may be an integer from 1 to 10. “y” may be an integer from 1 to 10. Accordingly, x may be 1, 2, 3, 4, 5, 6, 7, 8, 9 or 10. Accordingly, y may be 1, 2, 3, 4, 5, 6, 7, 8, 9 or 10. As purely illustrative examples, x or y may be an integer from 1 to 8, from 1 to 6, from 1 to 4, or from 1 to 2.

### “Ab”: Antibody

### Brentuximab

As outlined herein, the ADC of the invention can be described by having the formula Ab-(L-(D)ₓ)_{y}. The “Ab part” of the ADC of the invention is Brentuximab. Brentuximab, also known as cAC10 or SGN-30, is an antibody well known to a person skilled in the art (see e.g., Wahl et al. (2002), Cancer Res, 62:3736-3742). Brentuximab is a monoclonal antibody specifically binding to CD30 on the cell surface of target cells. The binding to CD30 initiates internalization of Brentuximab, which then traffics to the lysosomal compartment. Within the cell, the drug moiety of the ADC may be released from the monoclonal antibody, e.g., via cleavage or degradation of the linker. Brentuximab is comprised in the ADC of the invention.

CD30, also known as TNFRSF8, is a cell membrane protein of the tumor necrosis factor receptor family and tumor marker. This receptor is expressed by activated, but not by resting, T and B cells. TRAF2 and TRAF5 can interact with this receptor, and mediate the signal transduction that leads to the activation of NF-κB. It is a positive regulator of apoptosis, and also has been shown to limit the proliferative potential of autoreactive CD8 effector T cells and protect the body against autoimmunity. Two alternatively spliced transcript variants of this gene encoding distinct isoforms have been reported. CD30 is associated with anaplastic large cell lymphoma. It is expressed in embryonal carcinoma but not in seminoma and is thus a useful marker in distinguishing between these germ cell tumors. CD30 and CD15 are also expressed on Reed-Sternberg cells typical for Hodgkin's lymphoma.

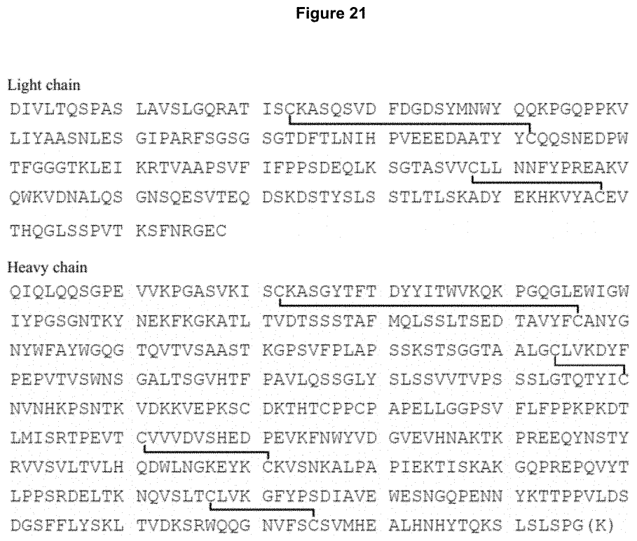

Each of the heavy chains of Brentuximab as the approved drug and as used herein has the amino acid sequence of SEQ ID NO: 1 (which is also shown in FIG. 21). It is also possible herein to use an antibody the heavy chain of which has a sequence identity of at least 95%, at least 96%, at least 97%, at least 98% or at least 99% to SEQ ID NO: 1. For example, and as shown in FIG. 21, the last residue K at position 447 can be omitted, if wanted. Such a heavy chain can be considered as a variant of the heavy chain of Brentuximab. Similarly, it is possible to delete, for example, the first two or three N-terminal residues of a heavy chain of Brentuximab. Such a heavy chain would have three or four mutations compared to Brentuximab and thus a sequence identity of about 99.2% (four amino acids difference) to 99.8% (one amino acid difference) to the Brentuximab heavy chain.

Each of the light chains of Brentuximab as the approved drug and as used herein has the amino acid sequence of SEQ ID NO: 2 (which is also shown in FIG. 21). It is also possible herein to use an antibody the light chain of which has a sequence identity of at least 95%, at least 96%, at least 97%, at least 98% or at least 99% to SEQ ID NO: 2. For example, the last residue C at position 218 can be omitted, if wanted. Such a light chain can be considered as a variant of the light chain of Brentuximab. Similarly, it is possible to delete, for example, the first two or three N-terminal residues of a light chain of Brentuximab. Such a light chain would have three or four mutations compared to Brentuximab and thus a sequence identity of 98.2% (four amino acids difference) to 99.5% (one amino acid difference) to the Brentuximab light chain.

In one particular embodiment, Brentuximab comprises heavy chains comprising the amino acid sequence of SEQ ID NO: 1 and light chains comprising the amino acid sequence of SEQ ID NO: 2. In one further particular embodiment, Brentuximab consists of heavy chains consisting of the amino acid sequence of SEQ ID NO: 1 and light chains consisting of the amino acid sequence of SEQ ID NO: 2.

Generally, when used herein, the terms “percent (%) identical” or “percent (%) identity,” in the context of two or more nucleic acids or polypeptide sequences, refers to the extent to which two or more sequences or subsequences that are the same. Two sequences are “identical” if they have the same sequence of amino acids or nucleotides over the region being compared. Two sequences are “substantially identical” if two sequences have a specified percentage of amino acid residues or nucleotides that are the same (i.e., 60% identity, optionally 65%, 70%, 75%, 80%, 85%, 90%, 95%, or 99% identity over a specified region, or, when not specified, over the entire sequence), when compared and aligned for maximum correspondence over a comparison window, or designated region as measured using one of the following sequence comparison algorithms or by manual alignment and visual inspection. Optionally, the identity exists over a region that is at least about 30 nucleotides (or 10 amino acids) in length, or more preferably over a region that is 100 to 500 or 1000 or more nucleotides (or 20, 50, 200 or more amino acids) in length.

The percentage of sequence homology or sequence identity can, for example, be determined herein using the program BLASTP, version blastp 2.2.5 (Nov. 16, 2002) (cf. Altschul et al., Nucleic Acids Res, 1997). In this embodiment the percentage of homology is based on the alignment of the entire polypeptide sequence (matrix: BLOSUM 62; gap costs: 11.1; cut-off value set to 10⁻³) including the propeptide sequences, preferably using the wild-type protein scaffold as reference in a pairwise comparison. It is calculated as the percentage of numbers of “positives” (homologous amino acids) indicated as result in the BLASTP program output divided by the total number of amino acids selected by the program for the alignment.

### TTL Recognition Sequence and Coupling of Unnatural Amino Acids to Brentuximab

In the ADC of the present invention, the Brentuximab can be fused to one or more TTL recognition sequence(s), e.g. at the C-terminus of the light chain, the heavy chain or both the light and the heavy chain. This TTL recognition sequence allows the TTL to conjugate an unnatural amino acid to the TTL recognition sequence at the C-terminus of the light and/or heavy chain of Brentuximab. Consequently, the Brentuximab comprised in the ADC of the invention comprises at the C-terminus of the light chains, the heavy chains or all of the heavy and light chains of the Brentuximab a recognition sequence for tubulin tyrosine ligase (“TTL recognition sequence”). In the ADC of the invention, an unnatural amino acid is attached to this TTL recognition sequence. Coupled to this unnatural amino acid is the drug moiety via the linker. “Tub-tag” when used herein, relates to a recognition sequence for TTL or a TTL recognition sequence, preferably SEQ ID NO: 4 or SEQ ID NO: 5.

As used herein and throughout the entire description, the term “Tubulin tyrosine ligase”, abbreviated sometimes herein as “TTL”, encompasses polypeptides that are capable of functionalizing polypeptides, i.e. covalently attaching an unnatural amino acid as defined herein to a polypeptide. For that action it is preferred that said polypeptide comprises a recognition sequence for TTL. Said term encompasses TTLs from eukaryotes, preferably mammals, more preferably from Canis Lupus. A preferred TTL is shown in SEQ ID No: 13. Also encompassed by said term is a TTL that has 70%, 80%, 90% or 95% or more identity over its entire amino acid sequence with the amino acid sequence of the TTL shown in SEQ ID No: 13. Preferably, such polypeptides having an amino acid sequence which shares an identity as described before have TTL activity. TTL activity can be tested as is known in the art or described herein. The percentage of sequence identity can, for example, be determined herein as described above. Preferably the amino acid sequence shown in SEQ ID No: 13 is used as reference in a pairwise comparison.

As used herein and throughout the entire description, the term “functionalizing” in all its grammatical forms as used herein means “covalently attaching an unnatural amino acid” to a polypeptide such as Brentuximab. Without wishing to be bound by a specific theory, it is envisaged that the TTL adds an unnatural amino acid as defined herein to the ultimate C-terminal amino acid of the TTL recognition sequence.

Methods for modifying proteins making use of the tubulin tyrosine ligase (TTL) have been described in WO 2016/066749 and WO 2017/186855, which are hereby incorporated by reference. For the TTL to attach a non-natural amino acid to a protein of interest such as the antibody of the ADC described herein, the TTL needs a recognition sequence. The ADC described herein may be modified to comprise a recognition sequence for tubulin-tyrosine ligase (TTL) at the C-terminus of the light, the heavy or both the light and the heavy chains of Brentuximab, comprising at least the amino acid sequence X₄X₃X₂X₁ (SEQ ID NO: 15). The term “recognition sequence” or “recognition motif” are used interchangeably herein and refer to a stretch of amino acids that is recognized by the TTL. Such recognition sequences are known in the art; see, e.g., Ruediger et al. (1994), Eur. J. Biochem. 220, 309-320 or Prota e al. (2013), J. Cell. Biol. 200, No. 3, 259-270. Moreover, the skilled person can easily test whether or not an amino acid sequence of interest is a TTL recognition sequence by applying, e.g., the assay “Tyrosination of peptides by TTL” described in Ruediger et al. “Recognized” by the TTL includes binding of the TTL to the recognition motif. The recognition motif advantageously comprises at least 4 amino acids which are designated X₄, X₃, X₂ and X₁ herein. In general, “X” can denote any amino acid unless indicated otherwise herein. Amino acids include, but are not limited, to the twenty “standard” amino acids: isoleucine (Ile, I), leucine (Leu, L), lysine (Lys, K), methionine (Met, M), phenylalanine (Phe, F), threonine (Thr, T), tryptophan (Trp, W), valine (Val, V), alanine (Ala, A), asparagine (Asn, N), aspartate (Asp, D), cysteine (Cys, C), glutamate (Glu, E), glutamine (Gln, Q), glycine (Gly, G), proline (Pro, P), serine (Ser, S), tyrosine (Tyr, Y), arginine (Arg, R) and histidine (His, H). The present invention also includes, without limitation, D-configuration amino acids, β-amino acids, amino acids having side chains as well as all non-natural amino acids known to one skilled in the art.

The recognition sequence for tubulin tyrosine ligase may have at least the amino acid sequence X₁X₂X₃X₄ (SEQ ID NO: 3), wherein X₁ and X₂ is any amino acid, X₃ is E, D or C and X₄ is E, preferably wherein X₂ is G, S, A, V, or F and/or wherein X₁ is E, D, A, K, or P. The recognition sequence may be EGEE (SEQ ID No. 4), preferably the recognition sequence is VDSVEGEGEEEGEE (SEQ ID No. 5), SVEGEGEEEGEE (SEQ ID No. 6), SADGEDEGEE (SEQ ID No. 7), SVEAEAEEGEE (SEQ ID No. 8), SYEDEDEGEE (SEQ ID No. 9), or SFEEENEGEE (SEQ ID No. 10). In one embodiment, the recognition sequence is VDSVEGEGEEEGEE (SEQ ID No. 5).

The number of the TTL recognition sequences present in the Brentuximab comprised in the ADC of the present invention determines the drug to antibody ratio (DAR). While the drug to antibody ratio (DAR) has a defined stoichiometric value for a specific conjugate molecule (e.g., y multiplied by x in the formula Ab-(L-(D)ₓ)_{y}), it is understood that the value will might be an average value when used to describe a sample containing many molecules, due to some degree of inhomogeneity, typically associated with the conjugation step. Therefore, in the context of any one of the antibody drug conjugates described herein above and below the average loading for a sample of an antibody drug conjugate is referred to as the drug to antibody ratio, or “DAR.” Preferably however, the present invention provides ADCs, in which substantially all or all ADCs have the required DAR or are, in other words, stoichiometrically defined.

The ADC of the present invention is based on Brentuximab, a monoclonal IgG antibody. IgG antibodies comprise or consist of two “light chains” and two “heavy chains”. Since the TTL recognition sequence can be fused only to the C-terminus of the light chain, the heavy chain, or both the light chain and the heavy chain, the maximum number of TTL recognition sequences of Brentuximab is limited to 4. In case either the heavy chain or the light chain comprises a TTL recognition sequence at the C-terminus, there are 2 TTL recognition sequences in the Brentuximab comprised in the ADC of the invention. In case the heavy chain and the light chain comprise a TTL recognition sequence at the C-terminus, there are 4 TTL recognition sequences in the Brentuximab comprised in the ADC of the invention.

In an ADC of the present invention, the TTL recognition sequence may be either directly fused to the C-terminus of a chain of the antibody Brentuximab. See in this context, the Brentuximab heavy chain of (SEQ ID NO: 12): in which the the TTL recognition sequence is directly fused (i.e. without any amino acid residue present inbetween) to the C-terminus of the heavy chain. Alternatively, a (peptide) linker may be arranged in between the TTL recognition sequence and a chain of the antibody. Thus it is possible that the Brentuximab light chain, the heavy chain, or both the light chain and the heavy chain may include an amino acid linker sequence that is arranged in between the TTL recognition sequence and the respective C-terminus. As illustrative example, see the light chain of SEQ ID NO: 11 in which the TTL recognition sequence is fused to the C-terminus of the Brentuximab light chain via a GGGGS (SEQ ID NO: 16) (G4S) linker. The amino acid linker may have any suitable length, as long as the function of the TTL recognition sequence is maintained. For example, the amino acid linker may comprise 1, 2, 3, 4, 5, 6, 7, 8, 9, 10, 11, 12, 13, 14 or 15 amino acids residues. See again the light chain of SEQ ID NO: 11 in which a GGGGS (SEQ ID NO: 16) (G4S) linker is arranged between the TTL recognition sequence and the C-terminus of the Brentuximab. It may also be possible to use other standard linkers such as (G4S)₂ (SEQ ID NO; 17) or (G4S)₃ (SEQ ID NO: 18) which are commonly used as linkers to fuse different chains of recombinant antibody molecules such as single-chain Fv fragments to each other. Thus, in these examples, the linker may have a length of 10 amino acid residues ((G4S)₂) (SEQ ID NO: 17) or of 15 amino acid residues ((G4S)₃) (SEQ ID NO 18). The linker is however by no means restricted to a length of up to 15 amino acid residues but, in accordance with the above disclosure, the amino acid linker sequence may comprise even more than 15 amino acids.

Also in accordance with the above disclosure, an ADC of the present invention may be constructed such that none of the antibody chains comprise a peptide linker inbetween the TTL recognition sequence and the respective antibody chains. In such an ADC, the TTL recognition sequence may be directly fused to both the light chains and the heavy chains of Brentuximab. An ADC of the present invention may however also be constructed such that one of the antibody chains comprise a peptide linker inbetween the TTL recognition sequence and the respective antibody chain. In such an ADC, the TTL recognition sequence may be fused to each of the light chains via a linker (for example, a GGGGS-(SEQ ID NO: 16) linker as in the light chain of SEQ ID NO: 11) while the TTL recognition sequence may be directly fused to each of the two heavy chains of Brentuximab. Finally, an ADC of the present invention may also be constructed such that both the antibody light chain and the antibody heavy chain comprise a peptide linker inbetween the TTL recognition sequence and the respective antibody chain. In such an ADC, the TTL recognition sequence may be fused to each of the two light chains and each of the two heavy chains of Brentuximab via a linker (for example, a GGGGS (SEQ ID NO 16)-linker, or a (G4S)₂ (SEQ ID NO: 17) linker).

In one embodiment, Brentuximab comprises a TTL recognition sequence at the C-termini of the light chains, i.e. 2 in total. The TTL recognition sequence may be fused to the C-termini of the light chains via an amino acid linker sequence (such as e.g. GGGGS (SEQ ID NO: 16)). The TTL recognition sequence may be bound directly to the C-termini of the light chains. The Brentuximab heavy chain may comprise a polypeptide having a sequence as depicted in SEQ ID NO: 1 and the Brentuximab light chain may comprise a polypeptide having a sequence as depicted in SEQ ID NO: 11. The Brentuximab heavy chain may comprise a polypeptide having a sequence as depicted in SEQ ID NO: 1 and the Brentuximab light chain may comprise a polypeptide having a sequence as depicted in SEQ ID NO: 14. The Brentuximab heavy chain may consist of a polypeptide having a sequence as depicted in SEQ ID NO: 1 and the Brentuximab light chain may consist of a polypeptide having a sequence as depicted in SEQ ID NO: 11. The Brentuximab heavy chain may consist of a polypeptide having a sequence as depicted in SEQ ID NO: 1 and the Brentuximab light chain may consist of a polypeptide having a sequence as depicted in SEQ ID NO: 14.

In one embodiment, Brentuximab comprises a TTL recognition sequence at the C-termini of the light and heavy chains, i.e. 4 in total. The TTL recognition sequence may be fused to the C-termini of the light chains via an amino acid linker sequence (such as e.g. GGGGS (SEQ ID NO: 16)), and the TTL recognition sequence may be bound directly to the C-termini of the heavy chains. The TTL recognition sequence may be bound to the C-termini of the light chains via an amino acid linker (such as e.g. GGGGS (SEQ ID NO: 16)), and the TTL recognition sequence may be bound to the C-termini of the heavy chains via an amino acid linker (such as e.g. GGGGS (SEQ ID NO: 16)). The TTL recognition sequence may be bound directly to the C-termini of the light chains, and the TTL recognition sequence may be bound directly to the C-termini of the heavy chains. The Brentuximab heavy chain may comprise a polypeptide having a sequence as depicted in SEQ ID NO: 12 and the Brentuximab light chain may comprise a polypeptide having a sequence as depicted in SEQ ID NO: 11. The Brentuximab heavy chain may comprise a polypeptide having a sequence as depicted in SEQ ID NO: 12 and the Brentuximab light chain may comprise a polypeptide having a sequence as depicted in SEQ ID NO: 14. The Brentuximab heavy chain may consist of a polypeptide having a sequence as depicted in SEQ ID NO: 12 and the Brentuximab light chain may consist of a polypeptide having a sequence as depicted in SEQ ID NO: 11. The Brentuximab heavy chain may consist of a polypeptide having a sequence as depicted in SEQ ID NO: 12 and the Brentuximab light chain may consist of a polypeptide having a sequence as depicted in SEQ ID NO: 14.

In one embodiment, Brentuximab comprises a TTL recognition sequence at the C-termini of the heavy chains, i.e. 2 in total. The TTL recognition sequence may be bound directly to the C-termini of the heavy chains. The TTL recognition sequence may be bound to the C-termini of the heavy chains via an amino acid linker sequence (such as e.g. GGGGS (SEQ ID NO: 16)). The Brentuximab heavy chain may comprise a polypeptide having a sequence as depicted in SEQ ID NO: 12 and the Brentuximab light chain may comprise a polypeptide having a sequence as depicted in SEQ ID NO: 2. Brentuximab heavy chain may consist of a polypeptide having a sequence as depicted in SEQ ID NO: 12 and the Brentuximab light chain may consist of a polypeptide having a sequence as depicted in SEQ ID NO: 2.

The number of the TTL recognition sequences is however not necessarily equal to the number of drug moieties conjugated to the unnatural amino acid, i.e. not necessarily equal to the DAR. Also encompassed by the present invention is that more than one drug moiety is coupled (via a linker) to the unnatural amino acids. E.g., 2, 3, 4, 5, 6, 7, 8, 9 or 10, preferably 2, drug moieties may be coupled to one unnatural amino acid via the linker. In this case, the linker may act as a scaffold, to which 2, 3, 4, 5, 6, 7, 8, 9 or 10, preferably 2, drug moieties may be coupled. The linker in turn is coupled to the unnatural amino acid. Exemplary linkers, which act as scaffold for two drug moieties, are structures 3 or 5. Accordingly, the antibody may be conjugated to two, four, six, or eight, preferably two or four drug moieties, more preferably two drug moieties.

### Unnatural Amino Acids

The unnatural amino acid is the anchor point for the linker in the “Ab” part of the ADC of the invention. In this regard, it is again referred to the “ADC formula” described herein Ab-(L-(D)ₓ)_{y}. Thus, the unnatural amino acid, which can be coupled to the C-terminus of a polypeptide having a TTL recognition sequence at its C-terminus such as the Brentuximab comprised in the ADC of the invention by TTL, comprises an functional group that can react with a functional group of the linker L to form a covalent bond.

In general, the unnatural amino acid can be seen as a tyrosine derivative. Preferably, the tyrosine derivative comprises an active group that allows the conjugation of the drug moiety via the linker.

The tyrosine derivative may (further) contain an unnatural (non-natural) functional group, which is preferably used for chemoselective or bioorthogonal modifications. The functional group may be suitable for click chemistry. The term “click chemistry” refers to a chemical philosophy introduced by Kolb, Finn and Sharpless in 2001 and encompasses a group of powerful linking reactions that are able to generate covalent bonds quickly and reliably by joining small units comprising reactive groups together. Click chemistry reactions are typically modular, wide in scope, give high chemical yields, generate inoffensive byproducts, are stereospecific, and/or can be carried using readily available starting materials and reagents out under simple, physiological reaction conditions. In addition, click chemistry reactions preferably use no toxic solvents or use a solvent that is benign or easily removed (preferably water), and/or provides simple product isolation by non-chromatographic methods (crystallisation or distillation). A distinct exothermic reaction makes a reactant “spring loaded”.

Click chemistry reactions comprise, e.g., cycloaddition reactions, especially from the 1,3-dipolar family, hetero-Diels-Alder reactions; nucleophilic ring-opening reactions, e.g. of strained heterocyclic electrophiles, such as epoxides, aziridines, cyclic sulfates, cyclic sulfamidates, aziridinium ions and episulfonium ions: carbonyl chemistry of the non-aldol type (e.g. the formation of oxime ethers, hydrazones and aromatic heterocycles); and addition to carbon-carbon multiple bonds; e.g. oxidation reactions, such as epoxidation, dihydroxylation, aziridination, and nitrosyl and sulfenyl halide additions but also certain Michael addition reactions. General principles of click chemistry reactions have been described by Kolb, Finn and Sharpless (2001). It is within the knowledge of the person skilled in the art to select a click chemistry reaction that is suitable for attaching a desired drug moiety via a linker to the tyrosine derivative or unnatural amino acid covalently bonded to the Brentuximab comprised in the ADC of the invention.

The term “click chemistry handle,” as used herein, refers to a reactant, or a reactive group, that can partake in a click chemistry reaction. Such a reactant or reactive group is preferably an unnatural (non-natural) functional group for a chemoselective or bioorthogonal modification; however, it may alternatively be a natural functional group for a chemoselective or bioorthogonal modification. For example, a strained alkyne, e.g., a cyclooctyne, is a click chemistry handle, since it can partake in a strain-promoted cycloaddition, e.g. strain-promoted azide-alkyne cycloaddition (SPAAC). In general, click chemistry reactions require at least two molecules comprising click chemistry handles that can react with each other. Such click chemistry handle pairs that are reactive with each other are sometimes referred to herein as “partner click chemistry handles”. For example, an azide is a partner click chemistry handle to a cyclooctyne or any other alkyne. In the context of the present invention, the click chemistry handle can preferably be selected from the group consisting of terminal alkyne, azide, strained alkyne, diene, dieneophile, alkoxyamine, carbonyl, β-Arylethylamine, phosphine, hydrazide, hydrazine, thiol, tetrazine, alkene, cyclooctene, norbornene, tetrazine, nitrone, cyanobenzothiazole, and cyclooctyne. Other suitable click chemistry handles are readily accessible to the person skilled in the art.

In the context of conjugation via click chemistry, the conjugation is via a covalent bond formed by the reaction of the click chemistry handles. In certain embodiments, the association is covalent, and the entities are said to be “conjugated” to one another.

It should be noted that the invention is not limited to the foregoing, exemplary click chemistry handles, and additional click chemistry handles, reactive click chemistry handle pairs, and reaction conditions for such click chemistry handle pairs will be apparent to those of skill in the art.

Other methods suitable for conjugating the linker to the unnatural amino acid comprised by the Brentuximab comprised in the ADC of the invention comprise Staudinger reactions (e.g. Staudinger-ligation, Staudinger-Phosphite reaction, Staudinger-Phosphonite reaction), strain-promoted cycloadditions, tetrazine ligations, inverse-electron demand Diels-Alder reactions, thiazolidine-forming reactions of aldehydes or ketones with 1,2-aminothiols, oxazolidine-forming reactions of aldehydes, oxime formation, hydrazone formation, or ketones with 1,2-aminoalcohols, acetal-forming reactions of aldehydes or ketones with 1,2-diols, Pictet-Spengler reactions, trapped-Knoevenagel ligations, tandem-Knoevenagel condensation, thiol addition to alkenes or alkynes, cross-metathesis, metal-catalyzed, in particular Pd-, Cu, Ni and Fe-catalyzed cross couplings with tyrosine-derivatives substituted with electron-withdrawing groups, preferably oxime formation.

It is envisaged that a drug moiety can be attached to the unnatural amino acid covalently bonded to Brentuximab via the linker, for example, by click chemistry or any other suitable method as described herein. A drug moiety may thus be conjugated to the unnatural amino acid via the linker by a non-peptidic bond, however, in the alternative it may also be conjugated to the unnatural amino acid via the linker by a peptidic-bond.

The unnatural amino acid may be a 2-substituted, 3-substituted or 4-substituted tyrosine or a tyrosine derivative substituted at the benzylic position. The 3- or 4-substituted tyrosine derivative may be 3-nitrotyrosine, 3-aminotyrosine, 3-azidotyrosine, 3-formyltyrosine, 3-acetyltyrosine, or 4-aminophenylalanine. The 3-substituted tyrosine may be 3-nitrotyrosine. The 3-substituted tyrosine may be 3-aminotyrosine. The 3-substituted tyrosine may be 3-azidotyrosine. The 3-substituted tyrosine may be 3-formyltyrosine. The 3-substituted tyrosine may be 3-acetyltyrosine. The substituted tyrosine might be a substituted phenylalanine. The 4-substituted tyrosine may be 4-aminophenylalanine. A particularly preferred unnatural amino acid is 3-formyltyrosine.

As used herein and throughout the entire description, the term “optionally substituted” or “substituted” indicates that one or more (such as 1 to the maximum number of hydrogen atoms bound to a group, e.g., 1, 2, 3, 4, 5, 6, 7, 8, 9, or up to 10, such as between 1 to 5, 1 to 4, or 1 to 3, or 1 or 2) hydrogen atom(s) may be replaced with a group different from hydrogen such as alkyl (preferably, C₁₋₆ alkyl), alkenyl (preferably, C₂₋₆ alkenyl), alkynyl (preferably, C₂₋₆ alkynyl), aryl (preferably, 3- to 14-membered aryl), heteroaryl (preferably, 3- to 14-membered heteroaryl), cycloalkyl (preferably, 3- to 14-membered cycloalkyl), heterocyclyl (preferably, 3- to 14-membered heterocyclyl), halogen, CN, azido, NO₂, OR⁷¹, N(R⁷²)(R⁷³), ON(R⁷²)(R⁷³), N⁺(O⁻) (R⁷²)(R⁷³), S(O)₀₂R⁷¹, S(O)O₂₀R⁷¹, OS(O)₀₂R⁷¹, OS(O)O₂₀R⁷¹, S(O)₀₂N(R⁷²)(R⁷³), OS(O)₀₂N(R⁷²)(R⁷³), N(R⁷¹)S(O)₀₂R⁷¹, NR⁷¹S(O)₀₂OR⁷¹, NR⁷¹S(O)₀₂N(R⁷²)(R⁷³), C(═W¹)R⁷¹, C(═W¹)W¹R⁷¹, W¹C(═W¹)R⁷¹, and —W¹C(═W¹)W¹R⁷¹; wherein R⁷¹, R⁷², and R⁷³ are independently selected from the group consisting of H, C₁₋₆ alkyl, C₂₋₆ alkenyl, C₂₆ alkynyl, 3- to 7-membered cycloalkyl, 5- or 6-membered aryl, 5- or 6-membered heteroaryl, and 3- to 7-membered heterocyclyl, wherein each of the alkyl, alkenyl, alkynyl, cycloalkyl, aryl, heteroaryl, and heterocyclyl groups is optionally substituted with one, two or three substituents selected from the group consisting of C₁₋₃ alkyl, halogen, CF₃, CN, azido, NO₂, OH, O(C₁₋₃ alkyl), S(C₁₋₃ alkyl), NH₂, NH(C₁₋₃ alkyl), N(C₁₋₃ alkyl)₂, NHS(O)₂(C₁₋₃ alkyl), S(O)₂NH_{2-z}(C₁₋₃ alkyl)_{z}, C(═O)OH, C(═O)O(C₁₋₃ alkyl), C(═O)NH_{2-z}(C₁₋₃ alkyl)_{z}, NHC(═O)(C₁₋₃ alkyl), NHC(═NH)NH_{2z}(C₁₋₃ alkyl)_{z}, and N(C₁₋₃ alkyl)C(═NH)NH_{2-z}(C₁₋₃ alkyl)_{z}, wherein z is 0, 1, or 2 and C₁₋₃ alkyl is methyl, ethyl, propyl or isopropyl; W¹ is independently selected from O, S, and NR⁸⁴, wherein R⁸⁴ is —H or C₁₋₃ alkyl.

As used herein and throughout the entire description, the term “alkyl” refers to a monoradical of a saturated straight or branched hydrocarbon. Preferably, the alkyl group comprises from 1 to 12 (such as 1 to 10) carbon atoms, i.e., 1, 2, 3, 4, 5, 6, 7, 8, 9, 10, 11, or 12 carbon atoms (such as 1, 2, 3, 4, 5, 6, 7, 8, 9, or 10 carbon atoms), more preferably 1 to 8 carbon atoms, such as 1 to 6 or 1 to 4 carbon atoms. In some embodiments, the alkyl group employed in the invention contains 1-20 carbon atoms (C₁₋₂₀ alkyl). In another embodiment, the alkyl group employed contains 1-15 carbon atoms (C₁₋₁₅ alkyl). In another embodiment, the alkyl group employed contains 1-10 carbon atoms (C₁₋₁₀ alkyl). In another embodiment, the alkyl group employed contains 1-8 carbon atoms (C₁₋₈ alkyl). In another embodiment, the alkyl group employed contains 1-6 carbon atoms (C₁₋₆ alkyl). In another embodiment, the alkyl group employed contains 1-5 carbon atoms (C₁₋₅-alkyl). In another embodiment, the alkyl group employed contains 1-4 carbon atoms (C₁₋₄ alkyl). In another embodiment, the alkyl group employed contains 1-3 carbon atoms (C₁₋₃ alkyl). In another embodiment, the alkyl group employed contains 1-2 carbon atoms (C₁₋₂ alkyl). Examples of alkyl radicals include, but are not limited to, methyl, ethyl, propyl, iso-propyl, butyl, iso-butyl, tert-butyl, n-pentyl, iso-pentyl, sec-pentyl, neo-pentyl, 1,2-dimethyl-propyl, iso-amyl, n-hexyl, iso-hexyl, sec-hexyl, n-heptyl, iso-heptyl, n-octyl, 2-ethyl-hexyl, n-nonyl, n-decyl, n-undecyl, n-dodecyl, and the like, which may bear one or more substituents. Alkyl group substituents include, but are not limited to, any of the substituents described herein, that result in the formation of a stable moiety. In some embodiments the alkyl chain is a linear. In some embodiments the alkyl chain is branched. In some embodiments the alkyl chain is substituted. In some embodiment the alkyl chain is unsubstituted. In some embodiments the alkyl chain is linear and substituted or unsubstituted. In some embodiments the alkyl chain is branched and substituted or unsubstituted.

As used herein and throughout the entire description, the term “alkylene” refers to a diradical of a saturated straight or branched hydrocarbon. Preferably, the alkylene comprises from 1 to 10 carbon atoms, i.e., 1, 2, 3, 4, 5, 6, 7, 8, 9, or 10 carbon atoms, more preferably 1 to 8 carbon atoms, such as 1 to 6 or 1 to 4 carbon atoms. Exemplary alkylene groups include methylene, ethylene (i.e., 1,1-ethylene, 1,2-ethylene), propylene (i.e., 1,1-propylene, 1,2-propylene (—CH(CH₃)CH₂—), 2,2-propylene (—C(CH₃)₂—), and 1,3-propylene), the butylene isomers (e.g., 1,1-butylene, 1,2-butylene, 2,2-butylene, 1,3-butylene, 2,3-butylene (cis or trans or a mixture thereof), 1,4-butylene, 1,1-iso-butylene, 1,2-iso-butylene, and 1,3-iso-butylene), the pentylene isomers (e.g., 1,1-pentylene, 1,2-pentylene, 1,3-pentylene, 1,4-pentylene, 1,5-pentylene, 1,1-iso-pentylene, 1,1-sec-pentyl, 1,1-neo-pentyl), the hexylenisomers (e.g., 1,1-hexylene, 1,2-hexylene, 1,3-hexylene, 1,4-hexylene, 1,5-hexylene, 1,6-hexylene, and 1,1-isohexylene), and the like. Alkylene groups may be cyclic or acyclic, branched or unbranched, substituted or unsubstituted. Alkylene group substituents include, but are not limited to, any of the substituents described herein, that result in the formation of a stable moiety.

As used herein and throughout the entire description, the term “halogen” or “halo” means fluoro, chloro, bromo, or iodo.

As used herein and throughout the entire description, the term “azido” means N3.

As used herein and throughout the entire description, the term “alkenyl” refers to a monoradical of an unsaturated straight or branched hydrocarbon having at least one carbon-carbon double bond. Generally, the maximal number of carbon-carbon double bonds in the alkenyl group can be equal to the integer which is calculated by dividing the number of carbon atoms in the alkenyl group by 2 and, if the number of carbon atoms in the alkenyl group is uneven, rounding the result of the division down to the next integer. For example, for an alkenyl group having 9 carbon atoms, the maximum number of carbon-carbon double bonds is 4. Preferably, the alkenyl group has 1 to 4, i.e., 1, 2, 3, or 4, carbon-carbon double bonds. Preferably, the alkenyl group comprises from 2 to 10 carbon atoms, i.e., 2, 3, 4, 5, 6, 7, 8, 9, or 10 carbon atoms, more preferably 2 to 8 carbon atoms, such as 2 to 6 carbon atoms or 2 to 4 carbon atoms. Thus, in a preferred embodiment, the alkenyl group comprises from 2 to 10 carbon atoms and 1, 2, 3, 4, or 5 carbon-carbon double bonds, more preferably it comprises 2 to 8 carbon atoms and 1, 2, 3, or 4 carbon-carbon double bonds, such as 2 to 6 carbon atoms and 1, 2, or 3 carbon-carbon double bonds or 2 to 4 carbon atoms and 1 or 2 carbon-carbon double bonds. In certain embodiments, the alkenyl group employed in the invention contains 2-20 carbon atoms (C₂₋₂₀ alkenyl). In some embodiments, the alkenyl group employed in the invention contains 2-15 carbon atoms (C₂₋₁₅ alkenyl). In another embodiment, the alkenyl group employed contains 2-10 carbon atoms (C₂₋₁₀ alkenyl). In still other embodiments, the alkenyl group contains 2-8 carbon atoms (C₂₋₈ alkenyl). In yet other embodiments, the alkenyl group contains 2-6 carbons (C₂₋₆ alkenyl). In yet other embodiments, the alkenyl group contains 2-5 carbons (C₂₋₅ alkenyl). In yet other embodiments, the alkenyl group contains 2-4 carbons (C₂₋₄ alkenyl). In yet other embodiments, the alkenyl group contains 2-3 carbons (C₂₋₃ alkenyl). In yet other embodiments, the alkenyl group contains 2 carbons (C₂ alkenyl). The carbon-carbon double bond(s) may be in cis (Z) or trans (E) configuration. Exemplary alkenyl groups include vinyl, 1-propenyl, 2-propenyl (i.e., allyl), 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, 1-heptenyl, 2-heptenyl, 3-heptenyl, 4-heptenyl, 5-heptenyl, 6-heptenyl, 1-octenyl, 2-octenyl, 3-octenyl, 4-octenyl, 5-octenyl, 6-octenyl, 7-octenyl, 1-nonenyl, 2-nonenyl, 3-nonenyl, 4-nonenyl, 5-nonenyl, 6-nonenyl, 7-nonenyl, 8-nonenyl, 1-decenyl, 2-decenyl, 3-decenyl, 4-decenyl, 5-decenyl, 6-decenyl, 7-decenyl, 8-decenyl, 9-decenyl, and the like. If an alkenyl group is attached to a nitrogen atom, the double bond cannot be alpha to the nitrogen atom. In some embodiments the alkenyl chain is a linear. In some embodiments the alkenyl chain is branched. In some embodiments the alkenyl chain is substituted. In some embodiment the alkenyl chain is unsubstituted. In some embodiments the alkenyl chain is linear and substituted or unsubstituted. In some embodiments the alkenyl chain is branched and substituted or unsubstituted. Alkenyl group substituents include, but are not limited to, any of the substituents described herein, that result in the formation of a stable moiety.

As used herein and throughout the entire description, the term “alkenylene” refers to a diradical of an unsaturated straight or branched hydrocarbon having at least one carbon-carbon double bond. Generally, the maximal number of carbon-carbon double bonds in the alkenylene group can be equal to the integer which is calculated by dividing the number of carbon atoms in the alkenylene group by 2 and, if the number of carbon atoms in the alkenylene group is uneven, rounding the result of the division down to the next integer. For example, for an alkenylene group having 9 carbon atoms, the maximum number of carbon-carbon double bonds is 4. Preferably, the alkenylene group has 1 to 4, i.e., 1, 2, 3, or 4, carbon-carbon double bonds. Preferably, the alkenylene group comprises from 2 to 10 carbon atoms, i.e., 2, 3, 4, 5, 6, 7, 8, 9, or 10 carbon atoms, more preferably 2 to 8 carbon atoms, such as 2 to 6 carbon atoms or 2 to 4 carbon atoms. Thus, in a preferred embodiment, the alkenylene group comprises from 2 to 10 carbon atoms and 1, 2, 3, 4, or 5 carbon-carbon double bonds, more preferably it comprises 2 to 8 carbon atoms and 1, 2, 3, or 4 carbon-carbon double bonds, such as 2 to 6 carbon atoms and 1, 2, or 3 carbon-carbon double bonds or 2 to 4 carbon atoms and 1 or 2 carbon-carbon double bonds. The carbon-carbon double bond(s) may be in cis (Z) or trans (E) configuration. Exemplary alkenylene groups include ethen-1,2-diyl, vinyliden, 1-propen-1,2-diyl, 1-propen-1,3-diyl, 1-propen-2,3-diyl, allyliden, 1-buten-1,2-diyl, 1-buten-1,3-diyl, 1-buten-1,4-diyl, 1-buten-2,3-diyl, 1-buten-2,4-diyl, 1-buten-3,4-diyl, 2-buten-1,2-diyl, 2-buten-1,3-diyl, 2-buten-1,4-diyl, 2-buten-2,3-diyl, 2-buten-2,4-diyl, 2-buten-3,4-diyl, and the like. If an alkenylene group is attached to a nitrogen atom, the double bond cannot be alpha to the nitrogen atom. Alkenylene groups may be cyclic or acyclic, branched or unbranched, substituted or unsubstituted. Alkenylene group substituents include, but are not limited to, any of the substituents described herein, that result in the formation of a stable moiety.

As used herein and throughout the entire description, the term “alkynyl” refers to a monoradical of an unsaturated straight or branched hydrocarbon having at least one carbon-carbon triple bond. Generally, the maximal number of carbon-carbon triple bonds in the alkynyl group can be equal to the integer which is calculated by dividing the number of carbon atoms in the alkynyl group by 2 and, if the number of carbon atoms in the alkynyl group is uneven, rounding the result of the division down to the next integer. For example, for an alkynyl group having 9 carbon atoms, the maximum number of carbon-carbon triple bonds is 4. Preferably, the alkynyl group has 1 to 4, i.e., 1, 2, 3, or 4, more preferably 1 or 2 carbon-carbon triple bonds. Preferably, the alkynyl group comprises from 2 to 10 carbon atoms, i.e., 2, 3, 4, 5, 6, 7, 8, 9, or 10 carbon atoms, more preferably 2 to 8 carbon atoms, such as 2 to 6 carbon atoms or 2 to 4 carbon atoms. Thus, in a preferred embodiment, the alkynyl group comprises from 2 to 10 carbon atoms and 1, 2, 3, 4, or 5 (preferably 1, 2, or 3) carbon-carbon triple bonds, more preferably it comprises 2 to 8 carbon atoms and 1, 2, 3, or 4 (preferably 1 or 2) carbon-carbon triple bonds, such as 2 to 6 carbon atoms and 1, 2 or 3 carbon-carbon triple bonds or 2 to 4 carbon atoms and 1 or 2 carbon-carbon triple bonds. In certain embodiments, the alkynyl group employed in the invention contains 2-20 carbon atoms (C₂₋₂₀ alkynyl). In some embodiments, the alkynyl group employed in the invention contains 2-15 carbon atoms (C₂₁₅ alkynyl). In another embodiment, the alkynyl group employed contains 2-10 carbon atoms (C₂₋₁₀ alkynyl). In still other embodiments, the alkynyl group contains 2-8 carbon atoms (C₂₋₈ alkynyl). In still other embodiments, the alkynyl group contains 2-6 carbon atoms (C₂₋₆ alkynyl). In still other embodiments, the alkynyl group contains 2-5 carbon atoms (C₂₋₅ alkynyl). In still other embodiments, the alkynyl group contains 2-4 carbon atoms (C₂₋₄ alkynyl). In still other embodiments, the alkynyl group contains 2-3 carbon atoms (C₂₋₃ alkynyl). In still other embodiments, the alkynyl group contains 2 carbon atoms (C₂ alkynyl). Exemplary alkynyl groups include ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, 5-hexynyl, 1-heptynyl, 2-heptynyl, 3-heptynyl, 4-heptynyl, 5-heptynyl, 6-heptynyl, 1-octynyl, 2-octynyl, 3-octynyl, 4-octynyl, 5-octynyl, 6-octynyl, 7-octynyl, 1-nonylyl, 2-nonynyl, 3-nonynyl, 4-nonynyl, 5-nonynyl, 6-nonynyl, 7-nonynyl, 8-nonynyl, 1-decynyl, 2-decynyl, 3-decynyl, 4-decynyl, 5-decynyl, 6-decynyl, 7-decynyl, 8-decynyl, 9-decynyl, and the like, which may bear one or more substituents. Alkynyl group substituents include, but are not limited to, any of the substituents described herein, that result in the formation of a stable moiety. If an alkynyl group is attached to a nitrogen atom, the triple bond cannot be alpha to the nitrogen atom. In some embodiments the alkynyl chain is a linear. In some embodiments the alkynyl chain is branched. In some embodiments the alkynyl chain is substituted. In some embodiment the alkynyl chain is unsubstituted. In some embodiments the alkynyl chain is linear and substituted or unsubstituted. In some embodiments the alkynyl chain is branched and substituted or unsubstituted.

As used herein and throughout the entire description, the term “alkynylene” refers to a diradical of an unsaturated straight or branched hydrocarbon having at least one carbon-carbon triple bond. Generally, the maximal number of carbon-carbon triple bonds in the alkynylene group can be equal to the integer which is calculated by dividing the number of carbon atoms in the alkynylene group by 2 and, if the number of carbon atoms in the alkynylene group is uneven, rounding the result of the division down to the next integer. For example, for an alkynylene group having 9 carbon atoms, the maximum number of carbon-carbon triple bonds is 4. Preferably, the alkynylene group has 1 to 4, i.e., 1, 2, 3, or 4, more preferably 1 or 2 carbon-carbon triple bonds. Preferably, the alkynylene group comprises from 2 to 10 carbon atoms, i.e., 2, 3, 4, 5, 6, 7, 8, 9, or 10 carbon atoms, more preferably 2 to 8 carbon atoms, such as 2 to 6 carbon atoms or 2 to 4 carbon atoms. Thus, in a preferred embodiment, the alkynylene group comprises from 2 to 10 carbon atoms and 1, 2, 3, 4, or 5 (preferably 1, 2, or 3) carbon-carbon triple bonds, more preferably it comprises 2 to 8 carbon atoms and 1, 2, 3, or 4 (preferably 1 or 2) carbon-carbon triple bonds, such as 2 to 6 carbon atoms and 1, 2 or 3 carbon-carbon triple bonds or 2 to 4 carbon atoms and 1 or 2 carbon-carbon triple bonds. Exemplary alkynylene groups include ethyn-1,2-diyl, 1-propyn-1,3-diyl, 1-propyn-3,3-diyl, 1-butyn-1,3-diyl, 1-butyn-1,4-diyl, 1-butyn-3,4-diyl, 2-butyn-1,4-diyl and the like. If an alkynylene group is attached to a nitrogen atom, the triple bond cannot be alpha to the nitrogen atom. Alkynylene groups may be cyclic or acyclic, branched or unbranched, substituted or unsubstituted. Alkynylene group substituents include, but are not limited to, any of the substituents described herein, that result in the formation of a stable moiety.

As used herein and throughout the entire description, the term “cycloalkyl” or “cycloaliphatic” or “carbocyclic” or “carbocycle” represents cyclic non-aromatic versions of “alkyl” and “alkenyl” with preferably 3 to 14 carbon atoms, such as 3 to 10 carbon atoms, i.e., 3, 4, 5, 6, 7, 8, 9, or 10 carbon atoms, more preferably 3 to 8 carbon atoms, even more preferably 3 to 7 carbon atoms. In certain embodiments, the cycloalkyl group employed in the invention contains 3-14 carbon atoms (C₃₋₁₄ cycloalkyl). In certain embodiments, the cycloalkyl group employed in the invention contains 3-12 carbon atoms (C₃₋₁₂ cycloalkyl). In another embodiment, the cycloalkyl group employed in the invention contains 3-10 carbon atoms (C₃₋₁₀ cycloalkyl). In another embodiment, the cycloalkyl group employed in the invention contains 3-8 carbon atoms (C₃₋₈ cycloalkyl). In another embodiment, the cycloalkyl group employed in the invention contains 3-7 carbon atoms (C₃₋₇ cycloalkyl). In another embodiment, the cycloalkyl group employed in the invention contains 3-6 carbon atoms (C₃₋₆ cycloalkyl). In another embodiment, the cycloalkyl group employed in the invention contains 3-5 carbon atoms (C₃₋₅ cycloalkyl). In another embodiment, the cycloalkyl group employed in the invention contains 3-4 carbon atoms (C₃₋₄ cycloalkyl). In another embodiment, the cycloalkyl group employed in the invention contains 3 carbon atoms (C₃ cycloalkyl). Exemplary cycloalkyl groups include cyclopropyl, cyclopropenyl, cyclobutyl, cyclobutenyl, cyclopentyl, cyclopentenyl, cyclohexyl, cyclohexenyl, cycloheptyl, cycloheptenyl, cyclooctyl, cyclooctenyl, cyclononyl, cyclononenyl, cylcodecyl, cylcodecenyl, and adamantyl. The term “cycloalkyl” is also meant to include bicyclic and tricyclic versions thereof. If bicyclic rings are formed it is preferred that the respective rings are connected to each other at two adjacent carbon atoms, however, alternatively the two rings are connected via the same carbon atom, i.e., they form a spiro ring system or they form “bridged” ring systems. Preferred examples of cycloalkyl include C₃-C₈-cycloalkyl, in particular cyclopropyl, cyclobutyl, cyclopentyl, cyclohexyl, cycloheptyl, cyclooctyl, spiro[3,3]heptyl, spiro[3,4]octyl, spiro[4,3]octyl, bicyclo[4.1.0]heptyl, bicyclo[3.2.0]heptyl, bicyclo[2.2.1]heptyl, bicyclo[2.2.2]octyl, bicyclo[5.1.0]octyl, and bicyclo[4.2.0]octyl. Cycloalkyl group substituents include, but are not limited to, any of the substituents described herein, that result in the formation of a stable moiety.

As used herein and throughout the entire description, the term “cyclopropylene” means a cyclopropyl group as defined above in which one hydrogen atom has been removed resulting in a diradical. The cyclopropylene may link two atoms or moieties via the same carbon atom (1,1-cyclopropylene, i.e., a geminal diradical) or via two carbon atoms (1,2-cyclopropylene).

As used herein and throughout the entire description, the term “aryl” or “aromatic ring” as used herein, refers to an aromatic mono- or polycyclic ring system having 3-20 ring atoms, of which all the ring atoms are carbon, and which may be substituted or unsubstituted. In certain embodiments of the present invention, “aryl” refers to a mono, bi, or tricyclic C₄-C₂₀ aromatic ring system having one, two, or three aromatic rings which include, but are not limited to, phenyl, biphenyl, naphthyl, and the like, which may bear one or more substituents. Preferably, the aryl group contains 3 to 14 (e.g., 5 to 10, such as 5, 6, or 10) carbon atoms, more preferably 6 to 10 carbon atoms, which can be arranged in one ring (e.g., phenyl) or two or more condensed rings (e.g., naphthyl). Exemplary aryl groups include cyclopropenylium, cyclopentadienyl, phenyl, indenyl, naphthyl, azulenyl, fluorenyl, anthryl, and phenanthryl. Preferably, “aryl” refers to a monocyclic ring containing 6 carbon atoms or an aromatic bicyclic ring system containing 10 carbon atoms. Preferred examples are phenyl and naphthyl. In certain embodiments, the aryl group employed in the invention contains 3-20 carbon atoms (C₃₋₂₀ aryl). In certain embodiments, the aryl group employed in the invention contains 3-18 carbon atoms (C₃₋₁₈ aryl). In another embodiment, the aryl group employed in the invention contains 3-16 carbon atoms (C₃₋₁₆ aryl). In another embodiment, the aryl group employed in the invention contains 6-16 carbon atoms (C₆₋₁₆ aryl). In another embodiment, the aryl group employed in the invention contains 7-16 carbon atoms (C₇₋₁₆ aryl). In another embodiment, the aryl group employed in the invention contains 6-14 carbon atoms (C₆-14 aryl). In another embodiment, the aryl group employed in the invention contains 7-14 carbon atoms (C₇₋₁₄ aryl). In another embodiment, the aryl group employed in the invention contains 6-12 carbon atoms (C₆₋₁₂ aryl)). In another embodiment, the aryl group employed in the invention contains 7-12 carbon atoms (C₇₋₁₂ aryl). In another embodiment, the aryl group employed in the invention contains 6-11 carbon atoms (C₆₋₁₁ aryl). In another embodiment, the aryl group employed in the invention contains 7-11 carbon atoms (C₇₋₁₁ aryl). In another embodiment, the aryl group employed in the invention contains 6-10 carbon atoms (C₆₋₁₀ aryl). In another embodiment, the aryl group employed in the invention contains 7-10 carbon atoms (C₇₋₁₀ aryl). In another embodiment, the aryl group employed in the invention contains 6-8 carbon atoms (C₆₋₈ aryl). In another embodiment, the aryl group employed in the invention contains 6 carbon atoms (C₆ aryl). In another embodiment, the aryl group employed in the invention contains 10 carbon atoms (C₁₀ aryl). In some embodiments Z is not a substituted monocyclic six-membered aryl or unsubstituted monocyclic six-membered aryl. In some embodiments Z is not a substituted phenyl or unsubstituted phenyl. In some embodiments Z is not a phenyl substituted with 1, 2, 3, 4 or 5 substituents selected from the group consisting of —NO₂, —N₃, halogen, —NH₂, hydroxyl, —OR¹¹ and —C(═O)R¹¹, wherein R¹¹ is hydrogen, substituted alkyl or substituted alkynyl. Aryl substituents include, but are not limited to, any of the substituents described herein, that result in the formation of a stable moiety.

As used herein and throughout the entire description, the term “arylene,” as used herein refers to an aryl biradical derived from an aryl group, as defined herein, by removal of two hydrogen atoms. Arylene groups may be substituted or unsubstituted. Arylene group substituents include, but are not limited to, any of the substituents described herein, that result in the formation of a stable moiety. Additionally, arylene groups may be incorporated as a linker group into an alkylene, alkenylene, alkynylene, heteroalkylene, heteroalkenylene, or heteroalkynylene group, as defined herein.

As used herein and throughout the entire description, the term “heteroaryl” or “heteroaromatic ring” means an aryl group as defined above in which one or more carbon atoms in the aryl group are replaced by heteroatoms of O, S, or N. Preferably, the heteroaryl group contains 3 to 14 carbon atoms. Preferably, heteroaryl refers to a five or six-membered aromatic monocyclic ring wherein 1, 2, or 3 carbon atoms are replaced by the same or different heteroatoms of O, N, or S. Alternatively, it means an aromatic bicyclic or tricyclic ring system wherein 1, 2, 3, 4, or 5 carbon atoms are replaced with the same or different heteroatoms of O, N, or S. Preferably, in each ring of the heteroaryl group the maximum number of O atoms is 1, the maximum number of S atoms is 1, and the maximum total number of O and S atoms is 2. In certain embodiments, the heteroaryl group employed in the invention is a five membered aromatic monocyclic ring wherein 1, 2, or 3 carbon atoms are replaced by the same or different heteroatoms of O, N, or S. In certain embodiments, the heteroaryl group employed in the invention is a five membered aromatic monocyclic ring wherein 1, 2, or 3 carbon atoms are replaced by the same or different heteroatoms of O. In certain embodiments, the heteroaryl group employed in the invention is a five membered aromatic monocyclic ring wherein 1, 2, or 3 carbon atoms are replaced by the same or different heteroatoms of O and N. In certain embodiments, the heteroaryl group employed in the invention is a five membered aromatic monocyclic ring wherein 1, 2, or 3 carbon atoms are replaced by the same or different heteroatoms of O and S. In certain embodiments, the heteroaryl group employed in the invention is a five membered aromatic monocyclic ring wherein 1, 2, or 3 carbon atoms are replaced by the same or different heteroatoms of N and S. In certain embodiments, the heteroaryl group employed in the invention is a six membered aromatic monocyclic ring wherein 1, 2, or 3 carbon atoms are replaced by the same or different heteroatoms of O, S or N. In certain embodiments, the heteroaryl group employed in the invention is a six membered aromatic monocyclic ring wherein 1, 2, or 3 carbon atoms are replaced by N. In certain embodiments, the heteroaryl group employed in the invention is an aromatic bicyclic system wherein 1, 2, 3, 4, or 5 carbon atoms are replaced with the same or different heteroatoms of O, N, or S. In certain embodiments, the heteroaryl group employed in the invention is an aromatic bicyclic system wherein 1 carbon atom is replaced with O. In certain embodiments, the heteroaryl group employed in the invention is an aromatic bicyclic system wherein 1 carbon atom is replaced with N. In some embodiments, the heteroaryl group is substituted or unsubstituted indolyl. In certain embodiments, the heteroaryl group employed in the invention is an aromatic bicyclic system wherein 2 carbon atoms are replaced with N. In some embodiments, the heteroaryl group is substituted or unsubstituted 7-azaindolyl. In some embodiments, the heteroaryl group is substituted or unsubstituted 6-azaindolyl. In some embodiments, the heteroaryl group is substituted or unsubstituted 5-azaindolyl. In some embodiments, the heteroaryl group is substituted or unsubstituted 4-azaindolyl. In some embodiments, the heteroaryl group is substituted or unsubstituted imidazolyl. In certain embodiments, the heteroaryl group employed in the invention is an aromatic bicyclic system wherein 3 carbon atoms are replaced with N, preferably a substituted or unsubstituted diazaindolyl group. Exemplary heteroaryl groups include furanyl, thienyl, oxazolyl, isoxazolyl, oxadiazolyl (1,2,5- and 1,2,3-), pyrrolyl, imidazolyl, pyrazolyl, triazolyl (1,2,3- and 1,2,4-), tetrazolyl, thiazolyl, isothiazolyl, thiadiazolyl (1,2,3- and 1,2,5-), pyridyl, pyrimidinyl, pyrazinyl, triazinyl (1,2,3-, 1,2,4-, and 1,3,5-), benzofuranyl (1- and 2-), indolyl, azaindolyl (4-, 5-6- and 7-), diazaindolyl, isoindolyl, benzothienyl (1- and 2-), 1H-indazolyl, benzimidazolyl, benzoxazolyl, indoxazinyl, benzisoxazolyl, benzothiazolyl, benzisothiazolyl, benzotriazolyl, quinolinyl, isoquinolinyl, benzodiazinyl, quinoxalinyl, quinazolinyl, benzotriazinyl (1,2,3- and 1,2,4-benzotriazinyl), pyridazinyl, phenoxazinyl, thiazolopyridinyl, pyrrolothiazolyl, phenothiazinyl, isobenzofuranyl, chromenyl, xanthenyl, phenoxathiinyl, pyrrolizinyl, indolizinyl, indazolyl, purinyl, quinolizinyl, phthalazinyl, naphthyridinyl (1,5-, 1,6-, 1,7-, 1,8-, and 2,6-), cinnolinyl, pteridinyl, carbazolyl, phenanthridinyl, acridinyl, perimidinyl, phenanthrolinyl (1,7-, 1,8-, 1,10-, 3,8-, and 4,7-), phenazinyl, oxazolopyridinyl, isoxazolopyridinyl, pyrrolooxazolyl, pyrrolopyrrolyl, and the like, which may bear one or more substituents. Heteroaryl substituents include, but are not limited to, any of the substituents described herein, that result in the formation of a stable moiety. Exemplary 5- or 6-membered heteroaryl groups include furanyl, thienyl, oxazolyl, isoxazolyl, oxadiazolyl (1,2,5- and 1,2,3-), pyrrolyl, imidazolyl, pyrazolyl, triazolyl (1,2,3- and 1,2,4-), thiazolyl, isothiazolyl, thiadiazolyl (1,2,3- and 1,2,5-), pyridyl, pyrimidinyl, pyrazinyl, triazinyl (1,2,3-, 1,2,4-, and 1,3,5-), and pyridazinyl. Exemplary bicyclic heteroaryl groups 7-azaindolyl, 6-azaindolyl, 5-azaindolyl, 4-azaindolyl, diazaindolyl and indolyl.

As used herein and throughout the entire description, the term “diazaindolyl” or “diazaindole” refers to a compound having an indole core structure, wherein 2 carbon atoms of the annulated phenyl ring are replaced by N. Preferably the carbon atoms 4, 5, 6 and/or 7 of the indole core are replaced by N. Preferably the carbon atoms 4 and 5 of the indole core are replaced by N. Preferably the carbon atoms 4 and 6 of the indole core are replaced by N. Preferably the carbon atoms 4 and 7 of the indole core are replaced by N. Preferably the carbon atoms 5 and 6 of the indole core are replaced by N. Preferably the carbon atoms 6 and 7 of the indole core are replaced by N. Preferably the carbon atoms 5 and 7 of the indole core are replaced by N. In some embodiments the diazaindolyl is substituted. In some embodiments the diazaindolyl is unsubstituted.

As used herein and throughout the entire description, the term “heteroarylene,” as used herein, refers to a biradical derived from a heteroaryl group, as defined herein, by removal of two hydrogen atoms. Heteroarylene groups may be substituted or unsubstituted. Additionally, heteroarylene groups may be incorporated as a linker group into an alkylene, alkenylene, alkynylene, heteroalkylene, heteroalkenylene, or heteroalkynylene group, as defined herein. Heteroarylene group substituents include, but are not limited to, any of the substituents described herein, that result in the formation of a stable moiety.

As used herein and throughout the entire description, the terms “arylalkyl” and “heteroarylalkyl” are meant to include those radicals in which an aryl group and heteroaryl group, respectively, is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like). Preferably the Arylalkyl is a substituted or unsubstituted (C₆-C₁₄)aryl(C₁-C₆)alkyl Preferably the Arylalkyl is a substituted or unsubstituted (C₆-C₁₀)aryl(C₁-C₆)alkyl. Preferably the Heteroarylalkyl is a substituted or unsubstituted (C₃-C₁₄)heteroaryl(C₁-C₆)alkyl. Preferably the Heteroarylalkyl is a substituted or unsubstituted (C₃-C₁₀)heteroaryl(C₁-C₆)alkyl. In some embodiments the alkyl chain is a linear. In some embodiments the alkyl chain is branched. In some embodiments the alkyl chain is substituted. In some embodiments the alkyl chain is unsubstituted. In some embodiments the alkyl chain is linear and substituted or unsubstituted. In some embodiments the alkyl chain is branched and substituted or unsubstituted.

As used herein and throughout the entire description, the term “heterocyclyl” or “heterocyclic ring” or “heterocycle refers to a cyclic heteroaliphatic group. A heterocyclic group refers to a non-aromatic, partially unsaturated or fully saturated, 3- to 10-membered ring system, which includes single rings of 3 to 8 atoms in size, and bi- and tri-cyclic ring systems which may include aromatic five- or six-membered aryl or heteroaryl groups fused to a non-aromatic ring. The heterocyclic group may be substituted or unsubstituted. These heterocyclic rings include those having from one to three heteroatoms independently selected from oxygen, sulfur, and nitrogen, in which the nitrogen and sulfur heteroatoms may optionally be oxidized and the nitrogen heteroatom may optionally be quaternized. In certain embodiments, the term heterocyclic refers to a non-aromatic 5-, 6-, or 7-membered ring or polycyclic group wherein at least one ring atom is a heteroatom selected from O, S, and N (wherein the nitrogen and sulfur heteroatoms may be optionally oxidized), and the remaining ring atoms are carbon, the radical being joined to the rest of the molecule via any of the ring atoms. Heterocycyl groups include, but are not limited to, a bi- or tri-cyclic group, comprising fused five, six, or seven-membered rings having between one and three heteroatoms independently selected from the oxygen, sulfur, and nitrogen, wherein (i) each 5-membered ring has 0 to 2 double bonds, each 6-membered ring has 0 to 2 double bonds, and each 7-membered ring has 0 to 3 double bonds, (ii) the nitrogen and sulfur heteroatoms may be optionally oxidized, (iii) the nitrogen heteroatom may optionally be quaternized, and (iv) any of the above heterocyclic rings may be fused to an aryl or heteroaryl ring. Preferably, in each ring of the heterocyclyl group the maximum number of O atoms is 1, the maximum number of S atoms is 1, and the maximum total number of O and S atoms is 2. The term “heterocyclyl” is also meant to encompass partially or completely hydrogenated forms (such as dihydro, tetrahydro or perhydro forms) of the above-mentioned heteroaryl groups. Exemplary heterocyclyl groups include morpholino, isochromanyl, chromanyl, pyrrolidinyl, imidazolidinyl, pyrazolidinyl, piperidinyl, piperazinyl, indolinyl, isoindolinyl, di- and tetrahydrofuranyl, di- and tetrahydrothienyl, di- and tetrahydrooxazolyl, di- and tetrahydroisoxazolyl, di- and tetrahydrooxadiazolyl (1,2,5- and 1,2,3-), dihydropyrrolyl, dihydroimidazolyl, dihydropyrazolyl, di- and tetrahydrotriazolyl (1,2,3- and 1,2,4-), di- and tetrahydrothiazolyl, di- and tetrahydrothiazolyl, di- and tetrahydrothiadiazolyl (1,2,3- and 1,2,5-), di- and tetrahydropyridyl, di- and tetrahydropyrimidinyl, di- and tetrahydropyrazinyl, di- and tetrahydrotriazinyl (1,2,3-, 1,2,4-, and 1,3,5-), di- and tetrahydrobenzofuranyl (1- and 2-), di- and tetrahydroindolyl, di- and tetrahydroisoindolyl, di- and tetrahydrobenzothienyl (1- and 2), di- and tetrahydro-1H-indazolyl, di- and tetrahydrobenzimidazolyl, di- and tetrahydrobenzoxazolyl, di- and tetrahydroindoxazinyl, di- and tetrahydrobenzisoxazolyl, di- and tetrahydrobenzothiazolyl, di- and tetrahydrobenzisothiazolyl, di- and tetrahydrobenzotriazolyl, di- and tetrahydroquinolinyl, di- and tetrahydroisoquinolinyl, di- and tetrahydrobenzodiazinyl, di- and tetrahydroquinoxalinyl, di- and tetrahydroquinazolinyl, di- and tetrahydrobenzotriazinyl (1,2,3- and 1,2,4-), di- and tetrahydropyridazinyl, di- and tetrahydrophenoxazinyl, di- and tetrahydrothiazolopyridinyl (such as 4,5,6-7-tetrahydro[1,3]thiazolo[5,4-c]pyridinyl or 4,5,6-7-tetrahydro[1,3]thiazolo[4,5-c]pyridinyl, e.g., 4,5,6-7-tetrahydro[1,3]thiazolo[5,4-c]pyridin-2-yl or 4,5,6-7-tetrahydro[1,3]thiazolo[4,5-c]pyridin-2-yl), di- and tetrahydropyrrolothiazolyl (such as 5,6-dihydro-4H-pyrrolo[3,4-d][1,3]thiazolyl), di- and tetrahydrophenothiazinyl, di- and tetrahydroisobenzofuranyl, di- and tetrahydrochromenyl, di- and tetrahydroxanthenyl, di- and tetrahydrophenoxathiinyl, di- and tetrahydropyrrolizinyl, di- and tetrahydroindolizinyl, di- and tetrahydroindazolyl, di- and tetrahydropurinyl, di- and tetrahydroquinolizinyl, di- and tetrahydrophthalazinyl, di- and tetrahydronaphthyridinyl (1,5-, 1,6-, 1,7-, 1,8-, and 2,6-), di- and tetrahydrocinnolinyl, di- and tetrahydropteridinyl, di- and tetrahydrocarbazolyl, di- and tetrahydrophenanthridinyl, di- and tetrahydroacridinyl, di- and tetrahydroperimidinyl, di- and tetrahydrophenanthrolinyl (1,7-, 1,8-, 1,10-, 3,8-, and 4,7-), di- and tetrahydrophenazinyl, di- and tetrahydrooxazolopyridinyl, di- and tetrahydroisoxazolopyridinyl, di- and tetrahydropyrrolooxazolyl, and di- and tetrahydropyrrolopyrrolyl. Exemplary 5- or 6-membered heterocyclyl groups include morpholino, pyrrolidinyl, imidazolidinyl, pyrazolidinyl, piperidinyl, piperazinyl, di- and tetrahydrofuranyl, di- and tetrahydrothienyl, di- and tetrahydrooxazolyl, di- and tetrahydroisoxazolyl, di- and tetrahydrooxadiazolyl (1,2,5- and 1,2,3-), dihydropyrrolyl, dihydroimidazolyl, dihydropyrazolyl, di- and tetrahydrotriazolyl (1,2,3- and 1,2,4-), di- and tetrahydrothiazolyl, di- and tetrahydroisothiazolyl, di- and tetrahydrothiadiazolyl (1,2,3- and 1,2,5-), di- and tetrahydropyridyl, di- and tetrahydropyrimidinyl, di- and tetrahydropyrazinyl, di- and tetrahydrotriazinyl (1,2,3-, 1,2,4-, and 1,3,5-), di- and tetrahydropyridazinyl and the like, which may bear one or more substituents. Preferably 2H-1-benzopyranyl (2H-chromenyl), benzodihydropyranyl (chromanyl), 4H-1-benzopyranyl (4H-chromenyl), 1H-2-benzopyranyl (1H-isochromenyl), isochromanyl, 3H-2-benzopyranyl (3H-isochromenyl), 1-benzopyran-4-on-yl (chromonyl), 4-chromanonyl, 1-benzopyran-2-on-yl (coumarinyl), dihydrocoumarinyl, 3-isochromanonyl, 2-coumaranon-yl. In some embodiments, the heterocyclyl group is substituted or unsubstituted 2H-1-benzopyranyl (2H-chromenyl). In some embodiments, the heterocyclyl group is substituted or unsubstituted benzodihydropyranyl (chromanyl). In some embodiments, the heterocyclyl group is substituted or unsubstituted 4H-1-benzopyranyl (4H-chromenyl). In some embodiments, the heterocyclyl group is substituted or unsubstituted 1H-2-benzopyranyl (1H-isochromenyl). In some embodiments, the heterocyclyl group is substituted or unsubstituted isochromanyl. In some embodiments, the heterocyclyl group is substituted or unsubstituted 3H-2-benzopyranyl (3H-isochromenyl). In some embodiments, the heterocyclyl group is substituted or unsubstituted 1-benzopyran-4-on-yl (chromonyl). In some embodiments, the heterocyclyl group is substituted or unsubstituted 4-chromanonyl. In some embodiments, the heterocyclyl group is substituted or unsubstituted 1-benzopyran-2-on-yl (coumarinyl). In some embodiments, the heterocyclyl group is substituted or unsubstituted dihydrocoumarinyl. In some embodiments, the heterocyclyl group is substituted or unsubstituted 3-isochromanonyl. In some embodiments, the heterocyclyl group is substituted or unsubstituted 2-coumaranon-yl. In some embodiments, the heterocyclyl group is a substituted or unsubstituted (C₃-C₁₄)heterocyclyl group, wherein 1, 2, 3, 4, or 5 carbon atoms are replaced with the same or different heteroatoms of O, N, or S. In some embodiments, the heterocyclyl group is a substituted or unsubstituted (C₃-C₁₄)heterocyclyl group, wherein 1, 2, 3, 4, or 5 carbon atoms are replaced with O. In some embodiments, the heterocyclyl group is a substituted or unsubstituted (C₃-C₁₄)heterocyclyl group, wherein 1, 2, 3, 4, or 5 carbon atoms are replaced with N. In some embodiments, the heterocyclyl group is a substituted or unsubstituted (C₃-C₁₄)heterocyclyl group, wherein 1, 2, 3, 4, or 5 carbon atoms are replaced with S. In some embodiments, the heterocyclyl group is a substituted or unsubstituted (C₉-C₁₀)heterocyclyl group, wherein 1, 2, 3, 4, or 5 carbon atoms are replaced with the same or different heteroatoms of O, N, or S. In some embodiments, the heterocyclyl group is a substituted or unsubstituted (C₉-C₁₀)heterocyclyl group, wherein 1, 2, 3, 4, or 5 carbon atoms are replaced with O. In some embodiments, the heterocyclyl group is a substituted or unsubstituted (C₉-C₁₀)heterocyclyl group, wherein 1, 2, 3, 4, or 5 carbon atoms are replaced with N. In some embodiments, the heterocyclyl group is a substituted or unsubstituted (C₉-C₁₀)heterocyclyl group, wherein 1, 2, 3, 4, or 5 carbon atoms are replaced with S. In some embodiments, the heterocyclyl group is a substituted or unsubstituted (C₁₀)heterocyclyl group, wherein 1, 2, 3, 4, or 5 carbon atoms are replaced with the same or different heteroatoms of O, N, or S. In some embodiments, the heterocyclyl group is a substituted or unsubstituted (C₁₀)heterocyclyl group, wherein 1, 2, 3, 4, or 5 carbon atoms are replaced with O. In some embodiments, the heterocyclyl group is a substituted or unsubstituted (C₁₀)heterocyclyl group, wherein 1, 2, 3, 4, or 5 carbon atoms are replaced with N. In some embodiments, the heterocyclyl group is a substituted or unsubstituted (C₁₀)heterocyclyl group, wherein 1, 2, 3, 4, or 5 carbon atoms are replaced with S.

As used herein and throughout the entire description, the term “heteroalkyl,” as used herein, refers to an alkyl moiety, as defined herein, which contain one or more heteroatoms (e.g., oxygen, sulfur, nitrogen, phosphorus, or silicon atoms) in between carbon atoms. The heteroalkyl may be substituted or unsubstituted. In certain embodiments, the heteroalkyl group contains 1-20 carbon atoms and 1-6 heteroatoms (C₁₋₂₀ heteroalkyl). In certain embodiments, the heteroalkyl group contains 1-10 carbon atoms and 1-4 heteroatoms (C₁₋₁₀ heteroalkyl). In certain embodiments, the heteroalkyl group contains 1-6 carbon atoms and 1-3 heteroatoms (C₁₋₆ heteroalkyl). In certain embodiments, the heteroalkyl group contains 1-5 carbon atoms and 1-3 heteroatoms (C₁₋₅ heteroalkyl). In certain embodiments, the heteroalkyl group contains 1-4 carbon atoms and 1-2 heteroatoms (C₁₋₄ heteroalkyl). In certain embodiments, the heteroalkyl group contains 1-3 carbon atoms and 1 heteroatom (C₁₋₃ heteroalkyl). In certain embodiments, the heteroalkyl group contains 1-2 carbon atoms and 1 heteroatom (C₁₋₂ heteroalkyl). The term “heteroalkylene,” as used herein, refers to a biradical derived from an heteroalkyl group, as defined herein, by removal of two hydrogen atoms. Heteroalkylene groups may be cyclic or acyclic, branched or unbranched, substituted or unsubstituted. In certain embodiments the heteroalkyl group is a substituted heteroalkyl group containing 1-6 carbon atoms and 1-3 heteroatoms (C₁₋₆ heteroalkyl). In certain embodiments the heteroalkyl group is an unsubstituted heteroalkyl group containing 1-6 carbon atoms and 1-3 heteroatoms (C₁₋₆ heteroalkyl). In some embodiments the heteroalkyl is an alkyl moiety wherein on methylene group is replaced by S. In some embodiments the heteroalkyl is an alkyl moiety wherein on methylene group is replaced by O. In some embodiments the heteroalkyl is an alkyl moiety wherein on methylene group is replaced by NR¹, wherein is selected from the group consisting of hydrogen, substituted or unsubstituted (C₁-C₆)alkyl, substituted or unsubstituted (C₂-C₆)alkenyl, substituted or unsubstituted (C₂-C₆)alkynyl, substituted or unsubstituted (C₃-C₈)cycloalkyl, substituted or unsubstituted (C₆-C₁₄)aryl and substituted or unsubstituted (C₃-C₁₄)heteroaryl. In some embodiments heteroalkyl is —CH₂SCH₃. In some embodiments heteroalkyl is —CH₂OCH₃.

As used herein and throughout the entire description, the Heteroalkylene group substituents include, but are not limited to, any of the substituents described herein, that result in the formation of a stable moiety.

As used herein and throughout the entire description, the term “heteroalkenyl,” as used herein, refers to an alkenyl moiety, as defined herein, which further contains one or more heteroatoms (e.g., oxygen, sulfur, nitrogen, phosphorus, or silicon atoms) in between carbon atoms. In certain embodiments, the heteroalkenyl group contains 2-20 carbon atoms and 1-6 heteroatoms (C₂₋₂₀ heteroalkenyl). In certain embodiments, the heteroalkenyl group contains 2-10 carbon atoms and 1-4 heteroatoms (C₂₋₁₀ heteroalkenyl). In certain embodiments, the heteroalkenyl group contains 2-6 carbon atoms and 1-3 heteroatoms (C₂₋₆ heteroalkenyl). In certain embodiments, the heteroalkenyl group contains 2-5 carbon atoms and 1-3 heteroatoms (C₂₋₅ heteroalkenyl). In certain embodiments, the heteroalkenyl group contains 2-4 carbon atoms and 1-2 heteroatoms (C₂₋₄ heteroalkenyl). In certain embodiments, the heteroalkenyl group contains 2-3 carbon atoms and 1 heteroatom (C₂₋₃ heteroalkenyl). The term “heteroalkenylene,” as used herein, refers to a biradical derived from an heteroalkenyl group, as defined herein, by removal of two hydrogen atoms. Heteroalkenylene groups may be cyclic or acyclic, branched or unbranched, substituted or unsubstituted. In certain embodiments the heteroalkenyl group is a substituted heteroalkenyl group containing 1-6 carbon atoms and 1-3 heteroatoms (C₁₋₆ heteroalkenyl). In certain embodiments the heteroalkenyl group is an unsubstituted heteroalkenyl group containing 1-6 carbon atoms and 1-3 heteroatoms (C₁₋₆ heteroalkenyl).

As used herein and throughout the entire description, the term “heteroalkynyl,” as used herein, refers to an alkynyl moiety, as defined herein, which further contains one or more heteroatoms (e.g., oxygen, sulfur, nitrogen, phosphorus, or silicon atoms) in between carbon atoms. In certain embodiments, the heteroalkynyl group contains 2-20 carbon atoms and 1-6 heteroatoms (C₂₋₂₀ heteroalkynyl). In certain embodiments, the heteroalkynyl group contains 2-10 carbon atoms and 1-4 heteroatoms (C₂₋₁₀ heteroalkynyl). In certain embodiments, the heteroalkynyl group contains 2-6 carbon atoms and 1-3 heteroatoms (C₂₋₆ heteroalkynyl). In certain embodiments, the heteroalkynyl group contains 2-5 carbon atoms and 1-3 heteroatoms (C₂₋₅ heteroalkynyl). In certain embodiments, the heteroalkynyl group contains 2-4 carbon atoms and 1-2 heteroatoms (C₂₋₄ heteroalkynyl). In certain embodiments, the heteroalkynyl group contains 2-3 carbon atoms and 1 heteroatom (C₂₋₃ heteroalkynyl). The term “heteroalkynylene,” as used herein, refers to a biradical derived from an heteroalkynyl group, as defined herein, by removal of two hydrogen atoms. Heteroalkynylene groups may be cyclic or acyclic, branched or unbranched, substituted or unsubstituted. In certain embodiments the heteroalkynyl group is a substituted heteroalkynyl group containing 1-6 carbon atoms and 1-3 heteroatoms (C₁₋₆ heteroalkynyl). In certain embodiments the heteroalkynyl group is an unsubstituted heteroalkynyl group containing 1-6 carbon atoms and 1-3 heteroatoms (C₁₋₆ heteroalkynyl).

An unnatural amino acid as described herein may also be described by having formula I:

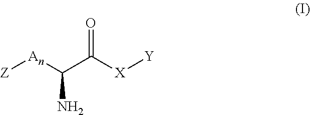

In some embodiments the compound having a formula according to Formula I may be characterized in that, X is O, NR¹ or S; Y is hydrogen or substituted or unsubstituted (C₁-C₆)alkyl; Z is selected from the group consisting of substituted or unsubstituted (C₁-C₆)alkyl, substituted or unsubstituted (C₂-C₆)alkenyl, substituted or unsubstituted (C₂-C₆)alkynyl, substituted or unsubstituted (C₃-C₈)cycloalkyl, substituted or unsubstituted (C₆-C₁₄)aryl, substituted or unsubstituted (C₆-C₁₄)aryl(C₁-C₆)alkyl, substituted or unsubstituted (C₃-C₁₄)heteroaryl, substituted or unsubstituted (C₃-C₁₄)heteroaryl(C₁-C₆)alkyl, substituted or unsubstituted (C₃-C₁₄)heterocyclyl, substituted or unsubstituted (C₁-C₆)heteroalkyl, substituted or unsubstituted (C₂-C₆)heteroalkenyl and substituted or unsubstituted (C₂-C₆)heteroalkynyl; R¹ is hydrogen or substituted or unsubstituted (C₁-C₆)alkyl, wherein A is a methylene group and n is 0 or 1, preferably n is 1.

In some embodiments the compound having a formula according to Formula I may be characterized in that, X is O, NR¹ or S; Y is hydrogen or substituted or unsubstituted (C₁-C₆)alkyl; Z is selected from the group consisting of substituted or unsubstituted (C₁-C₆)alkyl, substituted or unsubstituted (C₆-C₁₄)aryl, substituted or unsubstituted (C₃-C₁₄)heteroaryl, substituted or unsubstituted (C₃-C₁₄)Heterocyclyl and substituted or unsubstituted (C₁-C₆)heteroalkyl; R¹ is hydrogen or substituted or unsubstituted (C₁-C₆)alkyl, wherein A is a methylene group and n is 0 or 1, preferably n is 1.

In some embodiments the compound having a formula according to Formula I may be characterized in that, X is O; Y is hydrogen or substituted or unsubstituted (C₁-C₆)alkyl; Z is selected from the group consisting of substituted or unsubstituted (C₁-C₆)alkyl, substituted or unsubstituted (C₆-C₁₄)aryl, substituted or unsubstituted (C₃-C₁₄)heteroaryl, substituted or unsubstituted (C₃-C₁₄)heterocyclyl and substituted or unsubstituted (C₁-C₆)heteroalkyl, wherein A is a methylene group and n is 0 or 1, preferably n is 1.

In some embodiments the compound having a formula according to Formula I may be characterized in that, X is O, NR¹ or S; Y is hydrogen or substituted or unsubstituted (C₁-C₆)alkyl; Z is selected from the group consisting of substituted or substituted or unsubstituted (C₆-C₁₄)aryl, unsubstituted (C₃-C₁₄)heteroaryl and substituted or unsubstituted (C₃-C₁₄)heterocyclyl; R¹ is hydrogen or substituted or unsubstituted (C₁-C₆)alkyl, wherein A is a methylene group and n is 0 or 1, preferably n is 1.

In some embodiments the compound having a formula according to Formula I may be characterized in that, X is O; Y is hydrogen or substituted or unsubstituted (C₁-C₆)alkyl; Z is selected from the group consisting of substituted or unsubstituted (C₆-C₁₄)aryl, substituted or unsubstituted (C₃-C₁₄)heteroaryl and substituted or unsubstituted (C₃-C₁₄)heterocyclyl, wherein A is a methylene group and n is 0 or 1, preferably n is 1.

In some embodiments the compound having a formula according to Formula I may be characterized in that, X is O, NR¹ or S; Y is hydrogen or substituted or unsubstituted (C₁-C₆)alkyl; Z is selected from the group consisting of substituted or unsubstituted 2H-1-benzopyranyl (2H-chromenyl), substituted or unsubstituted benzodihydropyranyl (chromanyl), substituted or unsubstituted 4H-1-benzopyranyl (4H-chromenyl), substituted or unsubstituted 1H-2-benzopyranyl (1H-isochromenyl), substituted or unsubstituted isochromanyl, substituted or unsubstituted 3H-2-benzopyranyl (3H-isochromenyl), substituted or unsubstituted 1-benzopyran-4-on-yl (chromonyl), substituted or unsubstituted 4-chromanonyl, substituted or unsubstituted 1-benzopyran-2-on-yl (coumarinyl), substituted or unsubstituted dihydrocoumarinyl, substituted or unsubstituted 3-isochromanonyl, substituted or unsubstituted 2-coumaranon-yl, substituted or unsubstituted (C₁-C₆)alkyl, substituted or unsubstituted (C₁-C₆)heteroalkyl, substituted or unsubstituted imidazolyl, substituted or unsubstituted azaindolyl including 7-azaindolyl, 6-azaindolyl, 5-azaindolyl and 4-azaindolyl, substituted or unsubstituted diazaindolyl and substituted or unsubstituted indolyl; R¹ is hydrogen or substituted or unsubstituted (C₁-C₆)alkyl, wherein A is a methylene group and n is 0 or 1, preferably n is 1.

In some embodiments the compound having a formula according to Formula I may be characterized in that, X is O; Y is hydrogen or substituted or unsubstituted (C₁-C₆)alkyl; Z is selected from the group consisting of substituted or unsubstituted 2H-1-benzopyranyl (2H-chromenyl), substituted or unsubstituted benzodihydropyranyl (chromanyl), substituted or unsubstituted 4H-1-benzopyranyl (4H-chromenyl), substituted or unsubstituted 1H-2-benzopyranyl (1H-isochromenyl), substituted or unsubstituted isochromanyl, substituted or unsubstituted 3H-2-benzopyranyl (3H-isochromenyl), substituted or unsubstituted 1-benzopyran-4-on-yl (chromonyl), substituted or unsubstituted 4-chromanonyl, substituted or unsubstituted 1-benzopyran-2-on-yl (coumarinyl), substituted or unsubstituted dihydrocoumarinyl, substituted or unsubstituted 3-isochromanonyl, substituted or unsubstituted 2-coumaranon-yl, substituted or unsubstituted (C₁-C₆)alkyl, substituted or unsubstituted (C₁-C₆)heteroalkyl, substituted or unsubstituted imidazolyl, substituted or unsubstituted azaindolyl including 7-azaindolyl, 6-azaindolyl, 5-azaindolyl and 4-azaindolyl, substituted or unsubstituted diazaindolyl and substituted or unsubstituted indolyl, wherein A is a methylene group and n is 0 or 1, preferably n is 1.

In some embodiments the compound having a formula according to Formula I may be characterized in that, X is O, NR¹ or S; Y is hydrogen or substituted or unsubstituted (C₁-C₆)alkyl; Z is selected from the group consisting of substituted or unsubstituted 1-benzopyran-2-on-yl (coumarinyl), substituted or unsubstituted dihydrocoumarinyl, substituted or unsubstituted imidazolyl, substituted or unsubstituted azaindolyl including 7-azaindolyl, 6-azaindolyl, 5-azaindolyl and 4-azaindolyl, and substituted or unsubstituted indolyl; R¹ is hydrogen or substituted or unsubstituted (C₁-C₆)alkyl, wherein A is a methylene group and n is 0 or 1, preferably n is 1.

In some embodiments the compound having a formula according to Formula I may be characterized in that, X is O; Y is hydrogen or substituted or unsubstituted (C₁-C₆)alkyl; Z is selected from the group consisting of substituted or unsubstituted 1-benzopyran-2-on-yl (coumarinyl), substituted or unsubstituted dihydrocoumarinyl, substituted or unsubstituted imidazolyl, substituted or unsubstituted azaindolyl including 7-azaindolyl, 6-azaindolyl, 5-azaindolyl and 4-azaindolyl, and substituted or unsubstituted indolyl, wherein A is a methylene group and n is 0 or 1, preferably n is 1.

In some embodiments of the compound having a formula according to Formula I X is O. In some embodiments of the compound having a formula according to Formula I X is S. In some embodiments of the compound having a formula according to Formula I X is NR¹, wherein R¹ is selected from the group consisting of hydrogen, substituted or unsubstituted (C₁-C₆)alkyl, substituted or unsubstituted (C₂-C₆)alkenyl, substituted or unsubstituted (C₂-C₆)alkynyl, substituted or unsubstituted (C₃-C₈)cycloalkyl, substituted or unsubstituted (C₆-C₁₄)aryl and substituted or unsubstituted (C₃-C₁₄)heteroaryl, preferably hydrogen or substituted or unsubstituted (C₁-C₆)alkyl, more preferably hydrogen, more preferably substituted or unsubstituted (C₁-C₆)alkyl, even more preferably unsubstituted (C₁-C₆)alkyl.

In some embodiments of the compound having a formula according to Formula I Y is selected from the group consisting of hydrogen, substituted or unsubstituted alkyl, substituted or unsubstituted alkenyl, substituted or unsubstituted alkynyl, substituted or unsubstituted cycloalkyl, substituted or unsubstituted aryl, substituted or unsubstituted arylalkyl, substituted or unsubstituted heteroaryl, substituted or unsubstituted heteroarylalkyl, substituted or unsubstituted heterocyclyl, substituted or unsubstituted heteroalkyl, substituted or unsubstituted heteroalkenyl and substituted or unsubstituted heteroalkynyl. In some embodiments of the compound having a formula according to Formula I Y is selected from the group consisting of hydrogen, substituted or unsubstituted alkyl, substituted or unsubstituted alkynyl, substituted or unsubstituted cycloalkyl, substituted or unsubstituted aryl, substituted or unsubstituted arylalkyl, substituted or unsubstituted heteroaryl, substituted or unsubstituted heteroarylalkyl, substituted or unsubstituted Heterocyclyl and substituted or unsubstituted heteroalkyl. In some embodiments of the compound having a formula according to Formula I Y is selected from the group consisting of substituted or unsubstituted (C₁-C₆)alkyl, substituted or unsubstituted (C₂-C₆)alkenyl, substituted or unsubstituted (C₂-C₆)alkynyl, substituted or unsubstituted (C₃-C₈)cycloalkyl, substituted or unsubstituted (C₆-C₁₄)aryl, substituted or unsubstituted (C₆-C₄)aryl(C₁-C₆)alkyl, substituted or unsubstituted (C₃-C₁₄)heteroaryl, substituted or unsubstituted (C₃-C₁₄)heteroaryl(C₁-C₆)alkyl, substituted or unsubstituted (C₃-C₁₄)heterocyclyl, substituted or unsubstituted (C₁-C₆)heteroalkyl, substituted or unsubstituted (C₂-C₆)heteroalkenyl and substituted or unsubstituted (C₂-C₆)heteroalkynyl. In some embodiments of the compound having a formula according to Formula I Y is hydrogen or substituted or unsubstituted (C₁-C₆)alkyl. In some embodiments of the compound having a formula according to Formula I Y is hydrogen. In some embodiments of the compound having a formula according to Formula I Y is substituted or unsubstituted alkyl, preferably substituted or unsubstituted (C₁-C₆)alkyl. In some embodiments of the compound having a formula according to Formula I Y is substituted or unsubstituted alkenyl, preferably substituted or unsubstituted (C₂-C₆)alkenyl. In some embodiments of the compound having a formula according to Formula I Y is substituted or unsubstituted alkynyl, preferably substituted or unsubstituted (C₂-C₆)alkynyl. In some embodiments of the compound having a formula according to Formula I Y is substituted or unsubstituted cycloalkyl, preferably substituted or unsubstituted (C₃-C₈)cycloalkyl. In some embodiments of the compound having a formula according to Formula I Y is substituted or unsubstituted aryl, preferably substituted or unsubstituted (C₆-C₁₄)aryl. In some embodiments of the compound having a formula according to Formula I Y is substituted or unsubstituted Arylalkyl, preferably substituted or unsubstituted (C₆-C₁₄)aryl(C₁-C₆)alkyl. In some embodiments of the compound having a formula according to Formula I Y is substituted or unsubstituted heteroaryl, preferably substituted or unsubstituted (C₃-C₁₄)heteroaryl. In some embodiments of the compound having a formula according to Formula I Y is substituted or unsubstituted heteroarylalkyl, preferably substituted or unsubstituted (C₃-C₁₄)heteroaryl(C₁-C₆)alkyl. In some embodiments of the compound having a formula according to Formula I Y is substituted or unsubstituted heterocyclyl, preferably substituted or unsubstituted (C₃-C₁₄)heterocyclyl. In some embodiments of the compound having a formula according to Formula I Y is substituted or unsubstituted heteroalkyl, preferably substituted or unsubstituted (C₁-C₆)heteroalkyl. In some embodiments of the compound having a formula according to Formula I Y is substituted or unsubstituted heteroalkenyl, preferably substituted or unsubstituted (C₂-C₆)heteroalkenyl. In some embodiments of the compound having a formula according to Formula I Y is substituted or unsubstituted heteroalkynyl, preferably substituted or unsubstituted (C₂-C₆)heteroalkynyl.

In some embodiments of the compound having a formula according to Formula I Z is selected from the group consisting of hydrogen, substituted or unsubstituted alkyl, substituted or unsubstituted alkenyl, substituted or unsubstituted alkynyl, substituted or unsubstituted cycloalkyl, substituted or unsubstituted aryl, substituted or unsubstituted arylalkyl, substituted or unsubstituted heteroaryl, substituted or unsubstituted heteroarylalkyl, substituted or unsubstituted heterocyclyl, substituted or unsubstituted heteroalkyl, substituted or unsubstituted heteroalkenyl and substituted or unsubstituted heteroalkynyl; and with the proviso that Z is not a substituted or unsubstituted monocyclic six-membered aryl. In some embodiments of the compound having a formula according to Formula I Z is selected from the group consisting of hydrogen, substituted or unsubstituted alkyl, substituted or unsubstituted alkynyl, substituted or unsubstituted cycloalkyl, substituted or unsubstituted aryl, substituted or unsubstituted arylalkyl, substituted or unsubstituted heteroaryl, substituted or unsubstituted heteroarylalkyl, substituted or unsubstituted Heterocyclyl and substituted or unsubstituted heteroalkyl; and with the proviso that Z is not a substituted or unsubstituted monocyclic six-membered aryl. In some embodiments of the compound having a formula according to Formula I Z is selected from the group consisting of substituted or unsubstituted (C₁-C₆)alkyl, substituted or unsubstituted (C₂-C₆)alkenyl, substituted or unsubstituted (C₂-C₆)alkynyl, substituted or unsubstituted (C₃-C₈)cycloalkyl, substituted or unsubstituted (C₆-C₁₄)aryl, substituted or unsubstituted (C₆-C₁₄)aryl(C₁-C₆)alkyl, substituted or unsubstituted (C₃-C₁₄)heteroaryl, substituted or unsubstituted (C₃-C₁₄)heteroaryl(C₁-C₆)alkyl, substituted or unsubstituted (C₃-C₁₄)heterocyclyl, substituted or unsubstituted (C₁-C₆)heteroalkyl, substituted or unsubstituted (C₂-C₆)heteroalkenyl and substituted or unsubstituted (C₂-C₆)heteroalkynyl; and with the proviso that Z is not a substituted or unsubstituted monocyclic six-membered aryl. In some embodiments of the compound having a formula according to Formula I Z is hydrogen or substituted or unsubstituted (C₁-C₆)alkyl. In some embodiments of the compound having a formula according to Formula I Z is hydrogen. In some embodiments of the compound having a formula according to Formula I Z is substituted or unsubstituted alkyl, preferably substituted or unsubstituted (C₁-C₆)alkyl. In some embodiments of the compound having a formula according to Formula I Z is substituted or unsubstituted alkenyl, preferably substituted or unsubstituted (C₂-C₆)alkenyl. In some embodiments of the compound having a formula according to Formula I Z is substituted or unsubstituted alkynyl, preferably substituted or unsubstituted (C₂-C₆)alkynyl. In some embodiments of the compound having a formula according to Formula I Z is substituted or unsubstituted cycloalkyl, preferably substituted or unsubstituted (C₃-C₈)cycloalkyl. In some embodiments of the compound having a formula according to Formula I Z is substituted or unsubstituted aryl, preferably substituted or unsubstituted (C₆-C₁₄)aryl preferably with the proviso that Z is not a substituted or unsubstituted monocyclic six-membered aryl. In some embodiments of the compound having a formula according to Formula I Z is substituted or unsubstituted Arylalkyl, preferably substituted or unsubstituted (C₆-C₁₄)aryl(C₁-C₆)alkyl, preferably with the proviso that Z is not a substituted or unsubstituted monocyclic six-membered aryl. In some embodiments of the compound having a formula according to Formula I Z is substituted or unsubstituted heteroaryl, preferably substituted or unsubstituted (C₃-C₁₄)heteroaryl. In some embodiments of the compound having a formula according to Formula I Z is substituted or unsubstituted heteroarylalkyl, preferably substituted or unsubstituted (C₃-C₁₄)heteroaryl(C₁-C₆)alkyl. In some embodiments of the compound having a formula according to Formula I Z is substituted or unsubstituted Heterocyclyl, preferably substituted or unsubstituted (C₃-C₁₄)heterocyclyl. In some embodiments of the compound having a formula according to Formula I Z is substituted or unsubstituted heteroalkyl, preferably substituted or unsubstituted (C₁-C₆)heteroalkyl. In some embodiments of the compound having a formula according to Formula I Z is substituted or unsubstituted heteroalkenyl, preferably substituted or unsubstituted (C₂-C₆)heteroalkenyl. In some embodiments of the compound having a formula according to Formula I Z is substituted or unsubstituted heteroalkynyl, preferably substituted or unsubstituted (C₂-C₆)heteroalkynyl. In some embodiments of the compound having a formula according to Formula I Z is selected from the group consisting of substituted or unsubstituted 2H-1-benzopyranyl (2H-chromenyl), substituted or unsubstituted benzodihydropyranyl (chromanyl), substituted or unsubstituted 4H-1-benzopyranyl (4H-chromenyl), substituted or unsubstituted 1H-2-benzopyranyl (1H-isochromenyl), substituted or unsubstituted isochromanyl, substituted or unsubstituted 3H-2-benzopyranyl (3H-isochromenyl), substituted or unsubstituted 1-benzopyran-4-on-yl (chromonyl), substituted or unsubstituted 4-chromanonyl, substituted or unsubstituted 1-benzopyran-2-on-yl (coumarinyl), substituted or unsubstituted dihydrocoumarinyl, substituted or unsubstituted 3-isochromanonyl, substituted or unsubstituted 2-coumaranon-yl, substituted or unsubstituted (C₁-C₆)alkyl, substituted or unsubstituted (C₁-C₆)heteroalkyl, substituted or unsubstituted imidazolyl, substituted or unsubstituted azaindolyl including 7-azaindolyl, 6-azaindolyl, 5-azaindolyl and 4-azaindolyl and substituted or unsubstituted indolyl. In some embodiments of the compound having a formula according to Formula I Z is substituted or unsubstituted 2H-1-benzopyranyl (2H-chromenyl). In some embodiments of the compound having a formula according to Formula I Z is substituted or unsubstituted benzodihydropyranyl (chromanyl). In some embodiments of the compound having a formula according to Formula I Z is substituted or unsubstituted 4H-1-benzopyranyl (4H-chromenyl). In some embodiments of the compound having a formula according to Formula I Z is substituted or unsubstituted 1H-2-benzopyranyl (1H-isochromenyl). In some embodiments of the compound having a formula according to Formula I Z is substituted or unsubstituted isochromanyl. In some embodiments of the compound having a formula according to Formula I Z is substituted or unsubstituted 3H-2-benzopyranyl (3H-isochromenyl). In some embodiments of the compound having a formula according to Formula I Z is substituted or unsubstituted 1-benzopyran-4-on-yl (chromonyl). In some embodiments of the compound having a formula according to Formula I Z is substituted or unsubstituted 4-chromanonyl. In some embodiments of the compound having a formula according to Formula I Z is substituted or unsubstituted 1-benzopyran-2-on-yl (coumarinyl). In some embodiments of the compound having a formula according to Formula I Z is substituted or unsubstituted dihydrocoumarinyl. In some embodiments of the compound having a formula according to Formula I Z is substituted or unsubstituted 3-isochromanonyl. In some embodiments of the compound having a formula according to Formula I Z is substituted or unsubstituted 2-coumaranon-yl. In some embodiments of the compound having a formula according to Formula I Z is substituted or unsubstituted (C₁-C₆)alkyl. In some embodiments of the compound having a formula according to Formula I Z is substituted or unsubstituted (C₁-C₆)heteroalkyl. In some embodiments of the compound having a formula according to Formula I Z is substituted or unsubstituted imidazolyl. In some embodiments of the compound having a formula according to Formula I Z is substituted or unsubstituted azaindolyl including 7-azaindolyl, 6-azaindolyl, 5-azaindolyl and 4-azaindolyl. In some embodiments of the compound having a formula according to Formula I Z is substituted or unsubstituted indolyl. In some embodiments of the compound having a formula according to Formula I Z is not a substituted monocyclic six-membered aryl or unsubstituted monocyclic six-membered aryl. In some embodiments of the compound having a formula according to Formula I Z is not a substituted phenyl or unsubstituted phenyl. In some embodiments of the compound having a formula according to Formula I Z is not a phenyl substituted with 1, 2, 3, 4 or 5 substituents selected from the group consisting of —NO₂, —N₃, halogen, —NH₂, hydroxyl, —OR¹¹ and —C(═O)R¹¹, wherein R¹¹ is hydrogen, substituted alkyl or substituted alkynyl.

In some embodiments of the compound having a formula according to Formula I, A is a methylene group and n is 1. In some embodiments of the compound having a formula according to Formula I, A is a methylene group and n is 2.

As used herein and throughout the entire description, the term “aliphatic” includes both saturated and unsaturated, nonaromatic, straight chain (i.e., unbranched), branched, acyclic, and cyclic (i.e., carbocyclic) hydrocarbons, which are optionally substituted with one or more functional groups. As will be appreciated by one of ordinary skill in the art, “aliphatic” is intended herein to include, but is not limited to, alkyl, alkenyl, alkynyl, cycloalkyl, cycloalkenyl, and cycloalkynyl moieties. Thus, as used herein, the term “alkyl” includes straight, branched and cyclic alkyl groups. An analogous convention applies to other generic terms such as “alkenyl,” “alkynyl,” and the like. Furthermore, as used herein, the terms “alkyl,” “alkenyl,” “alkynyl,” and the like encompass both substituted and unsubstituted groups. In certain embodiments, as used herein, “aliphatic” is used to indicate those aliphatic groups (cyclic, acyclic, substituted, unsubstituted, branched or unbranched) having 1-20 carbon atoms (C₁₋₂₀ aliphatic). In certain embodiments, the aliphatic group has 1-10 carbon atoms (C₁₋₁₀ aliphatic). In certain embodiments, the aliphatic group has 1-6 carbon atoms (C₁₋₆ aliphatic). In certain embodiments, the aliphatic group has 1-5 carbon atoms (C₁₋₅ aliphatic). In certain embodiments, the aliphatic group has 1-4 carbon atoms (C₁₋₄ aliphatic). In certain embodiments, the aliphatic group has 1-3 carbon atoms (C₁₋₃ aliphatic). In certain embodiments, the aliphatic group has 1-2 carbon atoms (C₁₋₂ aliphatic). Aliphatic group substituents include, but are not limited to, any of the substituents described herein, that result in the formation of a stable moiety. In some embodiments, the aliphatic group is saturated or unsaturated, unbranched or branched alkyl, preferably (C₁-C₂₀)alkyl, more preferably (C₁-C₁₀)alkyl, even more preferably (C₁-C₆)alkyl.

As used herein and throughout the entire description, the term “heteroaliphatic” refers to an aliphatic moiety, as defined herein, which includes both saturated and unsaturated, nonaromatic, straight chain (i.e., unbranched), branched, acyclic, cyclic (i.e., heterocyclic), or polycyclic hydrocarbons, which are optionally substituted with one or more functional groups, and that further contains one or more heteroatoms (e.g., oxygen, sulfur, nitrogen, phosphorus, or silicon atoms) between carbon atoms. In certain embodiments, heteroaliphatic moieties are substituted by independent replacement of one or more of the hydrogen atoms thereon with one or more substituents. As will be appreciated by one of ordinary skill in the art, “heteroaliphatic” is intended herein to include, but is not limited to, heteroalkyl, heteroalkenyl, heteroalkynyl, heterocycloalkyl, heterocycloalkenyl, and heterocycloalkynyl moieties. Thus, the term “heteroaliphatic” includes the terms “heteroalkyl,” “heteroalkenyl,” “heteroalkynyl,” and the like. Furthermore, as used herein, the terms “heteroalkyl,” “heteroalkenyl,” “heteroalkynyl,” and the like encompass both substituted and unsubstituted groups. In certain embodiments, as used herein, “heteroaliphatic” is used to indicate those heteroaliphatic groups (cyclic, acyclic, substituted, unsubstituted, branched or unbranched) having 1-20 carbon atoms and 1-6 heteroatoms (C₁₋₂₀ heteroaliphatic). In certain embodiments, the heteroaliphatic group contains 1-10 carbon atoms and 1-4 heteroatoms (C₁₋₁₀ heteroaliphatic). In certain embodiments, the heteroaliphatic group contains 1-6 carbon atoms and 1-3 heteroatoms (C₁₋₆ heteroaliphatic). In certain embodiments, the heteroaliphatic group contains 1-5 carbon atoms and 1-3 heteroatoms (C₁₋₅ heteroaliphatic). In certain embodiments, the heteroaliphatic group contains 1-4 carbon atoms and 1-2 heteroatoms (C₁₋₄ heteroaliphatic). In certain embodiments, the heteroaliphatic group contains 1-3 carbon atoms and 1 heteroatom (C₁₋₃ heteroaliphatic). In certain embodiments, the heteroaliphatic group contains 1-2 carbon atoms and 1 heteroatom (C₁₋₂ heteroaliphatic). Heteroaliphatic group substituents include, but are not limited to, any of the substituents described herein, that result in the formation of a stable moiety. In some embodiments, the aliphatic group is saturated or unsaturated, unbranched or branched alkyl, preferably (C₁-C₂₀)heteroalkyl, more preferably (C₁-C₁₀)heteroalkyl, even more preferably (C₁-C₆)heteroalkyl.

### “L”: Linker

The present disclosure provides antibody drug conjugates, where Brentuximab, modified as described herein, is linked to a drug moiety. In accordance with the present disclosure, Brentuximab may be linked, via covalent attachment by a linker, to the drug moiety. As used herein, a “linker” is any chemical moiety that is capable of linking an antibody such as Brentuximab, antibody fragment (e.g., antigen binding fragments) or functional equivalent to another moiety, such as a drug moiety. In this regard, it is again referred to the “ADC formula” described herein: Ab-(L-(D)ₓ)_{y}. Accordingly, the drug moiety D can be linked to Brentuximab through a linker L. L is any chemical moiety that is capable of linking Brentuximab Ab to the drug moiety D. Preferably, the linker L attaches Brentuximab Ab to the drug moiety D through covalent bond(s). The linker reagent is a bifunctional or multifunctional moiety which can be used to link a drug moiety D and Brentuximab Ab to form antibody drug conjugates. Antibody drug conjugates can be prepared using a linker having a reactive functionality for binding to the drug moiety D and to the antibody Ab. The terms “linker reagent”, “cross-linking reagent”, “linker derived from a cross-linking reagent” and “linker” may be used interchangeably throughout the present disclosure.

Linkers can be susceptible to cleavage (cleavable linker) such as enzymatic cleavage, acid-induced cleavage, photo-induced cleavage and disulfide bond cleavage. Enzymatic cleavage includes, but is not limited to, protease-induced cleavage, peptidase-induced cleavage, esterase-induced cleavage, glycosidase-induced cleavage, phosphatase-induced cleavage, and sulfatase-induced cleavage, preferably at conditions under which the compound or the antibody remains active. Alternatively, linkers can be substantially resistant to cleavage (e.g., stable linker or non-cleavable linker). In some aspects, the linker may be a procharged linker, a hydrophilic linker, a PEG-based linker, or a dicarboxylic acid based linker. Accordingly, in some embodiments of any one of the antibody drug conjugates disclosed herein the linker (L) is selected from the group consisting of a cleavable linker, a non-cleavable linker, a hydrophilic linker, a PEG-based linker, a procharged linker and a dicarboxylic acid based linker. In some embodiments, L is a cleavable linker. In some embodiments, L is a non-cleavable linker. In some embodiments, L is a linker susceptible to enzymatic cleavage. In some embodiments, L is an acid-labile linker, photo-labile linker, peptidase cleavable linker, protease cleavable linker, esterase cleavable linker, glycosidase cleavable linker, phosphatase cleavable linker, sulfatase cleavable linker, a disulfide bond reducible linker, a hydrophilic linker, a procharged linker, a PEG-based linker, or a dicarboxylic acid based linker. Preferably a peptidase cleavable linker. Other preferred linkers are cleavable by a protease.

Non-cleavable linkers are any chemical moiety capable of linking a drug to an antibody in a stable, covalent manner and does not fall off under the categories listed above for cleavable linkers. Thus, non-cleavable linkers are substantially resistant to acid-induced cleavage, peptidase-induced cleavage, protease-induced cleavage, esterase-induced cleavage and disulfide bond cleavage. Furthermore, non-cleavable refers to the ability of the chemical bond in the linker or adjoining to the linker to withstand cleavage induced by an acid, photo labile-cleaving agent, a peptidase, a protease, an esterase, or a chemical or physiological compound that cleaves a disulfide bond, at conditions under which the drug or the antibody does not lose its activity.

Acid-labile linkers are linkers cleavable at acidic pH. For example, certain intracellular compartments, such as endosomes and lysosomes, have an acidic pH (pH 4-5), and provide conditions suitable to cleave acid-labile linkers.

Some linkers can be cleaved by peptidases, i.e. peptidase cleavable linkers. In this regard, certain peptides are readily cleaved inside or outside cells, see e.g. Trout et al., 79 Proc. Natl. Acad. Sci. USA, 626-629 (1982) and Umemoto et al. 43 Int. J. Cancer, 677-684 (1989). Peptides are composed of α-amino acids and peptidic bonds, which chemically are amide bonds between the carboxylate of one amino acid and the amino group of a second amino acid.

Some linkers can be cleaved by esterases, i.e. esterase cleavable linkers. In this regard, certain esters can be cleaved by esterases present inside or outside of cells. Esters are formed by the condensation of a carboxylic acid and an alcohol. Simple esters are esters produced with simple alcohols, such as aliphatic alcohols, and small cyclic and small aromatic alcohols.

Procharged linkers are derived from charged cross-linking reagents that retain their charge after incorporation into an antibody drug conjugate. Examples of procharged linkers can be found in US 2009/0274713.

The linker may be cleavable, preferably by a protease, more preferably by a cathepsin such as cathepsin B. The linker may comprise a valine-citrulline moiety, which can be cleaved by a cathepsin such as cathepsin B. The linker may comprise a hydroxylamine group and the unnatural amino acid comprises a formyl group ortho of a hydroxyl group in an aromatic ring such as 3-formyltyorsine, and wherein the hydroxylamine group of the linker forms an oxime with the formyl group of the unnatural amino acid after conjugation. The linker may comprise a cleavage site. A “cleavage site” is a sequence of amino acids, which is recognized by a protease or a peptidase and hydrolyzed by said protease or peptidase. Preferably, the cleavage site is a valine citrulline moiety.

In one embodiment, the Linker of the ADC of the invention has the formula: —Aₐ-Xₓ—W_{w}—Y_{y}, wherein: —A— is a Stretcher unit; a is 0 or 1; wherein —X— is a second Spacer unit; x is independently an integer ranging from 0 to 12; each —W— is independently an Amino Acid unit; w is independently an integer ranging from 0 to 12; —Y— is a first Spacer unit; and y is independently an integer ranging from 0 to 12, preferably 0, 1 or 2.

The Stretcher unit (-A-), when present, is capable of linking a Ligand unit, i.e. the unnatural amino acid of Brentuximab comprised in the ADC of the invention, to an amino acid unit (—W—). In this regard an unnatural amino acid has a functional group that can form a bond with a functional group of a Stretcher. Useful functional groups that can be present on the unnatural amino acid, either naturally or via chemical manipulation, include, but are not limited to, sulfhydryl (—SH), amino, azido, alkynyl, hydroxyl, carboxy, the anomeric hydroxyl group of a carbohydrate, formyl and carboxyl. In one aspect, the unnatural amino acid functional groups are sulfhydryl and amino. Preferably, the functional group of the unnatural amino acid is formyl. Sulfhydryl groups can be generated by reduction of an intramolecular disulfide bond of a Ligand. Alternatively, sulfhydryl groups can be generated by reaction of an amino group of a lysine moiety of the unnatural amino acid using 2-iminothiolane (Traut's reagent) or another sulfhydryl generating reagent.

The reactive group of the Stretcher may contain a reactive site that is reactive to a modified carbohydrate's (—CHO) group that can be present on the unnatural amino acid. For example, a carbohydrate can be mildly oxidized using a reagent such as sodium periodate or a unnatural formyltyrosine amino acid can be ligated to the ligand utilizing the TTL and the resulting (—CHO) unit can be condensed with a Stretcher that contains a functionality such as a hydrazide, an oxime, pyrazolone, thiopyrazolone a primary or secondary amine, a hydrazine, a thiosemicarbazone, a hydrazine carboxylate, and an arylhydrazide such as those described by Kaneko, T. et al. (1991) Bioconjugate Chem 2:133-41. Representative Stretcher units after coupling to the unnatural amino acid of this embodiment are depicted within the square brackets of Structures VIa, VIb, and VIc, wherein —W—, —Y—, —D, w and y are as defined above and L is the unnatural amino acid of Brentuximab comprised in the ADC of the invention.

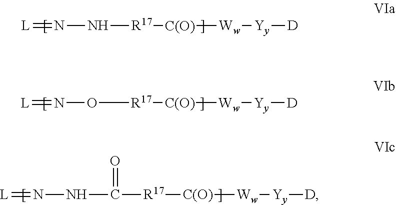

wherein R¹⁷ is selected from selected from the group consisting of substituted or unsubstituted alkyl, substituted or unsubstituted alkenyl, substituted or unsubstituted alkynyl, substituted or unsubstituted cycloalkyl, substituted or unsubstituted aryl, substituted or unsubstituted arylalkyl, substituted or unsubstituted heteroaryl, substituted or unsubstituted heteroarylalkyl, substituted or unsubstituted heterocyclyl, substituted or unsubstituted heteroalkyl, substituted or unsubstituted heteroalkenyl and substituted or unsubstituted heteroalkynyl.

The stretcher unit may also comprise one or more of the following:

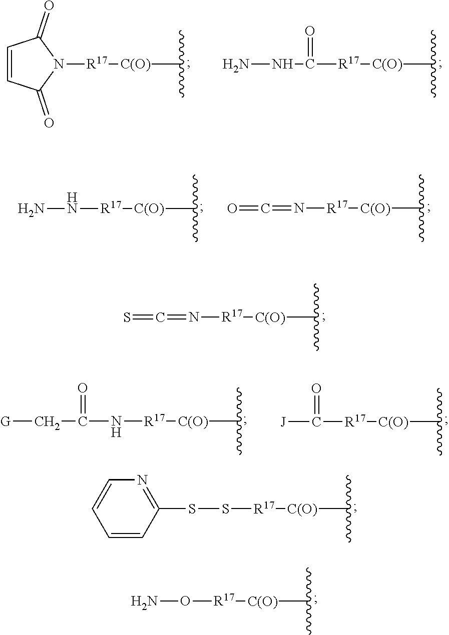

wherein G is selected from —Cl, —Br, —I, —O-mesyl and —O-tosyl;
wherein J is selected from —Cl, —Br, —I, —F, —OH, —O—N-succinimide, —O(4-nitrophenyl), —O— pentafluorophenyl, —O-tetrafluorophenyl and —O—C(O)—OR¹⁸; R¹⁸ is —C₁-C₈ alkyl or -aryl and wherein R¹⁷ is selected from selected from the group consisting of substituted or unsubstituted alkyl, substituted or unsubstituted alkenyl, substituted or unsubstituted alkynyl, substituted or unsubstituted cycloalkyl, substituted or unsubstituted aryl, substituted or unsubstituted arylalkyl, substituted or unsubstituted heteroaryl, substituted or unsubstituted heteroarylalkyl, substituted or unsubstituted heterocyclyl, substituted or unsubstituted heteroalkyl, substituted or unsubstituted heteroalkenyl and substituted or unsubstituted heteroalkynyl. The wavy lines indicate a covalent bond to the amino acid unit W, if present, the spacer unit W, if no amino acid is present and the spacer unit is present, or the drug moiety, if no amino acid unit or spacer unit is present.

The stretcher unit may comprise or consist of a structure as depicted in 1 before being coupled to the unnatural amino acid:

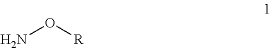

wherein R may indicate a covalent bond to the second spacer unit X, the amino acid unit W, the first spacer unit W, or the drug moiety.

In a linker as described herein, the stretcher unit may comprise or consist of a structure as depicted in 6 or 7 before being coupled to the unnatural amino acid:

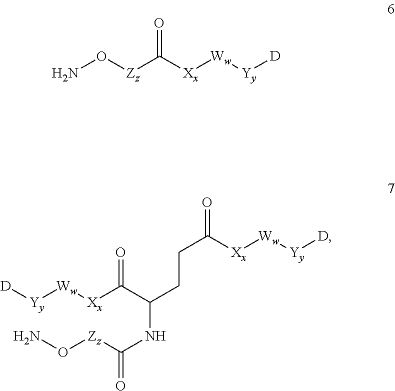

wherein Z is is selected from the group consisting of substituted or unsubstituted alkyl, substituted or unsubstituted alkenyl, substituted or unsubstituted alkynyl, substituted or unsubstituted cycloalkyl, substituted or unsubstituted aryl, substituted or unsubstituted arylalkyl, substituted or unsubstituted heteroaryl, substituted or unsubstituted heteroarylalkyl, substituted or unsubstituted heterocyclyl, substituted or unsubstituted heteroalkyl, substituted or unsubstituted heteroalkenyl and substituted or unsubstituted heteroalkynyl; z is independently an integer ranging from 0 to 12; wherein X is an optional second spacer unit as defined herein, preferably selected from the group consisting of substituted or unsubstituted alkyl, substituted or unsubstituted alkenyl, substituted or unsubstituted alkynyl, substituted or unsubstituted cycloalkyl, substituted or unsubstituted aryl, substituted or unsubstituted arylalkyl, substituted or unsubstituted heteroaryl, substituted or unsubstituted heteroarylalkyl, substituted or unsubstituted heterocyclyl, substituted or unsubstituted heteroalkyl, substituted or unsubstituted heteroalkenyl and substituted or unsubstituted heteroalkynyl, or a polyethylene glycol-based linker such as PEGᵢ, wherein i is an integer ranging from 2 to 12, preferably selected from the group consisting of 1, 2, 3, 4, 5, 6, 7, 8, 9, 10, 11 and 12, preferably 2; x is independently an integer ranging from 0 to 12;
wherein Y is an optional first spacer unit as defined herein; y is independently an integer ranging from 0 to 12;
wherein W is an optional amino acid unit as defined herein; w is independently an integer ranging from 0 to 12;
and
wherein D is a drug moiety,
preferably wherein the hydroxylamine of structure 6 or 7 is conjugated to the unnatural amino acid of Brentuximab comprised in the ADC of the invention, thereby forming an oxime after coupling.

The Amino Acid unit (—W—), when present, links the Stretcher unit to the Spacer unit if the Spacer unit is present, links the Stretcher unit to the Drug moiety if the Spacer unit is absent, and links the Ligand unit to the Drug moiety if the Stretcher unit and Spacer unit are absent.

W_{w}— is a dipeptide, tripeptide, tetrapeptide, pentapeptide, hexapeptide, heptapeptide, octapeptide, nonapeptide, decapeptide, undecapeptide or dodecapeptide unit. Each —W— unit independently has the formula denoted below in the square brackets, and w is an integer ranging from 0 to 12:

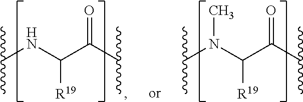

wherein R₁₉ is hydrogen, methyl, isopropyl, isobutyl, sec-butyl, benzyl, p-hydroxybenzyl, —CH₂OH, —CH(OH)CH₃, —CH₂CH₂SCH₃, —CH₂CONH₂, —CH₂COOH, —CH₂CH₂CONH₂, —CH₂CH₂COOH, —(CH₂)₃NHC(═NH)NH₂, —(CH₂)₃NH₂, —(CH₂)₃NHCOCH₃, —(CH₂)₃NHCHO, —(CH₂)₄NHC(═NH)NH₂, —(CH₂)₄NH₂, —(CH₂)₄NHCOCH₃, —(CH₂)₄NHCHO, —(CH₂)₃NHCONH₂, —(CH₂)₄NHCONH₂, —CH₂CH₂CH(OH)CH₂NH₂, 2-pyridylmethyl-, 3-pyridylmethyl-, 4-pyridylmethyl-, phenyl, cyclohexyl,

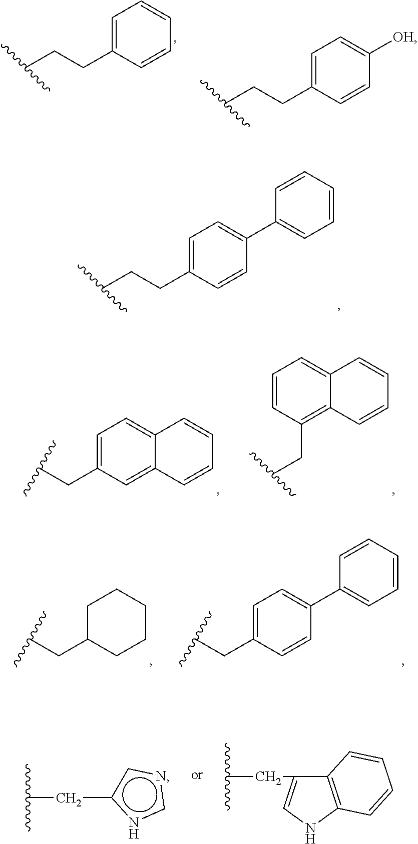

The Amino Acid unit can be enzymatically cleaved by one or more enzymes, including a tumor-associated protease, to liberate the Drug moiety (-D), which in one embodiment is protonated in vivo upon release to provide a Drug (D). Illustrative W_{w} units are represented by formulas (VII)-(IX):

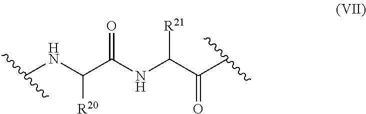

wherein R₂₀ and R₂₁ are as follows:

| R²⁰ | R²¹ |
|---|---|
| benzyl | (CH₂)₄NH₂; |
| methyl | (CH₂)₄NH₂: |
| isopropyl | (CH₂)₄NH₂: |
| isopropyl | (CH₂)₃NHCONH₂; |
| benzyl | (CH₂)₃NHCONH₂; |
| isobutyl | (CH₂)₃NHCONH₂; |
| [symbol] -butyl | (CH₂)₃NHCONH₂; |
| 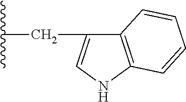 [SMILES: *Cc1c[nH]c2ccccc12] | (CH₂)₃NHCONH₂; |
| benzyl | methyl; and |
| benzyl | (CH₂)₃NHC(═NH)NH₂; |

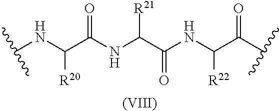

[symbol]

indicates data missing or illegible when filed

wherein R₂₀, R₂₁ and R₂₂ are as follows:

| | | |
|---|---|---|
| R²⁰ | R²¹ | R²² |
| benzyl | benzyl | (CH₂)₄NH₂; |
| isopropyl | benzyl | (CH₂)₄NH₂; and |
| H | benzyl | (CH₂)₄NH₂; |

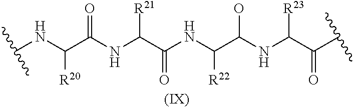

wherein R₂₀, R₂₁, R₂₂ and R₂₃ are as follows:

| R20 | R21 | R22 | R23 |
|---|---|---|---|
| H | benzyl | isobutyl | H; and |
| methyl | isobutyl | methyl | isobutyl. |

Further suitable linkers are disclosed in Salomon et al., Mol. Pharmaceutics 2019, 16, 12, 4817-4825, which is hereby incorporated by reference.

Exemplary Amino Acid units include, but are not limited to, units of formula (VII) where: R₂₀ is benzyl and R₂₁ is —(CH₂)₄NH₂; R₂₀ isopropyl and R₂₁ is —(CH₂)₄NH₂; R₂₀ isopropyl and R₂₁ is —(CH₂)₃NHCONH₂. Another exemplary Amino Acid unit is a unit of formula (VIII) wherein R₂₀ is benzyl, R₂₁ is benzyl, and R₂₂ is —(CH₂)₄NH₂.

Useful —W_{w}— units can be designed and optimized in their selectivity for enzymatic cleavage by a particular enzymes, for example, a tumor-associated protease. In one embodiment, a —W_{w}— unit is that whose cleavage is catalyzed by cathepsin B, C and D, or a plasmin protease (“tumor-associated proteases”).

In one embodiment, —W_{w}— is a dipeptide, tripeptide, tetrapeptide or pentapeptide.

When R₁₉, R₂₀, R₂₁, R₂₂ or R₂₃ is other than hydrogen, the carbon atom to which R₁₉, R₂₀, R₂₁, R₂₂ or R₂₃ is attached is chiral.

Each carbon atom to which R₁₉, R₂₀, R₂₁, R₂₂ or R₂₃ is attached is independently in the (S) or (R) configuration.

In one aspect of the Amino Acid unit, the Amino Acid unit is valine-citrulline. In another aspect, the Amino Acid unit is phenylalanine-lysine (i.e. fk). In yet another aspect of the Amino Acid unit, the Amino Acid unit is N-methylvalinecitrulline. In yet another aspect, the Amino Acid unit is 5-aminovaleric acid, homo phenylalanine lysine, tetraisoquinolinecarboxylate lysine, cyclohexylalanine lysine, isonepecotic acid lysine, beta-alanine lysine, glycine serine valine glutamine and isonepecotic acid.

In certain embodiments, the Amino Acid unit can comprise natural amino acids. In other embodiments, the Amino Acid unit can comprise non-natural amino acids.

The first Spacer unit (—Y—), when present, may link an Amino Acid unit to the Drug moiety when an Amino Acid unit is present. Alternately, the first Spacer unit may link the Stretcher unit to the Drug moiety when the Amino Acid unit is absent. The first Spacer unit may also link the Drug moiety to the Ligand unit when both the Amino Acid unit and Stretcher unit are absent. The second Spacer unit (—X—), when present, may link a Stretcher unit to the Amino Acid unit. Alternatively, the second spacer unit may link the stretcher unit to the first spacer unit, in case no amino acid unit is present. Alternatively, the second spacer unit may also link the stretcher unit to the drug moiety, in case no first spacer unit and no amino acid unit are present. Both spacer units can be identical or not identical. The disclosure for “spacer units” in general applies to both, the first and the second spacer unit. The first and the second spacer unit may be identical. The first and the second spacer unit may differ, wherein each is independently selected from the following examples of spacer units.

Spacer units are of two general types: self-immolative and non self-immolative. A non self-immolative Spacer unit is one in which part or all of the Spacer unit remains bound to the Drug moiety after cleavage, particularly enzymatic, of an Amino Acid unit from the Drug-Linker-Ligand Conjugate or the Drug-Linker Compound. Examples of a non selfimmolative Spacer unit include, but are not limited to a (glycine-glycine) Spacer unit and a glycine Spacer unit (both depicted in Scheme 1) (infra). When an Exemplary Compound containing a glycine-glycine Spacer unit or a glycine Spacer unit undergoes enzymatic cleavage via a tumor-cell associated-protease, a cancer-cell-associated protease or a lymphocyte-associated protease, a glycine-glycine-Drug moiety or a glycine-Drug moiety is cleaved from L—Aₐ—W_{w}—. In one embodiment, an independent hydrolysis reaction takes place within the target cell, cleaving the glycine-Drug moiety bond and liberating the Drug.

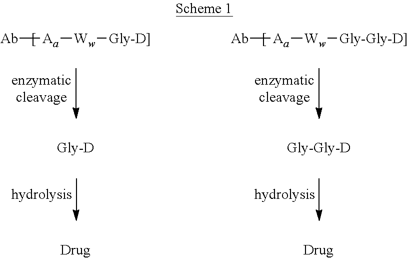

In another embodiment, a spacer unit is a p-aminobenzyl alcohol (PAB) unit (see Schemes 2 and 3) whose phenylene portion is substituted with Qₘ wherein Q is —C₁-C₈ alkyl, —O—(C₁-C₈ alkyl), -halogen, -nitro or -cyano; and m is an integer ranging from 0-4.

In one embodiment, a non self-immolative Spacer unit is -Gly-Gly-. In another embodiment, a non self immolative the Spacer unit is -Gly-.

In one embodiment, an ADC is provided in which the first Spacer unit is absent (y=0), or a pharmaceutically acceptable salt or solvate thereof. In one embodiment, an ADC is provided in which the second Spacer unit is absent (x=0), or a pharmaceutically acceptable salt or solvate thereof.

Alternatively, an Exemplary Compound containing a self-immolative Spacer unit can release —D without the need for a separate hydrolysis step. In this embodiment, a spacer unit is a PAB group that is linked to —W_{w}— via the amino nitrogen atom of the PAB group, and connected directly to —D via a carbonate, carbamate or ether group. Without being bound by any particular theory or mechanism, Scheme 2 depicts a possible mechanism of Drug release of a PAB group which is attached directly to —D via a carbamate or carbonate group espoused by Toki et al. (2002) J Org. Chem. 67:1866-1872.

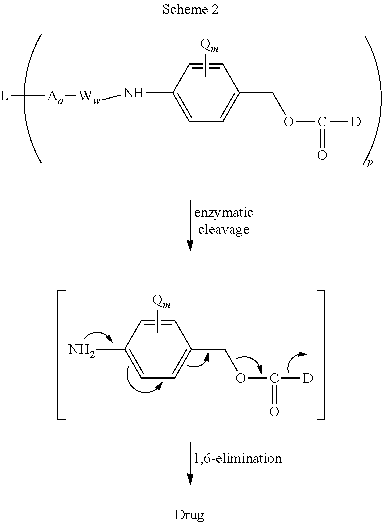

wherein Q is —C₁-C₈ alkyl, —O—(C₁-C₈ alkyl), -halogen, -nitro or -cyano; m is an integer ranging from 0-4; and p ranges from 1 to about 20.

Without being bound by any particular theory or mechanism, Scheme 3 depicts a possible mechanism of Drug release of a PAB group which is attached directly to —D via an ether or amine linkage.

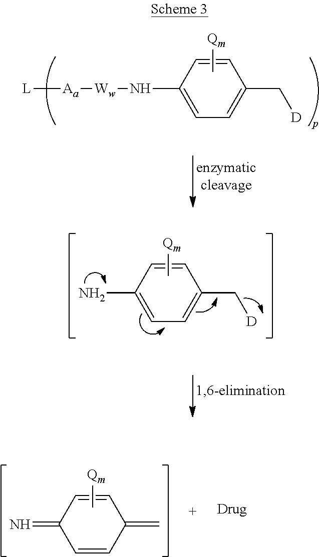

wherein Q is —C₁-C₈ alkyl, —O—(C₁-C₈ alkyl), -halogen, -nitro or -cyano; m is an integer ranging from 0-4; and p ranges from 1 to about 20.

Other examples of self-immolative spacers include, but are not limited to, aromatic compounds that are electronically similar to the PAB group such as 2-aminoimidazol-5-methanol derivatives (Hay et al. (1999) Bioorg. Med. Chem. Lett. 9:2237) and ortho or para-aminobenzylacetals. Spacers can be used that undergo cyclization upon amide bond hydrolysis, such as substituted and unsubstituted 4-aminobutyric acid amides (Rodrigues et al., Chemistry Biology, 1995, 2, 223), appropriately substituted bicyclo[2.2.1] and bicyclo[2.2.2] ring systems (Storm, et al., J. Amer. Chem. Soc., 1972, 94, 5815) and 2-aminophenylpropionic acid amides (Amsberry, et al., J. Org. Chem., 1990, 55, 5867). Elimination of amine-containing drugs that are substituted at the α-position of glycine (Kingsbury, et al., J. Med. Chem., 1984, 27, 1447) are also examples of self-immolative spacer useful in Exemplary Compounds.

In one embodiment, the Spacer unit is a branched bis(hydroxymethyl)styrene (BHMS) unit as depicted in Scheme 4, which can be used to incorporate and release multiple drugs.

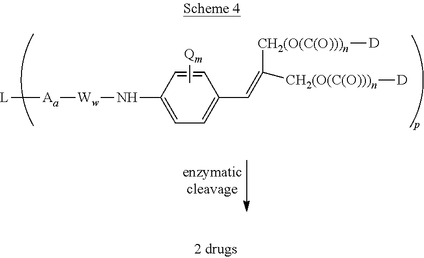

wherein Q is —C₁-C₈ alkyl, —O—(C₁-C₈ alkyl), -halogen, -nitro or -cyano; m is an integer ranging from 0-4; n is 0 or 1; and p ranges raging from 1 to about 20.

In one embodiment, the —D moieties are the same. In yet another embodiment, the —D moieties are different.

In one aspect, Spacer units are represented by Formulas (X)-(XII):

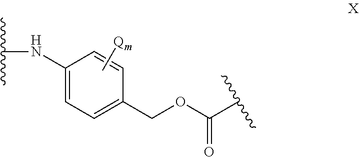

wherein Q is —C₁-C₈ alkyl, —O—(C₁-C₈ alkyl), -halogen, -nitro or -cyano; and m is an integer ranging from 0-4;

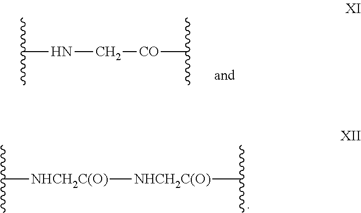

The first spacer unit (—X—) may be selected from the group consisting of substituted or unsubstituted alkyl, substituted or unsubstituted alkenyl, substituted or unsubstituted alkynyl, substituted or unsubstituted cycloalkyl, substituted or unsubstituted aryl, substituted or unsubstituted arylalkyl, substituted or unsubstituted heteroaryl, substituted or unsubstituted heteroarylalkyl, substituted or unsubstituted heterocyclyl, substituted or unsubstituted heteroalkyl, substituted or unsubstituted heteroalkenyl and substituted or unsubstituted heteroalkynyl, or a polyethylene glycol-based linker such as PEGᵢ, wherein i is an integer ranging from 2 to 12, preferably selected from the group consisting of 1, 2, 3, 4, 5, 6, 7, 8, 9, 10, 11 and 12, preferably 2; x is independently an integer ranging from 0 to 12.

The second spacer unit (—Y—) may be selected from the group consisting of substituted or unsubstituted alkyl, substituted or unsubstituted alkenyl, substituted or unsubstituted alkynyl, substituted or unsubstituted cycloalkyl, substituted or unsubstituted aryl, substituted or unsubstituted arylalkyl, substituted or unsubstituted heteroaryl, substituted or unsubstituted heteroarylalkyl, substituted or unsubstituted heterocyclyl, substituted or unsubstituted heteroalkyl, substituted or unsubstituted heteroalkenyl and substituted or unsubstituted heteroalkynyl, or a polyethylene glycol-based linker such as PEGᵢ, wherein i is an integer ranging from 2 to 12, preferably selected from the group consisting of 1, 2, 3, 4, 5, 6, 7, 8, 9, 10, 11 and 12, preferably 2; x is independently an integer ranging from 0 to 12.

The linker may have or comprise a structure as depicted in 1 before being coupled to the unnatural amino acid:

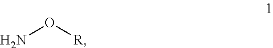

wherein R is one or more drug moieties, which are optionally coupled to the hydroxylamine of 1 by one or more cleavage sites, preferably wherein the hydroxylamine 1 is conjugated to the unnatural amino acid.

The linker may comprise or consist of a structure as depicted in 6 or 7 before being coupled to the unnatural amino acid:

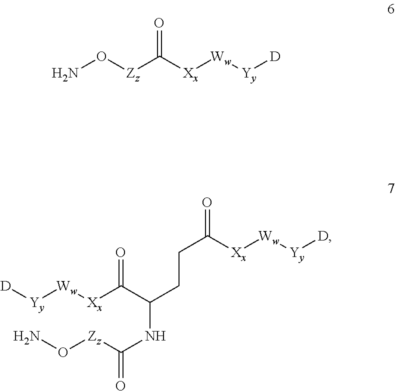

wherein Z is is selected from the group consisting of substituted or unsubstituted alkyl, substituted or unsubstituted alkenyl, substituted or unsubstituted alkynyl, substituted or unsubstituted cycloalkyl, substituted or unsubstituted aryl, substituted or unsubstituted arylalkyl, substituted or unsubstituted heteroaryl, substituted or unsubstituted heteroarylalkyl, substituted or unsubstituted heterocyclyl, substituted or unsubstituted heteroalkyl, substituted or unsubstituted heteroalkenyl and substituted or unsubstituted heteroalkynyl; z is independently an integer ranging from 0 to 12; wherein X is an optional second spacer unit as defined herein, preferably selected from the group consisting of substituted or unsubstituted alkyl, substituted or unsubstituted alkenyl, substituted or unsubstituted alkynyl, substituted or unsubstituted cycloalkyl, substituted or unsubstituted aryl, substituted or unsubstituted arylalkyl, substituted or unsubstituted heteroaryl, substituted or unsubstituted heteroarylalkyl, substituted or unsubstituted heterocyclyl, substituted or unsubstituted heteroalkyl, substituted or unsubstituted heteroalkenyl and substituted or unsubstituted heteroalkynyl, or a polyethylene glycol-based linker such as PEGᵢ, wherein i is an integer ranging from 2 to 12, preferably selected from the group consisting of 1, 2, 3, 4, 5, 6, 7, 8, 9, 10, 11 and 12, preferably 2; x is independently an integer ranging from 0 to 12;
wherein Y is an optional (first) spacer unit as defined herein; y is independently an integer ranging from 0 to 12;
wherein W is an optional amino acid unit as defined herein; w is independently an integer ranging from 0 to 12;
and
wherein D is a drug moiety,
preferably wherein the hydroxylamine of structure 6 or 7 is conjugated to the unnatural amino acid of Brentuximab comprised in the ADC of the invention, thereby forming an oxime after coupling.

The linker may have or comprise a structure as depicted in 2 or 3 before being coupled to the unnatural amino acid:

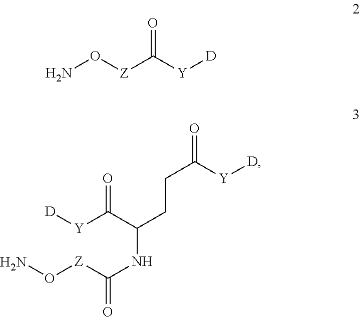

wherein Z is is selected from the group consisting of substituted or unsubstituted alkyl, substituted or unsubstituted alkenyl, substituted or unsubstituted alkynyl, substituted or unsubstituted cycloalkyl, substituted or unsubstituted aryl, substituted or unsubstituted arylalkyl, substituted or unsubstituted heteroaryl, substituted or unsubstituted heteroarylalkyl, substituted or unsubstituted heterocyclyl, substituted or unsubstituted heteroalkyl, substituted or unsubstituted heteroalkenyl and substituted or unsubstituted heteroalkynyl;
wherein D is one or more drug moieties; and
wherein Y is a cleavage site such as a cleavage site for a cathepsin such as cathepsin B; preferably wherein the hydroxylamine of structure 2 or 3 is conjugated to the unnatural amino acid.

The linker may have a structure as depicted in structure 4 or 5 before being coupled to the unnatural amino acid, wherein D is a drug moiety as described herein, preferably MMAE:

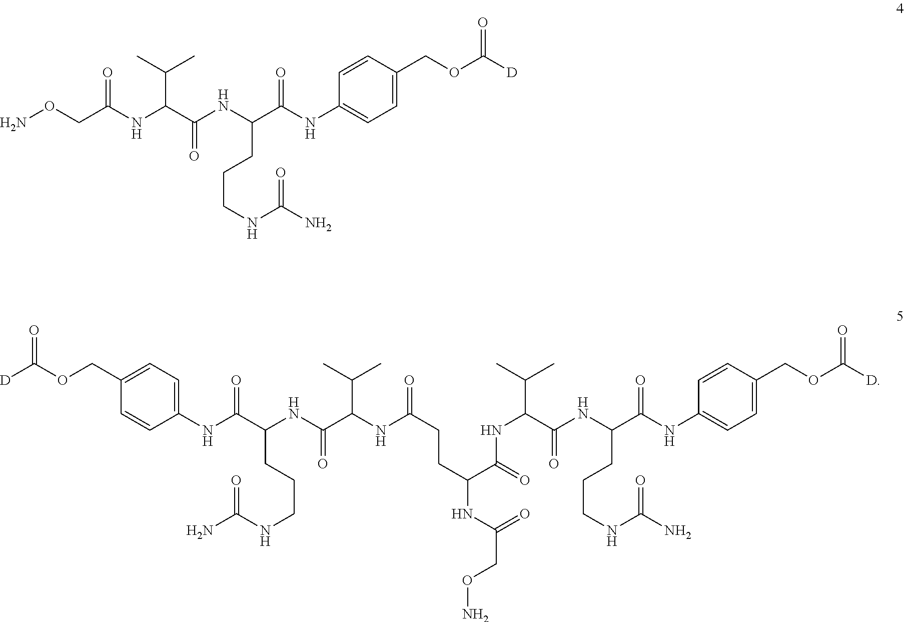

“Before coupling to the unnatural amino acid” as used herein describes a chemical entity such as the linker before it has formed a covalent bond with its counterpart, the unnatural amino acid. The same is however also true for linkers or drug moieties herein, which are not depicted as being covalently coupled to each other. The person skilled in the art can readily recognize the resulting structure formed as a result of the reaction of two chemical entities. In case of structures 1 to 5 as defined herein, the counterpart is the unnatural amino acid, which comprises a formyl group. The unnatural amino acid may be 3-formyltyrosine and the hydroxylamine group of the linker having any one of structures 1 to 5 may form an oxime/hydroxyimine with the 3-formyl group of the unnatural amino acid. The reaction mechanism is shown in the following:

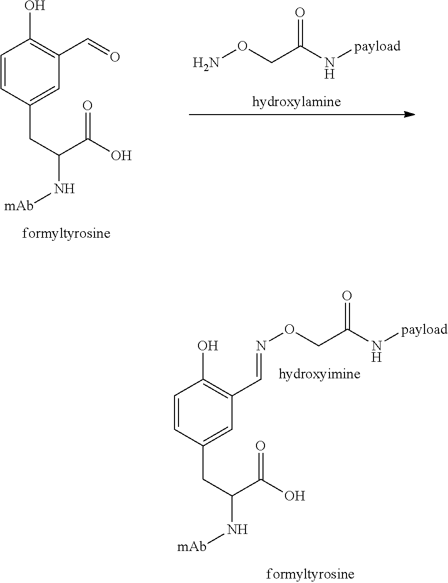

In this context, mAb corresponds to Brentuximab and the payload refers to the drug moiety as defined herein, which is coupled to the unnatural amino acid, here 3-formyltyrosine, via a linker as defined herein.

Suitable cross-linking reagents that form a non-cleavable linker between the drug moiety D and the Brentuximab Ab are well known in the art, and can form non-cleavable linkers that comprise a sulfur atom (such as SMCC) or those that are without a sulfur atom. Cross-linking reagents that form non-cleavable linkers between the drug moiety D, and the Brentuximab Ab comprise a maleimido- or haloacetyl-based moiety. According to the present disclosure, such non-cleavable linkers are said to be derived from maleimido- or haloacetyl-based moieties.

Cross-linking reagents comprising a maleimido-based moiety include but are not limited to, N-succinimidyl-4-(maleimidomethyl)cyclohexane-1-carboxylate (SMCC), sulfosuccinimidyl 4-(N-maleimidomethyl)cyclohexane-1-carboxylate (sulfo-SMCC), N-succinimidyl-4-(maleimidomethyl)cyclohexane-1-carboxy-(6-amidocaproate), which is a “long chain” analog of SMCC (LC-SMCC), κ-maleimidoundecanoic acid N-succinimidyl ester (KMUA), γ-maleimidobutyric acid N-succinimidyl ester (GMBS), c-maleimidocaproic acid N-succinimidyl ester (EMCS), m-maleimidobenzoyl-N-hydroxysuccinimide ester (MBS), N-(α-maleimidoacetoxy)-succinimide ester (AMSA), succinimidyl-6-(β-maleimidopropionamido)hexanoate (SMPH), N-succinimidyl-4-(p-maleimidophenyl)-butyrate (SMPB), N-(-p-maleomidophenyl)isocyanate (PMIP) and maleimido-based cross-linking reagents containing a polyethythene glycol spacer, such as maleimide-PEG-NHS, which is denoted herein also as MAL-PEG-NHS. These cross-linking reagents form non-cleavable linkers derived from maleimido-based moieties. Representative structures of maleimido-based cross-linking reagents are shown below.

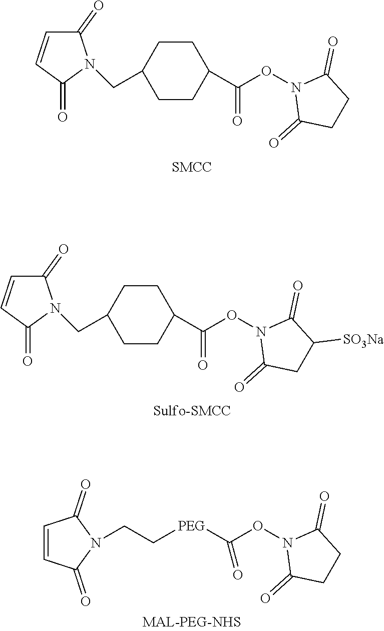

In some embodiments, the linker L is derived from N-succinimidyl-4-(maleimidomethyl)cyclohexanecarboxylate (SMCC), sulfosuccinimidyl 4-(N-maleimidomethyl) cyclohexane-1-carboxylate (sulfo-SMCC) or MAL-PEG-NHS.

Cross-linking reagents comprising a haloacetyl-based moiety include N-succinimidyl iodoacetate (SIA), N-succinimidyl(4-iodoacetyl)aminobenzoate (SIAB), N-succinimidyl bromoacetate (SBA) and N-succinimidyl 3-(bromoacetamido)propionate (SBAP). These cross-linking reagents form a non-cleavable linker derived from haloacetyl-based moieties. Representative structures of haloacetyl-based cross-linking reagents are shown below.

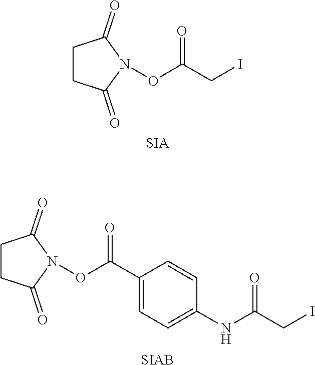

In some embodiments the linker L is derived from N-succinimidyl iodoacetate (SIA) or N-succinimidyl(4-iodoacetyl)aminobenzoate (SIAB).

Suitable cross-linking reagents that form a cleavable linker between the drug moiety D and the Brentuximab Ab are well known in the art. Disulfide containing linkers are linkers cleavable through disulfide exchange, which can occur under physiological conditions. According to the present disclosure, such cleavable linkers are said to be derived from disulfide-based moieties. Suitable disulfide cross-linking reagents include N-succinimidyl-3-(2-pyridyldithio)propionate (SPDP), N-succinimidyl-4-(2-pyridyldithio)pentanoate (SPP), N-succinimidyl-4-(2-pyridyldithio)butanoate (SPDB) and N-succinimidyl-4-(2-pyridyldithio)2-sulfo-butanoate (sulfo-SPDB), the structures of which are shown below. These disulfide cross-linking reagents form a cleavable linker derived from disulfide-based moieties.

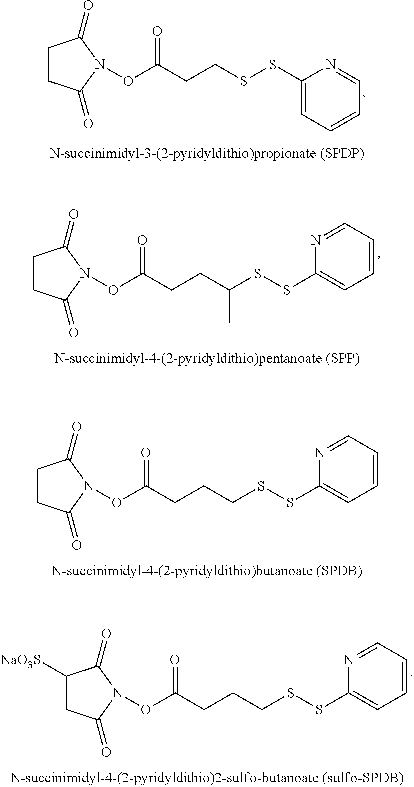

In some embodiments, the linker L is derived from N-succinimidyl-4-(2-pyridyldithio)butanoate (SPDB).

Suitable cross-linking reagents that form a charged linker between the drug moiety D and the Brentuximab Ab are known as procharged cross-linking reagents. In an embodiment, the linker L is derived from the procharged cross-linking reagent which is CX1-1. The structure of CX1-1 is shown below:

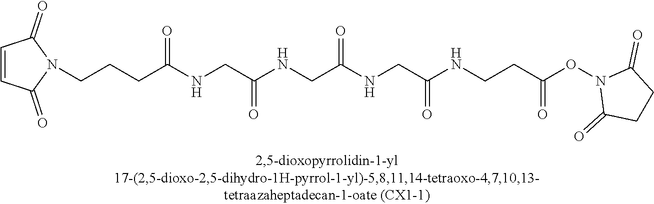 [SMILES: O=C(CCCN1C(=O)C=CC1=O)NCC(=O)NCC(=O)NCC(=O)NCCC(=O)ON1C(=O)CCC1=O]

Further linkers which may be suitably used in the Brentuximab drug conjugates disclosed herein are maleimidocaproyl (MC), maleimidocaproyl (MC) with a self-cleaving peptide, maleimidodiaminopropionyl (mDPR) with a self-cleaving peptide, and 4-(N-maleimidomethyl)-cyclohexane-1-carbonyl (MCC).

In some embodiments the linker is maleimidocaproyl (MC). Maleimidocaproyl has the following structure:

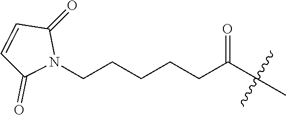 [SMILES: *C(=O)CCCCCN1C(=O)C=CC1=O]

wherein the wavy line indicates the covalent attachment of the carbonyl carbon atom of the linker to another part of the Brentuximab drug conjugate, in particular to the Brentuximab or antigen binding fragment (Ab) or to the drug moiety (D), preferably to the drug moiety (D). As non-limiting example, in an Brentuximab drug conjugate comprising an MC linker the Brentuximab or antigen binding fragment thereof (Ab) may be covalently attached to the maleimide moiety, and the drug moiety (D) may be covalently attached to the carbonyl carbon atom of the linker as indicated by the wavy line in the above structural formula.

The maleimidocaproyl linker may be, for example, derived from a cross-linking reagent which is an NHS ester having the following structure:

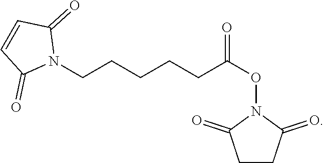 [SMILES: O=C(CCCCCN1C(=O)C=CC1=O)ON1C(=O)CCC1=O]

In some embodiments the linker is 4-(N-maleimidomethyl)-cyclohexane-1-carbonyl (MCC). MCC has the following structure:

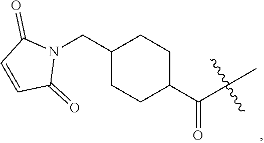 [SMILES: *C(=O)C1CCC(CN2C(=O)C=CC2=O)CC1]

wherein the wavy line indicates the covalent attachment of the carbonyl carbon atom of the linker to another part of the Brentuximab drug conjugate, in particular to the Brentuximab or antigen binding fragment (Ab) or to the drug moiety (D), preferably to the Brentuximab or antigen binding fragment (Ab). As non-limiting example, in an Brentuximab drug conjugate comprising an MCC linker the Brentuximab or antigen binding fragment thereof (Ab) may be covalently attached to the carbonyl carbon atom of the linker as indicated by the wavy line in the above structural formula, and the drug moiety (D) may be covalently attached to the maleimide moiety.

In some embodiments the linker is maleimidocaproyl (MC) with a self-cleaving peptide. Non-limiting examples of maleimidocaproyl linkers with a self-cleaving peptide are described below.

In some embodiments the linker is maleimidodiaminopropionyl (mDPR) with a self-cleaving peptide. Non-limiting examples of maleimidodiaminopropionyl (mDPR) linkers with a self-cleaving peptide are described below.

In some embodiments the linker (L) of any one of the Brentuximab drug conjugates Ab-(L-(D)ₓ)_{y} described herein is derived from a cross-linking reagent selected from the group consisting of N-succinimidyl-3-(2-pyridyldithio)propionate (SPDP), N-succinimidyl 4-(2-pyridyldithio)pentanoate (SPP), N-succinimidyl 4-(2-pyridyldithio)butanoate (SPDB), N-succinimidyl-4-(2-pyridyldithio)2-sulfo-butanoate (sulfo-SPDB), N-succinimidyl iodoacetate (SIA), N-succinimidyl(4-iodoacetyl)aminobenzoate (SIAB), maleimide-PEG-NHS, maleimidocaproyl (MC), maleimidocaproyl (MC) with a self-cleaving peptide, maleimidodiaminopropionyl (mDPR) with a self-cleaving peptide, 4-(N-maleimidomethyl)-cyclohexane-1-carbonyl (MCC), N-succinimidyl 4-(maleimidomethyl)cyclohexane-1-carboxylate (SMCC), N-sulfosuccinimidyl 4-(maleimidomethyl)cyclohexane-1-carboxylate (sulfo-SMCC) and 2,5-dioxopyrrolidin-1-yl 17-(2,5-dioxo-2,5-dihydro-1H-pyrrol-1-yl)-5,8,11,14-tetraoxo-4,7,10,13-tetraazahepta-decan-1-oate (CX1-1). In preferred embodiments the linker is derived from a cross-linking reagent selected from the group consisting of N-succinimidyl 4-(2-pyridyldithio)butanoate (SPDB), maleimidocaproyl (MC), maleimidocaproyl (MC) with a self-cleaving peptide, maleimidodiaminopropionyl (mDPR) with a self-cleaving peptide, 4-(N-maleimidomethyl)-cyclohexane-1-carbonyl (MCC), N-succinimidyl 4-(maleimidomethyl)cyclohexane-1-carboxylate (SMCC). Accordingly, in some embodiments the linker is derived from N-succinimidyl 4-(2-pyridyldithio)butanoate (SPDB).

In some embodiments the linker is derived from maleimidocaproyl (MC). In some embodiments the linker is derived from maleimidocaproyl (MC) with a self-cleaving peptide. In some embodiments the linker is derived from maleimidodiaminopropionyl (mDPR) with a self-cleaving peptide. In some embodiments the linker is derived from 4-(N-maleimidomethyl)-cyclohexane-1-carbonyl (MCC). In some embodiments the linker is derived from N-succinimidyl 4-(maleimidomethyl) cyclohexane-1-carboxylate (SMCC).

As set out herein above, the linker may be maleimidocaproyl (MC) with a self-cleaving peptide. In some embodiments the linker with a self-cleaving peptide is selected from the group consisting of maleimidocaproyl-valine-citrulline-p-aminobenzyloxycarbonyl (MC-VC-PAB), maleimidocaproyl-valine-alanine-p-aminobenzyloxycarbonyl (MC-VA-PAB), maleimidocaproyl-lysine-phenylalanine-p-aminobenzyloxycarbonyl (MC-KF-PAB), maleimidocaproyl-valine-lysine-p-aminobenzyloxycarbonyl (MC-VK-PAB). Maleimidocaproyl (MC) linkers with a self-cleaving peptide are, for example, disclosed in U.S. patent application publication US 2006/0074008, G. M. Dubowchik et al., Bioconjuate Chem. 2002, 13, 855-869, or S. O. Doronina et al., Nature Biotechnology, vol. 21, 778-784 (2003), the whole disclosure of these documents is incorporated herein by reference.

In some embodiments the linker with a self-cleaving peptide is maleimidocaproyl-valine-citrulline-p-aminobenzyloxycarbonyl (MC-VC-PAB). The MC-VC-PAB linker has the following structure:

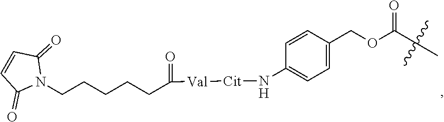

wherein the wavy line indicates the covalent attachment of the carbonyl carbon atom of the linker to another part of the Brentuximab drug conjugate, in particular to the Brentuximab or antigen binding fragment (Ab) or to the drug moiety (D), preferably to the drug moiety (D). As non-limiting example, in an Brentuximab drug conjugate comprising an MC-VC-PAB linker the Brentuximab or antigen binding fragment thereof (Ab) may be covalently attached to the maleimide moiety, and the drug moiety (D) may be covalently attached to the carbonyl carbon atom of the linker as indicated by the wavy line in the above structural formula. This linker and the following maleimidocaproyl linkers with a self-cleaving peptide which contain a para-aminobenzyloxycarbonyl group may, for example, derive from a cross-linking reagent in which a para-nitrophenoxy group is attached at the position indicated with the wavy line (see, for example, G. M. Dubowchik et al., Bioconjuate Chem. 2002, 13, 855-869, or S. O. Doronina et al., Nature Biotechnology, vol. 21, 778-784 (2003)). In case of an MC-VC-PAB linker the cross-linking reagent may thus have the structure:

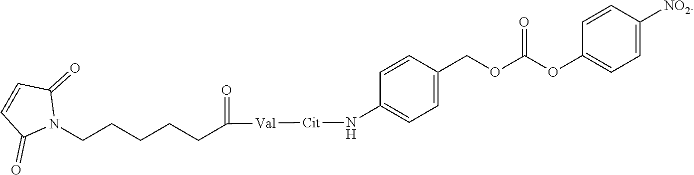

When reacting, for example, such cross-linking reagent with a drug moiety the para-nitrophenoxy group may be replaced by the drug moiety.

Without wishing to be bound by any theory, in the MC-VC-PAB linker and in the maleimidocaproyl linkers with a self-cleaving peptide which contain a para-aminobenzyloxycarbonyl group described in the following, cleavage by a protease may occur between the peptide part and the para-aminobenzyloxycarbonyl moiety (see, for example, G. M. Dubowchik et al., Bioconjuate Chem. 2002, 13, 855-869, or S. O. Doronina et al., Nature Biotechnology, vol. 21, 778-784 (2003)). The para-aminobenzyloxycarbonyl moiety may then act as a self-immolative group. In this regard, the term “self-immolative group” refers to a bifunctional chemical moiety that is capable of covalently linking together two spaced chemical moieties into a normally stable tripartite molecule. It will spontaneously separate from the second chemical moiety if its bond to the first moiety is cleaved.

In some embodiments the linker with a self-cleaving peptide is maleimidocaproyl-valine-alanine-p-aminobenzyloxycarbonyl (MC-VA-PAB). The MC-VA-PAB linker has the following structure:

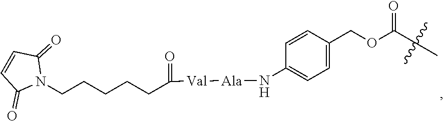

wherein the wavy line indicates the covalent attachment of the carbonyl carbon atom of the linker to another part of the Brentuximab drug conjugate, in particular to the Brentuximab or antigen binding fragment (Ab) or to the drug moiety (D), preferably to the drug moiety (D). As non-limiting example, in an Brentuximab drug conjugate comprising an MC-VA-PAB linker the Brentuximab or antigen binding fragment thereof (Ab) may be covalently attached to the maleimide moiety, and the drug moiety (D) may be covalently attached to the carbonyl carbon atom of the linker as indicated by the wavy line in the above structural formula.

In some embodiments the linker with a self-cleaving peptide is maleimidocaproyl-lysine-phenylalanine-p-aminobenzyloxycarbonyl (MC-KF-PAB). The MC-KF-PAB linker has the following structure:

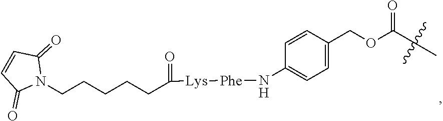

wherein the wavy line indicates the covalent attachment of the carbonyl carbon atom of the linker to another part of the Brentuximab drug conjugate, in particular to the Brentuximab or antigen binding fragment (Ab) or to the drug moiety (D), preferably to the drug moiety (D). As non-limiting example, in an Brentuximab drug conjugate comprising an MC-KF-PAB linker the Brentuximab or antigen binding fragment thereof (Ab) may be covalently attached to the maleimide moiety, and the drug moiety (D) may be covalently attached to the carbonyl carbon atom of the linker as indicated by the wavy line in the above structural formula.

In some embodiments the linker with a self-cleaving peptide is maleimidocaproyl-valine-lysine-p-aminobenzyloxycarbonyl (MC-VK-PAB). The MC-VK-PAB linker has the following structure:

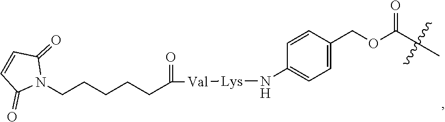

wherein the wavy line indicates the covalent attachment of the carbonyl carbon atom of the linker to another part of the Brentuximab drug conjugate, in particular to the Brentuximab or antigen binding fragment (Ab) or to the drug moiety (D), preferably to the drug moiety (D). As non-limiting example, in an Brentuximab drug conjugate comprising an MC-VK-PAB linker the Brentuximab or antigen binding fragment thereof (Ab) may be covalently attached to the maleimide moiety, and the drug moiety (D) may be covalently attached to the carbonyl carbon atom of the linker as indicated by the wavy line in the above structural formula.

As set out herein above, the linker may be maleimidodiaminopropionyl (mDPR) with a self-cleaving peptide. Such linker comprises a maleimidodiaminopropionyl moiety (mDPR) having the following structure:

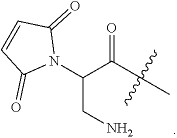 [SMILES: *C(=O)C(CN)N1C(=O)C=CC1=O]

In some embodiments the maleimidodiaminopropionyl (mDPR) linker with a self-cleaving peptide is selected from the group consisting of maleimidodiaminopropionyl-valine-citrulline-p-aminobenzyloxycarbonyl (mDPR-VC-PAB), maleimidodiaminopropionyl-valine-alanine-p-aminobenzyloxycarbonyl (mDPR-VA-PAB), maleimidodiaminopropionyl-lysine-phenylalanine-p-aminobenzyloxycarbonyl (mDPR-KF-PAB), maleimidodiaminopropionyl-valine-lysine-p-aminobenzyloxycarbonyl (mDPR-VK-PAB). Maleimidodiaminopropionyl (mDPR) linkers with a self-cleaving peptide are, for example, disclosed in U.S. patent application publication US 2013/0309256, the whole disclosure of which is incorporated herein by reference.

In some embodiments the linker with a self-cleaving peptide is maleimidodiaminopropionyl-valine-citrulline-p-aminobenzyloxycarbonyl (mDPR-VC-PAB). The mDPR-VC-PAB linker has the following structure:

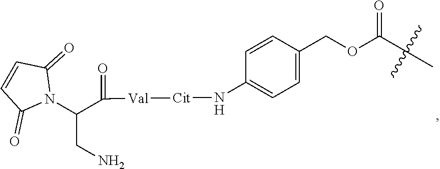

wherein the wavy line indicates the covalent attachment of the carbonyl carbon atom of the linker to another part of the Brentuximab drug conjugate, in particular to the Brentuximab or antigen binding fragment (Ab) or to the drug moiety (D), preferably to the drug moiety (D). As non-limiting example, in an Brentuximab drug conjugate comprising an mDPR-VC-PAB linker the Brentuximab or antigen binding fragment thereof (Ab) may be covalently attached to the maleimide moiety, and the drug moiety (D) may be covalently attached to the carbonyl carbon atom of the linker as indicated by the wavy line in the above structural formula. Without wishing to be bound by any theory, in the mDPR-VC-PAB linker and in the following maleimidodiaminopropionyl linkers with a self-cleaving peptide which contain a para-aminobenzyloxycarbonyl group, cleavage by a protease may occur between the peptide part and the para-aminobenzyloxycarbonyl moiety. The para-aminobenzyloxycarbonyl moiety may then act as a self-immolative group.

In some embodiments the linker with a self-cleaving peptide is maleimidodiaminopropionyl-valine-alanine-p-aminobenzyloxycarbonyl (mDPR-VA-PAB). The mDPR-VA-PAB linker has the following structure:

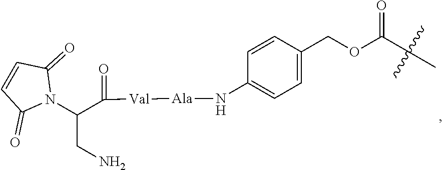

wherein the wavy line indicates the covalent attachment of the carbonyl carbon atom of the linker to another part of the Brentuximab drug conjugate, in particular to the Brentuximab or antigen binding fragment (Ab) or to the drug moiety (D), preferably to the drug moiety (D). As non-limiting example, in an Brentuximab drug conjugate comprising an mDPR-VA-PAB linker the Brentuximab or antigen binding fragment thereof (Ab) may be covalently attached to the maleimide moiety, and the drug moiety (D) may be covalently attached to the carbonyl carbon atom of the linker as indicated by the wavy line in the above structural formula.

In some embodiments the linker with a self-cleaving peptide is maleimidodiaminopropionyl-lysine-phenylalanine-p-aminobenzyloxycarbonyl (mDPR-KF-PAB). The mDPR-KF-PAB linker has the following structure:

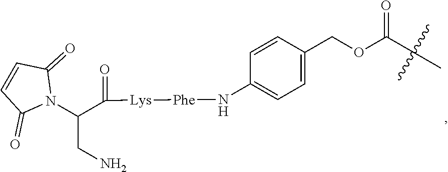

wherein the wavy line indicates the covalent attachment of the carbonyl carbon atom of the linker to another part of the Brentuximab drug conjugate, in particular to the Brentuximab or antigen binding fragment (Ab) or to the drug moiety (D), preferably to the drug moiety (D). As non-limiting example, in an Brentuximab drug conjugate comprising an mDPR-KF-PAB linker the Brentuximab or antigen binding fragment thereof (Ab) may be covalently attached to the maleimide moiety, and the drug moiety (D) may be covalently attached to the carbonyl carbon atom of the linker as indicated by the wavy line in the above structural formula.

In some embodiments the linker with a self-cleaving peptide is maleimidodiaminopropionyl-valine-lysine-p-aminobenzyloxycarbonyl (mDPR-VK-PAB). The mDPR-VK-PAB linker has the following structure:

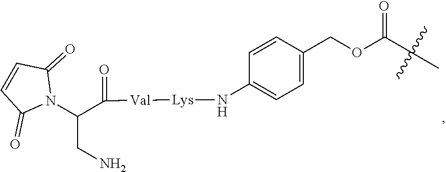

wherein the wavy line indicates the covalent attachment of the carbonyl carbon atom of the linker to another part of the Brentuximab drug conjugate, in particular to the Brentuximab or antigen binding fragment (Ab) or to the drug moiety (D), preferably to the drug moiety (D). As non-limiting example, in an Brentuximab drug conjugate comprising an mDPR-VK-PAB linker the Brentuximab or antigen binding fragment thereof (Ab) may be covalently attached to the maleimide moiety, and the drug moiety (D) may be covalently attached to the carbonyl carbon atom of the linker as indicated by the wavy line in the above structural formula.

In some embodiments a linker of an Brentuximab drug conjugate disclosed herein comprises a glucuronic acid modification. For linkers having a glucuronic acid modification see, for example, S. C. Jeffrey et al., Bioconjugate Chem. 2006, 17, 831-840, R. P. Lyon et al., Nature Biotechnology, vol. 33, 733-736 (2015), U.S. patent application publication US 2013/0309256, and international patent application publication WO 2015/057699, the whole disclosure of these documents incorporated herein by reference.

In some embodiments, said linker comprising a glucuronic acid modification may be maleimidocaproyl-glucuronide (MC-G) or maleimidodiaminopropionyl-glucuronide (mDPR-G).

Said maleimidocaproyl-glucuronide (MC-G) linker has the following structure:

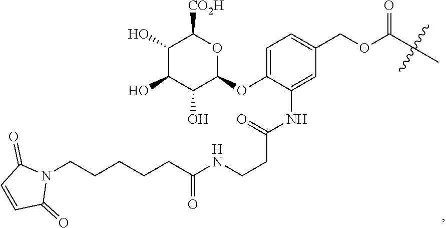 [SMILES: *C(=O)OCc1ccc(O[C@@H]2O[C@H](C(=O)O)[C@@H](O)[C@H](O)[C@H]2O)c(NC(=O)CCNC(=O)CCCCCN2C(=O)C=CC2=O)c1]

wherein the wavy line indicates the covalent attachment of the carbonyl carbon atom of the linker to another part of the Brentuximab drug conjugate, in particular to the Brentuximab or antigen binding fragment (Ab) or to the drug moiety (D), preferably to the drug moiety (D). As non-limiting example, in an Brentuximab drug conjugate comprising an MC-G linker the Brentuximab or antigen binding fragment thereof (Ab) may be covalently attached to the maleimide moiety, and the drug moiety (D) may be covalently attached to the carbonyl carbon atom of the linker as indicated by the wavy line in the above structural formula.

Said maleimidodiaminopropionyl-glucuronide (mDPR-G) linker has the following structure:

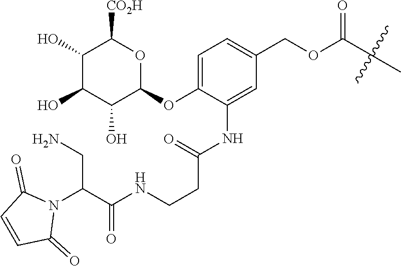 [SMILES: *C(=O)OCc1ccc(O[C@@H]2O[C@H](C(=O)O)[C@@H](O)[C@H](O)[C@H]2O)c(NC(=O)CCNC(=O)C(CN)N2C(=O)C=CC2=O)c1]

wherein the wavy line indicates the covalent attachment of the carbonyl carbon atom of the linker to another part of the Brentuximab drug conjugate, in particular to the Brentuximab or antigen binding fragment (Ab) or to the drug moiety (D), preferably to the drug moiety (D). As non-limiting example, in an Brentuximab drug conjugate comprising an mDPR-G linker the Brentuximab or antigen binding fragment thereof (Ab) may be covalently attached to the maleimide moiety, and the drug moiety (D) may be covalently attached to the carbonyl carbon atom of the linker as indicated by the wavy line in the above structural formula.

In some embodiments said linker comprising a glucuronic acid modification further comprises a polyethylene glycol modification. For linkers having a glucuronic acid modification and a polyethylene glycol modification see, for example, R. P. Lyon et al., Nature Biotechnology, vol. 33, 733-736 (2015) and international patent application publication WO 2015/057699, the whole disclosure of these documents incorporated herein by reference.

In some embodiments, said linker comprising a glucuronic acid modification and further comprising a polyethylene glycol modification may be maleimidocaproyl-glucuronide-polyethylene glycol (MC-G-PEG), maleimidodiaminopropionyl-glucuronide-polyethylene glycol (mDPR-G-PEG), or maleimidopropionyl-glucuronide-polyethylene glycol (MP-G-PEG).

Said maleimidocaproyl-glucuronide-polyethylene glycol (MC-G-PEG) linker has the following structure:

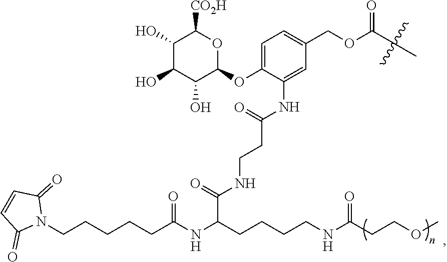

wherein n is an integer ranging from 6 to 72, 8 to 72, 12 to 72, 10 to 72, 12 to 36 or 38, 6 to 24 or 8 to 24; and wherein the wavy line indicates the covalent attachment of the carbonyl carbon atom of the linker to another part of the Brentuximab drug conjugate, in particular to the Brentuximab or antigen binding fragment (Ab) or to the drug moiety (D), preferably to the drug moiety (D). As non-limiting example, in an Brentuximab drug conjugate comprising an MC-G-PEG linker the Brentuximab or antigen binding fragment thereof (Ab) may be covalently attached to the maleimide moiety, and the drug moiety (D) may be covalently attached to the carbonyl carbon atom of the linker as indicated by the wavy line in the above structural formula.

Said maleimidodiaminopropionyl-glucuronide-polyethylene glycol (mDPR-G-PEG) linker has the following structure:

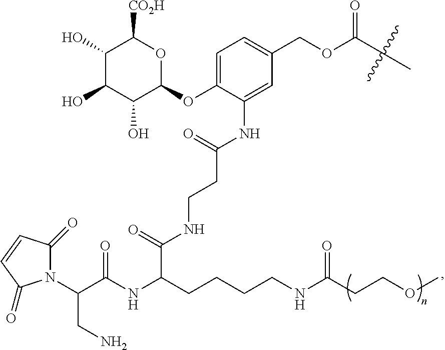

wherein n is an integer ranging from 6 to 72, 8 to 72, 10 to 72, 12 to 72, 12 to 36 or 38, 6 to 24 or 8 to 24; and wherein the wavy line indicates the covalent attachment of the carbonyl carbon atom of the linker to another part of the Brentuximab drug conjugate, in particular to the Brentuximab or antigen binding fragment (Ab) or to the drug moiety (D), preferably to the drug moiety (D). As non-limiting example, in an Brentuximab drug conjugate comprising an mDPR-G-PEG linker the Brentuximab or antigen binding fragment thereof (Ab) may be covalently attached to the maleimide moiety, and the drug moiety (D) may be covalently attached to the carbonyl carbon atom of the linker as indicated by the wavy line in the above structural formula.

Said maleimidopropionyl-glucuronide-polyethylene glycol (MP-G-PEG) linker has the following structure:

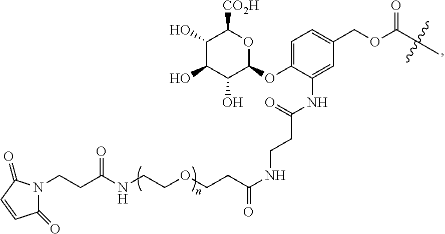

wherein n is an integer ranging from 6 to 72, 8 to 72, 10 to 72, 12 to 72, 12 to 36 or 38, 6 to 24, or 8 to 24; and wherein the wavy line indicates the covalent attachment of the carbonyl carbon atom of the linker to another part of the Brentuximab drug conjugate, in particular to the Brentuximab or antigen binding fragment (Ab) or to the drug moiety (D), preferably to the drug moiety (D). As non-limiting example, in an Brentuximab drug conjugate comprising an MP-G-PEG linker the Brentuximab or antigen binding fragment thereof (Ab) may be covalently attached to the maleimide moiety, and the drug moiety (D) may be covalently attached to the carbonyl carbon atom of the linker as indicated by the wavy line in the above structural formula.

Without wishing to be bound by any theory, release of the drug from an Brentuximab drug conjugate comprising said MC-G, mDPR-G, MC-G-PEG, mDPR-G-PEG or MP-G-PEG linker may initiated by cleavage of the glucuronic acid moiety by a glucuronidase (see, for example, S. C. Jeffrey et al., Bioconjugate Chem. 2006, 17, 831-840).

In some embodiments the linker is a platinum complex linker. For linkers of the platinum complex class see, for example, N. J. Sijbrandi et al, Cancer Res. 2016, 77(2), 257-267, D. Walboer et al., ChemMedChem 2015, 10, 797-803 or US Patent application US 2014/377174, the whole disclosure of these documents incorporated herein by reference.

In some embodiments, said platinum complex linker is an ethylenediamine platinum (II) linker having the following structure:

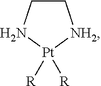

wherein the R independently stands for halogen atoms, for example Cl, Br, F or I, preferably Cl. As non-limiting example, in an Brentuximab drug conjugate comprising a platinum complex linker one Brentuximab or antigen binding fragment thereof (Ab) and one drug moiety (D) may be bound to the platinum complex.

In some embodiments, said platinum complex linker, in particular an ethylenediamine platinum (II) linker, and another part of the Brentuximab drug conjugate, in particular to the Brentuximab or antigen binding fragment (Ab) or to the drug moiety (D), preferably to the drug moiety (D), may be separated by a spacer. Suitable spacers may have different characteristics in length, composition or cleavability. In some embodiments, said spacer is 1-(2-(2-(2-aminoethoxy)ethoxy)ethyl)-3-(piperidin-4-ylmethyl)urea having the following formula:

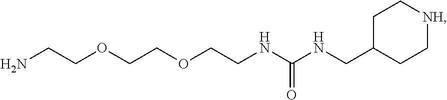 [SMILES: NCCOCCOCCNC(=O)NCC1CCNCC1]

wherein the NH₂ group of the spacer is bound to another part of the Brentuximab drug conjugate, in particular to the Brentuximab or antigen binding fragment (Ab) or to the drug moiety (D), preferably to the drug moiety (D), and the nitrogen atom of the piperidine group is bound to said platinum complex linker. The following structure shows a preferred embodiment of the use of such a spacer:

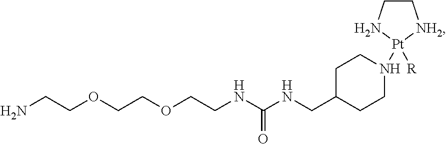

wherein the NH₂ group is bound to another part of the Brentuximab drug conjugate, in particular to the Brentuximab or antigen binding fragment (Ab) or to the drug moiety (D), preferably to the drug moiety (D), and R represents attachment to another part of the Brentuximab drug conjugate, in particular to the Brentuximab or antigen binding fragment (Ab) or to the drug moiety (D), preferably to the Brentuximab or antigen binding fragment (Ab). Preferably, the NH₂ group is covalently attached to the drug moiety (D) and said platinum complex linker is bound to the Brentuximab or antigen binding fragment (Ab) as indicated by R. In some embodiments, the spacer is 4-oxo-4-((piperidin-4-ylmethyl)amino)butanoyl having the following structure:

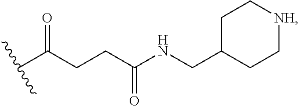 [SMILES: *C(=O)CCC(=O)NCC1CCNCC1]

wherein the wavy line indicates covalent attachment of the carbonyl carbon atom of the spacer to another part of the Brentuximab drug conjugate, in particular to the Brentuximab or antigen binding fragment (Ab) or to the drug moiety (D), preferably to the drug moiety (D), and wherein the nitrogen atom of the piperidine group is bound to said platinum complex linker. The following structure shows a preferred embodiment of the use of such a spacer:

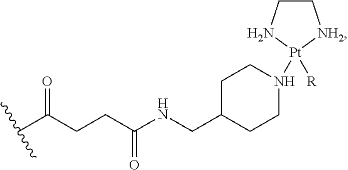

wherein the wavy line indicates the covalent attachment of the carbonyl carbon atom to another part of the Brentuximab drug conjugate, in particular to the Brentuximab or antigen binding fragment (Ab) or to the drug moiety (D), preferably to the drug moiety (D), and R represents attachment to another part of the Brentuximab drug conjugate, in particular the Brentuximab or antigen binding fragment (Ab) or the drug moiety (D), preferably the Brentuximab or antigen binding fragment (Ab). Preferably, the carbonyl carbon is covalently attached to the drug moiety (D) as indicated by the wavy line, and the platinum complex linker is bound to the Brentuximab or antigen binding fragment (Ab) as indicated by R.

In some embodiments the linker is a disulfide bridge replacing linker. The term “disulfide bridge replacing linker” as used throughout this disclosure denotes a linker which is capable of replacing a disulfide bridge resulting from two cysteins of an Brentuximab (Ab). Accordingly, in case such disulfide bridge replacing linker is bound to a drug moiety (D), a drug moiety (D) is introduced in the space between two cysteines of an Brentuximab (Ab). For disulfide bridge replacing linkers see, for example, M. E. B. Smith et al., J. Am. Chem. Soc. 2010, 132, 1960-1965, and international patent application WO 2013/173393, the whole disclosure of these documents incorporated herein by reference.

In some embodiments, said disulfide bridge replacing linker may be a maleimide linker having the following structure:

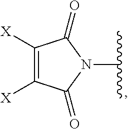

wherein X stands for a halogen atom, for example Cl, Br, F or I, preferably Cl or Br, more preferably Br, and wherein the wavy line indicates covalent attachment of the nitrogen atom to a drug moiety (D). For such linker see M. E. B. Smith et al., J. Am. Chem. Soc. 2010, 132, 1960-1965. A preferred embodiment is the dibromomaleimide having the following structure:

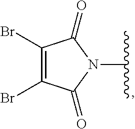 [SMILES: *N1C(=O)C(Br)=C(Br)C1=O]

wherein the wavy line indicates covalent attachment of a drug moiety (D) to the maleimide moiety. One or both bromine atoms may react with a thiol group, which is obtained by way of reduction from a disulfide bridge of an Brentuximab or antigen binding fragment (Ab), as, for example, shown in the following reaction scheme:

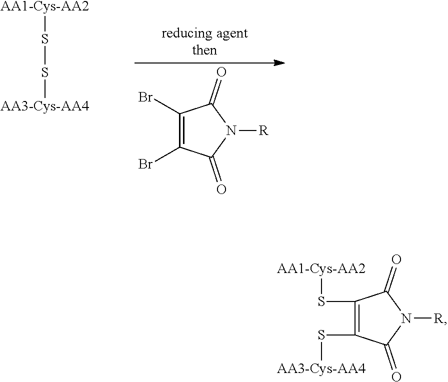

wherein AA1, AA2, AA3 and AA4 comprise peptide chains of the Brentuximab or antigen binding fragment (Ab). AA1, AA2, AA3 and AA4 may be part of a single peptide chain, or may not be part of a single peptide chain. In a preferred embodiment, the drug moiety is covalently attached to the maleimide moiety as indicated by R. Before reacting with the dibromomaleimide the disulfide bridges of the Brentuximab or antigen binding fragment (Ab) are reduced with a reducing agent to obtain free thiol groups. Suitable reducing agents comprise, for example, dithiothreitol (DTT), sodium dithionite, sodium thiosulfate, sodium sulfite or tris(2-carboxyethyl)phosphine (TCEP), with tris(2-carboxyethyl)phosphine (TCEP) being preferred. After the reduction step the dibromomaleimide is added to the Brentuximab (Ab), and said dibromomaleimide reacts with the free thiol groups to form a bridge between the two thiol groups of cysteines of said peptide chain or chains.

In some embodiments the disulfide bridge replacing linker comprises a fragment selected from the group consisting of:

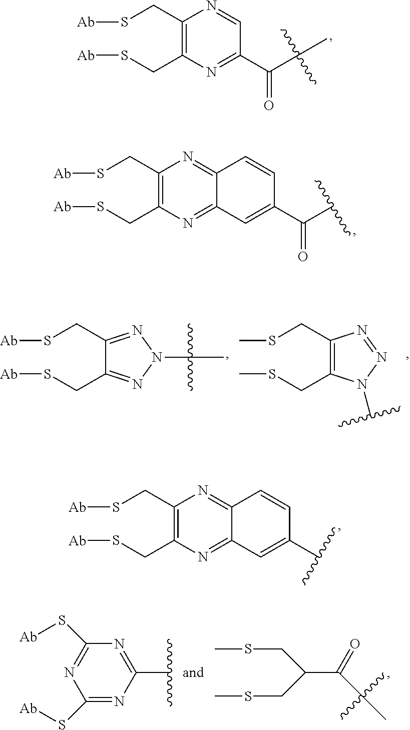

wherein Ab indicates attachment to a peptide chain of an Brentuximab or antigen binding fragment, and the wavy line indicates attachment to a drug moiety (D) of an Brentuximab drug conjugate. For such linkers see WO 2013/173393. Accordingly, in some embodiments the disulfide bridge replacing linker comprises a fragment

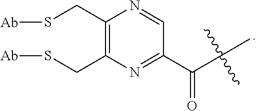

In some embodiments the disulfide bridge replacing linker comprises a fragment

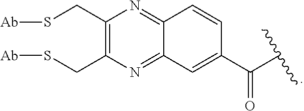

In some embodiments the disulfide bridge replacing linker comprises a fragment

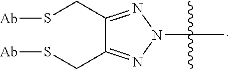

In some embodiments the disulfide bridge replacing linker comprises a fragment

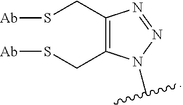

In some embodiments the disulfide bridge replacing linker comprises a fragment

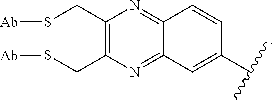

In some embodiments the disulfide bridge replacing linker comprises a fragment

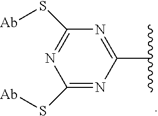

In some embodiments the disulfide bridge replacing linker comprises a fragment

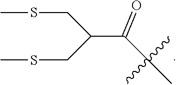 [SMILES: *C(=O)C(CSC)CSC ; "."]

Regarding the attachment of the drug moiety (D) indicated by the wavy line, in these embodiments the drug may be attached to the fragment through an optional spacer. Such spacer may be, for example, derived from an ethylene glycol oligomer, such as, for example,

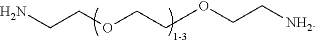

In some embodiments the linker is a glycolinker. The term “glycolinker” as used throughout this disclosure in general denotes a linker comprising a monosaccharide or oligosaccharide fragment. Glycolinkers are described for example, in F. S. Ekholm et al., ChemMedChem 2016, 11, 2501-2505, US patent application US 2016/0820797, US patent application US 2016/0257764, international patent application WO 2016/053107, US patent application US 2016/0106860 and international patent application WO 2016/001485, the whole disclosure of these documents incorporated herein by reference.

Accordingly, a glycolinker as used herein may comprise a fragment —G—, wherein G is a monosaccharide, or a linear or branched oligosaccharide comprising 2 to 20, preferably 2 to 12, more preferably 2 to 10, even more preferably 2 to 8, and most preferably 2 to 6 sugar moieties. Sugar moieties that may be present in a fragment —G— are known to a person skilled in the art, and include e.g. glucose (Glc), galactose (Gal), mannose (Man), fucose (Fuc), N-acetylglucosamine (GlcNAc), N-acetylgalactosamine (GalNAc), N-acetyl-neuraminic acid (NeuNAc) or sialic acid, xylose (Xyl).

In some embodiments a glycolinker may comprise the fragment

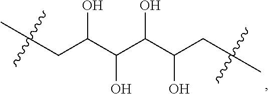 [SMILES: *CC(O)C(O)C(O)C(O)C* ; ","]

wherein the wavy lines indicate attachment to an Brentuximab or antigen binding fragment (Ab) and to a drug moiety (D) of an Brentuximab drug conjugate. For example, in some embodiments a glycolinker from galactose may be used which comprises the fragment

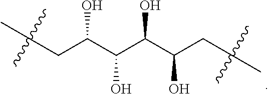 [SMILES: *C[C@@H](O)[C@H](O)[C@H](O)[C@@H](O)C* ; "."]

In some embodiments the fragment may be attached to the Brentuximab or antigen binding fragment (Ab) and/or to the drug moiety (D) through an optional spacer. For example, a glycolinker comprising a monosaccharide fragment and a spacer may have the following structure:

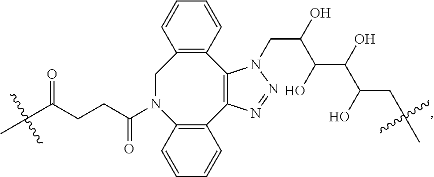 [SMILES: *CC(O)C(O)C(O)C(O)Cn1nnc2c1-c1ccccc1CN(C(=O)CCC(*)=O)c1ccccc1-2 ; ","]

wherein the wavy lines indicate attachment to an Brentuximab or antigen binding fragment (Ab) and a drug moiety (D) of an Brentuximab drug conjugate. For example, the Brentuximab or antigen binding fragment (Ab) may be attached to the glycolinker at the carbonyl carbon atom, and the drug moiety (D) may be attached to the monosaccharide fragment, thus resulting in the following structure:

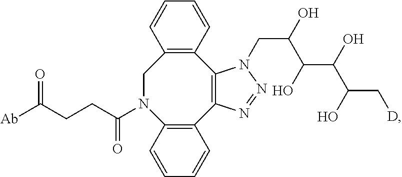

wherein Ab is the Brentuximab or antigen binding fragment and D is the drug moiety. In some embodiments, in case the monosaccharide fragment is galactose, the glycolinker may have the following structure:

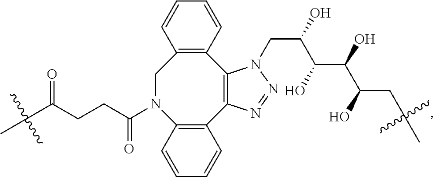 [SMILES: *C[C@@H](O)[C@H](O)[C@H](O)[C@@H](O)Cn1nnc2c1-c1ccccc1CN(C(=O)CCC(*)=O)c1ccccc1-2 ; ","]

wherein the wavy lines indicate attachment to an Brentuximab or antigen binding fragment (Ab) and a drug moiety (D) of an Brentuximab drug conjugate. For example, the Brentuximab or antigen binding fragment (Ab) may be attached to the glycolinker at the carbonyl carbon atom, and the drug moiety (D) may be attached to the monosaccharide fragment, thus resulting in the following structure:

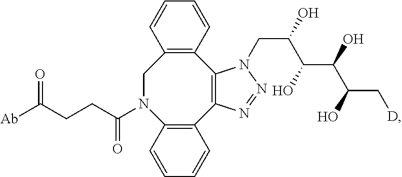

wherein Ab is the Brentuximab or antigen binding fragment (Ab) and D is the drug moiety. The glycolinkers of these embodiments are described, for example, in F. S. Ekholm et al., ChemMedChem 2016, 11, 2501-2505.

In some embodiments the glycolinker has the structure

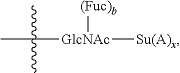

wherein the wavy line indicates attachment to an Brentuximab or antigen binding fragment (Ab), wherein Su(A)ₓ is a sugar derivative Su comprising x functional groups A, wherein A is independently selected from the group consisting of a thiol group or a precursor thereof, a halogen, a sulfonyloxy group, a halogenated acetamido group, a mercaptoacetamido group and a sulfonated hydroxyacetamido group, wherein x is 1, 2, 3 or 4, and wherein b is 0 or 1. Sugar derivative Su(A)ₓ is derived from a sugar or a sugar derivative Su, e.g. an amino sugar or an otherwise derivatized sugar. Examples of sugars and sugar derivatives include galactose (Gal), mannose (Man), glucose (Glc), N-acetylneuraminic acid or sialic acid (Sial) and fucose (Fuc). Sugar derivative Su(A)ₓ is preferably derived from galactose (Gal), mannose (Man), N-actylglucosamine (GlcNAc), fucose (Fuc) and N-acetylneuraminic acid (sialic acid Sia or NeuNAc), preferably from the group consisting of GlcNAc, Glc, Gal, and GalNAc. More preferably Su(A)ₓ is derived from Gal or GalNac, and most preferably Su(A)ₓ is derived from GalNAc. A drug moiety (D) of the Brentuximab drug conjugate may be attached to the glycolinker by way of reaction with the functional group A. The drug moiety may be attached to the glycolinker via an optional spacer, such as, for example, a spacer comprising a maleimide moiety. The glycolinkers of these embodiments are described, for example, in US patent application US 2016/0280797.

In some embodiments the glycolinker has the structure

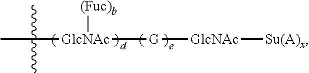

wherein the wavy line indicates attachment to an Brentuximab or antigen binding fragment (Ab), wherein Su(A)ₓ is a sugar derivative Su comprising x functional groups A, wherein A is independently selected from the group consisting of a thiol group or a precursor thereof, a halogen, a sulfonyloxy group, a halogenated acetamido group, a mercaptoacetamido group and a sulfonated hydroxyacetamido group, wherein x is 1, 2, 3 or 4, wherein b is 0 or 1, wherein d is 0 or 1, wherein e is 0 or 1, and wherein G is a monosaccharide, or a linear or branched oligosaccharide comprising 2 to 20 sugar moieties. Sugar derivative Su(A)ₓ is derived from a sugar or a sugar derivative Su, e.g. an amino sugar or an otherwise derivatized sugar. Examples of sugars and sugar derivatives include galactose (Gal), mannose (Man), glucose (Glc), N-acetylneuraminic acid or sialic acid (Sial) and fucose (Fuc). Sugar derivative Su(A)ₓ is preferably derived from galactose (Gal), mannose (Man), N-actylglucosamine (GlcNAc), fucose (Fuc) and N-acetylneuraminic acid (sialic acid Sia or NeuNAc), preferably from the group consisting of GlcNAc, Glc, Gal, and GalNAc. More preferably Su(A)ₓ is derived from Gal or GalNac, and most preferably Su(A)ₓ is derived from GalNAc. G represents a monosaccharide, or a linear or branched oligosaccharide comprising 2 to 20, preferably 2 to 12, more preferably 2 to 10, even more preferably 2 to 8 and most preferably 2 to 6 sugar moieties. Sugar moieties that may be present in fragment G are known to a person skilled in the art and include e.g. glucose (Glc), galactose (Gal), mannose (Man), fucose (Fuc), N-acetylglucosamine (GlcNAc), N-acetylgalactoseamine (GalNAc), N-acetyl-neuramininc acid (NeuNAc) or sialic acid, xylose (Xyl). A drug moiety (D) of the Brentuximab drug conjugate may be attached to the glycolinker by way of reaction with the functional group A. The drug moiety (D) may be attached to the glycolinker via an optional spacer, such as, for example, a spacer comprising a maleimide moiety. The glycolinkers of these embodiments are described, for example, in US patent application US 2016/0280797.

In some embodiments the linker is a methylene alkoxy carbamate linker. For methylene alkoxy carbamate linkers see, for example, R. V. Kolakowski et al., Angew. Chem. Int. Ed. 2016, 55, 7948-7951 or international patent application WO 2015/095755, the whole disclosure of these documents incorporated herein by reference.

In some embodiments, said methylene alkoxy carbamate linker has the following structure:

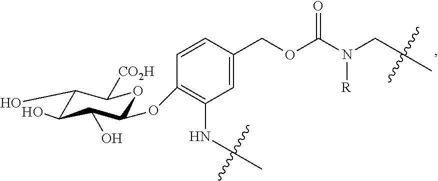

wherein the wavy lines indicate attachment to an Brentuximab or antigen binding fragment (Ab) and a drug moiety (D) of an Brentuximab drug conjugate, and the R group is selected from the group consisting of C1-C4 alkyl,

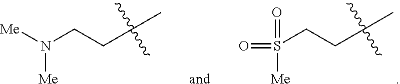 [SMILES: *CCN(C)C ; "and" ; *CCS(C)(=O)=O ; "."]

Preferably, R is C1-C4 alkyl, such as ethyl, or

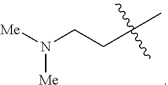 [SMILES: *CCN(C)C ; "."]

More preferably, R is

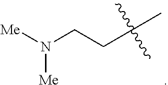 [SMILES: *CCN(C)C ; "."]

In some embodiments, the Brentuximab or antigen binding fragment (Ab) and the drug moiety (D) are attached to the methylene alkoxy carbamate linker as shown in the following structure:

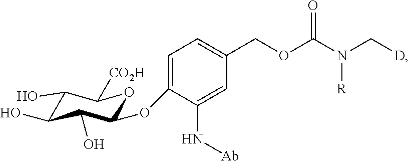

wherein Ab is the Brentuximab or antigen binding fragment and D is the drug moiety. In these embodiments the R group is selected from the group consisting of C1-C4 alkyl,

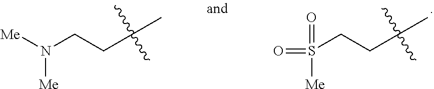 [SMILES: *CCN(C)C ; *CCS(C)(=O)=O]

Preferably, R is C1-C4 alkyl, such as ethyl, or

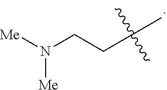 [SMILES: *CCN(C)C]

More preferably, R is

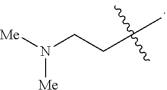 [SMILES: *CCN(C)C]

In some embodiments the Brentuximab or antigen binding fragment (Ab) and/or the drug moiety (D) are attached to the methylene alkoxy carbamate linker through an optional spacer. Such spacer may, for example, have the following structure:

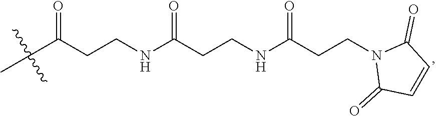 [SMILES: *C(=O)CCNC(=O)CCNC(=O)CCN1C(=O)C=CC1=O]

wherein the wavy line indicates attachment to the methylene alkoxy carbamate linker. This results in the following structure:

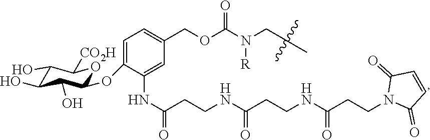

wherein the wavy line indicates covalent attachment of the linker to another part of the Brentuximab drug conjugate, in particular to the Brentuximab or antigen binding fragment (Ab) or to the drug moiety (D), preferably to the drug moiety (D). As non-limiting example, in an Brentuximab drug conjugate comprising such methylene alkoxy carbamate linker the Brentuximab or antigen binding fragment thereof (Ab) may be attached to the maleimide moiety, and the drug moiety (D) may be attached to the position indicated by the wavy line. In these embodiments the R group is selected from the group consisting of C1-C4 alkyl,

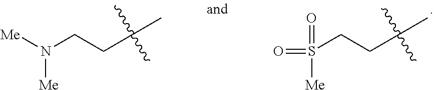 [SMILES: *CCN(C)C ; *CCS(C)(=O)=O]

Preferably, R is C1-C4 alkyl, such as ethyl, or

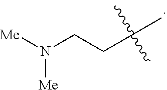 [SMILES: *CCN(C)C]

More preferably, R is

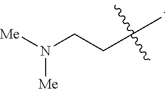 [SMILES: *CCN(C)C]

In some embodiments, the linker (L) is selected from -(Succinimid-3-yl-N)—CH₂CH₂—C(═O)—GGFG—NH—CH₂CH₂CH₂—C(═O)—, -(Succinimid-3-yl-N)—CH₂CH₂CH₂CH₂CH₂—C(═O)—GGFG—NH—CH₂CH₂CH₂—C(═O)—, -(Succinimid-3-yl-N)—CH₂CH₂CH₂CH₂CH₂—C(═O)—GGFG—NH—CH₂—O—CH₂—C(═O)—, -(Succinimid-3-yl-N)—CH₂CH₂CH₂CH₂CH₂—C(═O)—GGFG—NH—CH₂CH₂—O—CH₂—C(═O)—, -(Succinimid-3-yl-N)—CH₂CH₂—C(═O)—NH—CH₂CH₂O—CH₂CH₂O—CH₂CH₂—C(═O)—GGFG—NH—CH₂CH₂CH₂—C(═O)—, and -(Succinimid-3-yl-N)—CH₂CH₂—C(═O)—NH—CH₂CH₂O—CH₂CH₂O—CH₂CH₂O—CH₂CH₂O—CH₂CH₂—C(═O)—GGFG—NH—CH₂CH₂CH₂—C(═O)—, preferably -(Succinimid-3-yl-N)—CH₂CH₂CH₂CH₂CH₂—C(═O)—GGFG—NH—CH₂—O—CH₂—C(═O)—.

### “D”: Drug Moieties

The present disclosure provides antibody drug conjugates comprising a drug moiety. The term “drug moiety” or “payload”, both of which can be used interchangeably, as used herein refers to a chemical or biochemical moiety that is conjugated to an antibody or antigen binding fragment. In this regard, it is again referred to the “ADC formula” described herein Ab-(L-(D)ₓ)_{y}. Brentuximab can be conjugated to several identical or different drug moieties using any methods described herein or known in the art. In some preferred embodiments the drug moiety is an anti-cancer agent. Accordingly, the drug may be selected from the group consisting of camptothecins, Topoisomerase inhibitors, maytansinoids, calicheamycins, duocarmycins, tubulysins, amatoxins, dolastatins and auristatins such as monomethyl auristatin E (MMAE), pyrrolobenzodiazepine dimers, indolino-benzodiazepine dimers, radioisotopes, therapeutic proteins and peptides (or fragments thereof), nucleic acids, PROTACs, kinase inhibitors, MEK inhibitors, KSP inhibitors, and analogues or prodrugs thereof. In a preferred embodiment, the drug moiety is MMAE.

“Camptothecin” (CPT) as used herein relates to a topoisomerase poison. It was discovered in 1966 by M. E. Wall and M. C. Wani in systematic screening of natural products for anticancer drugs. It was isolated from the bark and stem of Camptotheca acuminata (Camptotheca, Happy tree), a tree native to China used as a cancer treatment in Traditional Chinese Medicine. The term Campthothecin may also comprise CPT analogoues. Four CPT analogues have been approved and are used in cancer chemotherapy today, topotecan, irinotecan, belotecan, and trastuzumab deruxtecan. Camptothecin has the following structure:

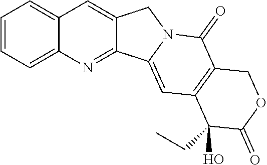 [SMILES: CC[C@@]1(O)C(=O)OCc2c1cc1n(c2=O)Cc2cc3ccccc3nc2-1]

The following analogues of CPT are also envisioned by the term CPT:

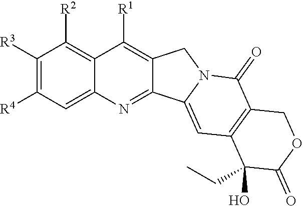

| Analogue | R¹ | R² | R³ | R⁴ |
|---|---|---|---|---|
| Topotecan | 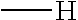 | 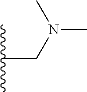 [SMILES: *CN(C)C] | 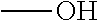 | 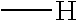 |
| Irinotecan (CPT-11) | 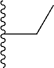 [SMILES: *CC] | 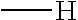 | 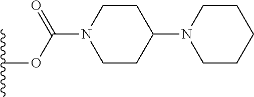 [SMILES: *OC(=O)N1CCC(N2CCCCC2)CC1] | 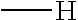 |
| Silatecan (DB-67, AR-67) | 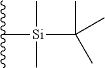 [SMILES: *[Si](C)(C)C(C)(C)C] | 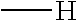 | 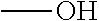 | 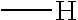 |
| Cositecan (BNP-1350) | 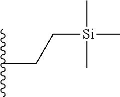 [SMILES: *CC[Si](C)(C)C] | 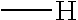 | 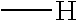 | 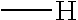 |
| Exatecan | 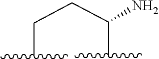 [SMILES: *CC[C@@H](*)N] | | 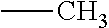 | 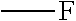 |
| Lurtotecan | 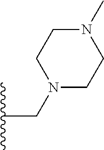 [SMILES: *CN1CCN(C)CC1] | 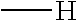 | 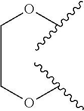 [SMILES: *OCCO*] | |
| Gimatecan (ST1481) | 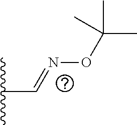 [SMILES: */C=N/OC(C)(C)C] | 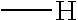 | 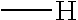 | 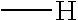 |
| Belotecan (CKD-602) | 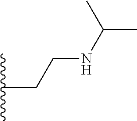 [SMILES: *CCNC(C)C] | 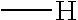 | 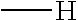 | 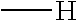 |
| Rubitecan | 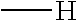 | 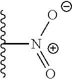 [SMILES: *[N+](=O)[O-]] | 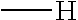 | 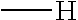 |

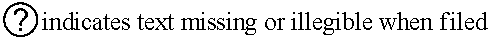

In some embodiments the drug moiety is a maytansinoid drug moiety, including those having the structure:

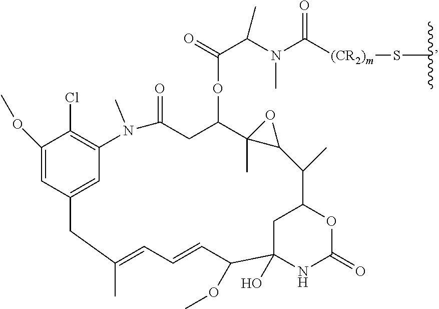

where the wavy line indicates the covalent attachment of the sulfur atom of the maytansinoid to a linker of an antibody drug conjugate. R at each occurrence is independently H or a C1-C6 alkyl. The alkylene chain attaching the amide group to the sulfur atom may be methanyl, ethanyl, or propanyl, i.e. m is 1, 2, or 3. (U.S. Pat. No. 633,410, U.S. Pat. No. 5,208,020, Chari et al. (1992) Cancer Res. 52; 127-131, Lui et al. (1996) Proc. Natl. Acad. Sci. 93:8618-8623).

All stereoisomers of the maytansinoid drug moiety are contemplated for the antibody drug conjugates disclosed herein, i.e. any combination of R and S configurations at the chiral carbons of the maytansinoid. In some embodiments the maytansinoid drug moiety has the following stereochemistry:

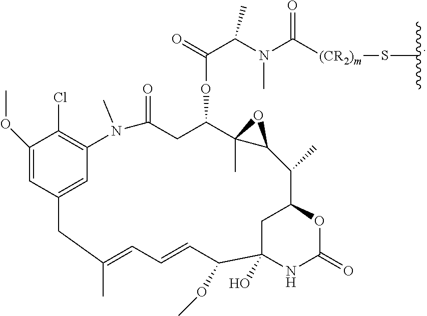

In some embodiments the maytansinoid drug moiety is N^{2′}-deacetyl-N^{2′}-(3-mercapto-1-oxopropyl)-maytansine (also known as DM1). DM1 is represented by the following structural structure:

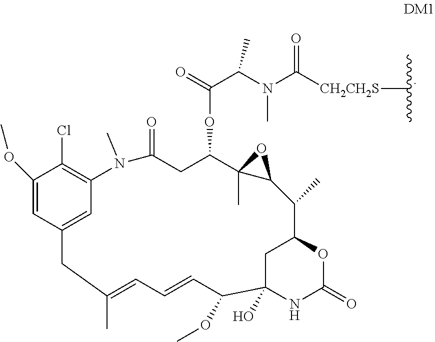

In some embodiments the maytansinoid drug moiety is N^{2′}-deacetyl-N^{2′}-(4-mercapto-1-oxopentyl)-maytansine (also known as DM3). DM3 is represented by the following structural structure:

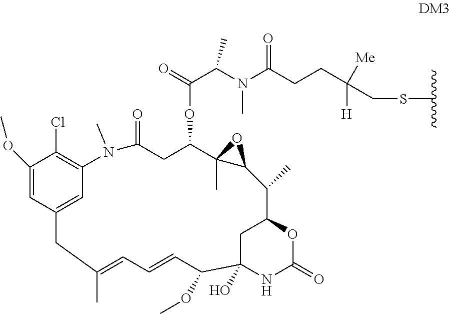 [SMILES: *SCC(C)CCC(=O)N(C)[C@@H](C)C(=O)O[C@H]1CC(=O)N(C)c2cc(cc(OC)c2Cl)C/C(C)=C/C=C/[C@@H](OC)[C@@]2(O)C[C@H](OC(=O)N2)[C@@H](C)[C@@H]2O[C@@]12C]

In some embodiments the maytansinoid drug moiety is N^{2′}-deacetyl-N^{2′}-(4-methyl-4-mercapto-1-oxopentyl)-maytansine (also known as DM4). DM4 is represented by the following structural structure:

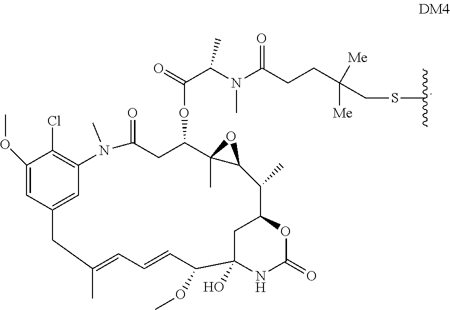 [SMILES: *SCC(C)(C)CCC(=O)N(C)[C@@H](C)C(=O)O[C@H]1CC(=O)N(C)c2cc(cc(OC)c2Cl)C/C(C)=C/C=C/[C@@H](OC)[C@@]2(O)C[C@H](OC(=O)N2)[C@@H](C)[C@@H]2O[C@@]12C]

Preferably, in antibody drug conjugates disclosed herein comprising a maytansinoid drug moiety the maytansinoid is N^{2′}-deacetyl-N^{2′}-(3-mercapto-1-oxopropyl)-maytansine (DM1) or N^{2′}′-deacetyl-N^{2′}-(4-mercapto-4-methyl-1-oxopentyl)-maytansine (DM4).

The drug moiety may be a calicheamicin. “Calicheamicins” as used herein relate to a class of enediyne antitumor antibiotics derived from the bacterium Micromonospora echinospora, with calicheamicin γ1 being the most notable. It was isolated originally in the mid-1980s from the chalky soil, or “caliche pits”, located in Kerrville, Tex. It is extremely toxic to all cells. Accordingly, the drug moiety may be Calicheamicin γ1 exemplified by the following structure:

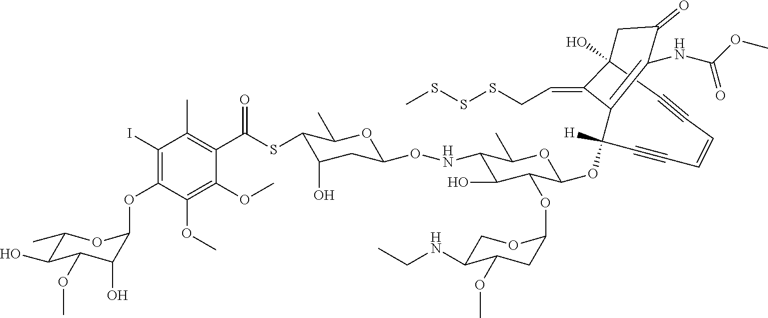 [SMILES: CCNC1COC(OC2C(O[C@H]3C#C/C=C\C#C[C@]4(O)CC(=O)C(NC(=O)OC)=C3/C4=C\CSSSC)OC(C)C(NOC3CC(O)C(SC(=O)c4c(C)c(I)c(OC5OC(C)C(O)C(OC)C5O)c(OC)c4OC)C(C)O3)C2O)CC1OC]

The drug moiety may be a duocarmycin. A “duocarmycin” as used herein describes a small-molecule, synthetic, DNA minor groove binding alkylating agent. Duocarmycins may be suitable to target solid tumors. They bind to the minor groove of DNA and alkylate the nucleobase adenine at the N3 position. The irreversible alkylation of DNA disrupts the nucleic acid architecture, which eventually leads to tumor cell death. Examples of duocarmycins include, but are not limited to, CC-1065, daunorubicin, mitomycin C, bleomycin, cyclocytidine, vincristine, vinblastine, methotrexate and Taxol and derivatives thereof.

The drug moiety may be a platinum-based antitumor agent such as cisplatin or derivatives thereof.

The drug moiety may be a tubulysin. Tubulysins have functions as being anti-microtubule, anti-mitotic, apoptosis inducer, anticancer, anti-angiogenic, and antiproliferative. Tubulysins are cytotoxic peptides, which include 9 members (A-I). Preferably, the tubulysin is Tubulysin A. Tubulysin A has potential application as an anticancer agent. It arrests cells in the G2/M phase. Tubulysin A has the following structure:

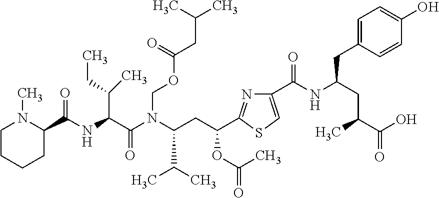 [SMILES: CC[C@H](C)[C@H](NC(=O)[C@H]1CCCCN1C)C(=O)N(COC(=O)CC(C)C)[C@H](C[C@@H](OC(C)=O)c1nc(C(=O)N[C@@H](Cc2ccc(O)cc2)C[C@H](C)C(=O)O)cs1)C(C)C]

The drug moiety may be an amatoxin. Amatoxin is the collective name of a subgroup of at least eight related toxic compounds found in several genera of poisonous mushrooms, most notably the death cap (Amanita phalloides) and several other members of the genus Amanita, as well as some Conocybe, Galerina and Lepiota mushroom species. Amatoxins are lethal in even small doses. The compounds have a similar structure, that of eight amino-acid residues arranged in a conserved macrobicyclic motif (an overall pentacyclic structure when counting the rings inherent in the proline and tryptophan-derived residues). All amatoxins are oligopeptides that are synthesized as 35-amino-acid proproteins, from which the final eight amino acids are cleaved by a prolyl oligopeptidase. The schematic amino acid sequence of amatoxins is Ile-Trp-Gly-Ile-Gly-Cys-Asn-Pro with cross-linking between Trp and Cys via the sulfoxide (S═O) moiety and hydroxylation in variants of the molecule. There are currently ten known amatoxins, which might be the drug moiety:

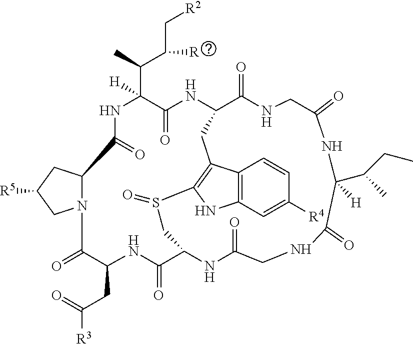

| Name | R¹ | R² | R³ | R⁴ | R⁵ |
|---|---|---|---|---|---|
| α-Amanitin | OH | OH | NH₂ | OH | OH |
| β-Amanitin | OH | OH | OH | OH | OH |
| γ-Amanitin | OH | H | NH₂ | OH | OH |
| ε-Amanitin | OH | H | OH | OH | OH |
| Amanullin | H | H | NH₂ | OH | OH |
| Amanullinic acid | H | H | OH | OH | OH |
| Amaninamide | OH | OH | NH₂ | H | OH |
| Amanin | OH | OH | OH | H | OH |
| Proamanullin | H | H | NH₂ | OH | H |

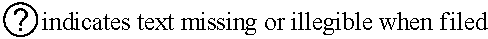

The drug moiety may be a dolastatin such as Dolastatin 10 or dolastatin 15. Both are marine natural products isolated from the Indian Ocean sea hare Dollabella auricularia. This potent antitumor agent is also isolated from the marine cyanobacterium Symploca sp. VP642 from Palau. Being a small linear peptide molecules, dolastatin 10 and 15 are considered anti-cancer drugs showing potency against breast and liver cancers, solid tumors and some leukemias. Preclinical research indicated potency in experimental antineoplastic and tubulin assembly systems. The dolastatins are mitotic inhibitors. They inhibit microtubule assembly by interfering with tubulin formation and thereby disrupt cell division by mitosis and induces apoptosis and Bcl-2 phosphorylation in several malignant cell types. Dolostatin 10 (N,N-Dimethyl-L-valyl-N-[(3R,4S,5S)-3-methoxy-1-{(2S)-2-[(1R,2R)-1-methoxy-2-methyl-3-oxo-3-{[(1S)-2-phenyl-1-(1,3-thiazol-2-yl)ethyl]amino}propyl]-1-pyrrolidinyl}-5-methyl-1-oxo-4-heptanyl]-N-methyl-L-valinamide) has the following structure:

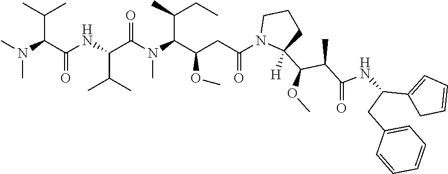 [SMILES: CC[C@H](C)[C@@H]([C@@H](CC(=O)N1CCC[C@@H]1[C@H](OC)[C@@H](C)C(=O)N[C@@H](Cc1ccccc1)C1=CC=CC1)OC)N(C)C(=O)[C@@H](NC(=O)[C@H](C(C)C)N(C)C)C(C)C]

Dolastatin 15 ((2S)-1-[(2S)-2-Benzyl-3-methoxy-5-oxo-2,5-dihydro-1H-pyrrol-1-yl]-3-methyl-1-oxo-2-butanyl N,N-dimethyl-L-valyl-L-valyl-N-methyl-L-valyl-L-prolyl-L-prol) has the following structure:

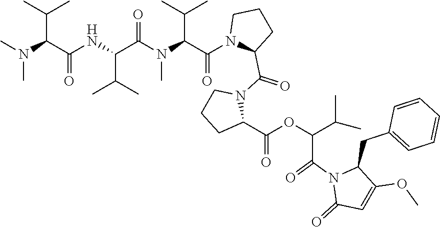 [SMILES: COC1=CC(=O)N(C(=O)C(OC(=O)[C@@H]2CCCN2C(=O)[C@@H]2CCCN2C(=O)[C@H](C(C)C)N(C)C(=O)[C@@H](NC(=O)[C@H](C(C)C)N(C)C)C(C)C)C(C)C)[C@H]1Cc1ccccc1]

In some embodiments of the antibody drug conjugates disclosed herein the drug moiety is an auristatin. Preferably, the auristatin is monomethyl auristatin F (MMAF) or monomethyl auristatin E (MMAE).

In some embodiments of the antibody drug conjugates described herein the drug moiety is monomethyl auristatin F (also known as MMAF). MMAF is represented by the following structural formula:

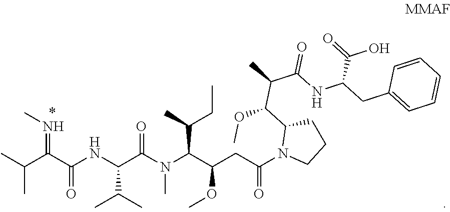 [SMILES: CC[C@H](C)[C@@H]([C@@H](CC(=O)N1CCC[C@H]1[C@H](OC)[C@@H](C)C(=O)N[C@@H](Cc1ccccc1)C(=O)O)OC)N(C)C(=O)[C@@H](NC(=O)/C(=N/C)C(C)C)C(C)C]

Monomethyl auristatin F (MMAF) may be bound to the linker via the nitrogen atom marked with an asterisk (*).

In some embodiments the auristatin drug moiety is monomethyl auristatin E (also known as MMAE). MMAE is represented by the following structural formula:

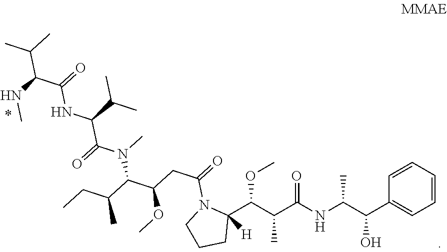 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@@H](NC)C(C)C)C(C)C]

Monomethyl auristatin E (MMAE) may be bound to the linker via the nitrogen atom marked with an asterisk (*).

These molecules noncompetitively inhibits binding of vincristine to tubulin (at a location known as the vinca/peptide region) but have been shown to bind to the RZX/MAY region.

The drug moiety may be a Pyrrolobenzodiazepine Dimer such as a compound having the following structure:

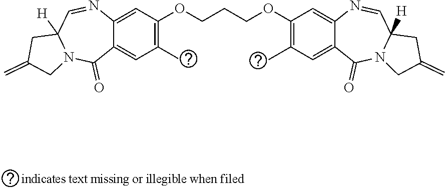

The drug moiety may be a Indolinobenzodiazepin Dimer such as a compound having the following structure:

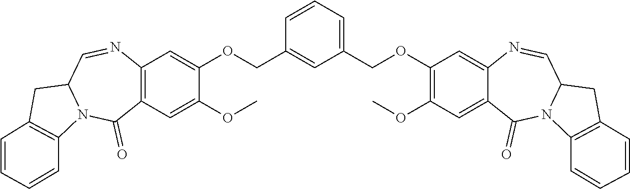 [SMILES: COc1cc2c(cc1OCc1cccc(COc3cc4c(cc3OC)C(=O)N3c5ccccc5CC3C=N4)c1)N=CC1Cc3ccccc3N1C2=O]

The drug moiety may be a radioisotope. Typical radioisotopes as described herein may relate to a small radiation source, usually a gamma or beta emitter such as iodine-125, iodine-131, iridium-192 or palladium-103.

The drug moiety may be a therapeutic protein or peptide or a fragment thereof. Typical examples are cytokines such as interleukines, ricin, diphtheria toxin, Pseudomonas exotoxin PE38.

The drug moiety may be a kinase inhibitor, preferably an inhibitor of a kinase associated with a pro-tumorigenic function. Exemplary kinase inhibitors include imatinib, nilotinib, dasatinib, bosutinib, ponatinib, gefitinib, erlotinib, afatinib, osimertinib, lapatinib, neratinib, sorafenib, sunitinib, pazopanib, axitinib, lenvatinib, cabozatinib, vandetanib, regorafenib, vemurafenib, dabrafenib, trametinib, cobimetinib, crizotinib, certinib, alectinib, brigatinib, lorlatinib, ibrutinib, acalibrutinib, midostaurin, ruxolitinib, idelalisib, copanlisib, palbociclib, ribociclib or abemaciclib.

The drug moiety may be a MEK inhibitor. A MEK inhibitor as used herein describes a chemical or drug that inhibits the mitogen-activated protein kinase kinase enzymes MEK1 and/or MEK2. They can be used to affect the MAPK/ERK pathway which is often overactive in some cancers. Hence MEK inhibitors have potential for treatment of some cancers, especially BRAF-mutated melanoma, and KRAS/BRAF mutated colorectal cancer. Typical MEK inhibitors include Trametinib (GSK1120212), Cobimetinib or XL518, Binimetinib (MEK162), Selumetinib, PD-325901, CI-1040, PD035901, or TAK-733.

The drug moiety may be a KSP (kinesin spindle protein) inhibitor. Examples of KSP inhibitors include Ispinesib (SB-715992), SB743921, AZ 3146, GSK923295, BAY 1217389, MPI-0479605 and ARQ 621.

The drug moiety may be a nucleic acid. A nucleic acid when used as a drug moiety may relate to a DNA/RNA molecule, e.g. an DNA/RNA molecule having an immune modulating function (preferably on innate immunity). Immune modulation can refer to increasing or decreasing an immune response, preferably decreasing. The DNA/RNA molecule may also be an siRNA, preferably designed to (specifically) modulate/manipulate (e.g. reduce) target protein expression (an exemplarily target can be dystrophia myotonica protein kinase (DMPK)).

The drug moiety may be a PROTAC. A proteolysis targeting chimera (PROTAC) is a heterobifunctional small molecule comprising two active domains and a linker capable of removing specific unwanted proteins. Rather than acting as a conventional enzyme inhibitor, a PROTAC preferably works by inducing selective intracellular proteolysis. PROTACs generally comprise two covalently linked protein-binding molecules: one capable of engaging an E3 ubiquitin ligase, and another that binds to a target protein meant for degradation. Recruitment of the E3 ligase to the target protein results in ubiquitination and subsequent degradation of the target protein by the proteasome. Exemplary PROTACS are described in Sakamoto et al. (2001), PNAS, 98(15):8554-9, hereby incorporated by reference.

### Methods of Producing ADC

Methods of producing the ADCs of the invention are known in the art. In particular, the introduction or addition of a recognition sequence for tubulin tyrosine ligase at the C-terminus of a polypeptide and contacting the polypeptide (here Brentuximab) with the non-natural amino acid in the presence of tubulin tyrosine ligase and the conjugation of the linker comprising a drug moiety to said ligated Brentuximab has already been described in WO 2016/066749, WO 2017/186855, Schumacher et al., Angew. Chem. Int. Ed. 2015, 54, 13787-13791, all of which are hereby incorporated by reference. The Materials and Methods section of the Examples also comprises guidance on how to produce or obtain the ADC of the invention.

Accordingly, the present invention further relates to a method of producing an ADC as defined herein, comprising

(a) introducing or adding at the C-terminus of the light chain, the heavy chain or both the light chain and the heavy chain of Brentuximab a recognition sequence for tubulin tyrosine ligase;
(b) contacting the Brentuximab obtained in step (a) in the presence of tubulin tyrosine ligase and a non-natural amino acid under conditions suitable for the tubulin tyrosine ligase to ligate said Brentuximab with said non-natural amino acid; and
(c) conjugating an optionally cleavable linker comprising a drug moiety to said ligated Brentuximab obtained in step (b).

The introduction or addition of a recognition sequence for TTL at the C-terminus of Brentuximab is done as described herein. For example, such a recognition sequence may be introduced or added by genetic engineering or by synthesis, either chemical protein synthesis or via synthetic biology.

The present invention further relates to an ADC obtainable by the method of producing an ADC as defined herein. The present invention further relates to an ADC obtained by the method of producing an ADC as defined herein.

### Pharmaceutical Compositions

The present invention further relates to a pharmaceutical composition comprising the ADC of the invention. A pharmaceutical composition according to the present invention may further comprise one or more pharmaceutically acceptable carriers. In a specific embodiment, the term “pharmaceutically acceptable” means approved by a regulatory agency or other generally recognized pharmacopoeia for use in animals, and more particularly in humans. Pharmaceutically acceptable carriers are well known in the art and include, for example, aqueous solutions such as water, 5% dextrose, or physiologically buffered saline or other solvents or vehicles such as glycols, glycerol, oils such as olive oil, or injectable organic esters that are suitable for administration to a human or non-human subject. Particular exemplary pharmaceutically acceptable carriers include (biodegradable) liposomes; microspheres made of the biodegradable polymer poly(D,L-lactic-coglycolic acid (PLGA), albumin microspheres; synthetic polymers (soluble); nanofibers, protein-DNA complexes; protein conjugates; erythrocytes; or virosomes. Various carrier based dosage forms comprise solid lipid nanoparticles (SLNs), polymeric nanoparticles, ceramic nanoparticles, hydrogel nanoparticles, copolymerized peptide nanoparticles, nanocrystals and nanosuspensions, nanocrystals, nanotubes and nanowires, functionalized nanocarriers, nanospheres, nanocapsules, liposomes, lipid emulsions, lipid microtubules/microcylinders, lipid microbubbles, lipospheres, lipopolyplexes, inverse lipid micelles, dendrimers, ethosomes, multicomposite ultrathin capsules, aquasomes, pharmacosomes, colloidosomes, niosomes, discomes, proniosomes, microspheres, microemulsions and polymeric micelles. Other suitable pharmaceutically acceptable carriers and excipients are inter alia described in Remington's Pharmaceutical Sciences, 15^{th} Ed., Mack Publishing Co., New Jersey (1991). See also, e.g., Remington: The Science and Practice of Pharmacy, 21^{st} edition; Lippincott Williams & Wilkins, 2005.

In some embodiments, a pharmaceutically acceptable carrier or composition is sterile. A pharmaceutical composition can comprise, in addition to the active agent, physiologically acceptable compounds that act, for example, as bulking agents, fillers, solubilizers, stabilizers, osmotic agents, uptake enhancers, etc. Physiologically acceptable compounds include, for example, carbohydrates, such as glucose, sucrose, lactose; dextrans; polyols such as mannitol; antioxidants, such as ascorbic acid or glutathione; preservatives; chelating agents; buffers; or other stabilizers or excipients.

The choice of a pharmaceutically acceptable carrier(s) and/or physiologically acceptable compound(s) can depend for example, on the nature of the active agent, e.g., solubility, compatibility (meaning that the substances can be present together in the composition without interacting in a manner that would substantially reduce the pharmaceutical efficacy of the pharmaceutical composition under ordinary use situations) and/or route of administration of the composition.

Pharmaceutical compositions of the invention comprise a therapeutically effective amount of the ADC described herein and can be structured in various forms, e.g. in solid, liquid, gaseous or lyophilized form and may be, inter alia, in the form of an ointment, a cream, transdermal patches, a gel, powder, a tablet, solution, an aerosol, granules, pills, suspensions, emulsions, capsules, syrups, liquids, elixirs, extracts, tincture or fluid extracts or in a form which is particularly suitable for topical or oral administration. A variety of routes are applicable for administration of the polypeptide of the invention, including, but not limited to, orally, topically, transdermally, subcutaneously, intravenously, intraperitoneally, intramuscularly or intraocularly. However, any other route may readily be chosen by the person skilled in the art if desired.

### Use in Methods of Treatment

As shown in Examples 4 and 5, the ADCs of the present invention can be used for the treatment, in particular the treatment of cancer. Accordingly, the present invention further relates to the ADC of the invention or the pharmaceutical composition of the invention for use in a method of treating a disease, optionally comprising the administration of an effective amount of the ADC of the invention or the pharmaceutical composition of the invention to a subject or patient in need thereof. Preferably, the disease is associated with overexpression of CD30. The disease may be a cancer associated with overexpression of CD30. More preferably, the disease is selected from the group consisting of lymphoma, such as Hodgkin's lymphoma (HL), non-Hodgkin lymphoma (NHL), anaplastic large-cell lymphoma (ALCL), large B-cell lymphoma, paediatric lymphoma, T-cell lymphoma and enteropathy-associated T-cell lymphoma (EATL), leukaemia, such as acute myeloid leukaemia (AML), acute lymphoblastic leukaemia (ALL) and mast cell leukaemia, germ cell cancer, graft-versus-host disease (GvHD) and lupus, in particular systemic lupus erythematosus (SLE), preferably Hodgkin Lymphoma (HL) or anaplastic large cell lymphoma (ALCL). The disease may be selected from the group consisting of peripheral T cell lymphoma—not otherwise specified (PTCL-NOS), angioimmunoblastic T-cell lymphoma (AITL), enteropathy associated T cell lymphoma (EATL), adult T-cell leukemia/lymphoma (ATLL), extranodal natural killer/T-cell lymphoma (ENKTCL), hepatosplenic and intestinal γ/δ-T cell lymphoma, nodal peripheral T-cell lymphoma with TFH phenotype, and follicular T cell lymphoma. The disease may be peripheral T cell lymphoma (PTCL), including anaplastic large cell lymphoma (ALCL). The disease may be cutaneous T cell lymphoma (CTCL), including primary cutaneous anaplastic large cell lymphoma (pcALCL). The disease may be Hodgkin lymphoma (HL).

The present invention also relates to the use of the ADC of the invention for the manufacture of a medicament for treating a disease. The present invention also relates to the use of the pharmaceutical composition of the invention for the manufacture of a medicament for treating a disease. Preferably, the disease is associated with overexpression of CD30. The disease may be a cancer associated with overexpression of CD30. More preferably, the disease is selected from the group consisting of lymphoma, such as Hodgkin's lymphoma (HL), non-Hodgkin lymphoma (NHL), anaplastic large-cell lymphoma (ALCL), large B-cell lymphoma, paediatric lymphoma, T-cell lymphoma and enteropathy-associated T-cell lymphoma (EATL), leukaemia, such as acute myeloid leukaemia (AML), acute lymphoblastic leukaemia (ALL) and mast cell leukaemia, germ cell cancer, graft-versus-host disease (GvHD) and lupus, in particular systemic lupus erythematosus (SLE), preferably Hodgkin Lymphoma (HL) or anaplastic large cell lymphoma (ALCL). The disease may be selected from the group consisting of peripheral T cell lymphoma—not otherwise specified (PTCL-NOS), angioimmunoblastic T-cell lymphoma (AITL), enteropathy associated T cell lymphoma (EATL), adult T-cell leukemia/lymphoma (ATLL), extranodal natural killer/T-cell lymphoma (ENKTCL), hepatosplenic and intestinal γ/γ-T cell lymphoma, nodal peripheral T-cell lymphoma with TFH phenotype, and follicular T cell lymphoma. The disease may be peripheral T cell lymphoma (PTCL), including anaplastic large cell lymphoma (ALCL). The disease may be cutaneous T cell lymphoma (CTCL), including primary cutaneous anaplastic large cell lymphoma (pcALCL). The disease may be Hodgkin lymphoma (HL).

The present invention also relates to a method of treating a disease, comprising the administration of an effective amount of the ADC of the invention to a subject or patient in need thereof. The present invention also relates to a method of treating a disease, comprising the administration of an effective amount of the pharmaceutical composition of the invention to a subject or patient in need thereof. Preferably, the disease is associated with overexpression of CD30. The disease may be a cancer associated with overexpression of CD30. More preferably, the disease is selected from the group consisting of lymphoma, such as Hodgkin's lymphoma (HL), non-Hodgkin lymphoma (NHL), anaplastic large-cell lymphoma (ALCL), large B-cell lymphoma, paediatric lymphoma, T-cell lymphoma and enteropathy-associated T-cell lymphoma (EATL), leukaemia, such as acute myeloid leukaemia (AML), acute lymphoblastic leukaemia (ALL) and mast cell leukaemia, germ cell cancer, graft-versus-host disease (GvHD) and lupus, in particular systemic lupus erythematosus (SLE), preferably Hodgkin Lymphoma (HL) or anaplastic large cell lymphoma (ALCL). The disease may be, bit is not limited to, peripheral T cell lymphoma—not otherwise specified (PTCL-NOS), angioimmunoblastic T-cell lymphoma (AITL), enteropathy associated T cell lymphoma (EATL), adult T-cell leukemia/lymphoma (ATLL), extranodal natural killer/T-cell lymphoma (ENKTCL), hepatosplenic and intestinal γ/δ-T cell lymphoma, nodal peripheral T-cell lymphoma with TFH phenotype, or follicular T cell lymphoma. The disease may also be peripheral T cell lymphoma (PTCL), including anaplastic large cell lymphoma (ALCL) or cutaneous T cell lymphoma (CTCL), including primary cutaneous anaplastic large cell lymphoma (pcALCL). The disease may also be Hodgkin lymphoma (HL).

The phrase “effective amount” refers to an amount of a therapeutic agent (e.g., the ADC of the invention) that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays. The exact amount will depend on the purpose of the treatment, and will be ascertainable by one skilled in the art using known techniques (see, for example, Lloyd (1999) The Art, Science and Technology of Pharmaceutical Compounding).

Further, the present invention relates to an antibody drug conjugate as disclosed herein for use in a method of treating cancer in a patient. The term “patient” means according to the invention a human being, a non-human primate or another animal, in particular a mammal such as a cow, horse, pig, sheep, goat, dog, cat or a rodent such as a mouse and rat. In a particularly preferred embodiment, the patient is a human being. Except when noted, the terms “patient” or “subject” are used herein interchangeably. The term “treatment” in all its grammatical forms includes therapeutic or prophylactic treatment. A “therapeutic or prophylactic treatment” comprises prophylactic treatments aimed at the complete prevention of clinical and/or pathological manifestations or therapeutic treatment aimed at amelioration or remission of clinical and/or pathological manifestations. The term “treatment” thus also includes the amelioration or prevention of diseases.

An ADC of the present invention may be administered at any dose that is therapeutically effective. The upper limit is usually a dose that is still safe to administer in terms of side effects. Typically, an ADC of the present invention may be administered at a(n effective) dose of 0.5-20 mg/kg. An ADC of the present invention may be administered at a(n effective) dose of 1-10 mg/kg. An ADC of the present invention may be administered at a(n effective) dose of 1-9 mg/kg. An ADC of the present invention may be administered at a(n effective) dose of 1.2-9 mg/kg. An ADC of the present invention may be administered at a(n effective) dose of 1.8-8 mg/kg. An ADC of the present invention may be administered at a(n effective) dose of 2-6 mg/kg. As illustrative examples, an ADC of the present invention may be administered at a(n effective) dose of 20 mg/kg, 18 mg/kg, 16 mg/kg, 14 mg/kg, 12 mg/kg, 10 mg/kg, 9 mg/kg, 8 mg/kg, 7 mg/kg, 6 mg/kg, 5 mg/kg, 4 mg/kg, 3 mg/kg, 2 mg/kg or 1 mg/kg. Other doses are possible if considered advantageous or necessary.

The ADC of the invention may be co-administered with other agents, in particular anticancer drugs, or compounds that enhance the effects of such agents. Co-administration comprises sequential and simultaneous administration. Suitable anticancer drugs include, e.g., one or more of the drug moieties described herein. The ADC of the invention may be co-administered with Cyclophosphamide, Doxorubicin and Prednisone (CHP scheme). The ADC of the invention may be co-administered in particular with anticancer drugs like immunomodulatory drugs, checkpoint inhibitors, chemotherapeutics (e.g. cyclophosphamide, doxorubicin hydrochloride (hydroxydaunorubicin), vincristine sulfate (Oncovin), and prednisone (CHOP scheme) Etoposide, Vincristine, Chlormethine), protein inhibitors.

By “tumor” is meant a group of cells or tissue that is formed by misregulated cellular proliferation, in particular cancer. Tumors may show partial or complete lack of structural organization and functional coordination with the normal tissue, and usually form a distinct mass of tissue, which may be either benign or malignant. In particular, the term “tumor” refers to a malignant tumor. According to one embodiment, the term “tumor” or “tumor cell” also refers to non-solid cancers and cells of non-solid cancers such as leukemia cells. According to another embodiment, respective non-solid cancers or cells thereof are not encompassed by the terms “tumor” and “tumor cell”.

By “metastasis” is meant the spread of cancer cells from its original site to another part of the body. The formation of metastasis is a very complex process and normally involves detachment of cancer cells from a primary tumor, entering the body circulation and settling down to grow within normal tissues elsewhere in the body. When tumor cells metastasize, the new tumor is called a secondary or metastatic tumor, and its cells normally resemble those in the original tumor. This means, for example, that, if breast cancer metastasizes to the lungs, the secondary tumor is made up of abnormal breast cells, not of abnormal lung cells. The tumor in the lung is then called metastatic breast cancer, not lung cancer.

Exemplary ADCs of the Invention

In the following table, an overview of exemplary ADCs is disclosed. In those exemplary ADCs, the unnatural amino acid is 3-formyl tyrosine and the drug moiety (D) is a drug moiety as defined herein, preferably MMAE:

| Brentuximab light chain | Brentuximab heavy chain | Linker |
|---|---|---|
| SEQ ID NO: 2 | SEQ ID NO: 12 (comprising | structure 4 |
| | TTL recognition sequence) | |
| SEQ ID NO: 2 | SEQ ID NO: 12 | structure 5 |
| SEQ ID NO: 11 (comprising | SEQ ID NO: 1 | structure 4 |
| TTL recognition sequence) | | |
| SEQ ID NO: 11 | SEQ ID NO: 1 | structure 5 |
| SEQ ID NO: 11 | SEQ ID NO: 12 | structure 4 |
| SEQ ID NO: 11 | SEQ ID NO: 12 | structure 5 |
| SEQ ID NO: 14 (comprising | SEQ ID NO: 1 | structure 4 |
| TTL recognition sequence) | | |
| SEQ ID NO: 14 | SEQ ID NO: 1 | structure 5 |
| SEQ ID NO: 14 | SEQ ID NO: 12 | structure 4 |
| SEQ ID NO: 14 | SEQ ID NO: 12 | structure 5 |

Structures 4 and 5 are repeated in the following:

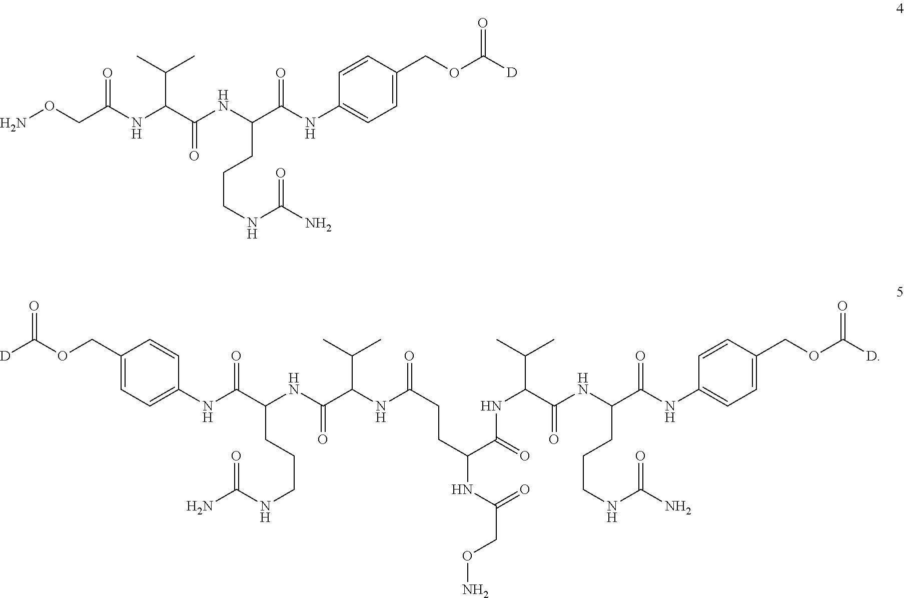

In some embodiments, the present invention relates to an antibody-drug conjugate (ADC) comprising:
(a) Brentuximab, wherein Brentuximab comprises at the C-terminus of each light chain a recognition sequence for tubulin-tyrosine ligase; each light chain, including the recognition sequence, has SEQ ID NO: 11; and each heavy chain of Brentuximab has SEQ ID NO: 1; and
(b) the C-terminus of the recognition sequence of each light chain is bound via an amide bond to a group having the following structure:

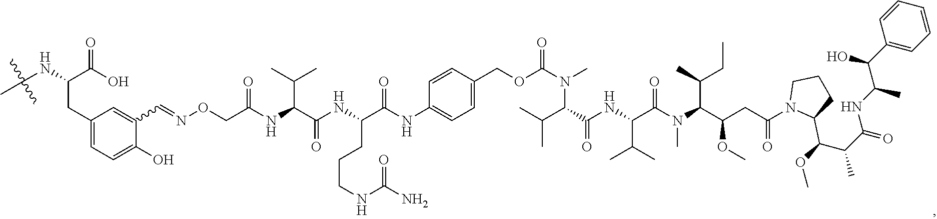 [SMILES: *N[C@@H](Cc1ccc(O)c(C=NOCC(=O)N[C@H](C(=O)N[C@@H](CCCNC(N)=O)C(=O)Nc2ccc(COC(=O)N(C)[C@H](C(=O)N[C@H](C(=O)N(C)[C@@H]([C@@H](C)CC)[C@@H](CC(=O)N3CCC[C@H]3[C@H](OC)[C@@H](C)C(=O)N[C@H](C)[C@@H](O)c3ccccc3)OC)C(C)C)C(C)C)cc2)C(C)C)c1)C(=O)O]

wherein the wavy line indicates attachment to the C-terminus of the recognition sequence of each light chain.

In some embodiments, the present invention relates to an antibody-drug conjugate (ADC) comprising:
(a) Brentuximab, wherein Brentuximab comprises at the C-terminus of each heavy chain a recognition sequence for tubulin-tyrosine ligase;
   each heavy chain, including the recognition sequence, has SEQ ID NO: 12; and each light chain of Brentuximab has SEQ ID NO: 2; and
(b) the C-terminus of the recognition sequence of each heavy chain is bound via an amide bond to a group having the following structure:

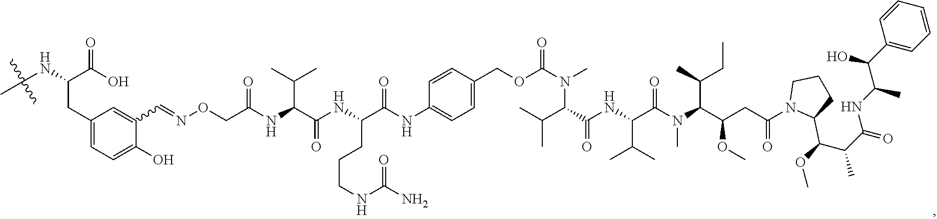 [SMILES: *N[C@@H](Cc1ccc(O)c(C=NOCC(=O)N[C@H](C(=O)N[C@@H](CCCNC(N)=O)C(=O)Nc2ccc(COC(=O)N(C)[C@H](C(=O)N[C@H](C(=O)N(C)[C@@H]([C@@H](C)CC)[C@@H](CC(=O)N3CCC[C@H]3[C@H](OC)[C@@H](C)C(=O)N[C@H](C)[C@@H](O)c3ccccc3)OC)C(C)C)C(C)C)cc2)C(C)C)c1)C(=O)O]

wherein the wavy line indicates attachment to the C-terminus of the recognition sequence of each heavy chain.

In some embodiments, the present invention relates to an antibody-drug conjugate (ADC) comprising:
(a) Brentuximab, wherein Brentuximab comprises at the C-terminus of each heavy chain a recognition sequence for tubulin-tyrosine ligase;
   each heavy chain, including the recognition sequence, has SEQ ID NO: 12; and wherein Brentuximab comprises at the C-terminus of each light chain a recognition sequence for tubulin-tyrosine ligase;
   each light chain, including the recognition sequence, has SEQ ID NO: 11; and
(b) the C-terminus of the recognition sequence of each heavy chain and the C-terminus of the recognition sequence of each light chain is bound via an amide bond to a group having the following structure:

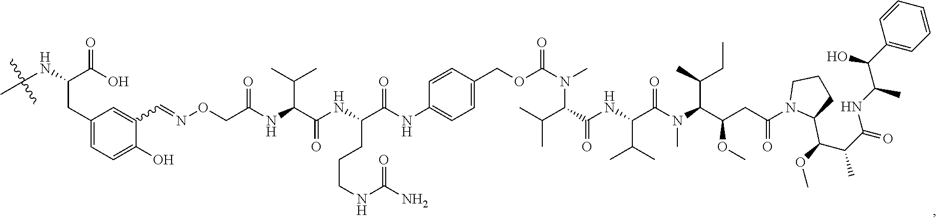 [SMILES: *N[C@@H](Cc1ccc(O)c(C=NOCC(=O)N[C@H](C(=O)N[C@@H](CCCNC(N)=O)C(=O)Nc2ccc(COC(=O)N(C)[C@H](C(=O)N[C@H](C(=O)N(C)[C@@H]([C@@H](C)CC)[C@@H](CC(=O)N3CCC[C@H]3[C@H](OC)[C@@H](C)C(=O)N[C@H](C)[C@@H](O)c3ccccc3)OC)C(C)C)C(C)C)cc2)C(C)C)c1)C(=O)O]

wherein the wavy line indicates attachment to the C-terminus of the recognition sequence of each heavy chain and to the C-terminus of the recognition sequence of each light chain.

In some embodiments, the present invention relates to an antibody-drug conjugate (ADC) comprising:
(a) Brentuximab, wherein Brentuximab comprises at the C-terminus of each light chain a recognition sequence for tubulin-tyrosine ligase;
   each light chain, including the recognition sequence, has SEQ ID NO: 11; and each heavy chain of Brentuximab has SEQ ID NO: 1; and
(b) the C-terminus of the recognition sequence of each light chain is bound via an amide bond to a group having the following structure:

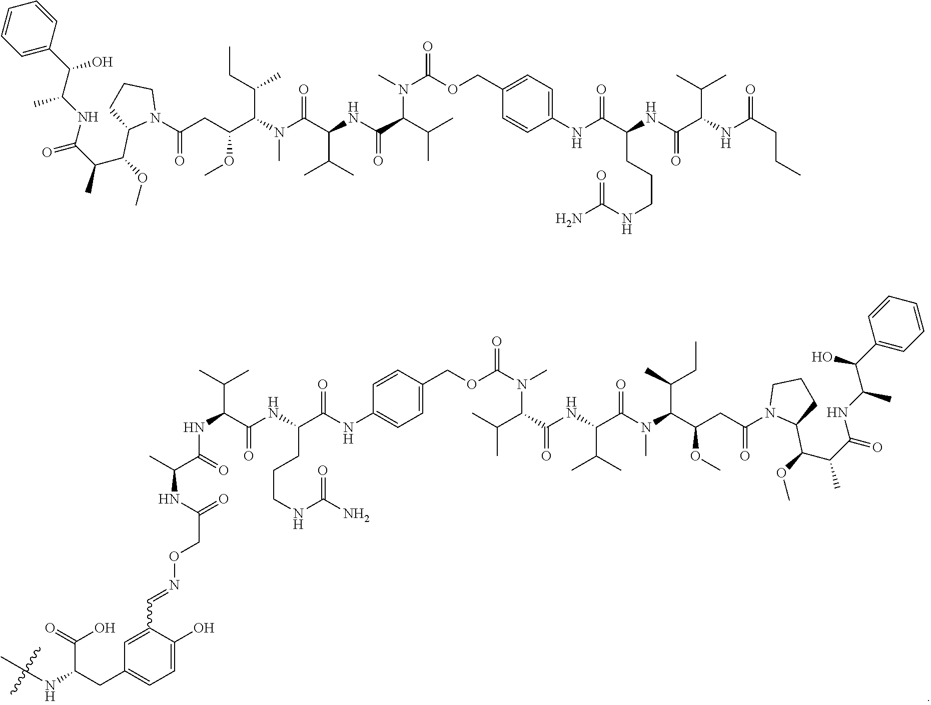 [SMILES: CCCC(=O)N[C@H](C(=O)N[C@@H](CCCNC(N)=O)C(=O)Nc1ccc(COC(=O)N(C)[C@H](C(=O)N[C@H](C(=O)N(C)[C@@H]([C@@H](C)CC)[C@@H](CC(=O)N2CCC[C@H]2[C@H](OC)[C@@H](C)C(=O)N[C@H](C)[C@@H](O)c2ccccc2)OC)C(C)C)C(C)C)cc1)C(C)C ; *N[C@@H](Cc1ccc(O)c(C=NOCC(=O)N[C@@H](C)C(=O)N[C@H](C(=O)N[C@@H](CCCNC(N)=O)C(=O)Nc2ccc(COC(=O)N(C)[C@H](C(=O)N[C@H](C(=O)N(C)[C@@H]([C@@H](C)CC)[C@@H](CC(=O)N3CCC[C@H]3[C@H](OC)[C@@H](C)C(=O)N[C@H](C)[C@@H](O)c3ccccc3)OC)C(C)C)C(C)C)cc2)C(C)C)c1)C(=O)O]

wherein the wavy line indicates attachment to the C-terminus of the recognition sequence of each light chain. In some embodiments, the present invention relates to an antibody-drug conjugate (ADC) comprising:
(a) Brentuximab, wherein Brentuximab comprises at the C-terminus of each light chain a recognition sequence for tubulin-tyrosine ligase;
   each light chain, including the recognition sequence, has SEQ ID NO: 14; and each heavy chain of Brentuximab has SEQ ID NO: 1; and
   (b) the C-terminus of the recognition sequence of each light chain is bound via an amide bond to a group having the following structure:

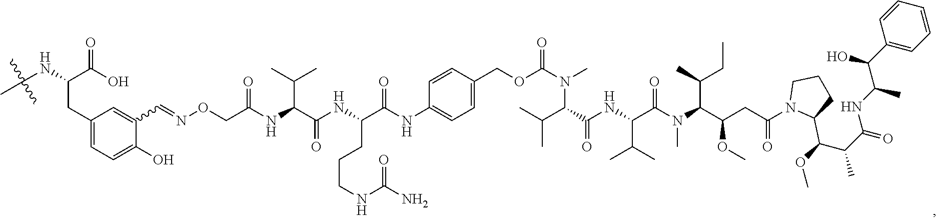 [SMILES: *N[C@@H](Cc1ccc(O)c(C=NOCC(=O)N[C@H](C(=O)N[C@@H](CCCNC(N)=O)C(=O)Nc2ccc(COC(=O)N(C)[C@H](C(=O)N[C@H](C(=O)N(C)[C@@H]([C@@H](C)CC)[C@@H](CC(=O)N3CCC[C@H]3[C@H](OC)[C@@H](C)C(=O)N[C@H](C)[C@@H](O)c3ccccc3)OC)C(C)C)C(C)C)cc2)C(C)C)c1)C(=O)O]

wherein the wavy line indicates attachment to the C-terminus of the recognition sequence of each light chain.

In some embodiments, the present invention relates to an antibody-drug conjugate (ADC) comprising:
(a) Brentuximab, wherein Brentuximab comprises at the C-terminus of each heavy chain a recognition sequence for tubulin-tyrosine ligase;
   each heavy chain, including the recognition sequence, has SEQ ID NO: 12; and
   wherein Brentuximab comprises at the C-terminus of each light chain a recognition sequence for tubulin-tyrosine ligase;
   each light chain, including the recognition sequence, has SEQ ID NO: 14; and
(b) the C-terminus of the recognition sequence of each heavy chain and the C-terminus of the recognition sequence of each light chain is bound via an amide bond to a group having the following structure:

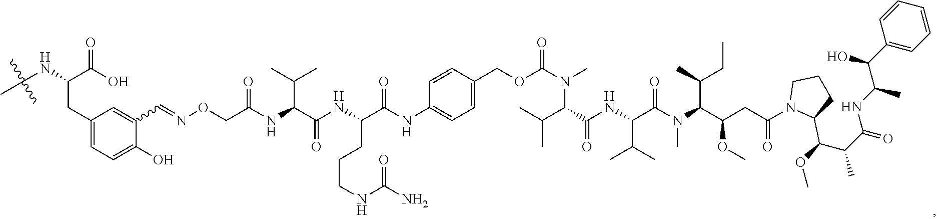 [SMILES: *N[C@@H](Cc1ccc(O)c(C=NOCC(=O)N[C@H](C(=O)N[C@@H](CCCNC(N)=O)C(=O)Nc2ccc(COC(=O)N(C)[C@H](C(=O)N[C@H](C(=O)N(C)[C@@H]([C@@H](C)CC)[C@@H](CC(=O)N3CCC[C@H]3[C@H](OC)[C@@H](C)C(=O)N[C@H](C)[C@@H](O)c3ccccc3)OC)C(C)C)C(C)C)cc2)C(C)C)c1)C(=O)O]

wherein the wavy line indicates attachment to the C-terminus of the recognition sequence of each heavy chain and to the C-terminus of the recognition sequence of each light chain.

In some embodiments, the present invention relates to an antibody-drug conjugate (ADC) comprising:
(a) Brentuximab, wherein Brentuximab comprises at the C-terminus of each light chain a recognition sequence for tubulin-tyrosine ligase;
   each light chain, including the recognition sequence, has SEQ ID NO: 14; and each heavy chain of Brentuximab has SEQ ID NO: 1; and
(b) the C-terminus of the recognition sequence of each light chain is bound via an amide bond to a group having the following structure:

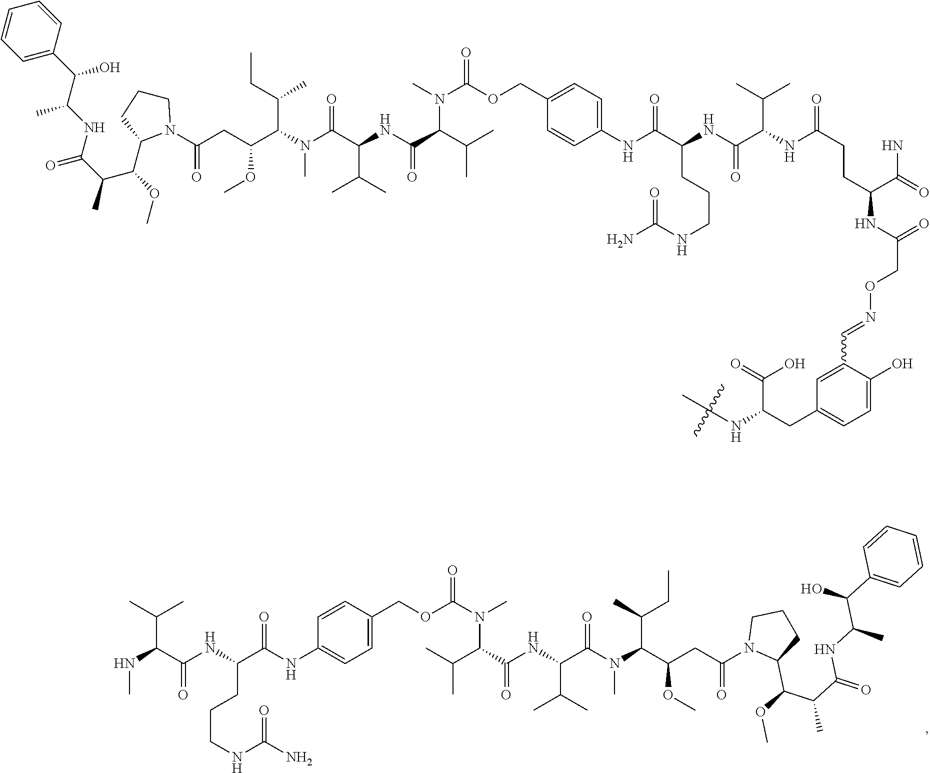 [SMILES: *N[C@@H](Cc1ccc(O)c(C=NOCC(=O)N[C@@H](CCC(=O)N[C@H](C(=O)N[C@@H](CCCNC(N)=O)C(=O)Nc2ccc(COC(=O)N(C)[C@H](C(=O)N[C@H](C(=O)N(C)[C@@H]([C@@H](C)CC)[C@@H](CC(=O)N3CCC[C@H]3[C@H](OC)[C@@H](C)C(=O)N[C@H](C)[C@@H](O)c3ccccc3)OC)C(C)C)C(C)C)cc2)C(C)C)C(N)=O)c1)C(=O)O ; CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC)C(C)C)cc1)C(C)C]

wherein the wavy line indicates attachment to the C-terminus of the recognition sequence of each light chain.

### Sequences

The present disclosure refers, inter alia, to the following sequences.

Brentuximab/cAC10 heavy chain (SEQ ID NO: 1):
Gln-Ile-Gln-Leu-Gln-Gln-Ser-Gly-Pro-Glu-Val-Val-Lys-Pro-Gly-Ala-Ser-Val-Lys-Ile-Ser-Cys-Lys-
Ala-Ser-Gly-Tyr-Thr-Phe-Thr-Asp-Tyr-Tyr-Ile-Thr-Trp-Val-Lys-Gln-Lys-Pro-Gly-Gln-Gly-Leu-Glu-
Trp-Ile-Gly-Trp-Ile-Tyr-Pro-Gly-Ser-Gly-Asn-Thr-Lys-Tyr-Asn-Glu-Lys-Phe-Lys-Gly-Lys-Ala-Thr-
Leu-Thr-Val-Asp-Thr-Ser-Ser-Ser-Thr-Ala-Phe-Met-Gln-Leu-Ser-Ser-Leu-Thr-Ser-Glu-Asp-Thr-
Ala-Val-Tyr-Phe-Cys-Ala-Asn-Tyr-Gly-Asn-Tyr-Trp-Phe-Ala-Tyr-Trp-Gly-Gln-Gly-Thr-Gln-Val-Thr-
Val-Ser-Ala-Ala-Ser-Thr-Lys-Gly-Pro-Ser-Val-Phe-Pro-Leu-Ala-Pro-Ser-Ser-Lys-Ser-Thr-Ser-Gly-
Gly-Thr-Ala-Ala-Leu-Gly-Cys-Leu-Val-Lys-Asp-Tyr-Phe-Pro-Glu-Pro-Val-Thr-Val-Ser-Trp-Asn-Ser-
Gly-Ala-Leu-Thr-Ser-Gly-Val-His-Thr-Phe-Pro-Ala-Val-Leu-Gln-Ser-Ser-Gly-Leu-Tyr-Ser-Leu-Ser-
Ser-Val-Val-Thr-Val-Pro-Ser-Ser-Ser-Leu-Gly-Thr-Gln-Thr-Tyr-Ile-Cys-Asn-Val-Asn-His-Lys-Pro-
Ser-Asn-Thr-Lys-Val-Asp-Lys-Lys-Val-Glu-Pro-Lys-Ser-Cys-Asp-Lys-Thr-His-Thr-Cys-Pro-Pro-
Cys-Pro-Ala-Pro-Glu-Leu-Leu-Gly-Gly-Pro-Ser-Val-Phe-Leu-Phe-Pro-Pro-Lys-Pro-Lys-Asp-Thr-
Leu-Met-Ile-Ser-Arg-Thr-Pro-Glu-Val-Thr-Cys-Val-Val-Val-Asp-Val-Ser-His-Glu-Asp-Pro-Glu-Val-
Lys-Phe-Asn-Trp-Tyr-Val-Asp-Gly-Val-Glu-Val-His-Asn-Ala-Lys-Thr-Lys-Pro-Arg-Glu-Glu-Gln-Tyr-
Asn-Ser-Thr-Tyr-Arg-Val-Val-Ser-Val-Leu-Thr-Val-Leu-His-Gln-Asp-Trp-Leu-Asn-Gly-Lys-Glu-Tyr-
Lys-Cys-Lys-Val-Ser-Asn-Lys-Ala-Leu-Pro-Ala-Pro-Ile-Glu-Lys-Thr-Ile-Ser-Lys-Ala-Lys-Gly-Gln-
Pro-Arg-Glu-Pro-Gln-Val-Tyr-Thr-Leu-Pro-Pro-Ser-Arg-Asp-Glu-Leu-Thr-Lys-Asn-Gln-Val-Ser-Leu-
Thr-Cys-Leu-Val-Lys-Gly-Phe-Tyr-Pro-Ser-Asp-Ile-Ala-Val-Glu-Trp-Glu-Ser-Asn-Gly-Gln-Pro-Glu-
Asn-Asn-Tyr-Lys-Thr-Thr-Pro-Pro-Val-Leu-Asp-Ser-Asp-Gly-Ser-Phe-Phe-Leu-Tyr-Ser-Lys-Leu-
Thr-Val-Asp-Lys-Ser-Arg-Trp-Gln-Gln-Gly-Asn-Val-Phe-Ser-Cys-Ser-Val-Met-His-Glu-Ala-Leu-His-
Asn-His-Tyr-Thr-Gln-Lys-Ser-Leu-Ser-Leu-Ser-Pro-Gly-Lys
Brentuximab/cAC10 light chain (SEQ ID NO: 2):
Asp-Ile-Val-Leu-Thr-Gln-Ser-Pro-Ala-Ser-Leu-Ala-Val-Ser-Leu-Gly-Gln-Arg-Ala-Thr-Ile-Ser-Cys-
Lys-Ala-Ser-Gln-Ser-Val-Asp-Phe-Asp-Gly-Asp-Ser-Tyr-Met-Asn-Trp-Tyr-Gln-Gln-Lys-Pro-Gly-
Gln-Pro-Pro-Lys-Val-Leu-Ile-Tyr-Ala-Ala-Ser-Asn-Leu-Glu-Ser-Gly-Ile-Pro-Ala-Arg-Phe-Ser-Gly-
Ser-Gly-Ser-Gly-Thr-Asp-Phe-Thr-Leu-Asn-Ile-His-Pro-Val-Glu-Glu-Glu-Asp-Ala-Ala-Thr-Tyr-Tyr-
Cys-Gln-Gln-Ser-Asn-Glu-Asp-Pro-Trp-Thr-Phe-Gly-Gly-Gly-Thr-Lys-Leu-Glu-Ile-Lys-Arg-Thr-Val-
Ala-Ala-Pro-Ser-Val-Phe-Ile-Phe-Pro-Pro-Ser-Asp-Glu-Gln-Leu-Lys-Ser-Gly-Thr-Ala-Ser-Val-Val-
Cys-Leu-Leu-Asn-Asn-Phe-Tyr-Pro-Arg-Glu-Ala-Lys-Val-Gln-Trp-Lys-Val-Asp-Asn-Ala-Leu-Gln-
Ser-Gly-Asn-Ser-Gln-Glu-Ser-Val-Thr-Glu-Gln-Asp-Ser-Lys-Asp-Ser-Thr-Tyr-Ser-Leu-Ser-Ser-Thr-
Leu-Thr-Leu-Ser-Lys-Ala-Asp-Tyr-Glu-Lys-His-Lys-Val-Tyr-Ala-Cys-Glu-Val-Thr-His-Gln-Gly-Leu-
Ser-Ser-Pro-Val-Thr-Lys-Ser-Phe-Asn-Arg-Gly-Glu-Cys
TTL recognition sequence (SEQ ID NO: 3):
Xaa₁-Xaa₂-Xaa₃-Glu,

wherein:
Xaa₁ is any amino acid; as illustrative examples, Xaa₁ may be Glu, Asp, Ala, Lys or Pro;
Xaa₂ is any amino acid; as illustrative example, Xaa₂ may be Gly, Ser, Ala, Val or Phe; and

### Xaa₃ is Glu, Asp or Cys.

TTL recognition sequence (SEQ ID NO: 4):
Glu-Gly-Glu-Glu
TTL recognition sequence (SEQ ID NO: 5):
Val-Asp-Ser-Val-Glu-Gly-Glu-Gly-Glu-Glu-Glu-Gly-Glu-Glu
TTL recognition sequence (SEQ ID NO: 6):
Ser-Val-Glu-Gly-Glu-Gly-Glu-Glu-Glu-Gly-Glu-Glu
TTL recognition sequence (SEQ ID NO: 7):
Ser-Ala-Asp-Gly-Glu-Asp-Glu-Gly-Glu-Glu
TTL recognition sequence (SEQ ID NO: 8):
Ser-Val-Glu-Ala-Glu-Ala-Glu-Glu-Gly-Glu-Glu
TTL recognition sequence (SEQ ID NO: 9):
Ser-Tyr-Glu-Asp-Glu-Asp-Glu-Gly-Glu-Glu
TTL recognition sequence (SEQ ID NO: 10):
Ser-Phe-Glu-Glu-Glu-Asn-Glu-Gly-Glu-Glu
Brentuximab light chain comprising Gly-Gly-Gly-Gly-Ser-Linker, and TTL recognition
sequence at C-terminus (SEQ ID NO: 11):
Asp-Ile-Val-Leu-Thr-Gln-Ser-Pro-Ala-Ser-Leu-Ala-Val-Ser-Leu-Gly-Gln-Arg-Ala-Thr-Ile-Ser-Cys-
Lys-Ala-Ser-Gln-Ser-Val-Asp-Phe-Asp-Gly-Asp-Ser-Tyr-Met-Asn-Trp-Tyr-Gln-Gln-Lys-Pro-Gly-
Gln-Pro-Pro-Lys-Val-Leu-Ile-Tyr-Ala-Ala-Ser-Asn-Leu-Glu-Ser-Gly-Ile-Pro-Ala-Arg-Phe-Ser-Gly-
Ser-Gly-Ser-Gly-Thr-Asp-Phe-Thr-Leu-Asn-Ile-His-Pro-Val-Glu-Glu-Glu-Asp-Ala-Ala-Thr-Tyr-Tyr-
Cys-Gln-Gln-Ser-Asn-Glu-Asp-Pro-Trp-Thr-Phe-Gly-Gly-Gly-Thr-Lys-Leu-Glu-Ile-Lys-Arg-Thr-Val-
Ala-Ala-Pro-Ser-Val-Phe-Ile-Phe-Pro-Pro-Ser-Asp-Glu-Gln-Leu-Lys-Ser-Gly-Thr-Ala-Ser-Val-Val-
Cys-Leu-Leu-Asn-Asn-Phe-Tyr-Pro-Arg-Glu-Ala-Lys-Val-Gln-Trp-Lys-Val-Asp-Asn-Ala-Leu-Gln-
Ser-Gly-Asn-Ser-Gln-Glu-Ser-Val-Thr-Glu-Gln-Asp-Ser-Lys-Asp-Ser-Thr-Tyr-Ser-Leu-Ser-Ser-Thr-
Leu-Thr-Leu-Ser-Lys-Ala-Asp-Tyr-Glu-Lys-His-Lys-Val-Tyr-Ala-Cys-Glu-Val-Thr-His-Gln-Gly-Leu-
Ser-Ser-Pro-Val-Thr-Lys-Ser-Phe-Asn-Arg-Gly-Glu-Cys-Gly-Gly-Gly-Gly-Ser-Val-Asp-Ser-Val-Glu-
Gly-Glu-Gly-Glu-Glu-Glu-Gly-Glu-Glu
Brentuximab heavy chain fused to TTL recognition sequence at C-terminus (SEQ ID NO: 12):
Gln-Ile-Gln-Leu-Gln-Gln-Ser-Gly-Pro-Glu-Val-Val-Lys-Pro-Gly-Ala-Ser-Val-Lys-Ile-Ser-Cys-Lys-
Ala-Ser-Gly-Tyr-Thr-Phe-Thr-Asp-Tyr-Tyr-Ile-Thr-Trp-Val-Lys-Gln-Lys-Pro-Gly-Gln-Gly-Leu-Glu-
Trp-Ile-Gly-Trp-Ile-Tyr-Pro-Gly-Ser-Gly-Asn-Thr-Lys-Tyr-Asn-Glu-Lys-Phe-Lys-Gly-Lys-Ala-Thr-
Leu-Thr-Val-Asp-Thr-Ser-Ser-Ser-Thr-Ala-Phe-Met-Gln-Leu-Ser-Ser-Leu-Thr-Ser-Glu-Asp-Thr-
Ala-Val-Tyr-Phe-Cys-Ala-Asn-Tyr-Gly-Asn-Tyr-Trp-Phe-Ala-Tyr-Trp-Gly-Gln-Gly-Thr-Gln-Val-Thr-
Val-Ser-Ala-Ala-Ser-Thr-Lys-Gly-Pro-Ser-Val-Phe-Pro-Leu-Ala-Pro-Ser-Ser-Lys-Ser-Thr-Ser-Gly-
Gly-Thr-Ala-Ala-Leu-Gly-Cys-Leu-Val-Lys-Asp-Tyr-Phe-Pro-Glu-Pro-Val-Thr-Val-Ser-Trp-Asn-Ser-
Gly-Ala-Leu-Thr-Ser-Gly-Val-His-Thr-Phe-Pro-Ala-Val-Leu-Gln-Ser-Ser-Gly-Leu-Tyr-Ser-Leu-Ser-
Ser-Val-Val-Thr-Val-Pro-Ser-Ser-Ser-Leu-Gly-Thr-Gln-Thr-Tyr-Ile-Cys-Asn-Val-Asn-His-Lys-Pro-
Ser-Asn-Thr-Lys-Val-Asp-Lys-Lys-Val-Glu-Pro-Lys-Ser-Cys-Asp-Lys-Thr-His-Thr-Cys-Pro-Pro-
Cys-Pro-Ala-Pro-Glu-Leu-Leu-Gly-Gly-Pro-Ser-Val-Phe-Leu-Phe-Pro-Pro-Lys-Pro-Lys-Asp-Thr-
Leu-Met-Ile-Ser-Arg-Thr-Pro-Glu-Val-Thr-Cys-Val-Val-Val-Asp-Val-Ser-His-Glu-Asp-Pro-Glu-Val-
Lys-Phe-Asn-Trp-Tyr-Val-Asp-Gly-Val-Glu-Val-His-Asn-Ala-Lys-Thr-Lys-Pro-Arg-Glu-Glu-Gln-Tyr-
Asn-Ser-Thr-Tyr-Arg-Val-Val-Ser-Val-Leu-Thr-Val-Leu-His-Gln-Asp-Trp-Leu-Asn-Gly-Lys-Glu-Tyr-
Lys-Cys-Lys-Val-Ser-Asn-Lys-Ala-Leu-Pro-Ala-Pro-Ile-Glu-Lys-Thr-Ile-Ser-Lys-Ala-Lys-Gly-Gln-
Pro-Arg-Glu-Pro-Gln-Val-Tyr-Thr-Leu-Pro-Pro-Ser-Arg-Asp-Glu-Leu-Thr-Lys-Asn-Gln-Val-Ser-Leu-
Thr-Cys-Leu-Val-Lys-Gly-Phe-Tyr-Pro-Ser-Asp-Ile-Ala-Val-Glu-Trp-Glu-Ser-Asn-Gly-Gln-Pro-Glu-
Asn-Asn-Tyr-Lys-Thr-Thr-Pro-Pro-Val-Leu-Asp-Ser-Asp-Gly-Ser-Phe-Phe-Leu-Tyr-Ser-Lys-Leu-
Thr-Val-Asp-Lys-Ser-Arg-Trp-Gln-Gln-Gly-Asn-Val-Phe-Ser-Cys-Ser-Val-Met-His-Glu-Ala-Leu-His-
Asn-His-Tyr-Thr-Gln-Lys-Ser-Leu-Ser-Leu-Ser-Pro-Gly-Lys-Val-Asp-Ser-Val-Glu-Gly-Glu-Gly-Glu-
Glu-Glu-Gly-Glu-Glu
TTL from Canis Lupus (SEQ ID NO: 13):
Met-Tyr-Thr-Phe-Val-Val-Arg-Asp-Glu-Asn-Ser-Ser-Val-Tyr-Ala-Glu-Val-Ser-Arg-Leu-Leu-Leu-Ala-
Thr-Gly-His-Trp-Lys-Arg-Leu-Arg-Arg-Asp-Asn-Pro-Arg-Phe-Asn-Leu-Met-Leu-Gly-Glu-Arg-Asn-
Arg-Leu-Pro-Phe-Gly-Arg-Leu-Gly-His-Glu-Pro-Gly-Leu-Met-Gln-Leu-Val-Asn-Tyr-Tyr-Arg-Gly-Ala-
Asp-Lys-Leu-Cys-Arg-Lys-Ala-Ser-Leu-Val-Lys-Leu-Ile-Lys-Thr-Ser-Pro-Asp-Leu-Ala-Glu-Ser-Cys-
Thr-Trp-Phe-Pro-Glu-Ser-Tyr-Val-Ile-Tyr-Pro-Thr-Asn-Leu-Lys-Thr-Pro-Val-Ala-Pro-Ala-Gln-Asn-
Gly-Ile-His-Pro-Pro-Ile-His-Asn-Ser-Arg-Thr-Asp-Glu-Arg-Glu-Phe-Phe-Leu-Ala-Ser-Tyr-Asn-Arg-
Lys-Lys-Glu-Asp-Gly-Glu-Gly-Asn-Val-Trp-Ile-Ala-Lys-Ser-Ser-Ala-Gly-Ala-Lys-Gly-Glu-Gly-Ile-
Leu-Ile-Ser-Ser-Glu-Ala-Thr-Glu-Leu-Leu-Asp-Phe-Ile-Asp-Asn-Gln-Gly-Gln-Val-His-Val-Ile-Gln-
Lys-Tyr-Leu-Glu-His-Pro-Leu-Leu-Leu-Glu-Pro-Gly-His-Arg-Lys-Phe-Asp-Ile-Arg-Ser-Trp-Val-Leu-
Val-Asp-His-Gln-Tyr-Asn-Ile-Tyr-Leu-Tyr-Lys-Glu-Gly-Val-Leu-Arg-Thr-Ala-Ser-Glu-Pro-Tyr-His-
Val-Asp-Asn-Phe-Gln-Asp-Lys-Thr-Cys-His-Leu-Thr-Asn-His-Cys-Ile-Gln-Lys-Glu-Tyr-Ser-Lys-
Asn-Tyr-Gly-Lys-Tyr-Glu-Glu-Gly-Asn-Glu-Met-Phe-Phe-Glu-Glu-Phe-Asn-Gln-Tyr-Leu-Ile-Ser-Ala-
Leu-Asn-Ile-Thr-Leu-Glu-Ser-Ser-Ile-Leu-Leu-Gln-Ile-Lys-His-Ile-Ile-Arg-Ser-Cys-Leu-Met-Ser-Val-
Glu-Pro-Ala-Ile-Ser-Thr-Arg-Tyr-Leu-Pro-Tyr-Gln-Ser-Phe-Gln-Leu-Phe-Gly-Phe-Asp-Phe-Met-Val-
Asp-Glu-Thr-Leu-Lys-Val-Trp-Leu-Ile-Glu-Val-Asn-Gly-Ala-Pro-Ala-Cys-Ala-Gln-Arg-Leu-Tyr-Ala-
Glu-Leu-Cys-Gln-Gly-Ile-Val-Asp-Ile-Ala-Ile-Ser-Ser-Val-Phe-Pro-Pro-Pro-Asp-Val-Glu-Pro-Gln-
His-Ile-Gln-Pro-Ala-Ala-Phe-Ile-Lys-Leu
Brentuximab light chain comprising TTL recognition sequence at C-terminus (SEQ ID NO: 14):
Asp-Ile-Val-Leu-Thr-Gln-Ser-Pro-Ala-Ser-Leu-Ala-Val-Ser-Leu-Gly-Gln-Arg-Ala-Thr-Ile-Ser-Cys-
Lys-Ala-Ser-Gln-Ser-Val-Asp-Phe-Asp-Gly-Asp-Ser-Tyr-Met-Asn-Trp-Tyr-Gln-Gln-Lys-Pro-Gly-
Gln-Pro-Pro-Lys-Val-Leu-Ile-Tyr-Ala-Ala-Ser-Asn-Leu-Glu-Ser-Gly-Ile-Pro-Ala-Arg-Phe-Ser-Gly-
Ser-Gly-Ser-Gly-Thr-Asp-Phe-Thr-Leu-Asn-Ile-His-Pro-Val-Glu-Glu-Glu-Asp-Ala-Ala-Thr-Tyr-Tyr-
Cys-Gln-Gln-Ser-Asn-Glu-Asp-Pro-Trp-Thr-Phe-Gly-Gly-Gly-Thr-Lys-Leu-Glu-Ile-Lys-Arg-Thr-Val-
Ala-Ala-Pro-Ser-Val-Phe-Ile-Phe-Pro-Pro-Ser-Asp-Glu-Gln-Leu-Lys-Ser-Gly-Thr-Ala-Ser-Val-Val-
Cys-Leu-Leu-Asn-Asn-Phe-Tyr-Pro-Arg-Glu-Ala-Lys-Val-Gln-Trp-Lys-Val-Asp-Asn-Ala-Leu-Gln-
Ser-Gly-Asn-Ser-Gln-Glu-Ser-Val-Thr-Glu-Gln-Asp-Ser-Lys-Asp-Ser-Thr-Tyr-Ser-Leu-Ser-Ser-Thr-
Leu-Thr-Leu-Ser-Lys-Ala-Asp-Tyr-Glu-Lys-His-Lys-Val-Tyr-Ala-Cys-Glu-Val-Thr-His-Gln-Gly-Leu-
Ser-Ser-Pro-Val-Thr-Lys-Ser-Phe-Asn-Arg-Gly-Glu-Cys-Val-Asp-Ser-Val-Glu-Gly-Glu-Gly-Glu-Glu-
Glu-Gly-Glu-Glu

### Items of the Invention

The invention further relates to the following items:
1. An antibody-drug conjugate (ADC) comprising:
   (a) Brentuximab, wherein Brentuximab comprises at the C-terminus of the light chains, the heavy chains or all of the heavy and light chains of the Brentuximab a recognition sequence for tubulin tyrosine ligase and a non-natural amino acid; and
   (b) at least one drug moiety;
   wherein a drug moiety is coupled to each of the non-natural amino acids via a linker.
2. The ADC of item 1, wherein the heavy chains of Brentuximab have an amino acid sequence that comprises SEQ ID NO: 1 or have a sequence identity of at least 95% to SEQ ID NO: 1 and/or wherein the light chains of Brentuximab have an amino acid sequence that comprises SEQ ID NO: 2 or have a sequence identity of at least 95% to SEQ ID NO: 2, preferably, Brentuximab consists of heavy chains consisting of the amino acid sequence of SEQ ID NO: 1 and light chains consisting of the amino acid sequence of SEQ ID NO: 2.
3. The ADC of any one of the preceding items, wherein the drug moiety is selected from the group consisting of camptothecins, maytansinoids, calicheamycins, duocarmycins, tubulysins, amatoxins, dolastatins and auristatins such as monomethyl auristatin E (MMAE), pyrrolobenzodiazepine dimers, indolino-benzodiazepine dimers, radioisotopes, therapeutic proteins and peptides (or fragments thereof), nucleic acids, PROTACs, kinase inhibitors, MEK inhibitors, KSP inhibitors, and analogues or prodrugs thereof, preferably the drug moiety is MMAE.
4. The ADC of any one of the preceding items, wherein the recognition sequence for tubulin tyrosine ligase has at least the amino acid sequence X₁X₂X₃X₄ (SEQ ID NO: 3), wherein X₁ and X₂ is any amino acid, X₃ is E, D or C and X₄ is E, preferably wherein X₂ is G, S, A, V, or F and/or wherein X₁ is E, D, A, K, or P.
5. The ADC of any one of the preceding items, wherein the recognition sequence is EGEE (SEQ ID No. 4), preferably wherein the recognition sequence is VDSVEGEGEEEGEE (SEQ ID No. 5), SVEGEGEEEGEE (SEQ ID No. 6), SADGEDEGEE (SEQ ID No. 7), SVEAEAEEGEE (SEQ ID No. 8), SYEDEDEGEE (SEQ ID No. 9), or SFEEENEGEE (SEQ ID No. 10).
6. The ADC of any one of the preceding items, wherein the unnatural amino acid is a 2-substituted, 3-substituted or 4-substituted tyrosine or a tyrosine derivative substituted at the benzylic position.
7. The ADC of item 6, wherein said 3- or 4-substituted tyrosine derivative is 3-nitrotyrosine, 3-aminotyrosine, 3-azidotyrosine, 3-formyltyrosine, 3-acetyltyrosine, or 4-aminophenylalanine, preferably the unnatural amino acid is 3-formyltyrosine.
8. The ADC of any one of the preceding items, wherein the linker is cleavable, preferably by a protease, more preferably by a cathepsin such as cathepsin B.
8a. The ADC of any one of items 1 to 7, wherein the linker is non-cleavable.
9. The ADC of any one of the preceding items, wherein the linker comprises a valine-citrulline moiety.
10. The ADC of any one of the preceding items, wherein the linker comprises a hydroxylamine group and the unnatural amino acid comprises a formyl group ortho of a hydroxyl group in an aromatic ring such as 3-formyltyorsine, and wherein the hydroxylamine group of the linker forms an oxime with the formyl group of the unnatural amino acid after conjugation.
11. The ADC of any one of the preceding items, wherein Brentuximab is conjugated to two, four, six, or eight, preferably two or four drug moieties, more preferably two drug moieties.
12. The ADC of any one of the preceding items, wherein the linker has a structure as depicted in structure 1 before being coupled to the unnatural amino acid:

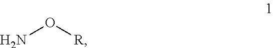

wherein R is one or more drug moieties, which are optionally coupled to the hydroxylamine of structure 1 by one or more cleavage sites, preferably wherein the hydroxylamine of structure 1 is conjugated to the unnatural amino acid.
13. The ADC of any one of the preceding items, wherein the linker has a structure as depicted in structure 2 or 3 before being coupled to the unnatural amino acid:

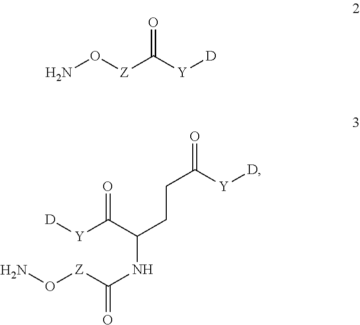

wherein Z is is selected from the group consisting of substituted or unsubstituted alkyl, substituted or unsubstituted alkenyl, substituted or unsubstituted alkynyl, substituted or unsubstituted cycloalkyl, substituted or unsubstituted aryl, substituted or unsubstituted arylalkyl, substituted or unsubstituted heteroaryl, substituted or unsubstituted heteroarylalkyl, substituted or unsubstituted heterocyclyl, substituted or unsubstituted heteroalkyl, substituted or unsubstituted heteroalkenyl and substituted or unsubstituted heteroalkynyl;
wherein D is one or more drug moieties; and
wherein Y is a cleavage site such as a cleavage site for a cathepsin such as cathepsin B; preferably wherein the hydroxylamine of structure 2 or 3 is conjugated to the unnatural amino acid.
14. The ADC of any one of the preceding items, wherein the linker has a structure as depicted in structure 4 or 5 before being coupled to the unnatural amino acid, wherein D is a drug moiety, preferably MMAE:

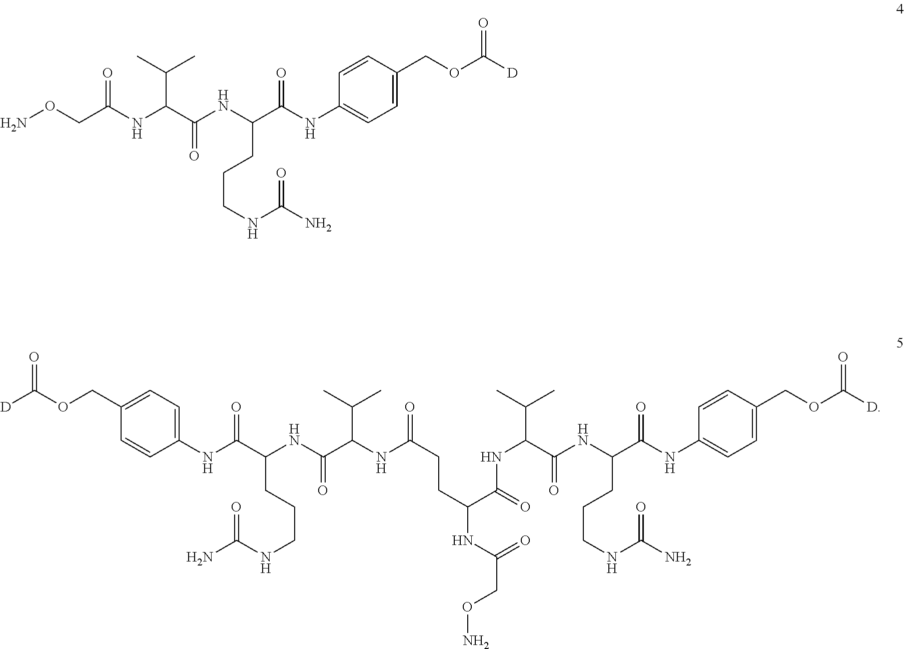

15. The ADC of item 14, wherein D is MMAE, wherein the unnatural amino acid is 3-formyltyrosine and the hydroxylamine group of the linker forms an oxime with the 3-formyl group of the unnatural amino acid.
16. An antibody-drug conjugate (ADC) comprising:
   (a) Brentuximab, wherein Brentuximab comprises at the C-terminus of each light chain a recognition sequence for tubulin-tyrosine ligase;
      each light chain, including the recognition sequence, has SEQ ID NO: 11; and
      each heavy chain of Brentuximab has SEQ ID NO: 1; and
   (b) the C-terminus of the recognition sequence of each light chain is bound via an amide bond to a group having the following structure:

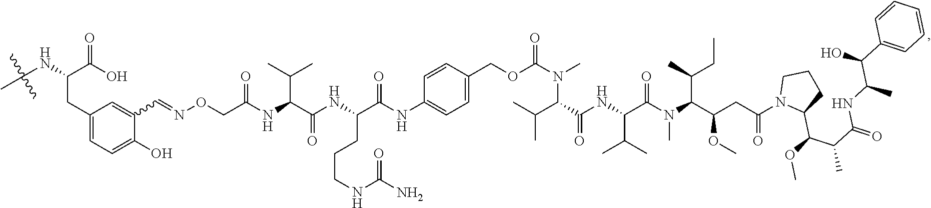 [SMILES: *N[C@@H](Cc1ccc(O)c(C=NOCC(=O)N[C@H](C(=O)N[C@@H](CCCNC(N)=O)C(=O)Nc2ccc(COC(=O)N(C)[C@H](C(=O)N[C@H](C(=O)N(C)[C@@H]([C@@H](C)CC)[C@@H](CC(=O)N3CCC[C@H]3[C@H](OC)[C@@H](C)C(=O)N[C@H](C)[C@@H](O)c3ccccc3)OC)C(C)C)C(C)C)cc2)C(C)C)c1)C(=O)O]

wherein the wavy line indicates attachment to the C-terminus of the recognition sequence of each light chain.
17. An antibody-drug conjugate (ADC) comprising:
   (a) Brentuximab, wherein Brentuximab comprises at the C-terminus of each heavy chain a recognition sequence for tubulin-tyrosine ligase;
      each heavy chain, including the recognition sequence, has SEQ ID NO: 12; and
      each light chain of Brentuximab has SEQ ID NO: 2; and
   (b) the C-terminus of the recognition sequence of each heavy chain is bound via an amide bond to a group having the following structure:

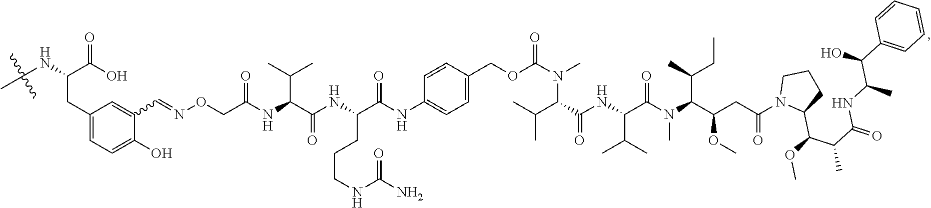 [SMILES: *N[C@@H](Cc1ccc(O)c(C=NOCC(=O)N[C@H](C(=O)N[C@@H](CCCNC(N)=O)C(=O)Nc2ccc(COC(=O)N(C)[C@H](C(=O)N[C@H](C(=O)N(C)[C@@H]([C@@H](C)CC)[C@@H](CC(=O)N3CCC[C@H]3[C@H](OC)[C@@H](C)C(=O)N[C@H](C)[C@@H](O)c3ccccc3)OC)C(C)C)C(C)C)cc2)C(C)C)c1)C(=O)O]

wherein the wavy line indicates attachment to the C-terminus of the recognition sequence of each heavy chain.
18. An antibody-drug conjugate (ADC) comprising:
   (a) Brentuximab, wherein Brentuximab comprises at the C-terminus of each heavy chain a recognition sequence for tubulin-tyrosine ligase;
      each heavy chain, including the recognition sequence, has SEQ ID NO: 12; and
      wherein Brentuximab comprises at the C-terminus of each light chain a recognition sequence for tubulin-tyrosine ligase;
      each light chain, including the recognition sequence, has SEQ ID NO: 11; and
   (b) the C-terminus of the recognition sequence of each heavy chain and the C-terminus of the recognition sequence of each light chain is bound via an amide bond to a group having the following structure:

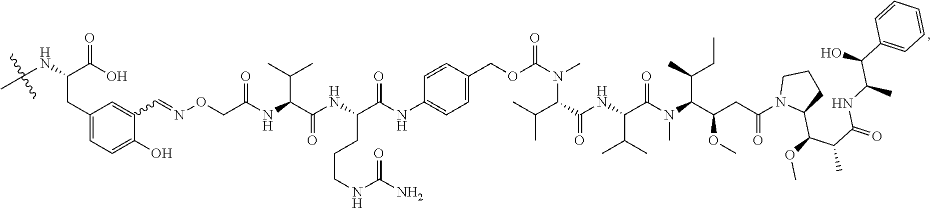 [SMILES: *N[C@@H](Cc1ccc(O)c(C=NOCC(=O)N[C@H](C(=O)N[C@@H](CCCNC(N)=O)C(=O)Nc2ccc(COC(=O)N(C)[C@H](C(=O)N[C@H](C(=O)N(C)[C@@H]([C@@H](C)CC)[C@@H](CC(=O)N3CCC[C@H]3[C@H](OC)[C@@H](C)C(=O)N[C@H](C)[C@@H](O)c3ccccc3)OC)C(C)C)C(C)C)cc2)C(C)C)c1)C(=O)O]

wherein the wavy line indicates attachment to the C-terminus of the recognition sequence of each heavy chain and to the C-terminus of the recognition sequence of each light chain.
19. An antibody-drug conjugate (ADC) comprising:
   (a) Brentuximab, wherein Brentuximab comprises at the C-terminus of each light chain a recognition sequence for tubulin-tyrosine ligase;
      each light chain, including the recognition sequence, has SEQ ID NO: 11; and
      each heavy chain of Brentuximab has SEQ ID NO: 1; and
   (b) the C-terminus of the recognition sequence of each light chain is bound via an amide bond to a group having the following structure:

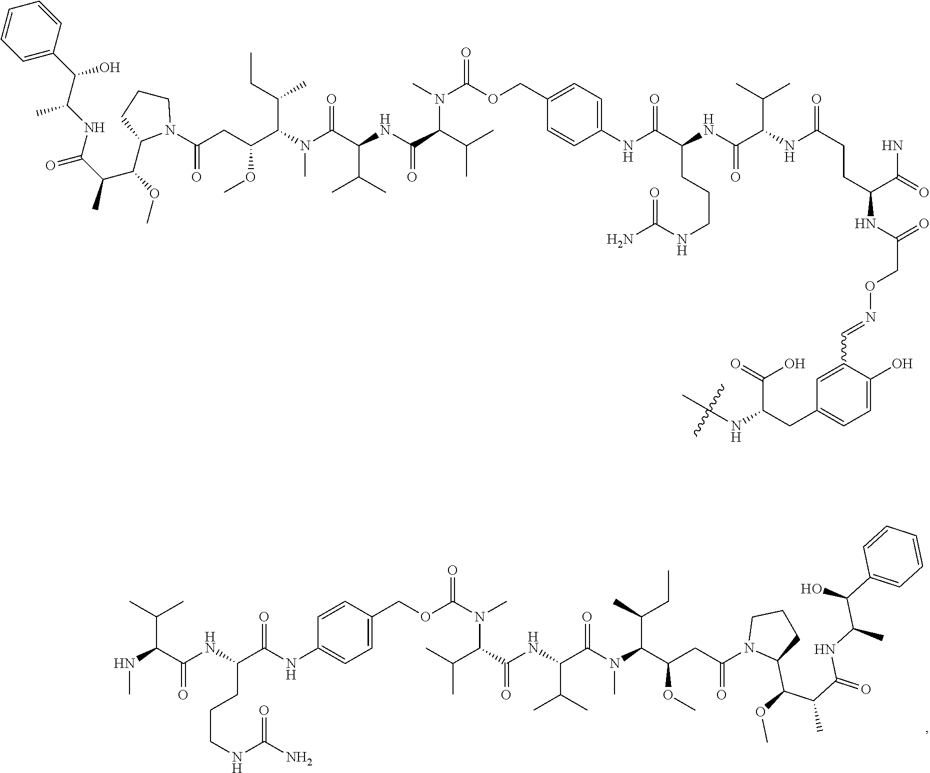 [SMILES: *N[C@@H](Cc1ccc(O)c(C=NOCC(=O)N[C@@H](CCC(=O)N[C@H](C(=O)N[C@@H](CCCNC(N)=O)C(=O)Nc2ccc(COC(=O)N(C)[C@H](C(=O)N[C@H](C(=O)N(C)[C@@H]([C@@H](C)CC)[C@@H](CC(=O)N3CCC[C@H]3[C@H](OC)[C@@H](C)C(=O)N[C@H](C)[C@@H](O)c3ccccc3)OC)C(C)C)C(C)C)cc2)C(C)C)C(N)=O)c1)C(=O)O ; CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC)C(C)C)cc1)C(C)C]

wherein the wavy line indicates attachment to the C-terminus of the recognition sequence of each light chain.
20. A method of producing an ADC as defined in any one of items 1-19, comprising
   (a) introducing or adding at the C-terminus of the light chain, the heavy chain or both the light chain and the heavy chain of Brentuximab a recognition sequence for tubulin tyrosine ligase;
   (b) contacting the Brentuximab obtained in step (a) in the presence of tubulin tyrosine ligase and a non-natural amino acid under conditions suitable for the tubulin tyrosine ligase to ligate said Brentuximab with said non-natural amino acid; and
   (c) conjugating an optionally cleavable linker comprising a drug moiety to said ligated Brentuximab obtained in step (b).
21. An ADC obtainable or being obtained by the method of item 20.
22. A pharmaceutical composition comprising the ADC of any one of items 1 to 19 or 21.
23. The ADC of any one items 1 to 19 or 21 or the pharmaceutical composition of item 22 for use in a method of treating a disease.
24. The ADC or the pharmaceutical composition for use of item 23, wherein the disease is associated with overexpression of CD30.
25. The ADC or the pharmaceutical composition for use of item 23 or 24, wherein the disease is selected from the group consisting of lymphoma, such as Hodgkin's lymphoma (HL), non-Hodgkin lymphoma (NHL), anaplastic large-cell lymphoma (ALCL), large B-cell lymphoma, paediatric lymphoma, T-cell lymphoma and enteropathy-associated T-cell lymphoma (EATL), leukaemia, such as acute myeloid leukaemia (AML), acute lymphoblastic leukaemia (ALL) and mast cell leukaemia, germ cell cancer, graft-versus-host disease (GvHD) and lupus, in particular systemic lupus erythematosus (SLE), preferably Hodgkin Lymphoma (HL) or anaplastic large cell lymphoma (ALCL).
25a. The ADC or the pharmaceutical composition for use of item 23 or 24, wherein the disease is selected from the group consisting of peripheral T cell lymphoma—not otherwise specified (PTCL-NOS), angioimmunoblastic T-cell lymphoma (AITL), enteropathy associated T cell lymphoma (EATL), adult T-cell leukemia/lymphoma (ATLL), extranodal natural killer/T-cell lymphoma (ENKTCL), hepatosplenic and intestinal γ/δ-T cell lymphoma, nodal peripheral T-cell lymphoma with TFH phenotype, and follicular T cell lymphoma.
25b. The ADC or the pharmaceutical composition for use of item 23 or 24, wherein the disease is peripheral T cell lymphoma (PTCL), including anaplastic large cell lymphoma (ALCL); or cutaneous T cell lymphoma (CTCL), including primary cutaneous anaplastic large cell lymphoma (pcALCL).
26. Use of an ADC of any one of items 1 to 19 or 21 for the manufacture of a medicament for treatment of a disease associated with overexpression of CD30.
27. The use of item 26, wherein the disease is selected from the group consisting of lymphoma, such as Hodgkin's lymphoma (HL), non-Hodgkin lymphoma (NHL), anaplastic large-cell lymphoma (ALCL), large B-cell lymphoma, paediatric lymphoma, T-cell lymphoma and enteropathy-associated T-cell lymphoma (EATL), leukaemia, such as acute myeloid leukaemia (AML), acute lymphoblastic leukaemia (ALL) and mast cell leukaemia, germ cell cancer, graft-versus-host disease (GvHD) and lupus, in particular systemic lupus erythematosus (SLE).
28. The use of item 27, wherein the disease is Hodgkin Lymphoma (HL) or anaplastic large cell lymphoma (ALCL).
28a. The use of item 26, wherein the disease is selected from the group consisting of peripheral T cell lymphoma—not otherwise specified (PTCL-NOS), angioimmunoblastic T-cell lymphoma (AITL), enteropathy associated T cell lymphoma (EATL), adult T-cell leukemia/lymphoma (ATLL), extranodal natural killer/T-cell lymphoma (ENKTCL), hepatosplenic and intestinal γ/δ-T cell lymphoma, nodal peripheral T-cell lymphoma with TFH phenotype, and follicular T cell lymphoma.
28b. The use of item 26, wherein the disease is peripheral T cell lymphoma (PTCL), including anaplastic large cell lymphoma (ALCL); or cutaneous T cell lymphoma (CTCL), including primary cutaneous anaplastic large cell lymphoma (pcALCL).
29. Use of the pharmaceutical composition of item 22 for the manufacture of a medicament for treatment of a disease associated with overexpression of CD30.
30. The use of item 29, wherein the disease is selected from the group consisting of lymphoma, such as Hodgkin's lymphoma (HL), non-Hodgkin lymphoma (NHL), anaplastic large-cell lymphoma (ALCL), large B-cell lymphoma, paediatric lymphoma, T-cell lymphoma and enteropathy-associated T-cell lymphoma (EATL), leukaemia, such as acute myeloid leukaemia (AML), acute lymphoblastic leukaemia (ALL) and mast cell leukaemia, germ cell cancer, graft-versus-host disease (GvHD) and lupus, in particular systemic lupus erythematosus (SLE).
31. The use of item 30, wherein the disease is Hodgkin Lymphoma (HL) or anaplastic large cell lymphoma (ALCL).
31a. The use of item 29, wherein the disease is selected from the group consisting of peripheral T cell lymphoma—not otherwise specified (PTCL-NOS), angioimmunoblastic T-cell lymphoma (AITL), enteropathy associated T cell lymphoma (EATL), adult T-cell leukemia/lymphoma (ATLL), extranodal natural killer/T-cell lymphoma (ENKTCL), hepatosplenic and intestinal γ/δ-T cell lymphoma, nodal peripheral T-cell lymphoma with TFH phenotype, and follicular T cell lymphoma.
31b. The use of item 29, wherein the disease is peripheral T cell lymphoma (PTCL), including anaplastic large cell lymphoma (ALCL); or cutaneous T cell lymphoma (CTCL), including primary cutaneous anaplastic large cell lymphoma (pcALCL).
32. A method of treating a disease associated with overexpression of CD30, comprising the administration of an effective amount of the ADC of any one of items 1 to 19 to a subject or patient in need thereof.
33. The method of item 32, wherein the disease is a cancer associated with overexpression of CD30.
34. The method of item 32 or 33, wherein the disease is selected from the group consisting of lymphoma, such as Hodgkin's lymphoma (HL), non-Hodgkin lymphoma (NHL), anaplastic large-cell lymphoma (ALCL), large B-cell lymphoma, paediatric lymphoma, T-cell lymphoma and enteropathy-associated T-cell lymphoma (EATL), leukaemia, such as acute myeloid leukaemia (AML), acute lymphoblastic leukaemia (ALL) and mast cell leukaemia, germ cell cancer, graft-versus-host disease (GvHD) and lupus, in particular systemic lupus erythematosus (SLE), preferably Hodgkin Lymphoma (HL) or anaplastic large cell lymphoma (ALCL).
35. The method of item 34, wherein the disease is Hodgkin Lymphoma (HL) or anaplastic large cell lymphoma (ALCL).
35a. The method of item 32 or 33, wherein the disease is selected from the group consisting of peripheral T cell lymphoma—not otherwise specified (PTCL-NOS), angioimmunoblastic T-cell lymphoma (AITL), enteropathy associated T cell lymphoma (EATL), adult T-cell leukemia/lymphoma (ATLL), extranodal natural killer/T-cell lymphoma (ENKTCL), hepatosplenic and intestinal γ/δ-T cell lymphoma, nodal peripheral T-cell lymphoma with TFH phenotype, and follicular T cell lymphoma.
35b. The method of item 32 or 33, wherein the disease is peripheral T cell lymphoma (PTCL), including anaplastic large cell lymphoma (ALCL); or cutaneous T cell lymphoma (CTCL), including primary cutaneous anaplastic large cell lymphoma (pcALCL).
36. The method of any one of items 32 to 35b, wherein the ADC is administered at a dose of 14 mg/kg, 12 mg/kg, 10 mg/kg, 9 mg/kg, 8 mg/kg, 7 mg/kg, 6 mg/kg, 5 mg/kg, 4 mg/kg, 3 mg/kg, 2 mg/kg or 1 mg/kg; or at a dose of 2-6 mg/kg.
37. A method of treating a disease associated with overexpression of CD30, comprising the administration of an effective amount of the pharmaceutical composition of item 22 to a subject or patient in need thereof.
38. The method of item 37, wherein the disease is a cancer associated with overexpression of CD30.
39. The method of item 37 or 38, wherein the disease is selected from the group consisting of lymphoma, such as Hodgkin's lymphoma (HL), non-Hodgkin lymphoma (NHL), anaplastic large-cell lymphoma (ALCL), large B-cell lymphoma, paediatric lymphoma, T-cell lymphoma and enteropathy-associated T-cell lymphoma (EATL), leukaemia, such as acute myeloid leukaemia (AML), acute lymphoblastic leukaemia (ALL) and mast cell leukaemia, germ cell cancer, graft-versus-host disease (GvHD) and lupus, in particular systemic lupus erythematosus (SLE), preferably Hodgkin Lymphoma (HL) or anaplastic large cell lymphoma (ALCL).
40. The method of item 39, wherein the disease is Hodgkin Lymphoma (HL) or anaplastic large cell lymphoma (ALCL).
40a. The method of item 37 or 38, wherein the disease is selected from the group consisting of peripheral T cell lymphoma—not otherwise specified (PTCL-NOS), angioimmunoblastic T-cell lymphoma (AITL), enteropathy associated T cell lymphoma (EATL), adult T-cell leukemia/lymphoma (ATLL), extranodal natural killer/T-cell lymphoma (ENKTCL), hepatosplenic and intestinal γ/δ-T cell lymphoma, nodal peripheral T-cell lymphoma with TFH phenotype, and follicular T cell lymphoma.
40b. The method of item 37 or 38, wherein the disease is peripheral T cell lymphoma (PTCL), including anaplastic large cell lymphoma (ALCL); or cutaneous T cell lymphoma (CTCL), including primary cutaneous anaplastic large cell lymphoma (pcALCL).
41. The method of any one of items 37 to 40b, wherein the ADC is administered at a dose of 14 mg/kg, 12 mg/kg, 10 mg/kg, 9 mg/kg, 8 mg/kg, 7 mg/kg, 6 mg/kg, 5 mg/kg, 4 mg/kg, 3 mg/kg, 2 mg/kg or 1 mg/kg; or at a dose of 2-6 mg/kg.

It is noted that as used herein, the singular forms “a”, “an”, and “the”, include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the present invention.

The term “and/or” wherever used herein includes the meaning of “and”, “or” and “all or any other combination of the elements connected by said term”.

The term “less than” or in turn “more than” does not include the concrete number.

For example, less than 20 means less than the number indicated. Similarly, more than or greater than means more than or greater than the indicated number, e.g. more than 80% means more than or greater than the indicated number of 80%.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”. When used herein “consisting of” excludes any element, step, or ingredient not specified.

The term “including” means “including but not limited to”. “Including” and “including but not limited to” are used interchangeably.

As used herein the terms “about”, “approximately” or “essentially” mean within 20%, preferably within 15%, preferably within 10%, and more preferably within 5% of a given value or range. It also includes the concrete number, i.e. “about 20” includes the number of 20.

It should be understood that this invention is not limited to the particular methodology, protocols, material, reagents, and substances, etc., described herein and as such can vary. The terminology used herein is for the purpose of describing particular embodiments only, and is not intended to limit the scope of the present invention, which is defined solely by the claims.

All publications cited throughout the text of this specification (including all patents, patent application, scientific publications, instructions, etc.), whether supra or infra, are hereby incorporated by reference in their entirety. Nothing herein is to be construed as an admission that the invention is not entitled to antedate such disclosure by virtue of prior invention. To the extent the material incorporated by reference contradicts or is inconsistent with this specification, the specification will supersede any such material.

The content of all documents and patent documents cited herein is incorporated by reference in their entirety.

### EXAMPLES

An even better understanding of the present invention and of its advantages will be evident from the following examples, offered for illustrative purposes only. The examples are not intended to limit the scope of the present invention in any way.

### Materials and Methods

### Chemicals, Materials, Reagents and Solvents

Chemicals and solvents were purchased from Merck (Merck group, Germany), TCI (Tokyo chemical industry CO., LTD., Japan) and Acros Organics (Thermo Fisher scientific, USA), Sigma-Aldrich (Merck group, Germany), Carl Roth (Carl Roth GmbH, Germany), Cytiva (Cytiva UK Limited, England) and used without further purification. Dry solvents were purchased from Acros Organics (Thermo Fisher scientific, USA).

### TTL Expression and Purification

As described previously (Schumacher et al. (2015), Angewandte Chemie (International ed. in English), 54:13787-13791, WO 2016/066749, WO 2017/186855), tubulin-tyrosine ligase (TTL) was expressed in E. coli BL21(DE3) with an N-terminal His- and SUMO3 solubility-Tag. Cultivation was carried out in LB medium supplemented with Kanamycin (30 μg/mL) at 37° C., 180 rpm until a OD₆₀₀ of 0.6-0.8 was reached. After cooling down the culture to 18° C., cells were induced with 0.5 mM IPTG and protein expression was accomplished at 18° C. for 18 h.

After harvesting the culture by centrifugation, lysis was performed via sonification in TTL binding buffer (20 mM Tris/HCl pH 8.2, 250 mM NaCl, 20 mM Imidazol, 3 mM β-Mercaptoethanol) supplemented with Lysozyme (100 μg/ml), DNAse (25 μg/ml) and PMSF (1 mM), then debris centrifugation was carried out at 20.000 g for 30 min and the supernatant was filtered.

His-SUMO3-TTL was purified using a 5 ml His-Trap HP (Cytiva) applying a linear gradient over 4 CV to elution buffer (20 mM Tris/HCl pH 8.2, 250 mM NaCl, 500 mM Imidazol, 3 mM β-Mercaptoethanol). Purified protein was desalted subsequently on a PD-10 Sephadex G-25M column (Cytiva) by buffer exchange to TTL storage buffer (20 mM MOPS/KCl pH 7.4, 100 mM KCl, 10 mM MgCl₂, 50 mM L-Arginine, 3 mM β-Mercaptoethanol). Protein aliquots were shock-frozen and stored at −80° C.

### Brentuximab Tub-Tag Expression and Purification

Recombinant expression of Brentuximab Tub-tag was carried out in suspension-adapted CHO cells. The seed was grown in chemically defined; animal-component free medium and the supernatant was harvested by centrifugation and subsequent filtration. Purification was achieved using protein A affinity chromatography (MabSelect SuRe, Cytiva). The pH of the HCCF was adjusted to 7.5 and the column was equilibrated with binding buffer (20 mM NaH₂PO₄, 50 mM NaCl, 1 mM EDTA pH 7.5). The flow rate was adjusted depending on the titer of the culture to achieve optimal residence time on the column. Elution was carried out with a step to elution buffer (100 mM Trisodium citrate, pH 3.0). After neutralization of the eluate and buffer exchange to storage buffer (PBS, 100 mM L-Arginine) the antibody was stored at 8-15 mg/mL, 2-8° C. (short-term storage) or shock-frozen and stored at −80° C. (long-term storage).

### Brentuximab Tub-Tag Conjugation Reaction Applying TTL.

TTL catalyzed ligation was performed in accordance to previous publications (Schumacher et al. (2015), Angewandte Chemie (International ed. in English), 54:13787-13791, WO 2016/066749, WO 2017/186855). In brief 3-formyl-L-tyrosine was ligated to Brentuximab Tub-tag mAbs in various volumes consisting of 20 mM MOPS/KCl, 100 mM KCl, 10 mM MgCl₂, 5 mM ATP, 5 mM 3-formyl-L-tyrosine, pH 7.0. After adjusting the pH value, Brentuximab Tub-tag and TTL were added and the reaction was carried out at 18° C. for 12 h for tyrosination of the antibody.

### Purification of Tyrosinated Brentuximab-Tub-Tag

The crude tyrosination reaction was diluted to lower the conductivity (<10 mS/cm). Subsequently the mixture was loaded to a 5 mL HiTrap Capto Q ImpRes column (Cytiva). After washing the column with 5 CV AEX binding buffer (14.4 mM Na₂HPO₄, 5.6 mM NaH₂PO₄, pH 4.5) and 5 CV 10% AEX elution buffer (14.4 mM Na₂HPO₄, 5.6 mM NaH₂PO₄, 1 M NaCl pH 4.5), the elution of tyrosinated Brentuximab Tub-tag was achieved with a linear gradient to 50% elution buffer over 10 CV. The protein concentration of the pooled product containing fractions was measured with an UV280 method (NanoPhotometer NP80, IMPLEN).

Oxime-Ligation after TTL-Reaction

Protein concentration of tyrosinated Brentuximab Tub-tag after purification via anion-exchange chromatography was adjusted and DMSO was added as co-solvent (2% final v/v). Oxime-ligation was carried out with a slight excess of 2 (HA-vc-PAB-MMAE) or 3 (HA-(VC-PAB-MMAE)₂) at 18° C., until completion of reaction. The structures of payloads 2 and 3 are depicted in Scheme 2.

### Purification of Brentuximab Tub-Tag ADCs

The crude Oxime-ligation was diluted with CEX binding buffer (1 to 1 v/v, 14.4 mM Na₂HPO₄, 5.6 mM NaH₂PO₄, pH 4.5) and loaded to a 5 mL HiTrap Capto S ImpAct column (Cytiva). After washing the column with 10 CV CEX wash buffer (14.4 mM Na₂HPO₄, 5.6 mM NaH₂PO₄, 0.1% Triton-x-114, pH 4.5) and 5 CV CEX binding buffer the elution of TUB-010 was achieved with a linear gradient to 100% CEX elution buffer (14.4 mM Na₂HPO₄, 5.6 mM NaH₂PO₄, 1 M NaCl pH 4.5) over 20 CV. The pH of the pooled fractions was adjusted to 7.2. Buffer exchange was conducted via TFF (Vivaflow 50R) to formulation buffer.

### Synthesis of 3-formyl-L-tyrosine

### N-[(tertbutyloxy)carbonyl]-L-tyrosine

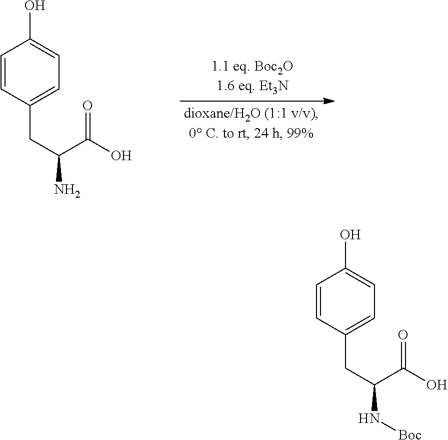 [SMILES: N[C@@H](Cc1ccc(O)cc1)C(=O)O ; CC(C)(C)OC(=O)N[C@@H](Cc1ccc(O)cc1)C(=O)O]

To a solution of 20 g L-Tyrosine (0.110 mol, 1 eq.) in 300 ml H₂O and 300 ml 1,4-Dioxane, 24.5 ml of triethylamine (0.176 mol, 1.6 eq.) was slowly added. The reaction mixture was cooled to 0° C. and 26.4 g of di-tert-butyl dicarbonate (0.121 mol, 1.1 eq.) were added portion-wise. Afterwards, the reaction mixture was allowed to slowly warm to room temperature overnight. The organic solvent was removed under reduced pressure and the solution acidified to pH 1 with 2 N HCl. The aqueous phase was extracted three times with 200 ml EtOAc, the organic fraction combined, dried over MgSO₄ and all volatiles removed under reduced pressure. The desired compound was obtained as a white solid (30.6 g, 0.109 mmol, 99%). Analytical data was in accordance with previously reported results (Schumacher et al. (2015), Angewandte Chemie (International ed. in English), 54:13787-13791).

### N-[(tertbutyloxy)carbonyl]-L-formyltyrosine

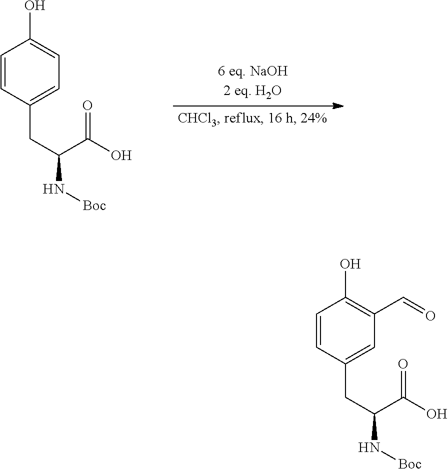 [SMILES: CC(C)(C)OC(=O)N[C@@H](Cc1ccc(O)cc1)C(=O)O ; CC(C)(C)OC(=O)N[C@@H](Cc1ccc(O)c(C=O)c1)C(=O)O]

To a suspension of 20 g N-[(tertbutyloxy)carbonyl]-L-tyrosine (0.071 mol, 1 eq.) in 300 ml Chloroform, 2.56 ml of H₂O (0.142 mol, 2 eq.) and 17.1 mg of powdered sodium hydroxide (0.427 mol, 6 eq.) were added portion wise. The reaction mixture was heated to reflux over night with vigorous stirring. The mixture was cooled to room temperature afterwards and diluted with 300 ml H₂O. The organic layer was separated and discarded. The aqueous layer was acidified to pH 1 with 2 N HCl and extracted three times with 200 ml EtOAc. The organic fractions were combined, dried over MgSO₄ and all volatiles removed under reduced pressure. The crude product was purified by column chromatography on silica (5% MeOH in CH₂Cl₂+0.5% formic acid) and obtained as yellowish solid. (5.26 g, 0.017 mol, 24%). Analytical data was in accordance with previously reported results (Schumacher et al. (2015), Angewandte Chemie (International ed. in English), 54:13787-13791).

### L-formyltyrosine TFA Salt

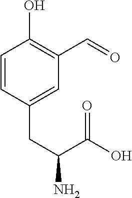 [SMILES: N[C@@H](Cc1ccc(O)c(C=O)c1)C(=O)O]

500 mg of N-[(tertbutyloxy)carbonyl]-L-formyltyrosine (1.616 mmol, 1 eq.) were suspended in 1 ml of CH₂Cl₂ and cooled to 0° C. 0.1 ml of H₂O were added, followed by a drop-wise addition of 1 ml trifluoroacetic acid. The reaction mixture was stirred for 3 hours at 0° C., all volatiles were removed in a nitrogen stream and the crude product was purified by preparative HPLC (Gilson Inc) with a VP 250/32 Macherey-Nagel Nucleodur C18 HTec Spum column (Macherey-Nagel GmbH) with the following gradient: (A=H₂O+0.1% TFA, B=MeCN (acetonitrile)+0.1% TFA, flow rate 30 ml/min, 5% B 0-5 min, 5-30% B 5-50 min, 99% B 50-60 min. The desired product was obtained as TFA salt as a yellowish powder after lyophilization. (397 mg, 1.228 mmol, 76%). Analytical data was in accordance with previously reported results (Schumacher et al. (2015), Angewandte Chemie (International ed. in English), 54:13787-13791).

### Synthesis of Hydroxylamine-MMAE (HA-VC-PAB-MMAE)

### (Fmoc-aminooxy) Acetic Acid (FMOC-HA-COOH)

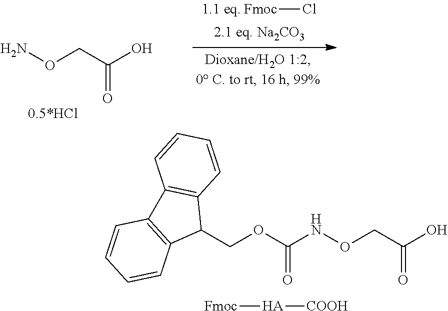

A 50-ml round-bottom flask was charged with 100 mg (0.915 mmol, 1.0 eq.) 0-(Carboxymethyl)hydroxylamine hemihydrochloride (Sigma-Aldrich), dissolved in 4 ml of water and 240 mg (2.264 mmol, 2.1 eq.) of sodium carbonate were added portion-wise. The solution was cooled to 0° C. and a solution of 260 mg (1.005 mmol, 1.1 eq.) Fluorenylmethyloxycarbonyl chloride (TCI-chemicals in 2 ml 1,4-Dioxane were added dropwise. The solution was allowed to warm to room temperature overnight. The dioxane was removed under reduced pressure, 50 ml of water were added, and the solution acidified with 2 mol/1 HCl. The aqueous phase was extracted three time with ethyl acetate and the combined organic phases were dried over MgSO₄. All volatiles were removed under reduced pressure and the desired compound obtained as a white solid. (283 mg, 99.0%) Analytical data was in accordance with previously reported results (Clave et al. (2008), Organic & Biomolecular Chemistry, 6:3065-3078).

### Fmoc-VC-PAB-PNP

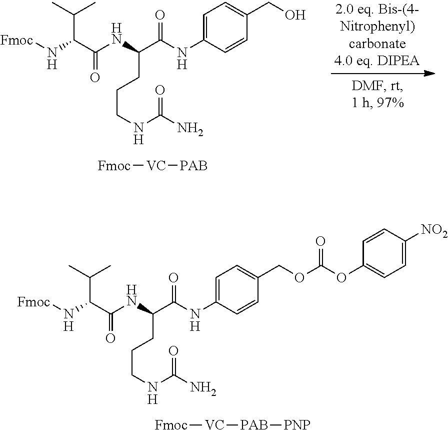

A 10-ml screw-cap vial was charged with 200 mg Fmoc-VC-PAB (0.333 mmol, 1.0 eq.), 202 mg Bis(4-nitrophenyl) carbonate (0.333 mmol, 1.0 eq.) and 1 ml of DMF. 232 μl of DIPEA (1.332 mmol, 4.0 eq.) were added and the reaction was stirred at room temperature for 1 h. The reaction mixture was poured into ice-cold diethylether and the filtrate collected via centrifugation. The filtrate was dissolved again in 1 ml of DMF and precipitate with ice-cold diethylether a second time. The precipitate was collected by centrifugation, dried under vacuum and the desired product was obtained as a yellowish powder. (247 mg, 97.0%). Analytical data was in accordance with previously reported results (WO2004010957).

### H₂N-VC-PAB-MMAE

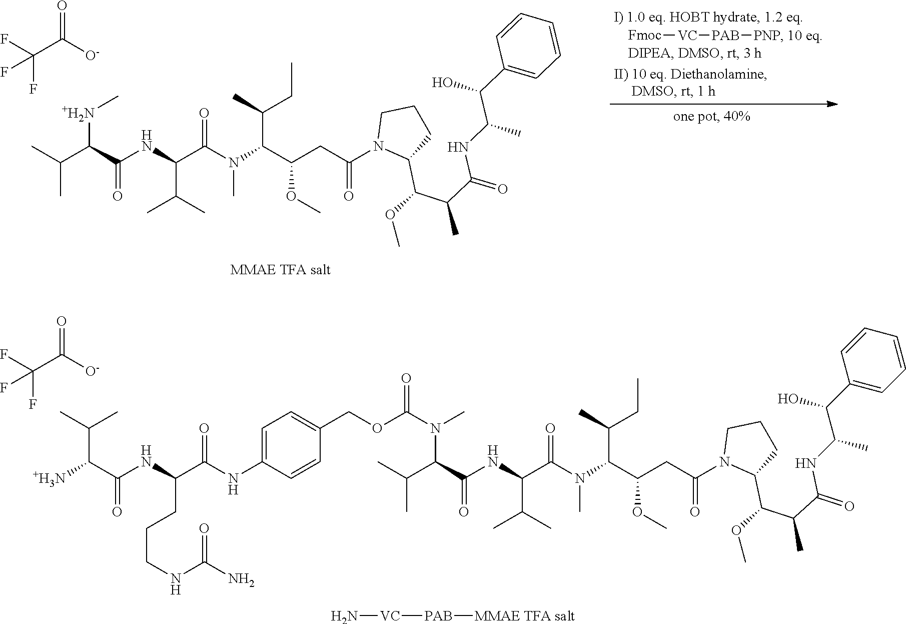

A 10-ml screw-cap vial was charged with 60 μl of a 1 mol/l solution of MMAE TFA salt in DMSO (50 mg, 0.06 mmol, 1.0 eq.), 180 μl of a 0.4 mol/L solution of Fmoc-VC-PAB-PNP solution in DMSO (0.072 mmol, 1.2 eq.) and 60 μl of a 1 mol/l solution of Hydroxybenzotriazole hydrate in DMSO (0.06 mmol, 1.0 eq.). 105 μl DIPEA (0.6 mmol, 10.0 eq.) were added and the yellow solution was stirred for three hours at room temperature. After full consumption of the MMAE starting material, monitored via UPLC/MS, 120 μl of a 50% (w/w) solution of diethanolamine in DMSO was added and the yellow solution further stirred for one hour. 1.5 ml of acetonitrile and 3 ml of water were added and the solution was purified via preparative HPLC (Gilson Inc) with a VP 250/32 Macherey-Nagel Nucleodur C18 HTec Spum column (Macherey-Nagel GmbH) with the following gradient: (A=H₂O+0.1% TFA, B=MeCN (acetonitrile)+0.1% TFA, flow rate 30 ml/min, 30% B 0-5 min, 30-60% B 5-25 min, 99% B 25-35 min. The desired product was obtained as TFA salt as a white powder after lyophilization. (29.5 mg, 39.7%). Analytical data was in accordance with previously reported results (Tang et al. (2016), Organic & Biomolecular Chemistry, 14(40): 9501-9518).

### HA-VC-PAB-MMAE

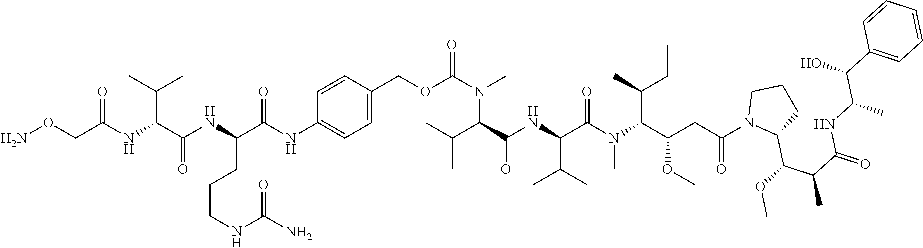 [SMILES: CC[C@H](C)[C@H]([C@H](CC(=O)N1CCC[C@@H]1[C@@H](OC)[C@H](C)C(=O)N[C@@H](C)[C@H](O)c1ccccc1)OC)N(C)C(=O)[C@H](NC(O)[C@@H](C(C)C)N(C)C(=O)OCc1ccc(NC(=O)[C@@H](CCCNC(N)=O)NC(=O)[C@H](NC(=O)CON)C(C)C)cc1)C(C)C]

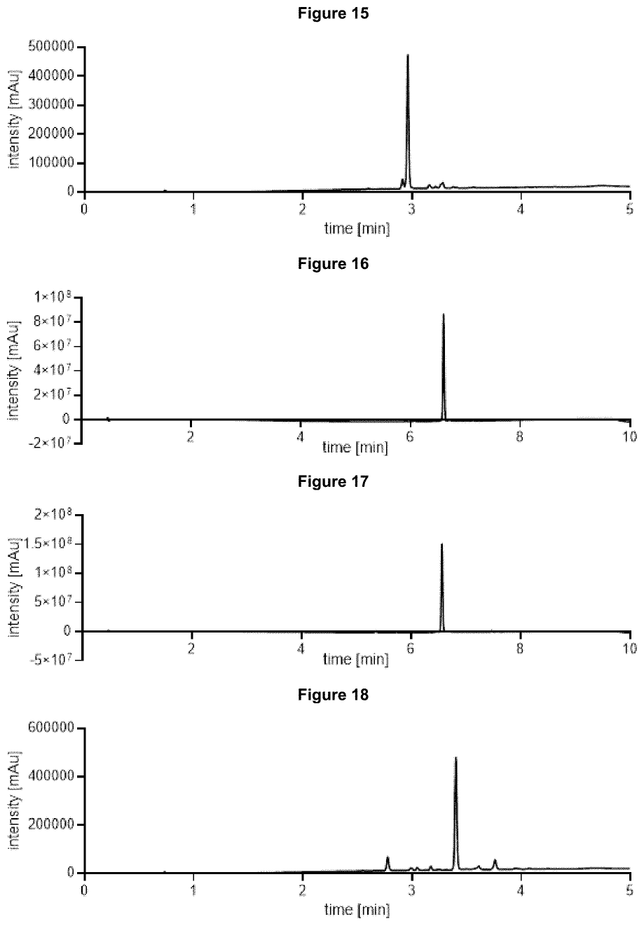

A 10-ml screw-cap vial was charged with 23.3 mg of H₂N-VC-PAB-MMAE (0.0188 mmol, 1.0 eq.), 38 μl of a 0.6 mol/L solution Fmoc-HA-COOH solution in DMSO (0.0226 mmol, 1.2 eq.) and 38 μl of a 0.6 mol/L solution Pybop in DMSO (0.0226 mmol, 1.2 eq.). 33 μl DIPEA (0.1880 mmol, 10.0 eq.) were added and the solution was stirred for two hours at room temperature. 150 μl of a 50% (w/w) solution of diethanolamine in DMSO was added and the solution further stirred for one hour. 1.5 ml of acetonitrile and 3 ml of water were added and the solution was purified via preparative HPLC (Gilson Inc) with a VP 250/32 Macherey-Nagel Nucleodur C18 HTec Spum column (Macherey-Nagel GmbH) with the following gradient: (A=H₂O+0.1% TFA, B=MeCN (acetonitrile)+0.1% TFA, flow rate 30 ml/min, 30% B 0-5 min, 30-99% B 5-60 min, 99% B 60-70 min. The desired product was obtained as TFA salt as a white powder after lyophilization. (20.1 mg, 81.6%). HR-MS for C₆₀H₉₈N₁₁O₁₄⁺[M+H]⁺ calcd.: 1196.7290, found 1196.7278. See FIG. 15 for an HPLC chromatogram of the purified substance.

### Synthesis of branched MMAE hydroxylamine (HA-(VC-PAB-MMAE)₂)

### BOC-Glu(OSu)₂

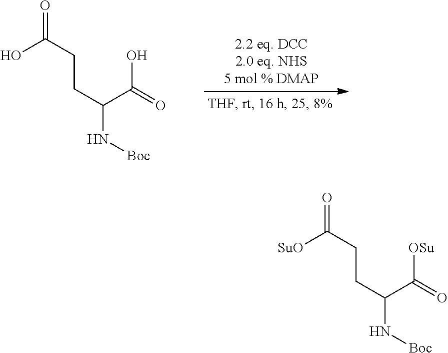

A 50-mL round-bottom flask was charged with 1.00 g Boc-Glu-OH (4.04 mmol, 1.00 eq.), 0.93 g N-Hydroxysuccinimide (8.08 mmol, 2.00 eq.) and 25 mg DMAP (0.20 mmol, 0.05 eq.), dissolved in 20 mL of dry THF. The suspension was cooled to 0° C. and 1.84 g Dicyclohexylcarbodiimide (8.88 mmol, 2.2 eq.) dissolved in 10 mL of dry THF were added drop-wise. The white suspension was allowed to warm to room-temperature over-night. The mixture was filtrated, the filtrate evaporated and the residue purified by flash-column-chromatography on silica gel. The desired product was obtained as colorless oil. (460 mg, 1.04 mmol, 25.8%). Analytical data was in accordance with previously reported results (Koshi et al., J. Am. Chem. Soc. 2008, 130, 1, 245-251).

### Boc-Glu-(VC-PAB-MMAE)₂

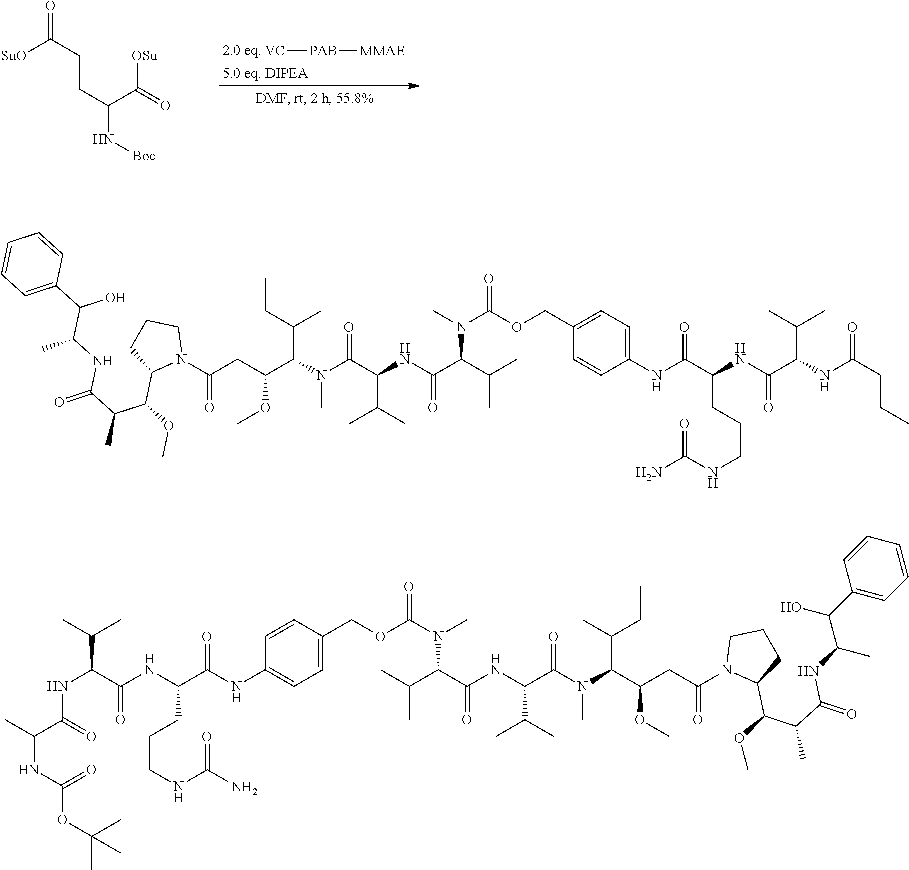

A 10-ml screw-cap vial was charged with 15.83 mg of H₂N—VC-PAB-MMAE TFA salt (0.012 mmol, 2.00 eq.), 2.84 mg Boc-Glu(OSu)₂ (0.06 mmol, 1.00 eq.) and 150 μL DMF. 4.5 μl DIPEA (0.0256 mmol, 4.0 eq.) were added and the solution was stirred for two hours at room temperature. 1.5 ml of acetonitrile and 3 ml of water were added and the solution was purified via preparative HPLC using a Gilson PLC 2020 system (Gilson Inc, WI, Middleton, USA) with a VP 250/21 Macherey-Nagel Nucleodur C18 HTec Spum column (Macherey-Nagel GmbH & Co. Kg, Germany) with the following gradient: (A=H₂O+0.1% TFA, B=MeCN (acetonitrile)+0.1% TFA, flow rate 10 ml/min, 30% B 0-5 min, 30-99% B 5-60 min, 99% B 60-70 min. The desired product was obtained as a white powder after lyophilization. (8.84 mg, 0.0036 mmol, 55.8%). HR-MS for C₁₂₆H₂₀₃N₂₁O₂₈²⁺[M+2H]²⁺ calcd.: 1229,7565, found 1229,7567. See FIG. 16 for an HPLC chromatogram of the purified substance.

### Boc-HA-Glu-(VC-PAB-MMAE)₂

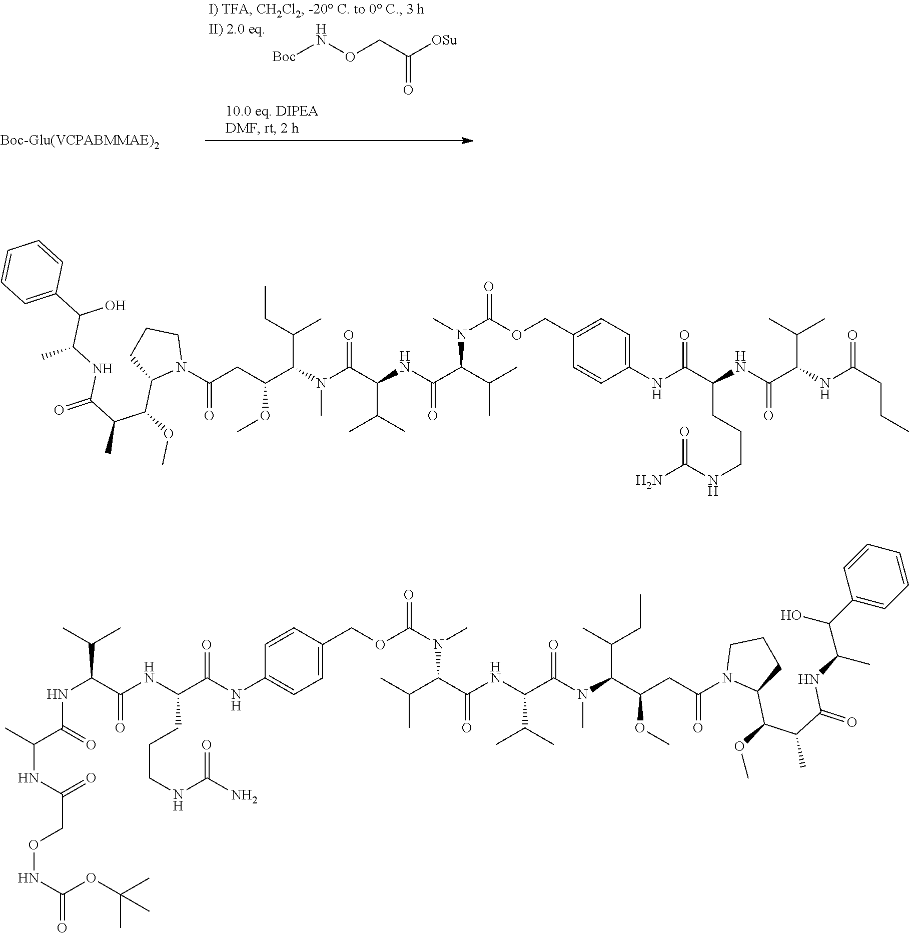

A 10-ml screw-cap vial was charged with 3.99 mg of Boc-Glu-(VC-PAB-MMAE)₂ (1.63 μmol, 1.00 eq.), dissolved in 100 μL CH₂Cl₂ and cooled to −20° C. 100 μL of Trifluoroacetic acid were added drop-wise. After 3 h at −20°, the solution was warmed to 0° C. and all volatiles were removed in an N₂-Stream. 0.94 mg Boc-HA-OSu (3.26 μmol, 2.00 eq.) dissolved in 100 μL DMF were added, followed by 2.8 μl DIPEA (0.163 μmol, 10.0 eq.) and the solution was stirred for two hours at room temperature. 1.5 ml of acetonitrile and 3 ml of water were added and the solution was purified via preparative HPLC using a Gilson PLC 2020 system (Gilson Inc, WI, Middleton, USA) with a VP 250/10 Macherey-Nagel Nucleodur C18 HTec Spum column (Macherey-Nagel GmbH & Co. Kg, Germany) with the following gradient: (A=H₂O+0.1% TFA, B=MeCN (acetonitrile)+0.1% TFA, flow rate 5 ml/min, 30% B 0-5 min, 30-99% B 5-60 min, 99% B 60-70 min. The desired product was obtained as a white powder after lyophilization. (2.11 mg, 0.748 μmol, 46%). HR-MS for C₁₂₈H₂₀₆N₂₂O₃₀²⁺ [M+2H]²⁺ calcd.: 1266,2647 found 1266,2644. See FIG. 17 for an HPLC chromatogram of the purified substance.

### HA-(VC-PAB-M MAE)₂

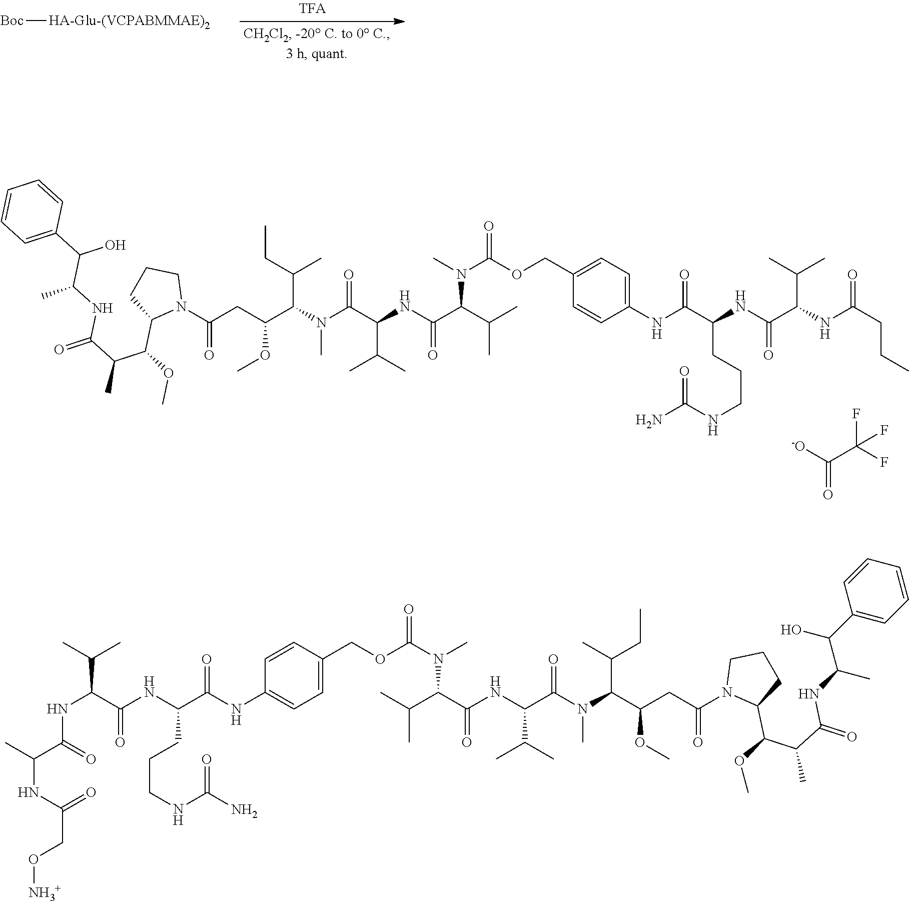

A 10-ml screw-cap vial was charged with 2.11 mg of Boc-Glu-(VC-PAB-MMAE)₂ (0.75 μmol), dissolved in 100 μL CH₂Cl₂ and cooled to −20° C. 100 μL of Trifluoroacetic acid were added drop-wise. After 3 h at −20°, the solution was warmed to 0° C. and all volatiles were removed in an N₂-Stream. The desired product was obtained as a white powder after lyophilization from 50% MeCN/H₂O. (1.90 mg, 0.748 μmol, quant.). HR-MS for C₁₂₃H₁₉₈N₂₂O₂₈²⁺ [M+2H]²⁺ calcd.: 1216,2384 found 1216,2326. See FIG. 18 for an HPLC chromatogram of the purified substance.

### Flash- and Thin Layer Chromatography

Flash column chromatography was performed, using NORMASIL 60® silica gel 40-63 μm (VWR international, USA). Glass TLC plates, silica gel 60 W coated with fluorescent indicator F254s were purchased from Merck (Merck Group, Germany). Spots were visualized by fluorescence depletion with a 254 nm lamp or manganese staining (10 g K₂CO₃, 1.5 g KMnO₄, 0.1 g NaOH in 200 ml H₂O), followed by heating.

### Preparative HPLC

Preparative HPLC was performed on a Gilson PLC 2020 system (Gilson Inc, WI, Middleton, USA) using a VP 250/32 Macherey-Nagel Nucleodur C18 HTec Spum column (Macherey-Nagel GmbH & Co. Kg, Germany). The following gradients were used: Method C: (A=H₂O+0.1% TFA (trifluoroacetic acid), B=MeCN (acetonitrile)+0.1% TFA, flow rate 30 ml/min, 5% B 0-5 min, 5-90% B 5-60 min, 90% B 60-65 min. Method D: (A=H₂O+0.1% TFA, B=MeCN++0.1% TFA), flow rate 30 ml/min, 5% B 0-5 min, 5-25% B 5-10 min, 25%-45% B 10-50 min, 45-90% 50-60 min, 90% B 60-65 min. Method E: 0.1% TFA, flow rate 18 ml/min, 5% B 0-5 min, 5-90% B 5-60 min, 90% B 60-65 min, using a VP 250/21 Macherey-Nagel Nucleodur C18 HTec Spum column (Macherey-Nagel GmbH & Co. Kg, Germany).

### HR-MS

High resolution ESI-MS spectra were recorded on a Waters H-class instrument equipped with a quaternary solvent manager, a Waters sample manager-FTN, a Waters PDA detector and a Waters column manager with an Acquity UPLC protein BEH C18 column (1.7 μm, 2.1 mm×50 mm). Samples were eluted with a flow rate of 0.3 mL/min. The following gradient was used: A: 0.01% FA in H₂O; B: 0.01% FA in MeCN. 5% B: 0-1 min; 5 to 95% B: 1-7 min; 95% B: 7 to 8.5 min. Mass analysis was conducted with a Waters XEVO G2-XS QT of analyzer.

### UPLC-UV/MS

UPLC-UV/MS traces were recorded on a Waters H-class instrument equipped with a quaternary solvent manager, a Waters autosampler, a Waters TUV detector and a Waters Acquity QDa detector with an Acquity UPLC BEH C18 1.7 μm, 2.1×50 mm RP column with a flow rate of 0.6 mL/min (Waters Corp., USA). The following gradient was used for purity analyses: A: 0.1% TFA in H₂O; B: 0.1% TFA in MeCN. 5% B 0-1.5 min, 5-95% B 1.5-11 min, 95% B 11-13 min, 5% B 13-15 min.

### Intact Protein MS

Intact proteins were analyzed using a Waters H-class instrument equipped with a quaternary solvent manager, a Waters sample manager-FTN, a Waters PDA detector and a Waters column manager with an Acquity UPLC protein BEH C4 column (300 Å, 1.7 μm, 2.1 mm×50 mm). Proteins were eluted with a flow rate of 0.3 mL/min. The following gradient was used: A: 0.01% FA in H₂O; B: 0.01% FA in MeCN. 5-95% B 0-6 min. Mass analysis was conducted with a Waters XEVO G2-XS QT of analyzer. Proteins were ionized in positive ion mode applying a cone voltage of 40 kV. Raw data was analyzed with MaxEnt 1.

### Resazurin Assay

HL60 and Karpas cell lines were cultured in RPMI-1640 supplemented with 10% FCS and 0.5% Penicillin-Streptomycin. Cells were seeded at a density of 5*10″3 cells/well in 96-well cell culture microplate. 1:4 serial dilutions of ADCs or antibodies were performed in cell culture medium starting at 3 μg/mL final concentration and transferred in duplicates to respective wells on the microplate. Plates were incubated for 96 h at 37° C. 5% CO₂. Subsequently, resazurin was added to a final concentration of 50 μM followed by incubation for 3-4 h at 37° C., 5% CO₂. Metabolic conversion of resazurin to resorufin is quantified by the fluorescent signal of resorufin (A_{Ex}=560 nm and λ_{EM}=590 nm) on a Tecan Infinite M1000 micro plate reader. Mean and standard deviation was calculated from duplicates, normalized to untreated control and plotted against antibody concentration. Data analysis was performed with MatLab R2016 software.

### Characterization and Stability Assessment of ADCs by A-SEC

ADCs were adjusted to a protein concentration of 1 mg/mL in PBS (Dulbeccos Phosphate Bufferd Saline, Sigma-Aldrich Merck KGaA) and filtered sterile (Ultrafree-MC Centrifugal filter units, Merck Millipore). Samples were stored at 4-8° C., 37° C. and 40° C. for up to 14 days. For samples stored at elevated temperatures, it was ensured that no condensate was formed. Before analysis via A-SEC the samples where centrifuged at 4° C., 4000×g for 4 minutes.

### In Vivo Xenograft Model

In vivo efficacy experiments were performed in accordance with animal welfare law and approved by local authorities. In brief, Karpas 299 cells were subcutaneously injected to CB17-Scid mice at day 0. Treatment was initiated when tumors reached a certain mean tumor volume. Following randomization of mice into treatment and control groups ADCs were administered as intravenous injection. Tumor volumes, body weights and general health conditions were recorded throughout the whole study.

### Pharmacokinetic Analysis

Experiments were performed in accordance with animal welfare law and approved by local authorities. The test items were administered to Sprague Dawley rats at day 1 by a single intravenous (bolus) injection. For the bioanalytic analysis blood was collected at various time points for up to 504 hours via the jugular vein with a target volume of 1 mL. Samples were kept at room temperature for at least 1 hour to allow clotting. The samples were centrifuged at 1500 g at 4° C. for 10 minutes. The resultant serum was stored at −20° C. PK parameters were calculated using Phoenix (WinNonlin) pharmacokinetic software 1.4 (Certara, 6.4) using a non-compartmental approach consistent with the intravenous (bolus) route of administration, with an in-vivo formation profile approach (extravascular) taken for free payload.

### Repeated Dose Toxicity Study

Experiments were performed in accordance with animal welfare law and approved by local authorities. Male and female Sprague Dawley rats (10/sex/group) were dosed with control (vehicle), Brentuximab Tub-tag ADC (10 mg/kg) or Brentuximab vedotin (10 mg/kg) weekly (days 1, 8, 15 & 22), with main dosing phase necropsy on day 26 (5/sex/group) and recovery phase necropsy on day 51 (5/sex/group). Clinical observations, body weight and food consumption were determined daily throughout the study. Pharmacokinetic samples were obtained prior to each dose, 15 minutes post each dose and at necropsy from each animal. All animals were necropsied, with organs weighed and retained in fixative. Clinical pathology (hematology, clinical chemistry and coagulation end-points) samples were also taken at necropsy.

### Example 1: Brentuximab Tub-Tad ADCs for the Treatment of CD30+ Indications

The Tub-tag technology makes use of a natural alpha-tubulin derived peptide that is highly polar and serves as a recognition sequence for the enzyme Tubulin tyrosine ligase (TTL), i.e. is a recognition sequence for tubulin tyrosine ligase within the meaning of this disclosure.

TTL recognizes the short hydrophilic peptide tag (Tub-tag) and catalyses the peptide bond formation with tyrosine derivatives and amino acid like building blocks. Here the Inventors demonstrate that the Tub-tag technology can be used to conjugate payloads to the antibody Brentuximab (cAC10) facilitating CD30 binding ADCs that surprisingly outcompete the approved Brentuximab based ADC Brentuximab vedotin in terms of stability, efficacy and toxicology.

The Inventors started their investigations by fusing (on the nucleic acid level) the recognition sequence for tubulin tyrosine ligase to the light chain (light chain SEQ ID NO: 11, heavy chain: SEQ ID NO: 1), heavy chain (light chain SEQ ID NO: 2, heavy chain: SEQ ID NO: 12) or light and heavy chain (light chain SEQ ID NO: 11, heavy chain: SEQ ID NO: 12) of the monoclonal antibody cAC10 (Brentuximab, Tub-tag fused to the light chain exemplarily shown in Scheme 2), optionally with the sequence of an Gly4Ser amino acid linker arranged between the recognition sequence for tubulin tyrosine ligase to the respective antibody chain.

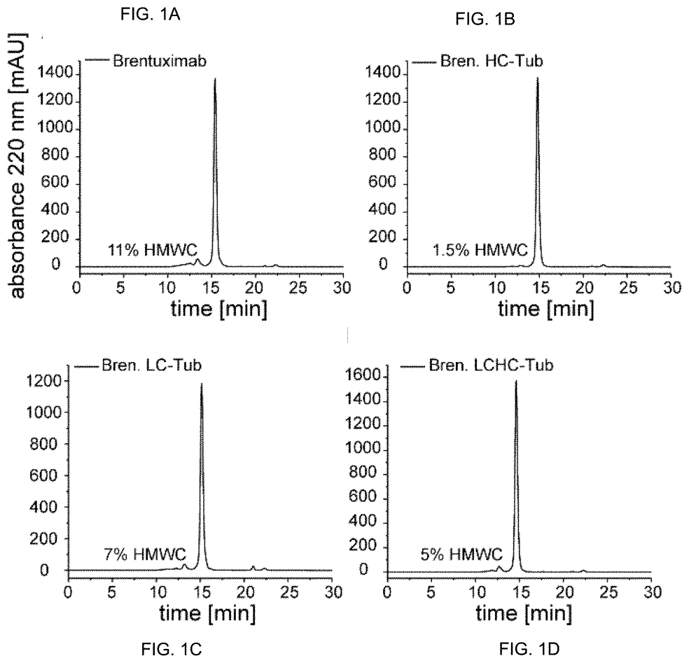

These Brentuximab variants were recombinantly expressed using established cell culture methods and subsequently successfully purified with Protein A chromatography. Analysis with size exclusion chromatography (SEC) showed an excellent purity of the Tub-tag antibodies and only minimal amount of high molecular weight species (HMWS) were observed (HMWS=1.5-7%). The results are shown in FIGS. 1A-1D. These results show that the antibodies are highly monomeric after expression and purification. As can be seen in FIG. 1B, attachment of the Tub-tag recognition sequence to the heavy chain of Brentuximab results in a decrease of high molecular weight species from 11% to only 1.5%, i.e. a decrease by a factor of 10.

FIG. 1A shows an analytical size exclusion chromatogram of unmodified Brentuximab, which is also denoted herein as just “Brentuximab”, after Protein A chromatography (PAC). “HMWC” stands for “High Molecular Weight Components”.

FIG. 1B shows an analytical size exclusion chromatogram of Brentuximab comprising TTL recognition sequences (Tub-tags) fused to the heavy chains (light chain: SEQ ID NO: 2, heavy chain: SEQ ID NO: 12), which is also denoted herein as “Bren. HC-Tub”, after Protein A chromatography (PAC).

FIG. 1C shows an analytical size exclusion chromatogram of Brentuximab comprising TTL recognition sequences (Tub-tags) fused to the light chains (light chain: SEQ ID NO: 11, heavy chain: SEQ ID NO: 1), which is also denoted herein as “Bren. LC-Tub”, after Protein A chromatography (PAC).

FIG. 1D shows an analytical size exclusion chromatogram of Brentuximab comprising TTL recognition sequences (Tub-tags) fused to the light chains and the heavy chains (light chain: SEQ ID NO: 11, heavy chain: SEQ ID NO: 12), which is also denoted herein as “Bren. LCHC-Tub”, after Protein A chromatography (PAC).

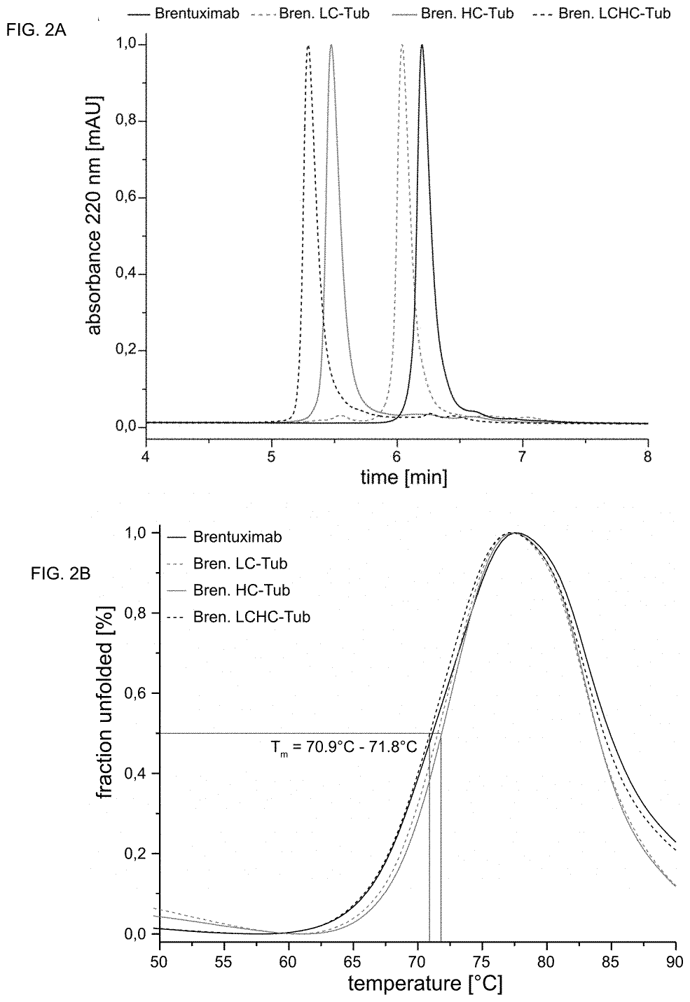

After showing the manufacturability of Brentuximab comprising at the C-terminus of the light chains, the heavy chains or all of the heavy and light chains of the Brentuximab a recognition sequence for tubulin tyrosine ligase (“Tub tag variants” or “Brentuximab tub-tags”), the Inventors determined the retention times by hydrophilic interaction chromatography (HIC), which is a common measure for hydrophobicity. This study revealed a higher hydrophilicity and surface polarity of all Tub-tag variants in comparison to the unmodified cAC10 (Brentuximab) (FIG. 2A).

FIG. 2A shows the analytical hydrophobic interaction chromatograms of Brentuximab (black), Bren. LC-Tub (grey, dotted), Bren. HC-Tub (grey) and Bren. LCHC-Tub (black, dotted). The chromatogram shows normalized absorption spectra recorded at 220 nm. The retention time is a measure for hydrophobicity. A longer retention time in the hydrophobic interaction chromatogram indicates a greater hydrophobicity. A shorter retention time indicates as greater hydrophilicity.

Since alterations of the antibody backbone can result in perturbation of structure and stability, the melting point (Tm) of Brentuximab and Tub-tag variants was measured with differential scanning fluorimetry (DSF). These experiments verified, that Tub-tag variants have the same melting point (Tm=70.9-71.8° C.) and thus the same stability against thermal stress as the unmodified mAb (Tm=71.0° C.) (FIG. 2B).

FIG. 2B shows the differential scanning fluorimetry curves for determination of the melting point (Tm) of Brentuximab, Bren. LC-Tub, Bren. HC-Tub and Bren. LCHC-Tub).

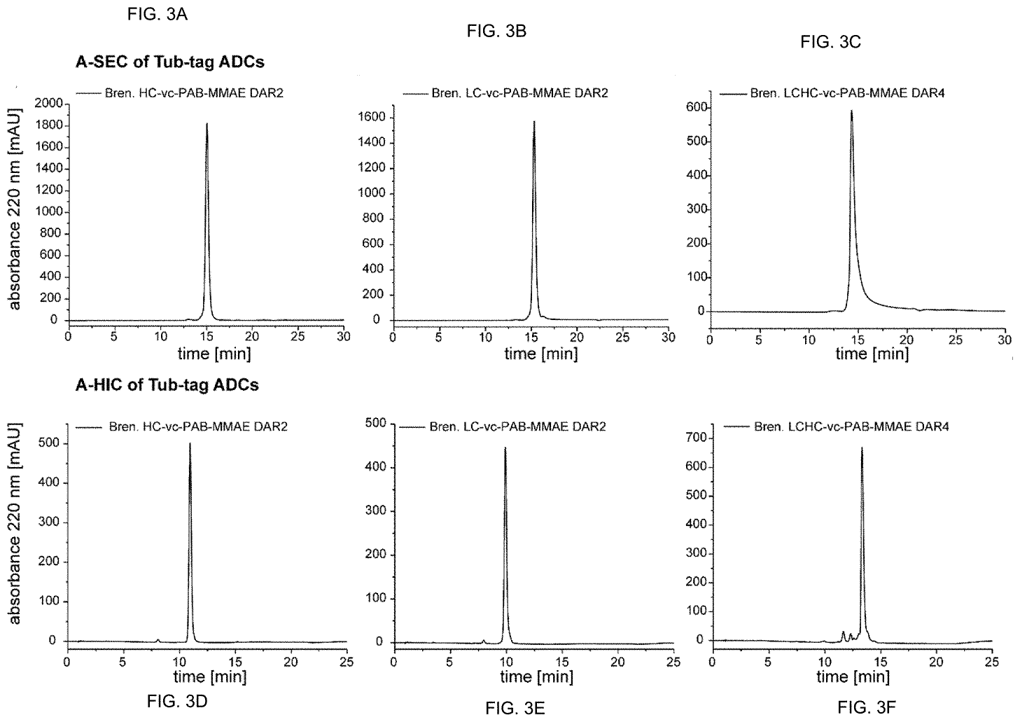

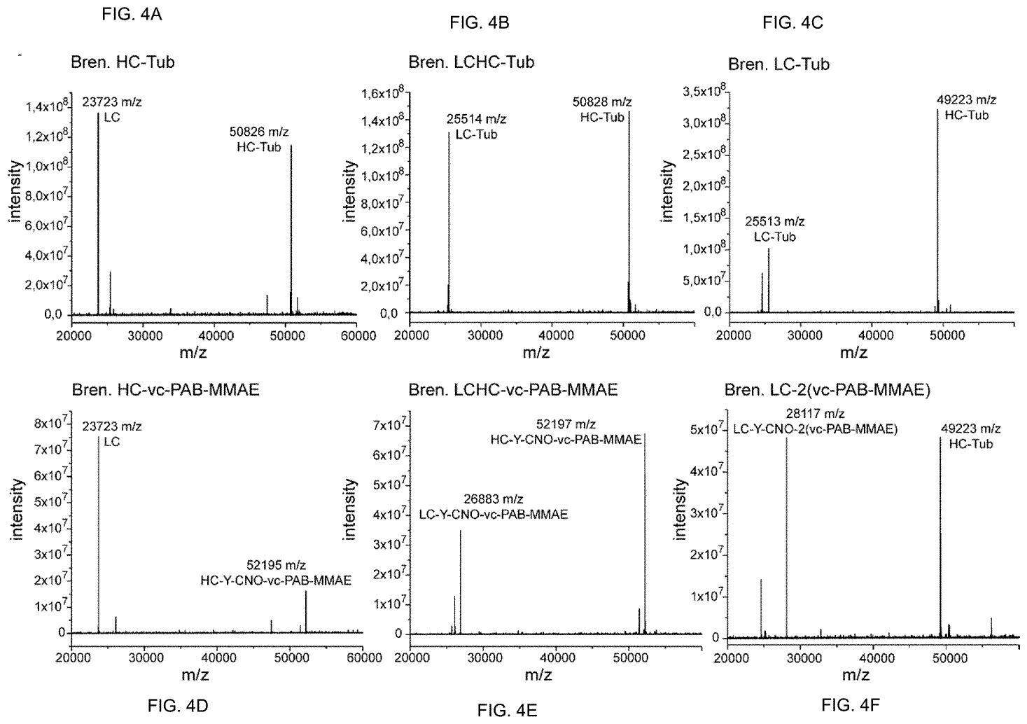

The Inventors tested different tyrosine-derivatives for enzymatic modification of the Tub-tag-antibodies, while 3-formyl-L-tyrosine turned out to be optimal for ADC manufacturing. In brief, the Inventors incubated the different Brentuximab Tub-tag variants with TTL to covalently attach 3-formyl-L-tyrosine to the terminal glutamic acid residue of the Tub-tag. In particular, the amino group of the 3-formyl-L-tyrosine was attached to the C-terminus of the Tub-tag. Thereafter, excess of 3-L-formyl-tyrosine and residual TTL were removed with Protein A chromatography or anion-exchange chromatography (AEX). Subsequently, a linker-payload (“linker-drug moiety”) was covalently attached by a bioorthogonal reaction. The purification of the resulting ADC was conducted with cation exchange chromatography (CEX) to remove excess-linker payload and to refine the purity of the ADC. The final step of the process involved formulation via tangential flow filtration (TFF) and sterile filtration. For production of ADCs with a drug to antibody ratio (DAR) of 2 we applied the linear payload 2 (depicted in Scheme 2, “structure 4” as defined in the description and claims conjugated with MMAE). For production of ADCs with DAR 4, we applied branched payload 3 (depicted in Scheme 2, “structure 5” as defined in the description and claims conjugated with MMAE). In particular, the H₂N—O— moiety of payload 2 and payload 3 reacted with the formyl group of the 3-formyl-L-tyrosine bound to the TTL recognition sequence to give an oxime having a —C═N—O— structure. This procedure yielded pure, homogenous ADCs, and the quality was controlled for example by means of SEC, HIC (FIGS. 3A-3F) and middle-up MS (FIGS. 4A-4F) showing excellent purity and homogeneity.

The following ADCs have been prepared and further investigated in the Examples (the payloads 2 and 3 are shown in Scheme 2):

| | | | | | Drug to |
|---|---|---|---|---|---|
| | Brentuximab | Brentuximab | Non-natural | | Antibody |
| ADC | light chain | heavy chain | amino acid | Payload | Ratio (DAR) |
| Bren. HC-2 | SEQ ID NO: 2 | SEQ ID NO: 12 | 3-formyl-L- | Payload 2 | 2 |
| | | (comprising | tyrosine | | |
| | | TTL | | | |
| | | recognition | | | |
| | | sequence) | | | |
| Bren. LC-2 | SEQ ID NO: 11 | SEQ ID NO: 1 | 3-formyl-L- | Payload 2 | 2 |
| | (comprising | | tyrosine | | |
| | TTL | | | | |
| | recognition | | | | |
| | sequence) | | | | |
| Bren. LCHC-2 | SEQ ID NO: 11 | SEQ ID NO: 12 | 3-formyl-L- | Payload 2 | 4 |
| | | | tyrosine | | |
| Bren. LC-3 | SEQ ID NO: 11 | SEQ ID NO: 1 | 3-formyl-L- | Payload 3 | 4 |
| | | | tyrosine | | |

FIGS. 3A-3F show an analysis of Brentuximab Tub-tag ADCs via SEC (FIGS. 3A to 3C) and HIC (FIGS. 3D to 3F). FIGS. 3A and 3D show the results for Bren. HC-vc-PAB-MMAE DAR2 (also denoted herein as “Bren. HC-2”), FIGS. 3B and 3E show the results for Bren. LC-vc-PAB-MMAE DAR2 (also denoted herein as “Bren. LC-2”), and FIGS. 3C and 3F show the results of Bren. LCHC-vc-PAB-MMAE DAR4 (also denoted herein as “Bren. LCHC-2”). Each of the Brentuximab variants was expressed, purified with PAC, and conjugated to payload 2 for generation of DAR 2 and payload 2 or payload 3 for generation of DAR 4 ADCs. After final polishing with HIC and buffer exchange, Tub-tag ADCs were analyzed in terms of aggregate content and DAR homogeneity. All Tub-tag ADCs contain a very low content of HMWS (<1%) and displayed an excellent DAR homogeneity.

FIGS. 4A-4F show an analysis of Brentuximab Tub-tag ADCs by middle-up protein MS after deglycosylation. Shown are deconvoluted spectra. FIG. 4A shows the result for Bren. HC-Tub; FIG. 4B shows the result for Bren. LCHC-Tub; FIG. 4C shows the result for Bren. LC-Tub, FIG. 4D shows the result for Bren. HC-vc-PAB-MMAE (also denoted herein as “Bren. HC-2”); FIG. 4E shows the result for Bren. LCHC-vc-PAB-MMAE (also denoted herein as “Bren. LCHC-2”); and FIG. 4F shows the result for Bren. LC-2(vc-PAB-MMAE) (also denoted herein as “Bren. LC-3”). The mass shift of 1369 m/z results from the incorporation of 3-formyl-L-tyrosine and oxime ligation with payload 2. After oxime ligation with payload 3 a mass difference of 2604 m/z was observed. These results are in accordance with the calculated values.

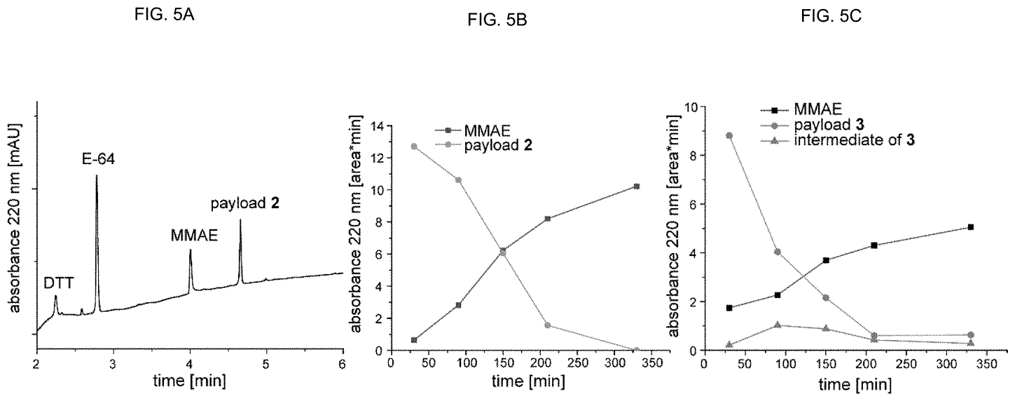

Prior to the conjugation with the antibody, payloads 2 and 3 have been analyzed for their susceptibility to cleavage by the protease cathepsin B (FIGS. 5A-5C). The results show that payloads 2 and 3 are cleaved by cathepsin B to release the free drug monomethyl auristatin E (MMAE).

FIGS. 5A-5C show in FIG. 5A an RP-HPLC analysis of the valine-citrulline (vc) containing payload 2 during digestion reaction with the protease cathepsin B (1:1000 for each vc moiety). Prior to analysis the reaction was stopped with E-64. The chromatogram after a reaction time of 150 min was recorded at 220 nm. The plot of FIG. 5B shows the increase of free MMAE over time (black) and the decrease of payload 2 (grey, circle) during reaction with cathepsin B. FIG. 5C shows the cleavage of payload 3. During the reaction of payload 3 with cathepsin B an intermediate is formed (grey, triangle) which contains one MMAE moiety.

### Example 2: In Vitro Stability of ADCs

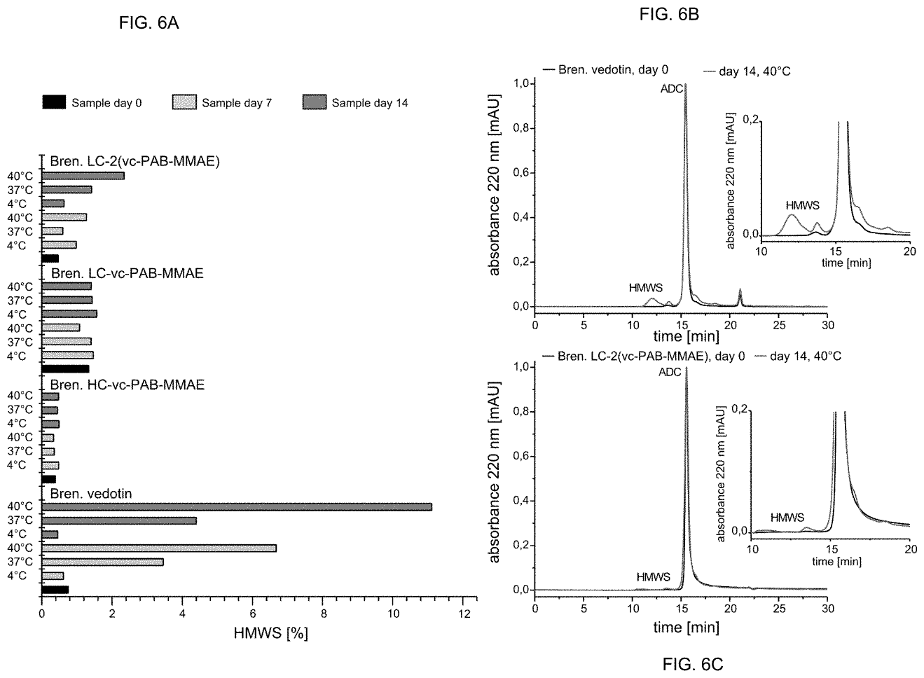

To elucidate the stability of Brentuximab ADCs described in Example 1, the Inventors performed thermal stress tests based on the fluorescent dye SYPRO Orange. The DSF experiment elucidated that Brentuximab Tub-tag ADCs possess a melting point similar to the corresponding mAb (Brentuximab Tm=71.0° C., Brentuximab Tub-tag ADCs Tm=70.9-72.9° C.). This illustrates the antibody-like stability of Brentuximab Tub-tag ADCs. Brentuximab vedotin, which was used for comparison displayed a lower melting point of 68.5° C. and the melting curve was covering a higher temperature range. In addition, a stability study of ADCs was carried out in buffer and mouse-plasma. Samples stored in buffer were separated via SEC to analyze the formation of HMWS (High Molecular Weight Species). No significant variance in the chromatogram was observed after two weeks of storage at 4° C. However, differences became visible during storage at elevated temperatures. After dissolving Brentuximab vedotin according to the manufacturer guidelines a HMWS of 0.74% was determined. This value increased during 14 days storage at 40° C. to 11.44%, representing a 15.5-fold increase of HMWS. In comparison to these values Brentuximab Tub-tag ADCs where very stable and a low 1.0-5.0 fold-increase of HMWS content was measured (FIG. 6A). Although Brentuximab LC-3 DAR 4 contains the same drug load as Brentuximab vedotin (DARav 4) and is therefore characterized by high hydrophobicity derived by the payload, a low increase of HMWS content was measured. After manufacturing the Tub-tag ADC Bren. LC-3 with DAR 4, a HMWS of 0.47% was determined. During the storage at 40° C. only a small increase in HMWS to 2.34% was observed. (FIG. 6C, for comparison the results for Brentuximab vedotin are shown in FIG. 6B).

FIGS. 6A-6C show the results of the storage of Brentuximab vedotin and Brentuximab Tub-tag ADCs at elevated temperatures. FIG. 6A displays the increase of HMWS during the course of the study for Bren. LC-2(vc-PAB-MMAE) (also denoted herein as “Bren. LC-3”); Bren. LC-vc-PAB-MMAE (also denoted herein as “Bren. LC-2”); Bren. HC-vc-PAB-MMAE (also denoted herein as “Bren. HC-2”); and Brentuximab vedotin. The highest increase of aggregate content can be seen for Brentuximab vedotin. In comparison, the HMWS content of Tub-tag ADCs remains almost constant. FIGS. 6B and 6C show SEC chromatograms showing normalized absorption spectra recorded at 220 nm for Brentuximab vedotin (average drug to antibody ratio (DARₐᵥ) 4) and Bren. LC conjugated with payload 3 (Bren. LC-3, DAR 4).

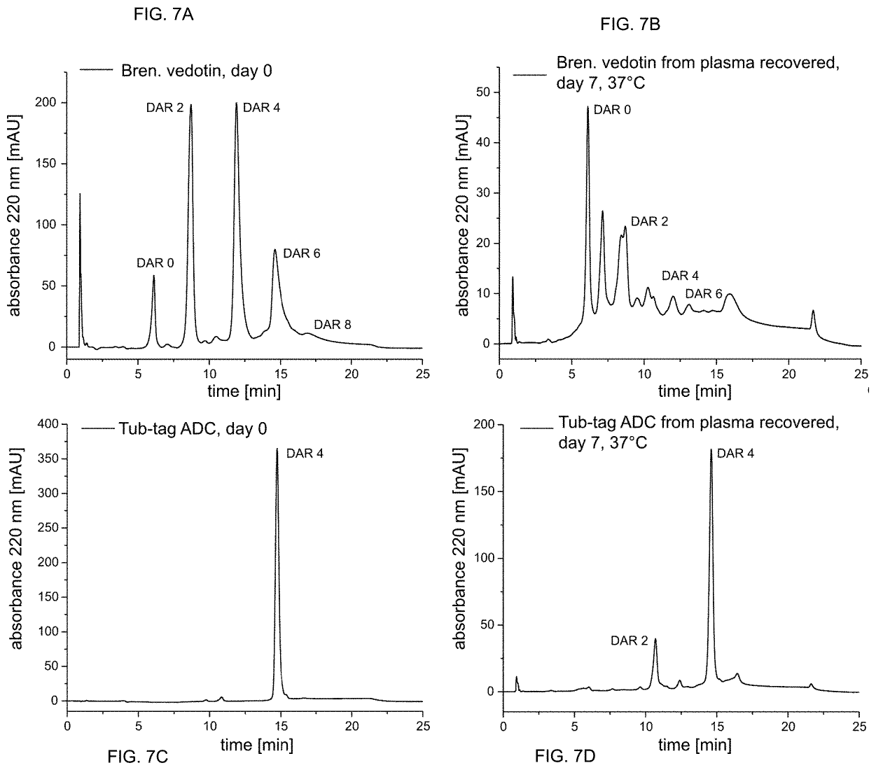

In addition, the stability of Brentuximab Tub-tag ADCs has been evaluated in mouse plasma showing excellent stability of Tub-tag ADCs. In contrast, Brentuximab vedotin was rapidly decomposed (FIGS. 7A-7D, Table 1). The rapid decomposition of maleimide-linked ADCs like Brentuximab vedotin has recently been shown and retro-Michael addition could be one explanation for our observations.

TABLE 1
Storage of Brentuximab vedotin and Brentuximab Tub-tag ADCs in
mouse-plasma for 7 days at 37° C.

| | DAR before | DARₐᵥ | Payload |
|---|---|---|---|
| | plasma | after plasma | lost |
| ADC | incubation | incubation n = 2 | [%] |
| Bren. HC-2 | 2 | 1.8 | 10 |
| Bren. LC-2 | 2 | 1.8 | 10 |
| Bren. LCHC-2 | 3.8 | 3.63 | 5 |
| Bren. LC-3 | 4 | 3.59 | 10 |
| Brentuximab vedotin | 4.02 | 1.53 | 62 |

FIGS. 7A-7D show an exemplary illustration of the stability of a Brentuximab Tub-tag ADC in mouse plasma compared to Brentuximab vedotin. FIGS. 7A and 7B show the result of storage of Brentuximab vedotin at 37° C. in mouse-plasma. FIGS. 7C and 7D show the result of storage of Bren. LC-3 DAR 4 at 37° C. in mouse-plasma.

### Example 3: In Vitro Efficacy of ADCs

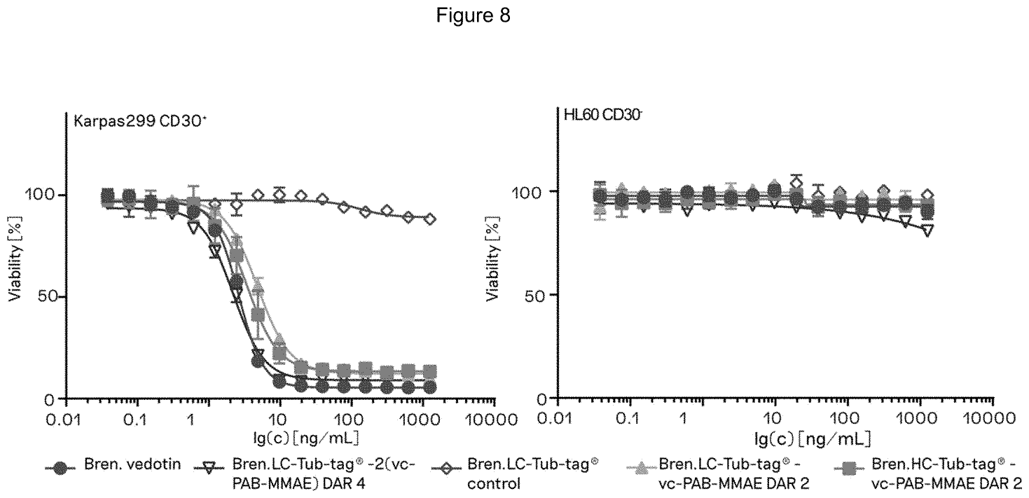

The Inventors then evaluated the in vitro efficacy of Brentuximab Tub-tag ADCs in a cell-based viability assay and compared the results with Brentuximab vedotin. The CD30-overexpressing cell line Karpas299 as well as the CD30-negative cell line HL60 were used for this resazurin assay (FIG. 8). The determined IC50 values for Brentuximab vedotin (IC50=2.08) in the antigen positive cell line Karpas299 were in line with the data from literature. Overall, the Brentuximab Tub-tag ADCs showed low IC50 values and a good in vitro efficacy (Table 2).

TABLE 2
IC₅₀ values for Brentuximab vedotin and Brentuximab Tub-tag ADCs
determined by a resazurin assay.

| ADC | IC₅₀Karpas299 Cells |
|---|---|
| Bren. HC-2 | 5.24 n = 1 * 2 |
| Bren. LC-2 | 5.63 n = 4 * 2 |
| Bren. LCHC-2 | 1.70 n = 2 * 2 |
| Bren. LC-3 | 1.06 n = 5 * 2 |
| Brentuximab vedotin | 2.08 n = 5 * 2 |

FIG. 8 shows the in vitro efficacy of Brentuximab Tub-tag ADCs in the CD30-overexpressing cell line Karpas299 and the CD30-negative cell line HL60.

### Example 4: In Vivo Efficacy of Brentuximab Tub-Tag ADCs

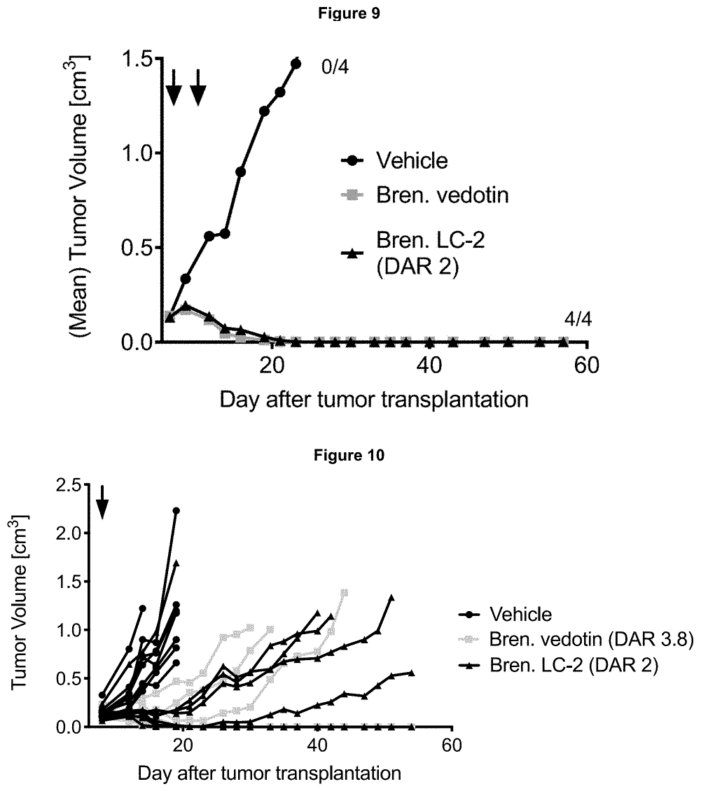

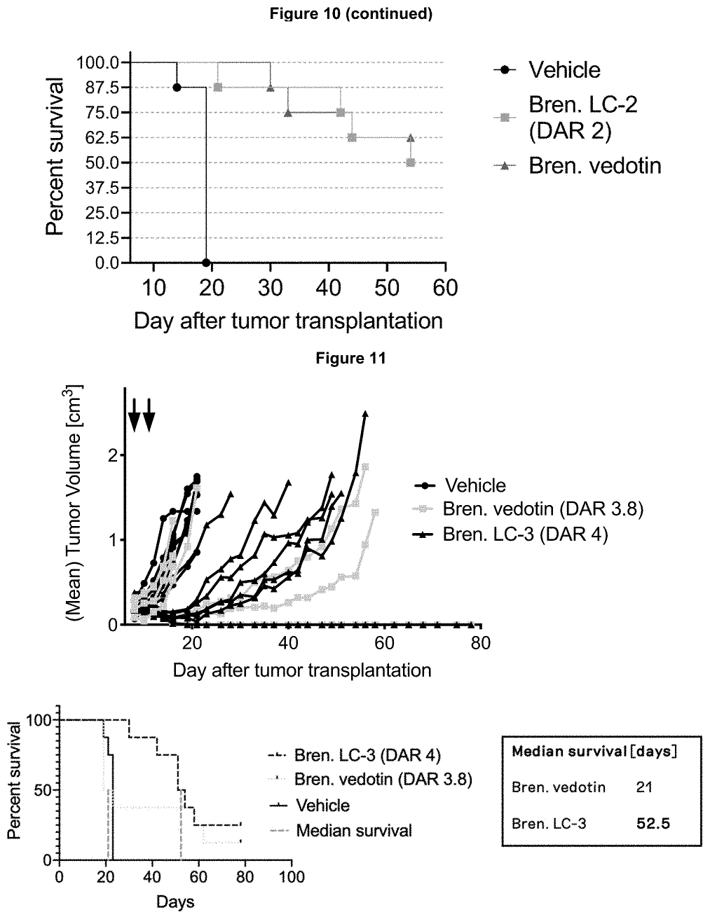

The in vivo efficacy of Brentuximab Tub-tag ADCs was evaluated with a Karpas299-derived tumor model in immune-deficient CB17-SCID mice. As can be seen in FIGS. 9 and 10, Bren. LC-2 showed an unexpected high in vivo efficacy that was similar to that of Brentuximab vedotin. This observation was made despite the fact that Brentuximab LC-2 is equipped with half of the drug loading as it is a DAR 2 MMAE ADC compared to Brentuximab vedotin being conjugated to 4 MMAE molecules. Furthermore, and as unexpected, Bren. LC-3 significantly outcompeted the in vivo efficacy of Brentuximab vedotin with an increase in Median survival by a factor of 2.5 (FIG. 11).

FIG. 9 shows the in vivo efficacy of Bren. LC-2 expressed in terms of tumor volume (cm³). For assessment of the in vivo efficacy of Bren. LC-2, mice, bearing a tumor volume between 100-150 mm³ were randomized and treatment was conducted with two injections of 1.5 mg/kg at day 7 and 10 after tumor transplantation.

FIG. 10 shows the in vivo efficacy of Bren. LC-2 expressed in terms of tumor volume (cm³) and percentage of survival of treated animals (Kaplan-Meier-Plot). For assessment of the in vivo efficacy of Bren. LC-2, mice, bearing a tumor volume between 100-150 mm³ were randomized and treatment was conducted with one single injection of 1.0 mg/kg.

FIG. 11 shows the in vivo efficacy of Bren. LC-3 expressed in terms of tumor volume (cm³) and percentage of survival of treated animals. For assessment of the in vivo efficacy of Bren. LC-3, mice, bearing a tumor volume between 100-150 mm³ were randomized and treatment was conducted with two injections of 0.5 mg/kg at day 8 and 11 after tumor transplantation.

### Example 5: Pharmacokinetics of Brentuximab Tub-Tad ADCs

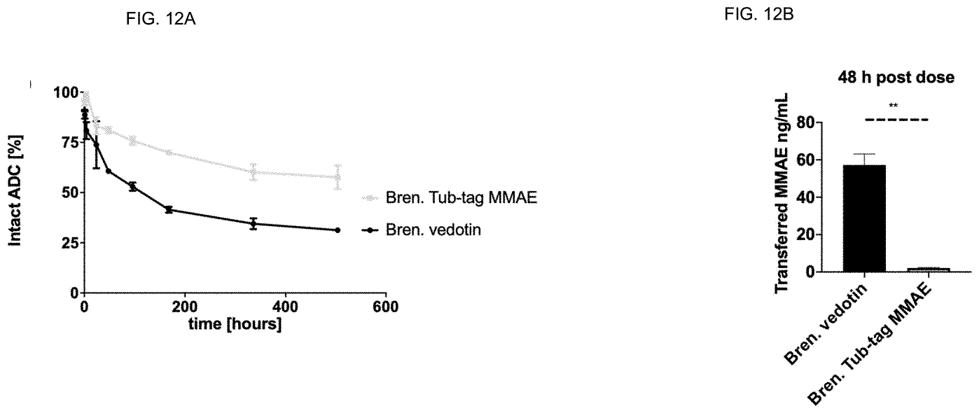

Motivated by the surprising findings that Brentuximab Tub-tag ADCs are characterized by high in vitro stability and superior in vivo efficacy, the Inventors wanted to elucidate whether the in vitro stability translates into living organisms and performed pharmacokinetic analytics in Sprague Dawley rats. Test items were administered to the animals at day 1 by a single intravenous (bolus) injection. To analyze the ADC content over time, ELISA-based assays have been developed detecting the antibody and the intact ADC (by measuring the payload linked to the antibody). The total antibody assay delivers useful information about antibody clearance, and detection differences in comparison to the intact ADC assay may account for payload loss from the antibody. Moreover, the Inventors developed an MS-based assay facilitating the determination of payload loss and payload transfer to blood proteins. The study showed that Brentuximab Tub-tag ADCs have excellent in vivo stability (FIG. 12A). Moreover, and in contrast to Brentuximab vedotin, Brentuximab Tub-tag ADCs are not characterized to enable payload being transferred and covalently linked to blood proteins (FIG. 12B).

FIGS. 12A-12B show a pharmacokinetic (PK) analysis of Brentuximab Tub-tag MMAE (Bren LC-2). In FIG. 12A the amount of intact ADC in comparison to Brentuximab vedotin is shown. In FIG. 12B the amount of transferred MMAE to blood proteins analyzed by MS-analysis is shown.

### Example 6: Repeated Dose Toxicity Study of TUB-010

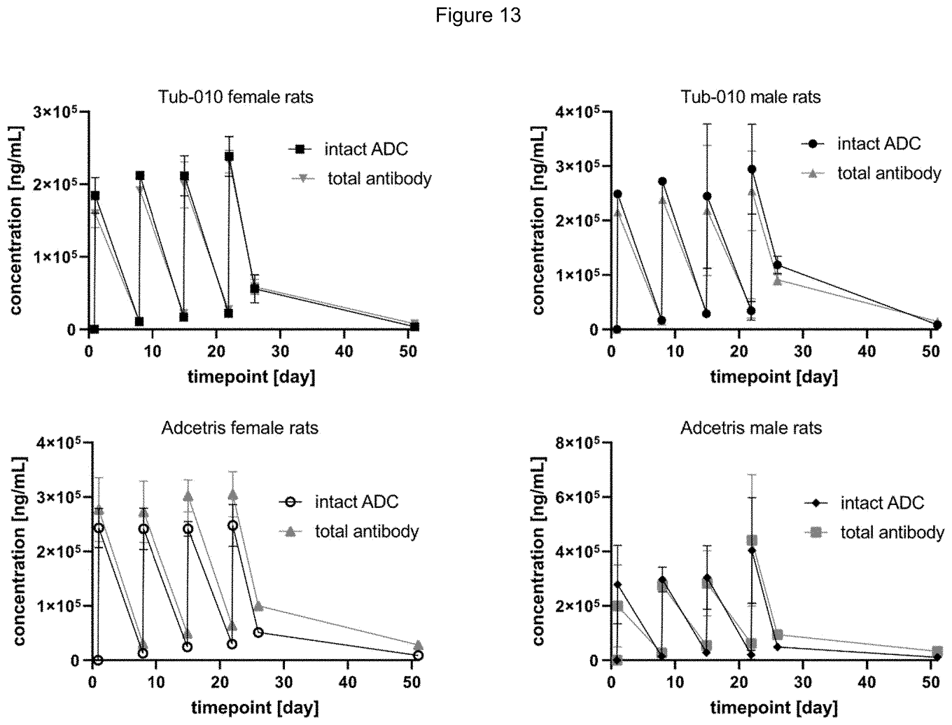

So far, Brentuximab Tub-tag ADCs have been shown to be characterized by increased in vivo efficacy and stability. Thus, the Inventors wondered whether the increased in vivo stability results in a beneficial toxicological profile of Brentuximab Tub-tag ADCs. This is advantageous because an improved toxicological profile in combination with improved efficacy can lead to a significantly widened therapeutic window and thus to a strong advantage for the treatment of patients. Therefore, the Inventors evaluated the toxicity and toxicokinetics of Brentuximab Tub-tag ADCs in Sprague Dawley male and female rats and compared the profile to that of Brentuximab vedotin. Male and female Sprague Dawley rats were dosed with control (vehicle), Brentuximab Tub-tag ADC (Bren LC-2) (10 mg/kg) or Brentuximab vedotin (10 mg/kg) weekly (days 1, 8, 15 & 22), with main dosing phase necropsy on day 26 (5/sex/group) and recovery phase necropsy on day 51 (5/sex/group). Clinical observations, body weight and food consumption were determined daily throughout the study. Pharmacokinetic samples were obtained prior to each dose, 15 minutes post each dose and at necropsy from each animal. All animals were necropsied, with organs weighed and retained in fixative. Clinical pathology (hematology, clinical chemistry, and coagulation endpoints) samples were also taken at necropsy. The mean serum concentrations of intact ADCs and total antibodies are shown in FIG. 13. The graphs clearly demonstrate that dosing with Bren LC-2 results in reasonable levels of serum concentrations in vivo after repeated doses. The high overlap between intact ADC and total antibody for Bren LC-2 again highlights the excellent stability of the constructs in vivo and differentiates them from the Brentuximab vedotin group.

FIG. 13 shows the mean serum concentrations of intact ADC and total antibody in male and female rats following an intravenous (bolus) administration at 10 mg/kg at day 1, 8, 15 and 22 for Brentuximab Tub-tag ADCs compared to that of Brentuximab vedotin.

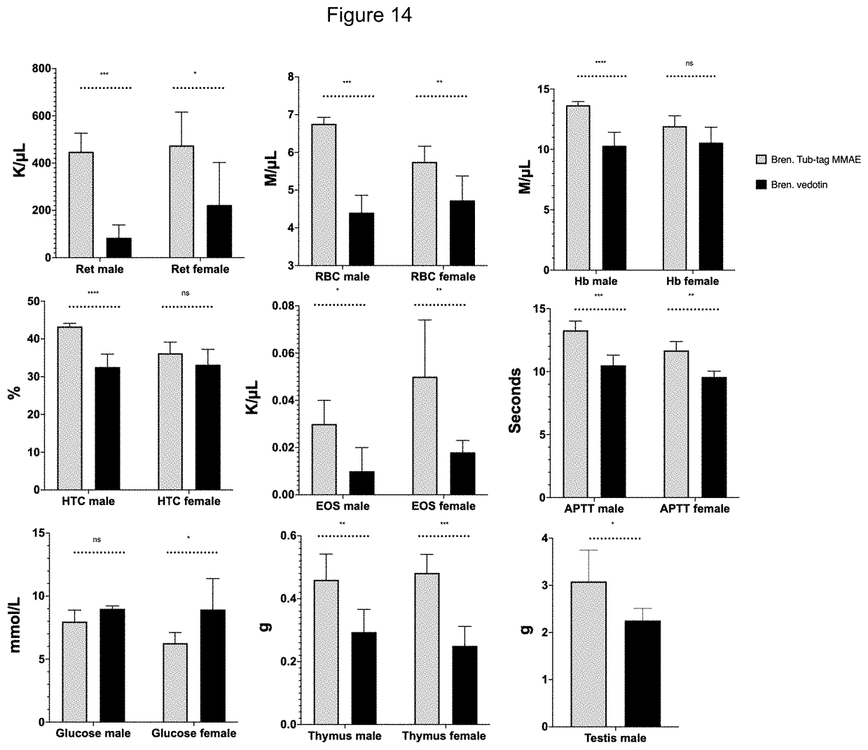

Contrary to Brentuximab vedotin, no impact on red blood cell populations was observed with Brentuximab Tub-tag ADC (Bren LC-2). This could offset the anemia (and potentially the thrombocytopenia) observed with Brentuximab vedotin in clinical practice. Expected MMAE driven testes changes were observed with both Bren LC-2 and Brentuximab vedotin, but were delayed in onset with Bren LC-2, with the stability of Brentuximab Tub-tag ADCs likely playing a role in the delayed onset in testicular toxicity. An exemplary toxicity profile of Bren. LC-2 is shown in FIG. 14.

FIG. 14 shows an exemplary toxicity profile of a Brentuximab Tub-tag ADC (Bren LC-2) (left bar) compared to that of Brentuximab vedotin (right bar) in male and female rats. Parameters depicted in FIG. 14 for the Brentuximab Tub-tag ADC Bren LC-2 are Reticulocytes in counts per microliter (RET in K/μl), Red Blood Cells in millions per microliter (RBC in M/μL), Hemoglobin in millions per microliter (Hb in M/μL), hematocrit in volume percent (HTC in %), Eosinophils in count per microliter (EOS in K/μl), activated partial thromboplastin time in seconds (APPT) (seconds), glucose levels in millimole per liter (mmol/L), thymus weight in gram (g) and testicle weight in gram (g). As reference, rats were dosed with a control (vehicle) (data not shown).

In particular, FIG. 14 shows:

Reticulocytes: Treatment with Brentuximab vedotin has a marked impact on the reticulocyte count, while no effect is observed under treatment with the Brentuximab Tub-tag ADC. Accordingly, treatment with the Brentuximab Tub-tag ADC is expected to result in less anemia in the clinic compared to treatment with Brentuximab vedotin (aligned with the red blood cell, hemoglobin and hematocrit data).

Red blood cells: Treatment with Brentuximab vedotin has a marked impact on the red blood cell count, while no marked effect is observed under treatment with the Brentuximab Tub-tag ADC. Accordingly, treatment with the Brentuximab Tub-tag ADC is expected to result in less anemia in the clinic compared to treatment with Brentuximab vedotin (aligned with the reticulocyte, hemoglobin and hematocrit data).

Hemoglobin: Treatment with Brentuximab vedotin reduces hemoglobin levels (mainly in males), while no marked effect is observed under treatment with the Brentuximab Tub-tag ADC. Accordingly, treatment with the Brentuximab Tub-tag ADC is expected to result in less anemia in the clinic compared to treatment with Brentuximab vedotin (aligned with the red blood cell, reticulocyte and hematocrit data).

Hematocrit: Treatment with Brentuximab vedotin reduces hematocrit (mainly in males), while no marked effect is observed under treatment with the Brentuximab Tub-tag ADC. Accordingly, treatment with the Brentuximab Tub-tag ADC is expected to result in less anemia in the clinic compared to treatment with Brentuximab vedotin (aligned with the red blood cell, hemoglobin and reticulocyte data).

Eosinophils: Treatment with Brentuximab vedotin results in lower eosinophils counts, compared to treatment with the Brentuximab Tub-tag ADC. Assuming this is a generalized effect on white blood cells, treatment with the Brentuximab Tub-tag ADC is expected to result in less neutropenia in the clinic compared to treatment with Brentuximab vedotin.

APTT: Treatment with Brentuximab vedotin results in lower APTT values compared to treatment with the Brentuximab Tub-tag ADC. As Brentuximab vedotin does not have any impact on clotting time/APTT in the clinic, in view of the provided data also the Brentuximab Tub-tag ADC is not expected to cause any concerns with regard to blood clotting time.

Glucose: Treatment with the Brentuximab vedotin results in a greater level of glucose than treatment with the Brentuximab Tub-tag ADC. Accordingly, treatment with the Brentuximab Tub-tag ADC results in lower glucose levels compared to treatment with Brentuximab vedotin. Treatment with the Brentuximab Tub-tag ADC is therefore less expected to result in hyperglycemia, which has been observed under treatment with Brentuximab vedotin in the clinic.

Thymus: Treatment with Brentuximab vedotin results in lower thymus weight, compared to treatment with the Brentuximab Tub-tag ADC. A thymus weight change observed under treatment with ADCs comprising MMAE is a typical target-independent/free toxin related toxicity, which results from cell depletion caused by MMAE. Accordingly, the Brentuximab Tub-tag ADC is expected to have a more favorable clinical safety profile in respect of a less target-independent/free MMAE related toxicity, as compared to Brentuximab vedotin.

Testis: Treatment with Brentuximab vedotin results in lower testis weight, compared to treatment with the Brentuximab Tub-tag ADC. A testicular weight change observed under treatment with ADCs comprising MMAE is a typical target-independent/free toxin related toxicity. Accordingly, the Brentuximab Tub-tag ADC is expected to have a more favorable clinical safety profile in respect of a less target-independent/free MMAE related toxicity compared to Brentuximab vedotin.

Overall, the provided data show that the Brentuximab Tub-tag ADC has a better safety profile compared to Brentuximab vedotin in the rat. Accordingly, in the clinic the Brentuximab Tub-tag ADC is less expected to result in particular dose-limiting toxicities of Brentuximab vedotin, including anemia, neutropenia and hyperglycemia. For the Brentuximab Tub-tag ADC, the lack of target-independent/free toxin related toxicities associated with Brentuximab vedotin can also offset characteristic changes like the peripheral neuropathy seen with many ADCs comprising MMAE, including Brentuximab vedotin.

### Example 7: Repeated Dose Toxicity Study of Brentuximab Tub-Tag MMAE in Cynomolgus Monkey

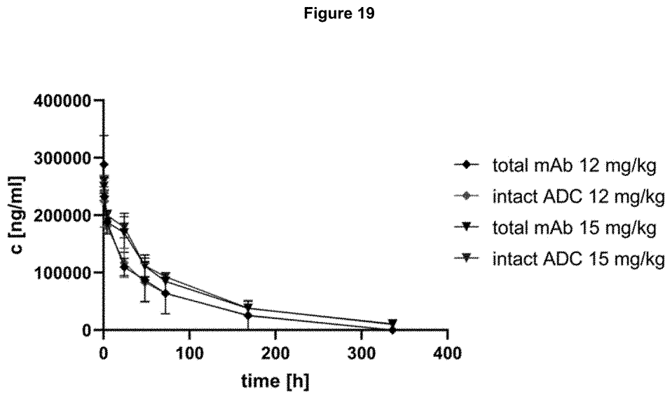

Next, the toxicity of Brentuximab Tub-tag MMAE (Bren. LC-2) was evaluated in Cynomolgus monkey. The aim of the study was to evaluate the toxicity of and toxicokinetic (TK) of Brentuximab Tub-tag MMAE (Bren. LC-2) in cynomolgus monkey. Brentuximab Tub-tag ADC was administered at 6, 12 & 15 mg/kg (Q3VVx4 for 6 mg/kg, Q3VVx2 for 12 & 15 mg/kg). Clinical condition (incl. bodyweight, food consumption & clinical observations), clinical pathology and immunophenotyping (T, B and NK cells) was evaluated throughout the study, with frequent sampling for TK and anti-drug antibody determination. All animals were subject to full necropsy with detailed macroscopic observations. Brentuximab Tub-tag MMAE (Bren. LC-2) has been clinically well tolerated at 6, 12 and 15 mg/kg with no impact on bodyweight. Adcetris, based on the FDA PharmTox review was poorly tolerated at 6 mg/kg in cynomolgus monkey (MTD was 3 mg/kg) with deaths and early euthanasia between 11 and 15 days following the first dose. In contrast, no mortality and no macroscopic or sever microscopic findings have been observed within this study for Brentuximab Tub-tag MMAE (Bren. LC-2). Reductions in red blood cells (RBC's), hemoglobin and neutrophils have been noted for Brentuximab Tub-tag MMAE (Bren. LC-2) at 12 & 15 mg/kg that were relatively consistent to those observed with Adcetris at 3 mg/kg (historical data). Moreover, the Brentuximab Tub-tag MMAE (Bren. LC-2) was characterized by high stability in toxicokinetic analysis as shown in FIG. 19.

To analyze the ADC content in cynomolgus monkey serum over time, ELISA-based assays have been developed detecting the antibody and the intact ADC content (by measuring the payload linked to the antibody; the same ELISA assay setup has been used within the pharmacokinetics and repeat dose toxicity study in rats with the difference, that the assay has been further validated for use in cynomolgus monkey serum). The total antibody assay delivers useful information about antibody clearance, and detection differences in comparison to the intact ADC assay may account for payload loss from the antibody. High overlap of intact ADC and total mAb curve in FIG. 19 shows minimal loss of payload and high ADC stability of Brentuximab Tub-tag MMAE.

FIG. 19 shows a toxicokinetic analysis of Brentuximab Tub-tag MMAE (i.e., Bren. LC-2) in cynomolgus monkey dosed with 12 & 15 mg/kg. Total amount of mAb and intact ADC was assessed by ELISA. High overlap of intact ADC and total mAb curves show high stability of Brentuximab Tub-tag MMAE at both dose levels. During the repeated dose studies of Brentuximab Tub-tag MMAE (Bren. LC-2), the body weight and different blood values, i.e. the concentration of lymphocytes, neutrophiles and white blood cells, of the cynomolgus monkeys were monitored.

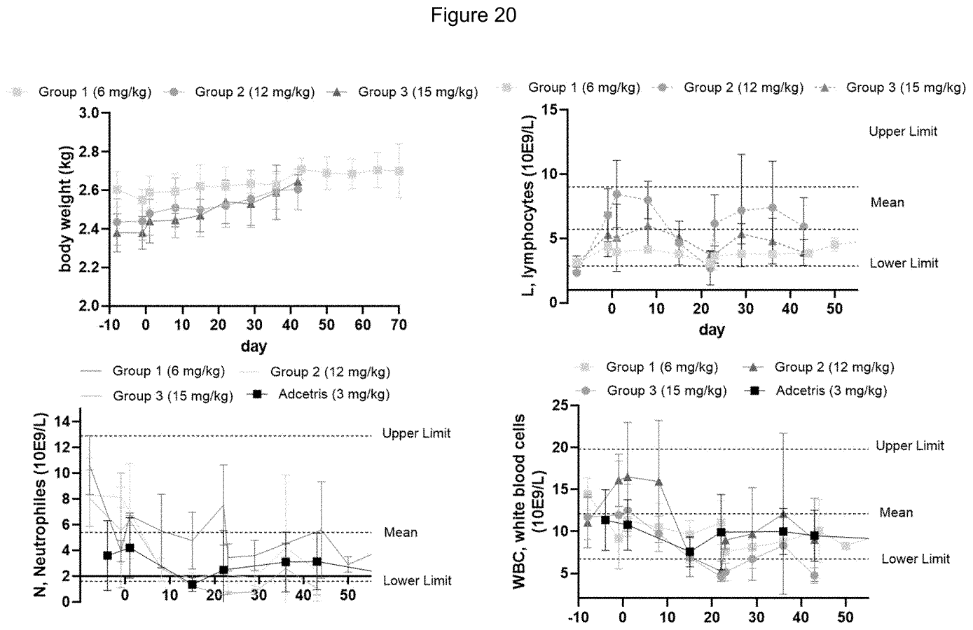

FIG. 20 shows the values obtained for the body weight and a selection of different blood values that have been collected throughout the repeated dose study of Brentuximab Tub-tag MMAE (i.e., Bren. LC-2) in cynomolgus monkey. In more detail, FIG. 20, upper panel on the left shows the body weight over time. FIG. 20, upper panel on the right shows the lymphocytes concentration over time. FIG. 20, lower panel on the left shows the neutrophiles concentration over time. FIG. 20, lower panel on the right shows the white blood cells concentration over time. The data is shown, in each case, as mean and error of three groups of two female animals dosed repeatedly with 6, 12 and 15 mg/kg Brentuximab Tub-tag MMAE (Bren. LC-2) in comparison with the results of the toxicity study for Brentuximab vedotin (Adcetris) described in the FDA report “Clinical Pharmacology And Biopharmaceutics Review(s) for application number 125388Orig1S00 (available at https://www.accessdata.fda.gov/drugsatfda_docs/nda/2011/125388orig1s000clinpharmr.pdf). Bodyweight is depicted in kilogram, lymphocytes in billion per liter, neutrophiles in billion per liter and white blood cells in billion per liter.

One of the dose-limiting toxicities of Brentuximab vedotin in the clinic is neutropenia. The dataset depicted in FIG. 20, lower panel on the left shows that Brentuximab Tub-tag MMAE (Bren. LC-2), advantageously, does not cause a higher amount of reduction in neutrophiles in direct comparison to Brentuximab vedotin, even though doses two times (6 mg/kg), four times (12 mg/kg) and five times (15 mg/kg) the dose of Brentuximab vedotin (3 mg/kg) were applied. Further, an increase in body weight was observed (FIG. 20, upper panel on the left). No significant changes of the lymphocytes concentration and white blood cells concentration were determined. (FIG. 20, upper panel on the right and lower panel on the right).

The observed combination of the neutrophils concentration, increasing body weights for all animals throughout the study, and no significant change in the white blood cells and lymphocytes concentration clearly shows a good tolerability of ACDs of the present disclosure, which exceeds the tolerability of Brentuximab vedotin. Thus, the data clearly suggests a good tolerability in the clinic, which exceeds the one of Brentuximab vedotin.

In summary, the presented results show that Brentuximab Tub-tag ADCs are characterized by unexpectedly high stability in vitro and in vivo that translates into superior in vivo efficacy and toxicology profiles.

What is claimed is:

1. An antibody-drug conjugate (ADC) comprising:
(a) Brentuximab, wherein Brentuximab comprises at the C-terminus of the light chains, the heavy chains or all of the heavy and light chains of the Brentuximab a recognition sequence for tubulin tyrosine ligase and a non-natural amino acid; and
(b) at least one drug moiety;
wherein a drug moiety is coupled to each of the non-natural amino acids via a linker.

2. The ADC of claim 1,
wherein the heavy chains of Brentuximab have an amino acid sequence that comprises or consists of SEQ ID NO: 1 or have a sequence identity of at least 95% to SEQ ID NO: 1; and/or
wherein the light chains of Brentuximab have an amino acid sequence that comprises or consists of SEQ ID NO: 2 or have a sequence identity of at least 95% to SEQ ID NO: 2; or
wherein Brentuximab consists of heavy chains consisting of the amino acid sequence of SEQ ID NO: 1 and light chains consisting of the amino acid sequence of SEQ ID NO: 2.

3. The ADC of claim 1, wherein the drug moiety is selected from the group consisting of camptothecins, maytansinoids, calicheamycins, duocarmycins, tubulysins, amatoxins, dolastatins and auristatins such as monomethyl auristatin E (MMAE), pyrrolobenzodiazepine dimers, indolino-benzodiazepine dimers, radioisotopes, therapeutic proteins and peptides (or fragments thereof), nucleic acids, PROTACs, kinase inhibitors, MEK inhibitors, KSP inhibitors, and analogues or prodrugs thereof.

4. The ADC of claim 1, wherein the recognition sequence for tubulin tyrosine ligase has at least the amino acid sequence X₁X₂X₃X₄ (SEQ ID NO: 3), wherein X₁ and X₂ is any amino acid, X₃ is E, D or C and X₄ is E; or
wherein X₂ is G, S, A, V, or F and/or wherein X₁ is E, D, A, K, or P; or
wherein the recognition sequence is EGEE (SEQ ID No. 4); or
wherein the recognition sequence is VDSVEGEGEEEGEE (SEQ ID No. 5), SVEGEGEEEGEE (SEQ ID No. 6), SADGEDEGEE (SEQ ID No. 7), SVEAEAEEGEE (SEQ ID No. 8), SYEDEDEGEE (SEQ ID No. 9), or SFEEENEGEE (SEQ ID No. 10).

5. The ADC of claim 1, wherein the unnatural amino acid is a 2-substituted, 3-substituted or 4-substituted tyrosine or a tyrosine derivative substituted at the benzylic position; or
wherein the unnatural amino acid is 3-nitrotyrosine, 3-aminotyrosine, 3-azidotyrosine, 3-formyltyrosine, 3-acetyltyrosine, or 4-aminophenylalanine.

6. The ADC of claim 1, wherein the linker is cleavable, such as by a protease like cathepsin B; or
wherein the linker comprises a valine-citrulline moiety.

7. The ADC of claim 1, wherein the linker comprises a hydroxylamine group and the unnatural amino acid comprises a formyl group ortho of a hydroxyl group in an aromatic ring, and wherein the hydroxylamine group of the linker forms an oxime with the formyl group of the unnatural amino acid after conjugation.

8. The ADC of claim 1, wherein Brentuximab is conjugated to two, four, six, or eight drug moieties.

9. The ADC of claim 1, wherein the linker has a structure as depicted in structure 1 before being coupled to the unnatural amino acid: 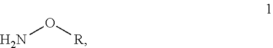
wherein R is one or more drug moieties, which are optionally coupled to the hydroxylamine of structure 1 by one or more cleavage sites.

10. The ADC of claim 9, wherein the hydroxylamine of structure 1 is conjugated to the unnatural amino acid.

11. The ADC of claim 1, wherein the linker has a structure as depicted in structure 2 or 3 before being coupled to the unnatural amino acid: 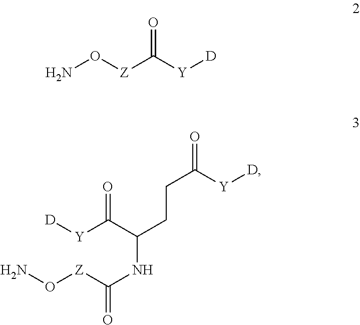
wherein Z is is selected from the group consisting of substituted or unsubstituted alkyl, substituted or unsubstituted alkenyl, substituted or unsubstituted alkynyl, substituted or unsubstituted cycloalkyl, substituted or unsubstituted aryl, substituted or unsubstituted arylalkyl, substituted or unsubstituted heteroaryl, substituted or unsubstituted heteroarylalkyl, substituted or unsubstituted heterocyclyl, substituted or unsubstituted heteroalkyl, substituted or unsubstituted heteroalkenyl and substituted or unsubstituted heteroalkynyl;
wherein D is one or more drug moieties; and
wherein Y is a cleavage site such as a cleavage site for a cathepsin such as cathepsin B.

12. The ADC of claim 11, wherein the hydroxylamine of structure 2 or 3 is conjugated to the unnatural amino acid.

13. The ADC of claim 1, wherein the linker has a structure as depicted in structure 4 or 5 before being coupled to the unnatural amino acid, wherein D is a drug moiety:

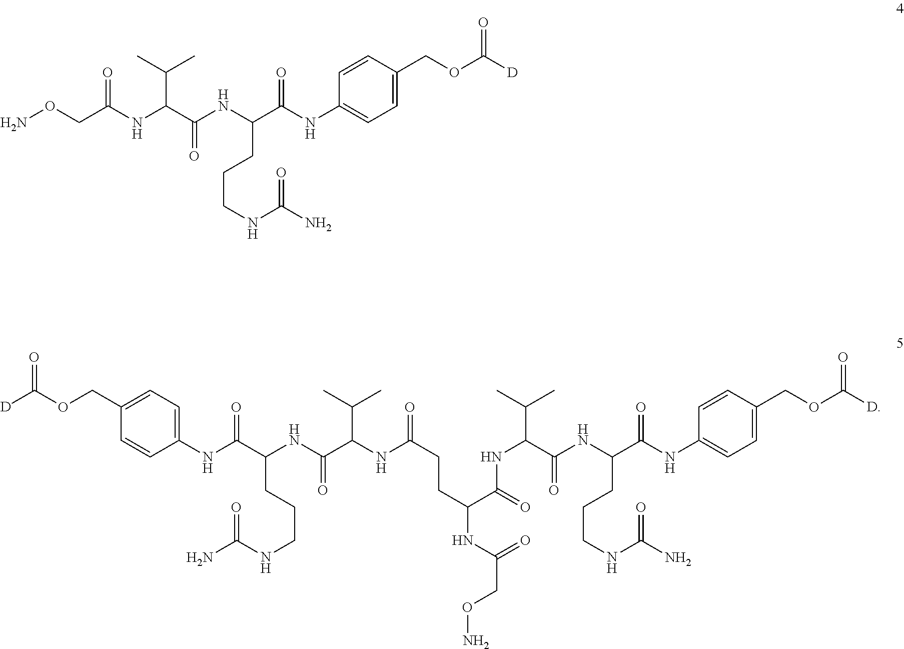

14. The ADC of claim 13, wherein the unnatural amino acid is 3-formyltyrosine and the hydroxylamine group of the linker forms an oxime with the 3-formyl group of the unnatural amino acid.

15. The ADC of claim 14, wherein the drug moiety is MMAE.

16. The ADC of claim 1, comprising:
(a) Brentuximab, wherein Brentuximab comprises at the C-terminus of each light chain a recognition sequence for tubulin-tyrosine ligase;
each light chain, including the recognition sequence, has SEQ ID NO: 11; and
each heavy chain of Brentuximab has SEQ ID NO: 1; and
(b) the C-terminus of the recognition sequence of each light chain is bound via an amide bond to a group having the following structure: 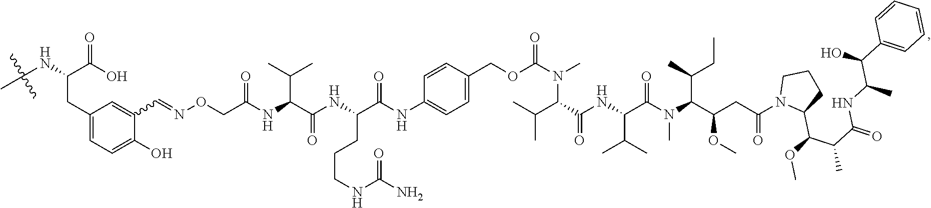 [SMILES: *N[C@@H](Cc1ccc(O)c(C=NOCC(=O)N[C@H](C(=O)N[C@@H](CCCNC(N)=O)C(=O)Nc2ccc(COC(=O)N(C)[C@H](C(=O)N[C@H](C(=O)N(C)[C@@H]([C@@H](C)CC)[C@@H](CC(=O)N3CCC[C@H]3[C@H](OC)[C@@H](C)C(=O)N[C@H](C)[C@@H](O)c3ccccc3)OC)C(C)C)C(C)C)cc2)C(C)C)c1)C(=O)O]
wherein the wavy line indicates attachment to the C-terminus of the recognition sequence of each light chain; or
(a) Brentuximab, wherein Brentuximab comprises at the C-terminus of each light chain a recognition sequence for tubulin-tyrosine ligase;
each light chain, including the recognition sequence, has SEQ ID NO: 11; and
each heavy chain of Brentuximab has SEQ ID NO: 1; and
(b) the C-terminus of the recognition sequence of each light chain is bound via an amide bond to a group having the following structure: 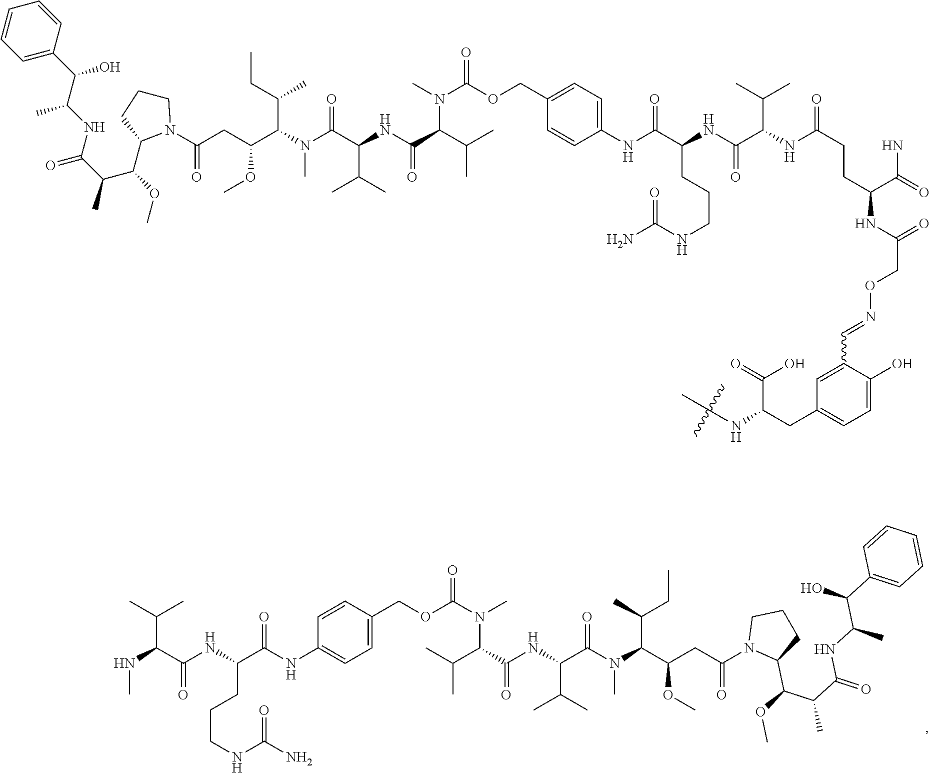 [SMILES: *N[C@@H](Cc1ccc(O)c(C=NOCC(=O)N[C@@H](CCC(=O)N[C@H](C(=O)N[C@@H](CCCNC(N)=O)C(=O)Nc2ccc(COC(=O)N(C)[C@H](C(=O)N[C@H](C(=O)N(C)[C@@H]([C@@H](C)CC)[C@@H](CC(=O)N3CCC[C@H]3[C@H](OC)[C@@H](C)C(=O)N[C@H](C)[C@@H](O)c3ccccc3)OC)C(C)C)C(C)C)cc2)C(C)C)C(N)=O)c1)C(=O)O ; CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC)C(C)C)cc1)C(C)C]
wherein the wavy line indicates attachment to the C-terminus of the recognition sequence of each light chain.

17. A method of producing an ADC as defined in claim 1, comprising
(a) introducing or adding at the C-terminus of the light chain, the heavy chain or both the light chain and the heavy chain of Brentuximab a recognition sequence for tubulin tyrosine ligase;
(b) contacting the Brentuximab obtained in step (a) in the presence of tubulin tyrosine ligase and a non-natural amino acid under conditions suitable for the tubulin tyrosine ligase to ligate said Brentuximab with said non-natural amino acid; and
(c) conjugating an optionally cleavable linker comprising a drug moiety to said ligated Brentuximab obtained in step (b).

18. A pharmaceutical composition comprising the ADC of claim 1.

19. A method of treating a disease associated with overexpression of CD30, comprising the administration of an effective amount of the ADC of claim 1 to a subject or patient in need thereof.

20. The method of claim 19, wherein the disease is a cancer associated with overexpression of CD30; or
wherein the disease is selected from the group consisting of lymphoma, such as Hodgkin's lymphoma (HL), non-Hodgkin lymphoma (NHL), anaplastic large-cell lymphoma (ALCL), large B-cell lymphoma, paediatric lymphoma, T-cell lymphoma and enteropathy-associated T-cell lymphoma (EATL), leukaemia, such as acute myeloid leukaemia (AML), acute lymphoblastic leukaemia (ALL) and mast cell leukaemia, germ cell cancer, graft-versus-host disease (GvHD) and lupus, in particular systemic lupus erythematosus (SLE), preferably Hodgkin Lymphoma (HL) or anaplastic large cell lymphoma (ALCL); or
wherein the disease is selected from the group consisting of peripheral T cell lymphoma—not otherwise specified (PTCL-NOS), angioimmunoblastic T-cell lymphoma (AITL), enteropathy associated T cell lymphoma (EATL), adult T-cell leukemia/lymphoma (ATLL), extranodal natural killer/T-cell lymphoma (ENKTCL), hepatosplenic and intestinal γ/δ-T cell lymphoma, nodal peripheral T-cell lymphoma with TFH phenotype, and follicular T cell lymphoma; or
wherein the disease is Hodgkin Lymphoma (HL) or anaplastic large cell lymphoma (ALCL); or
wherein the disease is peripheral T cell lymphoma (PTCL), including anaplastic large cell lymphoma (ALCL); or cutaneous T cell lymphoma (CTCL), including primary cutaneous anaplastic large cell lymphoma (pcALCL); or
wherein the ADC is administered at a dose of 14 mg/kg, 12 mg/kg, 10 mg/kg, 9 mg/kg, 8 mg/kg, 7 mg/kg, 6 mg/kg, 5 mg/kg, 4 mg/kg, 3 mg/kg, 2 mg/kg or 1 mg/kg; or at a dose of 2-6 mg/kg.
